SEQUENCE LISTING (332 sequences)

SEQ ID NO: 1 - protein, 8 aa, Artificial Sequence
  Synthetic
GFNIKDDY

SEQ ID NO: 2 - protein, 8 aa, Artificial Sequence
  Synthetic
IDPENGDT

SEQ ID NO: 3 - protein, 10 aa, Artificial Sequence
  Synthetic
TLWLRRGLDY

SEQ ID NO: 4 - protein, 11 aa, Artificial Sequence
  Synthetic
KSLLHSNGYTY

SEQ ID NO: 5 - protein, 3 aa, Artificial Sequence
  Synthetic
RMS

SEQ ID NO: 6 - protein, 9 aa, Artificial Sequence
  Synthetic
MQHLEYPFT

SEQ ID NO: 7 - protein, 117 aa, Artificial Sequence
  Synthetic
EVQLQQSGAELVRPGASVKLSCTASGFNIKDDYMYWVKQRPEQGLEWIGWIDPENGDTEYASKFQDKATVTADTSSNTAYLQLSSLTSEDTAVYYCTLWLRRGLDYWGQGTSVTVSS

SEQ ID NO: 8 - protein, 112 aa, Artificial Sequence
  Synthetic
DIVMTQAAPSVPVTPGESVSISCRSSKSLLHSNGYTYLFWFLQRPGQSPQLLIYRMSNLASGVPDRFSGSGSGTAFTLRISRVEAEDVGVYYCMQHLEYPFTFGGGTKLEIK

SEQ ID NO: 9 - protein, 9 aa, Artificial Sequence
  Synthetic
GYSITSGYY

SEQ ID NO: 10 - protein, 7 aa, Artificial Sequence
  Synthetic
ITFDGAN

SEQ ID NO: 11 - protein, 12 aa, Artificial Sequence
  Synthetic
TRSSYDYDVLDY

SEQ ID NO: 12 - protein, 6 aa, Artificial Sequence
  Synthetic
QDISNF

SEQ ID NO: 13 - protein, 3 aa, Artificial Sequence
  Synthetic
YTS

SEQ ID NO: 14 - protein, 9 aa, Artificial Sequence
  Synthetic
QQGHTLPYT

SEQ ID NO: 15 - protein, 119 aa, Artificial Sequence
  Synthetic
DVQLQESGPGLVKPSQSLSLTCSVTGYSITSGYYWNWIRQFPGNKLEWMGYITFDGANNYNPSLKNRISITRDTSKNQFFLKLTSVTTEDTATYYCTRSSYDYDVLDYWGQGTTLTVSS

SEQ ID NO: 16 - protein, 107 aa, Artificial Sequence
  Synthetic
DIQMTQTTSSLSASLGDRVTISCRASQDISNFLNWYQQRPDGTVKLLIYYTSRLHSGVPSRFSGSGSGTDFSLTVSNLEQEDIATYFCQQGHTLPYTFGGGTKLEIK

SEQ ID NO: 17 - protein, 8 aa, Artificial Sequence
  Synthetic
GYSFTDYC

SEQ ID NO: 18 - protein, 8 aa, Artificial Sequence
  Synthetic
IYPGSGNT

SEQ ID NO: 19 - protein, 13 aa, Artificial Sequence
  Synthetic
AREDYYPYHGMDY

SEQ ID NO: 20 - protein, 10 aa, Artificial Sequence
  Synthetic
ESVDGYDNSF

SEQ ID NO: 21 - protein, 3 aa, Artificial Sequence
  Synthetic
RAS

SEQ ID NO: 22 - protein, 9 aa, Artificial Sequence
  Synthetic
QQSSEDPWT

SEQ ID NO: 23 - protein, 120 aa, Artificial Sequence
  Synthetic
QIQLQQSGPELVRPGASVKISCKASGYSFTDYCINWVNQRPGQGLEWIGWIYPGSGNTRYSERFKGKATLTVDTSSNTAYMQLSSLTSEDSAVYFCAREDYYPYHGMDYWGQGTSVTVSS

SEQ ID NO: 24 - protein, 111 aa, Artificial Sequence
  Synthetic
DIVLTQSPTSLAVSLGQRATISCRASESVDGYDNSFMHWYQQKPGQPPKLLIFRASNLESGIPARFSGSGSRTDFTLTINPVEAADVATYYCQQSSEDPWTFGGGTKLEIK

SEQ ID NO: 25 - protein, 8 aa, Artificial Sequence
  Synthetic
GYTFTSYW

SEQ ID NO: 26 - protein, 8 aa, Artificial Sequence
  Synthetic
IFPNSGRT

SEQ ID NO: 27 - protein, 11 aa, Artificial Sequence
  Synthetic
AREGNFGSLDY

SEQ ID NO: 28 - protein, 5 aa, Artificial Sequence
  Synthetic
SNLNY

SEQ ID NO: 29 - protein, 3 aa, Artificial Sequence
  Synthetic
DTS

SEQ ID NO: 30 - protein, 9 aa, Artificial Sequence
  Synthetic
QQWSRNPLT

SEQ ID NO: 31 - protein, 118 aa, Artificial Sequence
  Synthetic
QVHLQQPGAELVKPGASVKMSCKASGYTFTSYWITWVKQRPGQGLEWIGDIFPNSGRTNYDEKFKSKATLTVDTSSSTAYMQLSSLTSEDSAVYFCAREGNFGSLDYWGQGTTLTVSS

SEQ ID NO: 32 - protein, 106 aa, Artificial Sequence
  Synthetic
QIVLTQSPAIMSASPGEKVTMTCSANSNLNYMNWYHQKSGTSPKRWIYDTSKLASGVPARFSASGSGTSYSLTISSMEAEDAATYYCQQWSRNPLTFGAGTRLELK

SEQ ID NO: 33 - protein, 10 aa, Artificial Sequence
  Synthetic
GFSLNTYDVG

SEQ ID NO: 34 - protein, 7 aa, Artificial Sequence
  Synthetic
IWWNDDK

SEQ ID NO: 35 - protein, 11 aa, Artificial Sequence
  Synthetic
TLYSYDGGFAY

SEQ ID NO: 36 - protein, 7 aa, Artificial Sequence
  Synthetic
SSVSSSY

SEQ ID NO: 37 - protein, 3 aa, Artificial Sequence
  Synthetic
STS

SEQ ID NO: 38 - protein, 9 aa, Artificial Sequence
  Synthetic
HQYHRSPYT

SEQ ID NO: 39 - protein, 119 aa, Artificial Sequence
  Synthetic
QVTLKESGPGMLQPSQTLSLTCSFSGFSLNTYDVGVGWIRQPSGKGLEWLANIWWNDDKYYNSALKSRLTISKDTSNNQVFLKISSVDTADTATYYCTLYSYDGGFAYWGQGTLVTVSA

SEQ ID NO: 40 - protein, 108 aa, Artificial Sequence
  Synthetic
QIVLTQSPAIMSASLGERVTMTCTASSSVSSSYLHWYQQKPGSSPKLWIYSTSNLASGVPARFSGSGSGTSYSLTISSMEAEDAATYYCHQYHRSPYTFGGGTKLEIK

SEQ ID NO: 41 - protein, 8 aa, Artificial Sequence
  Synthetic
GYSFTGYN

SEQ ID NO: 42 - protein, 8 aa, Artificial Sequence
  Synthetic
INPYYGST

SEQ ID NO: 43 - protein, 15 aa, Artificial Sequence
  Synthetic
ARGDYGYDEGTWFAY

SEQ ID NO: 44 - protein, 12 aa, Artificial Sequence
  Synthetic
QSLLNSRTRKNY

SEQ ID NO: 45 - protein, 3 aa, Artificial Sequence
  Synthetic
WAS

SEQ ID NO: 46 - protein, 9 aa, Artificial Sequence
  Synthetic
KQSYNLPFT

SEQ ID NO: 47 - protein, 122 aa, Artificial Sequence
  Synthetic
EIQMKQSGAELVKPGASVKISCKASGYSFTGYNMNWVKQSHGKSLEWIGNINPYYGSTGYNQKFKGKATLTVDKSSSTAYMQLNSLTSEDSAVYYCARGDYGYDEGTWFAYWGQGTLVTVSA

SEQ ID NO: 48 - protein, 113 aa, Artificial Sequence
  Synthetic
DIVMSQSPSSLAVSAGEKVTMSCKSSQSLLNSRTRKNYLAWYQQKPEQSPKLLIYWASTRESGVPDRFTGSGSGTDFTLTISSVQAEDLAVYYCKQSYNLPFTFGSGTKLEIK

SEQ ID NO: 49 - protein, 8 aa, Artificial Sequence
  Synthetic
GYTFTRHW

SEQ ID NO: 50 - protein, 8 aa, Artificial Sequence
  Synthetic
IYPGSGRT

SEQ ID NO: 51 - protein, 13 aa, Artificial Sequence
  Synthetic
ARDGYLYINYFDY

SEQ ID NO: 52 - protein, 5 aa, Artificial Sequence
  Synthetic
SSVSF

SEQ ID NO: 53 - protein, 9 aa, Artificial Sequence
  Synthetic
FQGSGYPYT

SEQ ID NO: 54 - protein, 120 aa, Artificial Sequence
  Synthetic
QVQLQQPGAELVKPGASVKMSCKASGYTFTRHWITWVKQRPGQGLEWIGDIYPGSGRTNYNEKFKSTATLTVDTSSSTAYMQLSSLTSEDSAVYYCARDGYLYINYFDYWGQGTTLTVSS

SEQ ID NO: 55 - protein, 106 aa, Artificial Sequence
  Synthetic
ENVLTQSPAIMSASPGEKVTMTCSASSSVSFMHWFQQKSSTSPKLWIYDTSKLASGVPGRFSGSGSGSSYSLTISSMAAEDVATYYCFQGSGYPYTFGGGTKLEIK

SEQ ID NO: 56 - protein, 8 aa, Artificial Sequence
  Synthetic
GFNIVDDY

SEQ ID NO: 57 - protein, 8 aa, Artificial Sequence
  Synthetic
IYPENADT

SEQ ID NO: 58 - protein, 11 aa, Artificial Sequence
  Synthetic
TTATGTGWFAY

SEQ ID NO: 59 - protein, 11 aa, Artificial Sequence
  Synthetic
QSLLDSDGKTY

SEQ ID NO: 60 - protein, 3 aa, Artificial Sequence
  Synthetic
LVS

SEQ ID NO: 61 - protein, 9 aa, Artificial Sequence
  Synthetic
WQGTHFPWT

SEQ ID NO: 62 - protein, 118 aa, Artificial Sequence
  Synthetic
EVQLQQSGAELVRPGASVKLSCTASGFNIVDDYMHWVKQRPEQGLEWIGWIYPENADTEYASKFQGKATITADTSSNTAYLQLSSLTSEDTAVYYCTTATGTGWFAYWGQGTLVTVSA

SEQ ID NO: 63 - protein, 112 aa, Artificial Sequence
  Synthetic
DVVMTQTPLTLSVTIGQPASISCKSSQSLLDSDGKTYLNWLFQRPGQSPKRLIYLVSKLDSGVPDRFTGSGSGTDFTLKISRVETEDLGVYYCWQGTHFPWTFGGGAKLEIK

SEQ ID NO: 64 - protein, 8 aa, Artificial Sequence
  Synthetic
GYTFSNYW

SEQ ID NO: 65 - protein, 8 aa, Artificial Sequence
  Synthetic
ILPGSGST

SEQ ID NO: 66 - protein, 11 aa, Artificial Sequence
  Synthetic
ARRGAYGNFHY

SEQ ID NO: 67 - protein, 7 aa, Artificial Sequence
  Synthetic
SSISSSN

SEQ ID NO: 68 - protein, 3 aa, Artificial Sequence
  Synthetic
GTS

SEQ ID NO: 69 - protein, 9 aa, Artificial Sequence
  Synthetic
QQWRSYPYT

SEQ ID NO: 70 - protein, 118 aa, Artificial Sequence
  Synthetic
QVQLQQSGAELMKPGASVKISCKATGYTFSNYWIEWVKQRPGHGLEWIGEILPGSGSTNYNENFKGKATFTADTSSNTAYMQLSSLTSEDSAVYYCARRGAYGNFHYWGQGTTLTVSS

SEQ ID NO: 71 - protein, 108 aa, Artificial Sequence
  Synthetic
EIVLTQSPALMAASPGEKVTITCSVSSSISSSNLHWYQQKSETSPKPWIYGTSNLASGVPVRFSGSGSGTSYSLTISSMEAEDAATYYCQQWRSYPYTFGGGTKLEIK

SEQ ID NO: 72 - protein, 8 aa, Artificial Sequence
  Synthetic
GYTFTDYN

SEQ ID NO: 73 - protein, 8 aa, Artificial Sequence
  Synthetic
INPNYDTT

SEQ ID NO: 74 - protein, 16 aa, Artificial Sequence
  Synthetic
ARSGYYGSSYYWHFDV

SEQ ID NO: 75 - protein, 12 aa, Artificial Sequence
  Synthetic
QSLLYSSNQKNY

SEQ ID NO: 76 - protein, 9 aa, Artificial Sequence
  Synthetic
QQYYNYPFT

SEQ ID NO: 77 - protein, 123 aa, Artificial Sequence
  Synthetic
EVQLQQFGAELVKPGASVKISCKASGYTFTDYNMAWVKESHGKSLEWIGDINPNYDTTSYNQKFKGKATLTVDKSSSTAHMELRSLTSEGTAVYYCARSGYYGSSYYWHFDVWGTGTTVTVSS

SEQ ID NO: 78 - protein, 113 aa, Artificial Sequence
  Synthetic
DIVMSQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYNYPFTFGSGTKLEIK

SEQ ID NO: 79 - protein, 8 aa, Artificial Sequence
  Synthetic
GFNIKDYY

SEQ ID NO: 80 - protein, 8 aa, Artificial Sequence
  Synthetic
IDPESGDT

SEQ ID NO: 81 - protein, 9 aa, Artificial Sequence
  Synthetic
YGHDYRVDC

SEQ ID NO: 82 - protein, 11 aa, Artificial Sequence
  Synthetic
QSLVHSNGNTY

SEQ ID NO: 83 - protein, 3 aa, Artificial Sequence
  Synthetic
KVS

SEQ ID NO: 84 - protein, 9 aa, Artificial Sequence
  Synthetic
SQSTHIPWT

SEQ ID NO: 85 - protein, 116 aa, Artificial Sequence
  Synthetic
EVQLQQSGAELVRSGASVKLSCTASGFNIKDYYMHWVKQRPEQGLEWIGWIDPESGDTEYAPKFQGRATMTADTSSNTAYMQLSSLTSEDTAVYYCYGHDYRVDCWGQGTSVTVSS

SEQ ID NO: 86 - protein, 112 aa, Artificial Sequence
  Synthetic
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHIPWTFGGGTKLEIK

SEQ ID NO: 87 - protein, 8 aa, Artificial Sequence
  Synthetic
INPNYDST

SEQ ID NO: 88 - protein, 9 aa, Artificial Sequence
  Synthetic
QQYYHYPFT

SEQ ID NO: 89 - protein, 123 aa, Artificial Sequence
  Synthetic
EVQLQQFGAELVKPGASVKISCKASGYTFTDYNMGWVKQSHGKSLEWIGDINPNYDSTSYTQKFKGKATLTVDKSSSTAYMELRSLTSEDTAVYYCARSGYYGSSYYWHFDVWGTGTTVTVSS

SEQ ID NO: 90 - protein, 113 aa, Artificial Sequence
  Synthetic
DIVMSQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYHYPFTFGSGTKLEIK

SEQ ID NO: 91 - protein, 8 aa, Artificial Sequence
  Synthetic
GFSLTNYG

SEQ ID NO: 92 - protein, 7 aa, Artificial Sequence
  Synthetic
IWNDGSA

SEQ ID NO: 93 - protein, 11 aa, Artificial Sequence
  Synthetic
ARHESSNPFAY

SEQ ID NO: 94 - protein, 6 aa, Artificial Sequence
  Synthetic
QSIGTS

SEQ ID NO: 95 - protein, 3 aa, Artificial Sequence
  Synthetic
SAS

SEQ ID NO: 96 - protein, 9 aa, Artificial Sequence
  Synthetic
QQNNRWPYT

SEQ ID NO: 97 - protein, 117 aa, Artificial Sequence
  Synthetic
QVQLKESGPGLVAPSQSLSITCTVSGFSLTNYGVHWVRQPPGKGLEWLVVIWNDGSATYNSALESRLSISKDNSKSQVFLKMNSLQTDDTAMYYCARHESSNPFAYWGQGTLVTVSA

SEQ ID NO: 98 - protein, 107 aa, Artificial Sequence
  Synthetic
DILLTQSPAILSVSPGERVSFSCRASQSIGTSIHWYQQRTNGSPRLLIKSASESIAGIPSRFSGSGSGTDFTLSINSVESEDIADYYCQQNNRWPYTFGGGTKLEIK

SEQ ID NO: 99 - protein, 8 aa, Artificial Sequence
  Synthetic
DFNIKDDY

SEQ ID NO: 100 - protein, 8 aa, Artificial Sequence
  Synthetic
IDPANGNT

SEQ ID NO: 101 - protein, 6 aa, Artificial Sequence
  Synthetic
ALGYTY

SEQ ID NO: 102 - protein, 11 aa, Artificial Sequence
  Synthetic
QSLLHSYGKTY

SEQ ID NO: 103 - protein, 9 aa, Artificial Sequence
  Synthetic
LQTTHFPQT

SEQ ID NO: 104 - protein, 113 aa, Artificial Sequence
  Synthetic
EVQLQQSGAELVRPGASVKLSCTASDFNIKDDYIHWVKQRPEQGLEWIGRIDPANGNTKYAPKFQDKATITADTSSNTAYLQLSSLTSEDTAVYYCALGYTYWGQGTTLTVSS

SEQ ID NO: 105 - protein, 112 aa, Artificial Sequence
  Synthetic
DVVMTQTPLTLSVTIGQPASISCKSSQSLLHSYGKTYLNWLLQRPGQSPKLLIYLVSKLESGVPDRFSGSGSGTDFTLKISRVEAEDLGVYYCLQTTHFPQTFGGGTKLEIK

SEQ ID NO: 106 - protein, 8 aa, Artificial Sequence
  Synthetic
GFTFSDYG

SEQ ID NO: 107 - protein, 8 aa, Artificial Sequence
  Synthetic
INSGSSTI

SEQ ID NO: 108 - protein, 13 aa, Artificial Sequence
  Synthetic
ARPGDYDNYAMDY

SEQ ID NO: 109 - protein, 6 aa, Artificial Sequence
  Synthetic
QDVSVA

SEQ ID NO: 110 - protein, 3 aa, Artificial Sequence
  Synthetic
WAY

SEQ ID NO: 111 - protein, 10 aa, Artificial Sequence
  Synthetic
QQHYNTPPWT

SEQ ID NO: 112 - protein, 120 aa, Artificial Sequence
  Synthetic
EVQLVESGGDLVKPGGSLKLSCAASGFTFSDYGMHWVRQGPEKGLEWVAYINSGSSTIYYADTVKGRFTISRDNAKNTLFLQMTSLRSEDTAMYYCARPGDYDNYAMDYWGQGTSVTVSS

SEQ ID NO: 113 - protein, 108 aa, Artificial Sequence
  Synthetic
DIVMTQSHKFLSTSVGDRVSITCKASQDVSVAVAWYQQKPGQSPKLLIYWAYTRHTGVPDRFTGSGSGTEYTLTISSVQAEDLALYYCQQHYNTPPWTFGGGTKLEIK

SEQ ID NO: 114 - protein, 8 aa, Artificial Sequence
  Synthetic
IDPENADT

SEQ ID NO: 115 - protein, 9 aa, Artificial Sequence
  Synthetic
YAWDYSMDY

SEQ ID NO: 116 - protein, 9 aa, Artificial Sequence
  Synthetic
SQNTHVPYT

SEQ ID NO: 117 - protein, 116 aa, Artificial Sequence
  Synthetic
EVQLQQSGAELVRSGASVKLSCTASGFNIKDYYIHWVKQRPEQGLEWIGWIDPENADTEYAPKFQGKATMTPDTSSNTAYLQLSSLTSEDTAVYYCYAWDYSMDYWGQGTSVTVSS

SEQ ID NO: 118 - protein, 112 aa, Artificial Sequence
  Synthetic
DVVMTQTPLSLSVSLGDQASISCRSSQSLVHSNGNTYLHWYLQKPGQSPKLLIYKVSNRFSGVPDRFSGSGSGTDFILKISRVEAEDLGVYFCSQNTHVPYTFGGGTRLEIK

SEQ ID NO: 119 - protein, 8 aa, Artificial Sequence
  Synthetic
GFTFTDYG

SEQ ID NO: 120 - protein, 8 aa, Artificial Sequence
  Synthetic
IYPSSGNS

SEQ ID NO: 121 - protein, 12 aa, Artificial Sequence
  Synthetic
ARSTYYGSPIDY

SEQ ID NO: 122 - protein, 6 aa, Artificial Sequence
  Synthetic
QDVDTT

SEQ ID NO: 123 - protein, 9 aa, Artificial Sequence
  Synthetic
QQYSTYPLT

SEQ ID NO: 124 - protein, 119 aa, Artificial Sequence
  Synthetic
QVQLQQSGTELARPGASVKLSCKASGFTFTDYGINWVKQRTGQGLEWIGEIYPSSGNSYYNEKFKAKATLTADKSSSTAYMELRSLTSEDSAVYFCARSTYYGSPIDYWGQGTTLTVSS

SEQ ID NO: 125 - protein, 107 aa, Artificial Sequence
  Synthetic
DIVMTQSHKFMSTPVGDRVSITCKASQDVDTTVAWYQQKPGQSPKLLIYWASTRQIGVPDRFTGSGSGTDFTLTISNVQSEDLADYFCQQYSTYPLTFGGGTKLEIK

SEQ ID NO: 126 - protein, 8 aa, Artificial Sequence
  Synthetic
GFSLTSYA

SEQ ID NO: 127 - protein, 7 aa, Artificial Sequence
  Synthetic
IWTGGGT

SEQ ID NO: 128 - protein, 13 aa, Artificial Sequence
  Synthetic
ARIYDGYYRYFDV

SEQ ID NO: 129 - protein, 6 aa, Artificial Sequence
  Synthetic
QSVSND

SEQ ID NO: 130 - protein, 3 aa, Artificial Sequence
  Synthetic
YAS

SEQ ID NO: 131 - protein, 9 aa, Artificial Sequence
  Synthetic
QQDYSSPWT

SEQ ID NO: 132 - protein, 119 aa, Artificial Sequence
  Synthetic
QVQLKESGPGLVAPSQSLSITCTVSGFSLTSYAITWVRQSPGKGLEWLGLIWTGGGTNYNSALKSRLSISKDNSKSQVFLKMNSLQTDDTARYYCARIYDGYYRYFDVWGTGTTVTVSS

SEQ ID NO: 133 - protein, 107 aa, Artificial Sequence
  Synthetic
RIVLTQTPKFLLVSAGDRVTMTCKASQSVSNDVAWYQQKPGQSPKLLIYYASNRYTGVPDRFTGSGYGTDFTFTISTVQAEDLAVYFCQQDYSSPWTFGGGTKLEIK

SEQ ID NO: 134 - protein, 9 aa, Artificial Sequence
  Synthetic
NVLTMPTAY

SEQ ID NO: 135 - protein, 9 aa, Artificial Sequence
  Synthetic
QQYYSYPYT

SEQ ID NO: 136 - protein, 116 aa, Artificial Sequence
  Synthetic
EVQLQQSGAELVRSGASVKLSCTASGFNIKDYYMHWVKQRPEQGLDWIGWIDPENGDTEYAPKFQGKATMTADTSSNTAYLQLSSLTSEDTAVYYCNVLTMPTAYWGQGTLVTVSA

SEQ ID NO: 137 - protein, 113 aa, Artificial Sequence
  Synthetic
DIVMSQSPSSLAVSVGEKVIMSCKSSQSLLYSSNQKNYLAWYQQKPGQSPKLLIYWASTRESGVPDRFTGSGSGTDFTLTISSVKAEDLAVYYCQQYYSYPYTFGGGTKLEIK

SEQ ID NO: 138 - protein, 8 aa, Artificial Sequence
  Synthetic
GFTFSSYG

SEQ ID NO: 139 - protein, 8 aa, Artificial Sequence
  Synthetic
ITSGGSYT

SEQ ID NO: 140 - protein, 7 aa, Artificial Sequence
  Synthetic
ALWSLDY

SEQ ID NO: 141 - protein, 5 aa, Artificial Sequence
  Synthetic
SSLSY

SEQ ID NO: 142 - protein, 9 aa, Artificial Sequence
  Synthetic
HQRRSYPWT

SEQ ID NO: 143 - protein, 114 aa, Artificial Sequence
  Synthetic
EVQLMESGGDLVKPGGSLKLSCAASGFTFSSYGLSWVRQTPDKRLEWVATITSGGSYTYYPDSVKGRFTISRDNARNTLYLQMFSLKSEDTAMYYCALWSLDYWGQGTTLTVSS

SEQ ID NO: 144 - protein, 106 aa, Artificial Sequence
  Synthetic
QIVLTQSPAIMSASPGEKVTMTCSANSSLSYMHWYQQKPGTSPKRWIYDTSELASGVPARFSGSGSGTSYSLTISSMEAEDAATYYCHQRRSYPWTFGGGTKLEIK

SEQ ID NO: 145 - protein, 5 aa, Artificial Sequence
  Synthetic
DDYMY

SEQ ID NO: 146 - protein, 17 aa, Artificial Sequence
  Synthetic
WIDPENGDTEYASKFQD

SEQ ID NO: 147 - protein, 8 aa, Artificial Sequence
  Synthetic
WLRRGLDY

SEQ ID NO: 148 - protein, 16 aa, Artificial Sequence
  Synthetic
RSSKSLLHSNGYTYLF

SEQ ID NO: 149 - protein, 7 aa, Artificial Sequence
  Synthetic
RMSNLAS

SEQ ID NO: 150 - protein, 7 aa, Artificial Sequence
  Synthetic
GFNIKDD

SEQ ID NO: 151 - protein, 3 aa, Artificial Sequence
  Synthetic
ENG

SEQ ID NO: 152 - protein, 6 aa, Artificial Sequence
  Synthetic
LRRGLD

SEQ ID NO: 153 - protein, 12 aa, Artificial Sequence
  Synthetic
SKSLLHSNGYTY

SEQ ID NO: 154 - protein, 6 aa, Artificial Sequence
  Synthetic
HLEYPF

SEQ ID NO: 155 - protein, 6 aa, Artificial Sequence
  Synthetic
SGYYWN

SEQ ID NO: 156 - protein, 16 aa, Artificial Sequence
  Synthetic
YITFDGANNYNPSLKN

SEQ ID NO: 157 - protein, 10 aa, Artificial Sequence
  Synthetic
SSYDYDVLDY

SEQ ID NO: 158 - protein, 11 aa, Artificial Sequence
  Synthetic
RASQDISNFLN

SEQ ID NO: 159 - protein, 7 aa, Artificial Sequence
  Synthetic
YTSRLHS

SEQ ID NO: 160 - protein, 8 aa, Artificial Sequence
  Synthetic
GYSITSGY

SEQ ID NO: 161 - protein, 3 aa, Artificial Sequence
  Synthetic
FDG

SEQ ID NO: 162 - protein, 8 aa, Artificial Sequence
  Synthetic
SYDYDVLD

SEQ ID NO: 163 - protein, 7 aa, Artificial Sequence
  Synthetic
SQDISNF

SEQ ID NO: 164 - protein, 6 aa, Artificial Sequence
  Synthetic
GHTLPY

SEQ ID NO: 165 - protein, 5 aa, Artificial Sequence
  Synthetic
DYCIN

SEQ ID NO: 166 - protein, 17 aa, Artificial Sequence
  Synthetic
WIYPGSGNTRYSERFKG

SEQ ID NO: 167 - protein, 11 aa, Artificial Sequence
  Synthetic
EDYYPYHGMDY

SEQ ID NO: 168 - protein, 15 aa, Artificial Sequence
  Synthetic
RASESVDGYDNSFMH

SEQ ID NO: 169 - protein, 7 aa, Artificial Sequence
  Synthetic
RASNLES

SEQ ID NO: 170 - protein, 7 aa, Artificial Sequence
  Synthetic
GYSFTDY

SEQ ID NO: 171 - protein, 3 aa, Artificial Sequence
  Synthetic
GSG

SEQ ID NO: 172 - protein, 9 aa, Artificial Sequence
  Synthetic
DYYPYHGMD

SEQ ID NO: 173 - protein, 11 aa, Artificial Sequence
  Synthetic
SESVDGYDNSF

SEQ ID NO: 174 - protein, 6 aa, Artificial Sequence
  Synthetic
SSEDPW

SEQ ID NO: 175 - protein, 330 aa, Homo sapiens [sequence omitted]

SEQ ID NO: 176 - protein, 330 aa, Artificial Sequence
  Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 177 - protein, 107 aa, Artificial Sequence [sequence omitted]

SEQ ID NO: 178 - protein, 447 aa, Artificial Sequence
  Synthetic
EVQLQQSGAELVRPGASVKLSCTASGFNIKDDYMYWVKQRPEQGLEWIGWIDPENGDTEYASKFQDKATVTADTSSNTAYLQLSSLTSEDTAVYYCTLWLRRGLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 179 - protein, 219 aa, Artificial Sequence
  Synthetic
DIVMTQAAPSVPVTPGESVSISCRSSKSLLHSNGYTYLFWFLQRPGQSPQLLIYRMSNLASGVPDRFSGSGSGTAFTLRISRVEAEDVGVYYCMQHLEYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 180 - protein, 449 aa, Artificial Sequence
  Synthetic
DVQLQESGPGLVKPSQSLSLTCSVTGYSITSGYYWNWIRQFPGNKLEWMGYITFDGANNYNPSLKNRISITRDTSKNQFFLKLTSVTTEDTATYYCTRSSYDYDVLDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 181 - protein, 214 aa, Artificial Sequence
  Synthetic
DIQMTQTTSSLSASLGDRVTISCRASQDISNFLNWYQQRPDGTVKLLIYYTSRLHSGVPSRFSGSGSGTDFSLTVSNLEQEDIATYFCQQGHTLPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 182 - protein, 450 aa, Artificial Sequence
  Synthetic
QIQLQQSGPELVRPGASVKISCKASGYSFTDYCINWVNQRPGQGLEWIGWIYPGSGNTRYSERFKGKATLTVDTSSNTAYMQLSSLTSEDSAVYFCAREDYYPYHGMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 183 - protein, 218 aa, Artificial Sequence
  Synthetic
DIVLTQSPTSLAVSLGQRATISCRASESVDGYDNSFMHWYQQKPGQPPKLLIFRASNLESGIPARFSGSGSRTDFTLTINPVEAADVATYYCQQSSEDPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 184 - protein, 108 aa, Artificial Sequence
  Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 185 - protein, 225 aa, Artificial Sequence
  Synthetic
EVQLQQSGAELVRPGASVKLSCTASGFNIKDDYMYWVKQRPEQGLEWIGWIDPENGDTEYASKFQDKATVTADTSSNTAYLQLSSLTSEDTAVYYCTLWLRRGLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 186 - protein, 227 aa, Artificial Sequence
  Synthetic
DVQLQESGPGLVKPSQSLSLTCSVTGYSITSGYYWNWIRQFPGNKLEWMGYITFDGANNYNPSLKNRISITRDTSKNQFFLKLTSVTTEDTATYYCTRSSYDYDVLDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 187 - protein, 228 aa, Artificial Sequence
  Synthetic
QIQLQQSGPELVRPGASVKISCKASGYSFTDYCINWVNQRPGQGLEWIGWIYPGSGNTRYSERFKGKATLTVDTSSNTAYMQLSSLTSEDSAVYFCAREDYYPYHGMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 188 - protein, 8 aa, Artificial Sequence
  Synthetic
GYSFTSYW

SEQ ID NO: 189 - protein, 8 aa, Artificial Sequence
  Synthetic
IYPGDSDT

SEQ ID NO: 190 - protein, 15 aa, Artificial Sequence
  Synthetic
ARFPYDSSGYYSFDY

SEQ ID NO: 191 - protein, 6 aa, Artificial Sequence
  Synthetic
QSISSY

SEQ ID NO: 192 - protein, 3 aa, Artificial Sequence
  Synthetic
AAS

SEQ ID NO: 193 - protein, 9 aa, Artificial Sequence
  Synthetic
QQSYSTPLT

SEQ ID NO: 194 - protein, 5 aa, Artificial Sequence
  Synthetic
SYWIG

SEQ ID NO: 195 - protein, 18 aa, Artificial Sequence
  Synthetic
IIYPGDSDTRYSPSFQGQ

SEQ ID NO: 196 - protein, 13 aa, Artificial Sequence
  Synthetic
FPYDSSGYYSFDY

SEQ ID NO: 197 - protein, 11 aa, Artificial Sequence
  Synthetic
RASQSISSYLN

SEQ ID NO: 198 - protein, 7 aa, Artificial Sequence
  Synthetic
AASSLQS

SEQ ID NO: 199 - protein, 7 aa, Artificial Sequence
  Synthetic
GYSFTSY

SEQ ID NO: 200 - protein, 3 aa, Artificial Sequence
  Synthetic
GDS

SEQ ID NO: 201 - protein, 11 aa, Artificial Sequence
  Synthetic
PYDSSGYYSFD

SEQ ID NO: 202 - protein, 7 aa, Artificial Sequence
  Synthetic
SQSISSY

SEQ ID NO: 203 - protein, 6 aa, Artificial Sequence
  Synthetic
SYSTPL

SEQ ID NO: 204 - protein, 122 aa, Artificial Sequence
  Synthetic
QVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARFPYDSSGYYSFDYWGQGTLVTVSS

SEQ ID NO: 205 - protein, 107 aa, Artificial Sequence [sequence omitted]

SEQ ID NO: 206 - protein, 243 aa, Artificial Sequence
  Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKEGKSSGSGSESKASQVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARFPYDSSGYYSFDYWGQGTLVTVSS

SEQ ID NO: 207 - protein, 231 aa, Artificial Sequence
  Synthetic
PKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 208 - protein, 481 aa, Artificial Sequence
  Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKEGKSSGSGSESKASQVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARFPYDSSGYYSFDYWGQGTLVTVSSDIEGRMDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 209 - protein, 481 aa, Artificial Sequence
  Synthetic
PKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKDIEGRMDDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKEGKSSGSGSESKASQVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARFPYDSSGYYSFDYWGQGTLVTVSS

SEQ ID NO: 210 - protein, 453 aa, Artificial Sequence
  Synthetic
QVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARFPYDSSGYYSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 211 - protein, 452 aa, Artificial Sequence
  Synthetic
QVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARFPYDSSGYYSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 212 - protein, 214 aa, Artificial Sequence
  Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 213 - protein, 232 aa, Artificial Sequence
  Synthetic
QVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARFPYDSSGYYSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCP

SEQ ID NO: 214 - protein, 19 aa, Artificial Sequence
  Synthetic
MGWSCIILFLVATATGVHS

SEQ ID NO: 215 - protein, 14 aa, Artificial Sequence
  Synthetic
EGKSSGSGSESKAS

SEQ ID NO: 216 - protein, 5 aa, Artificial Sequence
  Synthetic
SYWMH

SEQ ID NO: 217 - protein, 17 aa, Artificial Sequence
  Synthetic
EINPTNGRTNYIEKFKS

SEQ ID NO: 218 - protein, 7 aa, Artificial Sequence
  Synthetic
GTRAYHY

SEQ ID NO: 219 - protein, 11 aa, Artificial Sequence
  Synthetic
RASDNLYSNLA

SEQ ID NO: 220 - protein, 7 aa, Artificial Sequence
  Synthetic
DATNLAD

SEQ ID NO: 221 - protein, 9 aa, Artificial Sequence
  Synthetic
QHFWGTPLT

SEQ ID NO: 222 - protein, 7 aa, Artificial Sequence
  Synthetic
GYTFTSY

SEQ ID NO: 223 - protein, 6 aa, Artificial Sequence
  Synthetic
NPTNGR

SEQ ID NO: 224 - protein, 6 aa, Artificial Sequence
  Synthetic
TSYWMH

SEQ ID NO: 225 - protein, 13 aa, Artificial Sequence
  Synthetic
WIGEINPTNGRTN

SEQ ID NO: 226 - protein, 6 aa, Artificial Sequence
  Synthetic
ARGTRA

SEQ ID NO: 227 - protein, 7 aa, Artificial Sequence
  Synthetic
YSNLAWY

SEQ ID NO: 228 - protein, 10 aa, Artificial Sequence
  Synthetic
LLVYDATNLA

SEQ ID NO: 229 - protein, 8 aa, Artificial Sequence
  Synthetic
QHFWGTPL

SEQ ID NO: 230 - protein, 116 aa, Artificial Sequence
  Synthetic
QVQLQQPGAELVKPGASVKLSCKASGYTFTSYWMHWVKQRPGQGLEWIGEINPTNGRTNYIEKFKSKATLTVDKSSSTAYMQLSSLTSEDSAVYYCARGTRAYHYWGQGTSVTVSS

SEQ ID NO: 231 - protein, 107 aa, Artificial Sequence
  Synthetic
DIQMTQSPASLSVSVGETVTITCRASDNLYSNLAWYQQKQGKSPQLLVYDATNLADGVPSRFSGSGSGTQYSLKINSLQSEDFGTYYCQHFWGTPLTFGAGTKLELK

SEQ ID NO: 232 - protein, 9 aa, Artificial Sequence
  Synthetic
QHFAGTPLT

SEQ ID NO: 233 - protein, 8 aa, Artificial Sequence
  Synthetic
QHFAGTPL

SEQ ID NO: 234 - protein, 116 aa, Artificial Sequence
  Synthetic
EVQLVQSGAEVKKPGASVKVSCKASGYTFTSYWMHWVRQAPGQRLEWIGEINPTNGRTNYIEKFKSRATLTVDKSASTAYMELSSLRSEDTAVYYCARGTRAYHYWGQGTMVTVSS

SEQ ID NO: 235 - protein, 107 aa, Artificial Sequence
  Synthetic
DIQMTQSPSSLSASVGDRVTITCRASDNLYSNLAWYQQKPGKSPKLLVYDATNLADGVPSRFSGSGSGTDYTLTISSLQPEDFATYYCQHFWGTPLTFGQGTKVEIK

SEQ ID NO: 236 - protein, 446 aa, Artificial Sequence
  Synthetic
QVQLQQPGAELVKPGASVKLSCKASGYTFTSYWMHWVKQRPGQGLEWIGEINPTNGRTNYIEKFKSKATLTVDKSSSTAYMQLSSLTSEDSAVYYCARGTRAYHYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 237 - protein, 214 aa, Artificial Sequence
  Synthetic
DIQMTQSPASLSVSVGETVTITCRASDNLYSNLAWYQQKQGKSPQLLVYDATNLADGVPSRFSGSGSGTQYSLKINSLQSEDFGTYYCQHFWGTPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 238 - protein, 446 aa, Artificial Sequence
  Synthetic
EVQLVQSGAEVKKPGASVKVSCKASGYTFTSYWMHWVRQAPGQRLEWIGEINPTNGRTNYIEKFKSRATLTVDKSASTAYMELSSLRSEDTAVYYCARGTRAYHYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 239 - protein, 214 aa, Artificial Sequence
  Synthetic
DIQMTQSPSSLSASVGDRVTITCRASDNLYSNLAWYQQKPGKSPKLLVYDATNLADGVPSRFSGSGSGTDYTLTISSLQPEDFATYYCQHFWGTPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 240 - protein, 226 aa, Artificial Sequence
  Synthetic
QVQLQQPGAELVKPGASVKLSCKASGYTFTSYWMHWVKQRPGQGLEWIGEINPTNGRTNYIEKFKSKATLTVDKSSSTAYMQLSSLTSEDSAVYYCARGTRAYHYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCP

SEQ ID NO: 241 - protein, 226 aa, Artificial Sequence
  Synthetic
EVQLVQSGAEVKKPGASVKVSCKASGYTFTSYWMHWVRQAPGQRLEWIGEINPTNGRTNYIEKFKSRATLTVDKSASTAYMELSSLRSEDTAVYYCARGTRAYHYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCP

SEQ ID NO: 242 - protein, 760 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 243 - protein, 760 aa, Macaca mulatta
MMDQARSAFSNLFGGEPLSYTRFSLARQVDGDNSHVEMKLGVDEEENTDNNTKPNGTKPKRCGGNICYGTIAVIIFFLIGFMIGYLGYCKGVEPKTECERLAGTESPAREEPEEDFPAAPRLYWDDLKRKLSEKLDTTDFTSTIKLLNENLYVPREAGSQKDENLALYIENQFREFKLSKVWRDQHFVKIQVKDSAQNSVIIVDKNGGLVYLVENPGGYVAYSKAATVTGKLVHANFGTKKDFEDLDSPVNGSIVIVRAGKITFAEKVANAESLNAIGVLIYMDQTKFPIVKADLSFFGHAHLGTGDPYTPGFPSFNHTQFPPSQSSGLPNIPVQTISRAAAEKLFGNMEGDCPSDWKTDSTCKMVTSENKSVKLTVSNVLKETKILNIFGVIKGFVEPDHYVVVGAQRDAWGPGAAKSSVGTALLLKLAQMFSDMVLKDGFQPSRSIIFASWSAGDFGSVGATEWLEGYLSSLHLKAFTYINLDKAVLGTSNFKVSASPLLYTLIEKTMQDVKHPVTGRSLYQDSNWASKVEKLTLDNAAFPFLAYSGIPAVSFCFCEDTDYPYLGTTMDTYKELVERIPELNKVARAAAEVAGQFVIKLTHDTELNLDYERYNSQLLLFLRDLNQYRADVKEMGLSLQWLYSARGDFFRATSRLTTDFRNAEKRDKFVMKKLNDRVMRVEYYFLSPYVSPKESPFRHVFWGSGSHTLSALLESLKLRRQNNSAFNETLFRNQLALATWTIQGAANALSGDVWDIDNEF

SEQ ID NO: 244 - protein, 760 aa, Macaca fascicularis
MMDQARSAFSNLFGGEPLSYTRFSLARQVDGDNSHVEMKLGVDEEENTDNNTKANGTKPKRCGGNICYGTIAVIIFFLIGFMIGYLGYCKGVEPKTECERLAGTESPAREEPEEDFPAAPRLYWDDLKRKLSEKLDTTDFTSTIKLLNENLYVPREAGSQKDENLALYIENQFREFKLSKVWRDQHFVKIQVKDSAQNSVIIVDKNGGLVYLVENPGGYVAYSKAATVTGKLVHANFGTKKDFEDLDSPVNGSIVIVRAGKITFAEKVANAESLNAIGVLIYMDQTKFPIVKADLSFFGHAHLGTGDPYTPGFPSFNHTQFPPSQSSGLPNIPVQTISRAAAEKLFGNMEGDCPSDWKTDSTCKMVTSENKSVKLTVSNVLKETKILNIFGVIKGFVEPDHYVVVGAQRDAWGPGAAKSSVGTALLLKLAQMFSDMVLKDGFQPSRSIIFASWSAGDFGSVGATEWLEGYLSSLHLKAFTYINLDKAVLGTSNFKVSASPLLYTLIEKTMQDVKHPVTGRSLYQDSNWASKVEKLTLDNAAFPFLAYSGIPAVSFCFCEDTDYPYLGTTMDTYKELVERIPELNKVARAAAEVAGQFVIKLTHDTELNLDYERYNSQLLLFLRDLNQYRADVKEMGLSLQWLYSARGDFFRATSRLTTDFRNAEKRDKFVMKKLNDRVMRVEYYFLSPYVSPKESPFRHVFWGSGSHTLSALLESLKLRRQNNSAFNETLFRNQLALATWTIQGAANALSGDVWDIDNEF

SEQ ID NO: 245 - protein, 763 aa, Mus musculus
MMDQARSAFSNLFGGEPLSYTRFSLARQVDGDNSHVEMKLAADEEENADNNMKASVRKPKRFNGRLCFAAIALVIFFLIGFMSGYLGYCKRVEQKEECVKLAETEETDKSETMETEDVPTSSRLYWADLKTLLSEKLNSIEFADTIKQLSQNTYTPREAGSQKDESLAYYIENQFHEFKFSKVWRDEHYVKIQVKSSIGQNMVTIVQSNGNLDPVESPEGYVAFSKPTEVSGKLVHANFGTKKDFEELSYSVNGSLVIVRAGEITFAEKVANAQSFNAIGVLIYMDKNKFPVVEADLALFGHAHLGTGDPYTPGFPSFNHTQFPPSQSSGLPNIPVQTISRAAAEKLFGKMEGSCPARWNIDSSCKLELSQNQNVKLIVKNVLKERRILNIFGVIKGYEEPDRYVVVGAQRDALGAGVAAKSSVGTGLLLKLAQVFSDMISKDGFRPSRSIIFASWTAGDFGAVGATEWLEGYLSSLHLKAFTYINLDKVVLGTSNFKVSASPLLYTLMGKIMQDVKHPVDGKSLYRDSNWISKVEKLSFDNAAYPFLAYSGIPAVSFCFCEDADYPYLGTRLDTYEALTQKVPQLNQMVRTAAEVAGQLIIKLTHDVELNLDYEMYNSKLLSFMKDLNQFKTDIRDMGLSLQWLYSARGDYFRATSRLTTDFHNAEKTNRFVMREINDRIMKVEYHFLSPYVSPRESPFRHIFWGSGSHTLSALVENLKLRQKNITAFNETLFRNQLALATWTIQGVANALSGDIWNIDNEF

SEQ ID NO: 246 - protein, 197 aa, Artificial Sequence
  Synthetic
FVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKE

SEQ ID NO: 247 - protein, 7 aa, Artificial Sequence
  Synthetic
DIEGRMD

SEQ ID NO: 248 - protein, 7 aa, Artificial Sequence
  Synthetic
ASSLNIA

SEQ ID NO: 249 - protein, 12 aa, Artificial Sequence
  Synthetic
SKTFNTHPQSTP

SEQ ID NO: 250 - protein, 12 aa, Artificial Sequence
  Synthetic
TARGEHKEEELI

SEQ ID NO: 251 - protein, 9 aa, Artificial Sequence
  Synthetic
CQAQGQLVC

SEQ ID NO: 252 - protein, 9 aa, Artificial Sequence
  Synthetic
CSERSMNFC

SEQ ID NO: 253 - protein, 9 aa, Artificial Sequence
  Synthetic
CPKTRRVPC

SEQ ID NO: 254 - protein, 20 aa, Artificial Sequence
  Synthetic
WLSEAGPVVTVRALRGTGSW

SEQ ID NO: 255 - protein, 9 aa, Artificial Sequence
  Synthetic
CMQHSMRVC

SEQ ID NO: 256 - protein, 7 aa, Artificial Sequence
  Synthetic
DDTRHWG

SEQ ID NO: 257 - protein, 5 aa, Artificial Sequence
  Synthetic
  misc_feature (3)..(3): Xaa can be any naturally occurring amino acid
LPXTG

SEQ ID NO: 258 - RNA, 21 nt, Artificial Sequence
  Synthetic
  misc_feature (1)..(1): modified by 2′Fluoro
  misc_feature (2)..(2): modified by 2′OMe
  misc_feature (3)..(3): modified by 2′Fluoro
  misc_feature (4)..(4): modified by 2′OMe
  misc_feature (5)..(5): modified by 2′Fluoro
  misc_feature (6)..(6): modified by 2′OMe
  misc_feature (7)..(7): modified by 2′Fluoro
  misc_feature (8)..(8): modified by 2′OMe
  misc_feature (9)..(9): modified by 2′Fluoro
  misc_feature (10)..(10): modified by 2′OMe
  misc_feature (11)..(11): modified by 2′Fluoro
  misc_feature (12)..(12): modified by 2′OMe
  misc_feature (13)..(13): modified by 2′Fluoro
  misc_feature (14)..(14): modified by 2′OMe
  misc_feature (15)..(15): modified by 2′Fluoro
  misc_feature (16)..(16): modified by 2′OMe
  misc_feature (17)..(17): modified by 2′Fluoro
  misc_feature (18)..(18): modified by 2′OMe
  misc_feature (19)..(19): modified by 2′Fluoro
  misc_feature (20)..(20): modified by 2′OMe
  misc_feature (21)..(21): modified by 2′Fluoro
uccuaugacuguagauuuuau

SEQ ID NO: 259 - RNA, 23 nt, Artificial Sequence
  Synthetic
  misc_feature (1)..(1): modified by 2′OMe
  misc_feature (2)..(2): modified by 2′Fluoro
  misc_feature (3)..(3): modified by 2′OMe
  misc_feature (4)..(4): modified by 2′Fluoro
  misc_feature (5)..(5): modified by 2′OMe
  misc_feature (6)..(6): modified by 2′Fluoro
  misc_feature (7)..(7): modified by 2′OMe
  misc_feature (8)..(8): modified by 2′Fluoro
  misc_feature (9)..(9): modified by 2′OMe
  misc_feature (10)..(10): modified by 2′Fluoro
  misc_feature (11)..(11): modified by 2′OMe
  misc_feature (12)..(12): modified by 2′Fluoro
  misc_feature (13)..(13): modified by 2′OMe
  misc_feature (14)..(14): modified by 2′Fluoro
  misc_feature (15)..(15): modified by 2′OMe
  misc_feature (16)..(16): modified by 2′Fluoro
  misc_feature (17)..(17): modified by 2′OMe
  misc_feature (18)..(18): modified by 2′Fluoro
  misc_feature (19)..(19): modified by 2′OMe
  misc_feature (20)..(20): modified by 2′Fluoro
  misc_feature (21)..(21): modified by 2′OMe
  misc_feature (21)..(23): phosphorothioate linkage
  misc_feature (22)..(22): modified by 2′Fluoro
  misc_feature (23)..(23): modified by 2′OMe
auaaaaucuacagucauaggaau

SEQ ID NO: 260 - RNA, 21 nt, Artificial Sequence
  Synthetic
  misc_feature (1)..(1): modified by 2′Fluoro
  misc_feature (2)..(2): modified by 2′OMe
  misc_feature (3)..(3): modified by 2′Fluoro
  misc_feature (4)..(4): modified by 2′OMe
  misc_feature (5)..(5): modified by 2′Fluoro
  misc_feature (6)..(6): modified by 2′OMe
  misc_feature (7)..(7): modified by 2′Fluoro
  misc_feature (8)..(8): modified by 2′OMe
  misc_feature (9)..(9): modified by 2′Fluoro
  misc_feature (10)..(10): modified by 2′OMe
  misc_feature (11)..(11): modified by 2′Fluoro
  misc_feature (12)..(12): modified by 2′OMe
  misc_feature (13)..(13): modified by 2′Fluoro
  misc_feature (14)..(14): modified by 2′OMe
  misc_feature (15)..(15): modified by 2′Fluoro
  misc_feature (16)..(16): modified by 2′OMe
  misc_feature (17)..(17): modified by 2′Fluoro
  misc_feature (18)..(18): modified by 2′OMe
  misc_feature (19)..(19): modified by 2′Fluoro
  misc_feature (20)..(20): modified by 2′OMe
  misc_feature (21)..(21): modified by 2′Fluoro
uguaauaaccauaucuaccuu

SEQ ID NO: 261 - RNA, 23 nt, Artificial Sequence
  Synthetic
  misc_feature (1)..(1): modified by 2′OMe
  misc_feature (2)..(2): modified by 2′Fluoro
  misc_feature (3)..(3): modified by 2′OMe
  misc_feature (4)..(4): modified by 2′Fluoro
  misc_feature (5)..(5): modified by 2′OMe
  misc_feature (6)..(6): modified by 2′Fluoro
  misc_feature (7)..(7): modified by 2′OMe
  misc_feature (8)..(8): modified by 2′Fluoro
  misc_feature (9)..(9): modified by 2′OMe
  misc_feature (10)..(10): modified by 2′Fluoro
  misc_feature (11)..(11): modified by 2′OMe
  misc_feature (12)..(12): modified by 2′Fluoro
  misc_feature (13)..(13): modified by 2′OMe
  misc_feature (14)..(14): modified by 2′Fluoro
  misc_feature (15)..(15): modified by 2′OMe
  misc_feature (16)..(16): modified by 2′Fluoro
  misc_feature (17)..(17): modified by 2′OMe
  misc_feature (18)..(18): modified by 2′Fluoro
  misc_feature (19)..(19): modified by 2′OMe
  misc_feature (20)..(20): modified by 2′Fluoro
  misc_feature (21)..(21): modified by 2′OMe
  misc_feature (21)..(23): phosphorothioate linkage
  misc_feature (22)..(22): modified by 2′Fluoro
  misc_feature (23)..(23): modified by 2′OMe
aagguagauaugguuauuacaaa

SEQ ID NO: 262 - protein, 8 aa, Artificial Sequence
  Synthetic
IDPETGDT

SEQ ID NO: 263 - protein, 17 aa, Artificial Sequence
  Synthetic
WIDPETGDTEYASKFQD

SEQ ID NO: 264 - skipped

SEQ ID NO: 265 - protein, 17 aa, Artificial Sequence
  Synthetic
WIDPESGDTEYASKFQD

SEQ ID NO: 266 - protein, 8 aa, Artificial Sequence
  Synthetic
GYSFTDYY

SEQ ID NO: 267 - protein, 5 aa, Artificial Sequence
  Synthetic
DYYIN

SEQ ID NO: 268 - protein, 8 aa, Artificial Sequence
  Synthetic
GYSFTDYD

SEQ ID NO: 269 - protein, 5 aa, Artificial Sequence
  Synthetic
DYDIN

SEQ ID NO: 270 - protein, 3 aa, Artificial Sequence
  Synthetic
ETG

SEQ ID NO: 271 - protein, 3 aa, Artificial Sequence
  Synthetic
ESG

SEQ ID NO: 272 - protein, 25 aa, Artificial Sequence [sequence omitted]

SEQ ID NO: 273 - protein, 13 aa, Artificial Sequence
  Synthetic
WVRQAPGKGLEWV

SEQ ID NO: 274 - protein, 30 aa, Artificial Sequence
  Synthetic
RFTISRDNSKNTLYLQMNSLRAEDTAVYYC

SEQ ID NO: 275 - protein, 11 aa, Artificial Sequence
  Synthetic
WGQGTLVTVSS

SEQ ID NO: 276 - protein, 25 aa, Artificial Sequence [sequence omitted]

SEQ ID NO: 277 - protein, 13 aa, Artificial Sequence
  Synthetic
WVRQAPGQGLEWM

SEQ ID NO: 278 - protein, 30 aa, Artificial Sequence
  Synthetic
RVTITADTSTSTAYMELSSLRSEDTAVYYC

SEQ ID NO: 279 - protein, 23 aa, Artificial Sequence
  Synthetic
DIVMTQSPDSLAVSLGERATINC

SEQ ID NO: 280 - protein, 25 aa, Artificial Sequence
  Synthetic
QVQLQESGPGLVKPSQTLSLTCTVS

SEQ ID NO: 281 - protein, 13 aa, Artificial Sequence
  Synthetic
WIRQPPGKGLEWI

SEQ ID NO: 282 - protein, 30 aa, Artificial Sequence
  Synthetic
RVTISVDTSKNQFSLKLSSVTAADTAVYYC

SEQ ID NO: 283 - protein, 15 aa, Artificial Sequence
  Synthetic
WYQQKPGQPPKLLIY

SEQ ID NO: 284 - protein, 23 aa, Artificial Sequence
  Synthetic
DIQMTQSPSSLSASVGDRVTITC

SEQ ID NO: 285 - protein, 15 aa, Artificial Sequence
  Synthetic
WYQQKPGKAPKLLIY

SEQ ID NO: 286 - protein, 32 aa, Artificial Sequence
  Synthetic
GVPSRFSGSGSGTDFTLTISSLQPEDFATYYC

SEQ ID NO: 287 - protein, 10 aa, Artificial Sequence
  Synthetic
FGQGTKVEIK

SEQ ID NO: 288 - protein, 23 aa, Artificial Sequence
  Synthetic
DIVMTQSPLSLPVTPGEPASISC

SEQ ID NO: 289 - protein, 15 aa, Artificial Sequence
  Synthetic
WYLQKPGQSPQLLIY

SEQ ID NO: 290 - protein, 32 aa, Artificial Sequence
  Synthetic
GVPDRFSGSGSGTDFTLKISRVEAEDVGVYYC

SEQ ID NO: 291 - protein, 32 aa, Artificial Sequence
  Synthetic
GVPDRFSGSGSGTDFTLTISSLQAEDFAVYYC

SEQ ID NO: 292-299 - skipped (8 entries)

SEQ ID NO: 300 - protein, 447 aa, Artificial Sequence
  Synthetic
EVQLQQSGAELVRPGASVKLSCTASGFNIKDDYMYWVKQRPEQGLEWIGWIDPETGDTEYASKFQDKATVTADTSSNTAYLQLSSLTSEDTAVYYCTLWLRRGLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 301 - protein, 447 aa, Artificial Sequence
  Synthetic
EVQLQQSGAELVRPGASVKLSCTASGFNIKDDYMYWVKQRPEQGLEWIGWIDPESGDTEYASKFQDKATVTADTSSNTAYLQLSSLTSEDTAVYYCTLWLRRGLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 302 - protein, 450 aa, Artificial Sequence
  Synthetic
QIQLQQSGPELVRPGASVKISCKASGYSFTDYYINWVNQRPGQGLEWIGWIYPGSGNTRYSERFKGKATLTVDTSSNTAYMQLSSLTSEDSAVYFCAREDYYPYHGMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 303 - protein, 450 aa, Artificial Sequence
  Synthetic
QIQLQQSGPELVRPGASVKISCKASGYSFTDYDINWVNQRPGQGLEWIGWIYPGSGNTRYSERFKGKATLTVDTSSNTAYMQLSSLTSEDSAVYFCAREDYYPYHGMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 304 - protein, 225 aa, Artificial Sequence
  Synthetic
EVQLQQSGAELVRPGASVKLSCTASGFNIKDDYMYWVKQRPEQGLEWIGWIDPETGDTEYASKFQDKATVTADTSSNTAYLQLSSLTSEDTAVYYCTLWLRRGLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 305 - protein, 225 aa, Artificial Sequence
  Synthetic
EVQLQQSGAELVRPGASVKLSCTASGFNIKDDYMYWVKQRPEQGLEWIGWIDPESGDTEYASKFQDKATVTADTSSNTAYLQLSSLTSEDTAVYYCTLWLRRGLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 306 - protein, 228 aa, Artificial Sequence
  Synthetic
QIQLQQSGPELVRPGASVKISCKASGYSFTDYYINWVNQRPGQGLEWIGWIYPGSGNTRYSERFKGKATLTVDTSSNTAYMQLSSLTSEDSAVYFCAREDYYPYHGMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 307 - protein, 228 aa, Artificial Sequence
  Synthetic
QIQLQQSGPELVRPGASVKISCKASGYSFTDYDINWVNQRPGQGLEWIGWIYPGSGNTRYSERFKGKATLTVDTSSNTAYMQLSSLTSEDSAVYFCAREDYYPYHGMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 308 - protein, 230 aa, Artificial Sequence
  Synthetic
QVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARFPYDSSGYYSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 309 - protein, 10 aa, Artificial Sequence
  Synthetic
GYTFTNYWMH

SEQ ID NO: 310 - protein, 17 aa, Artificial Sequence
  Synthetic
EINPINGRSNYGERFKT

SEQ ID NO: 311 - protein, 7 aa, Artificial Sequence
  Synthetic
GTRAMHY

SEQ ID NO: 312 - protein, 11 aa, Artificial Sequence
  Synthetic
RTSENIYNNLA

SEQ ID NO: 313 - protein, 7 aa, Artificial Sequence
  Synthetic
AATNLAD

SEQ ID NO: 314 - protein, 10 aa, Artificial Sequence
  Synthetic
QHFWGTPLTF

SEQ ID NO: 315 - protein, 116 aa, Artificial Sequence
  Synthetic
QVQLQQPGAELVKPGASVKLSCKASGYTFTNYWMHWVKQRPGQGLEWIGEINPINGRSNYGERFKTKATLTVDKSSSTAYMQLSSLTSEDSAVYYCARGTRAMHYWGQGTSVTVSS

SEQ ID NO: 316 - protein, 107 aa, Artificial Sequence
  Synthetic
DIQMTQSPASLSVSVGETVTITCRTSENIYNNLAWYQQKQGKSPQLLVYAATNLADGVPSRFSGSGSGTQYSLKINSLQSEDFGNYYCQHFWGTPLTFGAGTKLELK

SEQ ID NO: 317 - protein, 9 aa, Artificial Sequence
  Synthetic
YTFTNYWMH

SEQ ID NO: 318 - protein, 17 aa, Artificial Sequence
  Synthetic
EINPINGRSNYAQKFQG

SEQ ID NO: 319 - protein, 17 aa, Artificial Sequence
  Synthetic
EINPINGRSNYAEKFQG

SEQ ID NO: 320 - protein, 17 aa, Artificial Sequence
  Synthetic
EINPIQGRSNYAEKFQG

SEQ ID NO: 321 - protein, 9 aa, Artificial Sequence
  Synthetic
QHFWGTPLT

SEQ ID NO: 322 - protein, 7 aa, Artificial Sequence
  Synthetic
AATNLAE

SEQ ID NO: 323 - protein, 11 aa, Artificial Sequence
  Synthetic
RTSENIYSNLA

SEQ ID NO: 324 - protein, 7 aa, Artificial Sequence
  Synthetic
AGTNLAD

SEQ ID NO: 325 - protein, 116 aa, Artificial Sequence
  Synthetic
QVQLVQSGAEVKKPGASVKVSCKASGYTFTNYWMHWVRQAPGQGLEWMGEINPINGRSNYAQKFQGRVTLTVDTSISTAYMELSRLRSDDTAVYYCARGTRAMHYWGQGTLVTVSS

SEQ ID NO: 326 - protein, 116 aa, Artificial Sequence
  Synthetic
QVQLVQSGAEVKKPGASVKVSCKASGYTFTNYWMHWVRQAPGQGLEWIGEINPINGRSNYAEKFQGRVTLTVDTSSSTAYMELSRLRSDDTAVYYCARGTRAMHYWGQGTLVTVSS

SEQ ID NO: 327 - protein, 116 aa, Artificial Sequence
  Synthetic
QVQLVQSGAEVKKPGASVKVSCKASGYTFTNYWMHWVRQAPGQGLEWMGEINPIQGRSNYAEKFQGRVTLTVDTSSSTAYMELSSLRSEDTATYYCARGTRAMHYWGQGTLVTVSS

SEQ ID NO: 328 - protein, 116 aa, Artificial Sequence
  Synthetic
QVQLVQSGAEVKKPGASVKVSCKASGYTFTNYWMHWVRQAPGQGLEWMGEINPINGRSNYAEKFQGRVTLTVDTSSSTAYMELSSLRSEDTATYYCARGTRAMHYWGQGTLVTVSS

SEQ ID NO: 329 - protein, 107 aa, Artificial Sequence
  Synthetic
DIQMTQSPSSLSASVGDRVTITCRTSENIYNNLAWYQQKPGKSPKLLIYAATNLADGVPSRFSGSGSGTDYTLTISSLQPEDFATYYCQHFWGTPLTFGGGTKVEIK

SEQ ID NO: 330 - protein, 107 aa, Artificial Sequence
  Synthetic
DIQMTQSPSSLSASVGDRVTITCRTSENIYNNLAWYQQKPGKAPKLLIYAATNLADGVPSRFSGSGSGTDYTLTISSLQPEDFATYYCQHFWGTPLTFGGGTKVEIK

SEQ ID NO: 331 - protein, 107 aa, Artificial Sequence
  Synthetic
DIQMTQSPSSLSASVGDRVTITCRTSENIYNNLAWYQQKPGKAPKLLIYAATNLAEGVPSRFSGSGSGTDYTLTISSLQPEDFATYYCQHFWGTPLTFGGGTKVEIK

SEQ ID NO: 332 - protein, 107 aa, Artificial Sequence
  Synthetic
DIQMTQSPSSLSASVGDRVTITCRTSENIYSNLAWYQQKPGKAPKLLIYAGTNLADGVPSRFSGSGSGTDYTLTISSLQPEDFANYYCQHFWGTPLTFGGGTKVEIK

(19) United States
(12) Patent Application Publication
(10) Pub. No.: US 2023/0117883 A1
(43) Pub. Date: Apr. 20, 2023

(54) MUSCLE-TARGETING COMPLEXES AND USES THEREOF IN TREATING MUSCLE ATROPHY

(71) Applicant: Dyne Therapeutics, Inc., Waltham, MA (US)

(72) Inventors: Romesh R. Subramanian, Framingham, MA (US); Mohammed T. Qatanani, Waltham, MA (US); Timothy Weeden, Waltham, MA (US); Cody A. Desjardins, Waltham, MA (US); Brendan Quinn, Boston, MA (US)

(73) Assignee: Dyne Therapeutics, Inc., Waltham, MA (US)

(21) Appl. No.: 17/794,768

(22) PCT Filed: Jan. 8, 2021
(86) PCT No.: PCT/US2021/012662

§ 371 Date: Jul. 22, 2022

Related U.S. Application Data
(60) Provisional application No. 62/965,762, filed on Jan. 24, 2020.
(60) Provisional application No. 62/968,398, filed on Jan. 31, 2020.
(60) Provisional application No. 63/055,529, filed on Jul. 23, 2020.

(51) Int. Cl. A61K 47/68; C07K 16/28; C12N 15/113

(52) U.S. Cl. CPC A61K 47/6807; C07K 16/2881; C12N 15/1137; C12N 15/1136; C07K 2317/92; C12N 2310/14; C12N 2310/315; C12N 2310/341; C12N 2310/3513; C12N 2320/32; C12N 2310/3233

(57) ABSTRACT

Aspects of the disclosure relate to complexes comprising a muscle-targeting agent covalently linked to a molecular payload. In some embodiments, the muscle-targeting agent specifically binds to an internalizing cell surface receptor on muscle cells. In some embodiments, the molecular payload inhibits activity of a pro-atrophy gene. In some embodiments, the molecular payload is an oligonucleotide, such as an antisense oligonucleotide or RNAi oligonucleotide.

### RELATED APPLICATIONS

This application claims the benefit under 35 U.S.0 § 119(e) of the filing date of U.S. Provisional Application No. 63/055,529, entitled “MUSCLE-TARGETING COMPLEXES AND USES THEREOF IN TREATING MUSCLE ATROPHY”, filed Jul. 23, 2020; U.S. Provisional Application No. 62/968,398, entitled “MUSCLE-TARGETING COMPLEXES AND USES THEREOF IN TREATING MUSCLE ATROPHY”, filed Jan. 31, 2020; and U.S. Provisional Application No. 62/965,762, entitled “MUSCLE-TARGETING COMPLEXES AND USES THEREOF IN TREATING MUSCLE ATROPHY” filed Jan. 24, 2020; the contents of each of which are incorporated herein by reference in their entirety.

### FIELD OF THE INVENTION

The present application relates to targeting complexes for delivering molecular payloads (e.g., oligonucleotides) to cells and uses thereof, particularly uses relating to treatment of disease.

### REFERENCE TO SEQUENCE LISTING SUBMITTED AS A TEXT FILE VIA EFS-WEB

The instant application contains a sequence listing which has been submitted in ASCII format via EFS-Web and is hereby incorporated by reference in its entirety. Said ASCII copy, created on Jan. 8, 2021, is named D082470034W000-SEQ-ZJG and is 231 kilobytes in size.

### BACKGROUND

Muscle atrophy can result from numerous conditions, including, for example, AIDS, cancer, sepsis, burn injury, anorexia, congestive heart failure, renal failure, chronic obstructive pulmonary disease, and muscle disuse. In some cases, muscle atrophy results in life-threatening complications. Although numerous factors have been implicated in regulating muscle atrophy, effective treatments are limited.

### SUMMARY

According to some aspects, the disclosure provides complexes that target muscle cells for purposes of delivering molecular payloads to those cells for treating muscle atrophy. In some embodiments, complexes provided herein are particularly useful for delivering molecular payloads that inhibit the expression or activity of a pro-atrophy gene (e.g., a gene listed in Table 1), e.g., in a subject having, suspected of having or at risk of muscle atrophy. In some embodiments, complexes provided herein comprise muscle-targeting agents (e.g., muscle targeting antibodies) that specifically bind to receptors on the surface of muscle cells for purposes of delivering molecular payloads to the muscle cells. In some embodiments, the complexes are taken up into the cells via a receptor (e.g., transferrin receptor) mediated internalization, following which the molecular payload may be released to perform a function inside the cells. For example, complexes engineered to deliver oligonucleotides may release the oligonucleotides such that the oligonucleotides can inhibit gene expression (e.g., of one or more pro-atrophy genes provided in Table 1) in the muscle cells. In some embodiments, the oligonucleotides are released by endosomal cleavage of covalent linkers connecting oligonucleotides and muscle-targeting agents of the complexes.

Some aspects of the present disclosure provide complexes comprising an anti-transferrin receptor antibody covalently linked to a molecular payload configured for to inhibit expression or activity of a pro-atrophy gene. In some embodiments, the anti-TfR antibody comprises a heavy chain complementarity determining region 1 (CDR-H1), a heavy chain complementarity determining region 2 (CDR-H2), a heavy chain complementarity determining region 3 (CDR-H3), a light chain complementarity determining region 1 (CDR-L1), a light chain complementarity determining region 2 (CDR-L2), a light chain complementarity determining region 3 (CDR-L3) of any of the anti-TfR antibodies listed in Table 2, 4, and 7.

In some embodiments, the antibody comprises a CDR-H1, a CDR-H2, and a CDR-H3 of a heavy chain variable region (VH) comprising the amino acid sequence of SEQ ID NO: 15, and a CDR-L1, a CDR-L2, and a CDR-L3 of a light chain variable region (VL) comprising the amino acid sequence of SEQ ID NO: 16. In some embodiments, the antibody comprises a CDR-H1, a CDR-H2, and a CDR-H3 of a VH comprising the amino acid sequence of SEQ ID NO: 204, and a CDR-L1, a CDR-L2, and a CDR-L3 of a VL comprising the amino acid sequence of SEQ ID NO: 205. In some embodiments, the antibody comprises CDR-H1, a CDR-H2, and a CDR-H3 of a VH comprising the amino acid sequence of SEQ ID NO: 7, and a CDR-L1, a CDR-L2, and a CDR-L3 of a VL comprising the amino acid sequence of SEQ ID NO: 8. In some embodiments, the antibody comprises CDR-H1, a CDR-H2, and a CDR-H3 of a VH comprising the amino acid sequence of SEQ ID NO: 23, and a CDR-L1, a CDR-L2, and a CDR-L3 of a VL comprising the amino acid sequence of SEQ ID NO: 24.

In some embodiments, the antibody comprises: a CDR-H1 of SEQ ID NO: 155, a CDR-H2 of SEQ ID NO: 156, a CDR-H3 of SEQ ID NO: 157, a CDR-L1 of SEQ ID NO: 158, a CDR-L2 of SEQ ID NO: 159, and a CDR-L3 of SEQ ID NO: 14. In some embodiments, the antibody comprises a CDR-H1 of SEQ ID NO: 194, a CDR-H2 of SEQ ID NO: 195, a CDR-H3 of SEQ ID NO: 196, a CDR-L1 of SEQ ID NO: 197, a CDR-L2 of SEQ ID NO: 198, and a CDR-L3 of SEQ ID NO: 193. In some embodiments, the antibody comprises a CDR-H1 of SEQ ID NO: 145, a CDR-H2 of SEQ ID NO: 146, SEQ ID NO: 263, or SEQ ID NO: 265, a CDR-H3 of SEQ ID NO: 147, a CDR-L1 of SEQ ID NO: 148, a CDR-L2 of SEQ ID NO: 149, and a CDR-L3 of SEQ ID NO: 6. In some embodiments, the antibody comprises a CDR-H1 of SEQ ID NO: 165, SEQ ID NO: 267, or SEQ ID NO: 269, a CDR-H2 of SEQ ID NO: 166, a CDR-H3 of SEQ ID NO: 167, a CDR-L1 of SEQ ID NO: 168, a CDR-L2 of SEQ ID NO: 169, and a CDR-L3 of SEQ ID NO: 22.

In some embodiments, the antibody comprises human or humanized framework regions with the CDR-H1, the CDR-H2, the CDR-H3 of a VH as set forth in SEQ ID NO: 15, and the CDR-L1, the CDR-L2, the CDR-L3 of a VL as set forth in SEQ ID NO: 16. In some embodiments, the antibody comprises human or humanized framework regions with the CDR-H1, the CDR-H2, the CDR-H3 of a VH as set forth in SEQ ID NO: 204, and the CDR-L1, the CDR-L2, the CDR-L3 of a VL as set forth in SEQ ID NO: 205. In some embodiments, the antibody comprises human or humanized framework regions with the CDR-H1, the CDR-H2, the CDR-H3 of a VH as set forth in SEQ ID NO: 7, and the CDR-L1, the CDR-L2, the CDR-L3 of a VL as set forth in SEQ ID NO: 8. In some embodiments, the antibody comprises human or humanized framework regions with the CDR-H1, the CDR-H2, the CDR-H3 of a VH as set forth in SEQ ID NO: 23, and the CDR-L1, the CDR-L2, the CDR-L3 of a VL as set forth in SEQ ID NO: 24.

In some embodiments, the antibody comprises a VH comprising an amino acid sequence at least 80% identical to SEQ ID NO: 15, and a VL comprising an amino acid sequence at least 80% identical to SEQ ID NO: 16. In some embodiments, the antibody comprises a VH comprising an amino acid sequence at least 80% identical to SEQ ID NO: 204, and a VL comprising an amino acid sequence at least 80% identical to SEQ ID NO: 205, optionally wherein the antibody comprises a VH comprising the amino acid sequence of SEQ ID NO: 204 and a VL comprising the amino acid sequence of SEQ ID NO: 205. In some embodiments, the antibody comprises a VH comprising an amino acid sequence at least 80% identical to SEQ ID NO: 7, and a VL comprising an amino acid sequence at least 85% identical to SEQ ID NO: 8. In some embodiments, the antibody comprises a VH comprising an amino acid sequence at least 80% identical to SEQ ID NO: 23, and a VL comprising an amino acid sequence at least 80% identical to SEQ ID NO: 24.

In some embodiments, the equilibrium dissociation constant (KD) of binding of the antibody to the transferrin receptor is in a range from 10⁻¹¹M to 10⁻⁶ M.

In some embodiments, the antibody is selected from the group consisting of a full-length IgG, a Fab fragment, a F(ab′) fragment, a F(ab′)2 fragment, a scFv, and a Fv, optionally wherein the antibody is a Fab′ fragment.

In some embodiments, the molecular payload is an oligonucleotide.

In some embodiments, the oligonucleotide comprises at least one modified internucleoside linkage.

In some embodiments, the at least one modified internucleoside linkage is a phosphorothioate linkage.

In some embodiments, the oligonucleotide comprises one or more modified nucleotides.

In some embodiments, the one or more modified nucleotides are 2′-modified nucleotides.

In some embodiments, the 2′ modified nucleotide is selected from the group consisting of: 2′-O-methyl (2′-O-Me), 2′-fluoro (2′-F), 2′-O-methoxyethyl (2′-MOE), and 2′,4′-bicyclic nucleosides, further optionally wherein the 2′,4′-bicyclic nucleoside is selected from: locked nucleic acid (LNA), ethylene-bridged nucleic acid (ENA), and (S)-constrained ethyl-bridged nucleic acid (cEt).

In some embodiments, the oligonucleotide is a gapmer oligonucleotide that directs RNAse H-mediated cleavage of an mRNA transcript encoded by the muscle disease gene in a cell.

In some embodiments, the oligonucleotide is a mixmer oligonucleotide.

In some embodiments, the oligonucleotide is an RNAi oligonucleotide that promotes RNAi-mediated cleavage of a mRNA transcript encoded by the muscle disease gene.

In some embodiments, the oligonucleotide is a phosphorodiamidate morpholino oligomer (PMO).

In some embodiments, the antibody is covalently linked to the molecular payload via a cleavable linker, optionally wherein the cleavable linker comprises a valine-citrulline dipeptide sequence.

Other aspects of the present disclosure provide methods of delivering a molecular payload to a cell expressing transferrin receptor. In some embodiments, the methods comprise contacting the cell with the complex described herein.

Other aspects of the present disclosure provide methods of inhibiting expression or activity of a pro-atrophy gene in a cell. In some embodiments, the methods comprise contacting the cell with the complex described herein in an amount effective for promoting internalization of the molecular payload to the cell.

Further provided herein are methods of treating a subject having muscle atrophy. In some embodiments, the method comprises administering to the subject an effective amount of the complex described herein, e.g., wherein the muscle disease is a disease listed in Table 1.

### BRIEF DESCRIPTION OF THE DRAWINGS

FIG. 1 depicts a non-limiting schematic showing the effect of transfecting cells with an siRNA.

FIG. 2 depicts a non-limiting schematic showing the activity of a muscle targeting complex comprising an siRNA.

FIGS. 3A-3B depict non-limiting schematics showing the activity of a muscle targeting complex comprising an siRNA in mouse muscle tissues (gastrocnemius and heart) in vivo, relative to control experiments. (N=4 C57BL/6 WT mice)

FIGS. 4A-4E depict non-limiting schematics showing the tissue selectivity of a muscle targeting complex comprising an siRNA.

FIGS. 5A-5L depict non-limiting schematics showing the ability of a muscle targeting complex (DTX-C-012) comprising an anti-transferrin receptor antibody (a 15G11 antibody) to reduce gene expression levels in cynomolgus monkey muscle tissues in vivo, relative to a vehicle experiment and compared to a naked ASO (control DMPK-ASO). (N=3 male cynomolgus monkeys)

FIGS. 6A-6B depict non-limiting schematics showing the ability of a muscle targeting complex (DTX-C-012) comprising an anti-transferrin receptor antibody (a 15G11 antibody) to reduce gene expression levels in cynomolgus monkey smooth muscle tissues in vivo, relative to a vehicle experiment and compared to a naked ASO (control DMPK-ASO). (N=3 male cynomolgus monkeys).

FIGS. 7A-7D depict non-limiting schematics showing the tissue selectivity of a muscle targeting complex (DTX-C-012) comprising an anti-transferrin receptor antibody (a 15G11 antibody). The muscle targeting complex does not reduce gene expression levels in cynomolgus monkey liver, kidney, brain, or spleen tissues in vivo, relative to a vehicle experiment. (N=3 male cynomolgus monkeys).

FIG. 8 shows normalized mRNA tissue expression levels across several tissue types in cynomolgus monkeys. (N=3 male cynomolgus monkeys)

FIG. 9 shows that a single dose of a muscle targeting complex (DTX-C-012) comprising an anti-transferrin receptor antibody (a 15G11 antibody) is safe and tolerated in cynomolgus monkeys. (N=3 male cynomolgus monkeys).

FIG. 10 is a graph showing DMPK knock down efficiency in non-human primate (NHP) cells or cells from human DM1 patients (DM1) of conjugates containing selected anti-TfR1 antibodies covalently conjugated to an antisense oligonucleotide targeting DMPK.

FIGS. 11A-11B show binding of the different anti-TfR1 antibody formats to human (FIG. 11A) or cyno (FIG. 11B) transferrin receptor 1.

FIG. 12 shows binding of the different anti-TfR1 antibody formats to human transferrin receptor 2. An anti-TfR2 monoclonal antibody (OTI1B1) was used as control. None of the tested antibodies binds to TfR2.

FIG. 13 is a graph showing DMPK knock down efficiency in non-human primate (NHP) cells or cells from human DM1 patients (DM1) of conjugates containing an anti-TfR1 antibody described herein covalently conjugated to an antisense oligonucleotide targeting DMPK.

FIGS. 14A-14B show binding of oligonucleotide-conjugated or unconjugated anti-TfR to human TfR1 (hTfR1) and cynomolgus monkey TfR1 (cTfR1), as measured by ELISA. The anti-TfR is the one shown in Table 7. FIG. 14A shows the binding of the anti-TfR alone (EC50 26.6 nM) or in conjugates with a DMPK targeting oligo (EC50 8.2 nM) to hTfR1. FIG. 14B shows the binding of the anti-TfR alone (EC50 33.6 nM) or in conjugates with a DMPK targeting oligo (EC50 5.3 nM) to cTfR1.

FIG. 15 shows the quantified cellular uptake of anti-TfR Fab conjugates into rhabdomyosarcoma (RD) cells. The molecular payload in the tested conjugates are DMPK-targeting oligonucleotides and the uptake of the conjugates were facilitated by indicated anti-TfR Fabs. Conjugates having a negative control Fab (anti-mouse TfR) or a positive control Fab (anti-human TfR1) are also included this assay. Cells were incubated with indicated conjugate at a concentration of 100 nM for 4 hours. Cellular uptake was measured by mean Cypher5e fluorescence.

FIG. 16. shows DMPK expression in RD cells treated with various concentrations of conjugates containing an anti-TfR antibody (the anti-TfR in Table 7) conjugated to a DMPK-targeting oligonucleotide (control DMPK-ASO). The duration of treatment was 3 days. Control DMPK-ASO delivered using transfection agents were used as control.

FIG. 17 shows the serum stability of the linker used for linking an anti-TfR antibody and a molecular payload (e.g., an oligonucleotide) in various species over time after intravenous administration.

### DETAILED DESCRIPTION

Aspects of the disclosure relate to a recognition that while certain molecular payloads (e.g., oligonucleotides, peptides, small molecules) can have beneficial effects in muscle cells, it has proven challenging to effectively target such cells. As described herein, the present disclosure provides complexes comprising muscle-targeting agents covalently linked to molecular payloads in order to overcome such challenges. In some embodiments, the complexes are particularly useful for delivering molecular payloads that inhibit the expression or activity of target genes in muscle cells, e.g., in a subject having or suspected of having muscle atrophy. For example, in some embodiments, complexes are provided for inhibiting expression or activity of a pro-atrophy gene provided in Table 1. For example, in some embodiments, a complex comprises an oligonucleotide configured to inhibit expression of a FBXO32. In some embodiments, a complex comprises an oligonucleotide configured to inhibit expression of a MSTN. In some embodiments, a complex comprises an oligonucleotide configured to inhibit expression of a TRIM63. In some embodiments, a complex comprises an oligonucleotide configured to inhibit expression of an INHBA.

Further aspects of the disclosure, including a description of defined terms, are provided below.

### I. Definitions

Administering: As used herein, the terms “administering” or “administration” means to provide a complex to a subject in a manner that is physiologically and/or (e.g., and) pharmacologically useful (e.g., to treat a condition in the subject).

Approximately: As used herein, the term “approximately” or “about,” as applied to one or more values of interest, refers to a value that is similar to a stated reference value. In certain embodiments, the term “approximately” or “about” refers to a range of values that fall within 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Antibody: As used herein, the term “antibody” refers to a polypeptide that includes at least one immunoglobulin variable domain or at least one antigenic determinant, e.g., paratope that specifically binds to an antigen. In some embodiments, an antibody is a full-length antibody. In some embodiments, an antibody is a chimeric antibody. In some embodiments, an antibody is a humanized antibody. However, in some embodiments, an antibody is a Fab fragment, a F(ab′) fragment, a F(ab′)2 fragment, a Fv fragment or a scFv fragment. In some embodiments, an antibody is a nanobody derived from a camelid antibody or a nanobody derived from shark antibody. In some embodiments, an antibody is a diabody. In some embodiments, an antibody comprises a framework having a human germline sequence. In another embodiment, an antibody comprises a heavy chain constant domain selected from the group consisting of IgG, IgG1, IgG2, IgG2A, IgG2B, IgG2C, IgG3, IgG4, IgAl, IgA2, IgD, IgM, and IgE constant domains. In some embodiments, an antibody comprises a heavy (H) chain variable region (abbreviated herein as VH), and/or (e.g., and) a light (L) chain variable region (abbreviated herein as VL). In some embodiments, an antibody comprises a constant domain, e.g., an Fc region. An immunoglobulin constant domain refers to a heavy or light chain constant domain. Human IgG heavy chain and light chain constant domain amino acid sequences and their functional variations are known. With respect to the heavy chain, in some embodiments, the heavy chain of an antibody described herein can be an alpha (α), delta (Δ), epsilon (ε), gamma (γ) or mu (μ) heavy chain. In some embodiments, the heavy chain of an antibody described herein can comprise a human alpha (α), delta (Δ), epsilon (ε), gamma (γ) or mu (μ) heavy chain. In a particular embodiment, an antibody described herein comprises a human gamma 1 CH1, CH2, and/or (e.g., and) CH3 domain. In some embodiments, the amino acid sequence of the VH domain comprises the amino acid sequence of a human gamma (γ) heavy chain constant region, such as any known in the art. Non-limiting examples of human constant region sequences have been described in the art, e.g., see U.S. Pat. No. 5,693,780 and Kabat E A et al., (1991) supra. In some embodiments, the VH domain comprises an amino acid sequence that is at least 70%, 75%, 80%, 85%, 90%, 95%, 98%, or at least 99% identical to any of the variable chain constant regions provided herein. In some embodiments, an antibody is modified, e.g., modified via glycosylation, phosphorylation, sumoylation, and/or (e.g., and) methylation. In some embodiments, an antibody is a glycosylated antibody, which is conjugated to one or more sugar or carbohydrate molecules. In some embodiments, the one or more sugar or carbohydrate molecule are conjugated to the antibody via N-glycosylation, O-glycosylation, C-glycosylation, glypiation (GPI anchor attachment), and/or (e.g., and) phosphoglycosylation. In some embodiments, the one or more sugar or carbohydrate molecule are monosaccharides, disaccharides, oligosaccharides, or glycans. In some embodiments, the one or more sugar or carbohydrate molecule is a branched oligosaccharide or a branched glycan. In some embodiments, the one or more sugar or carbohydrate molecule includes a mannose unit, a glucose unit, an N-acetylglucosamine unit, an N-acetylgalactosamine unit, a galactose unit, a fucose unit, or a phospholipid unit. In some embodiments, an antibody is a construct that comprises a polypeptide comprising one or more antigen binding fragments of the disclosure linked to a linker polypeptide or an immunoglobulin constant domain. Linker polypeptides comprise two or more amino acid residues joined by peptide bonds and are used to link one or more antigen binding portions. Examples of linker polypeptides have been reported (see e.g., Holliger, P., et al. (1993) Proc. Natl. Acad. Sci. USA 90:6444-6448; Poljak, R. J., et al. (1994) Structure 2:1121-1123). Still further, an antibody may be part of a larger immunoadhesion molecule, formed by covalent or noncovalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058).

CDR: As used herein, the term “CDR” refers to the complementarity determining region within antibody variable sequences. A typical antibody molecule comprises a heavy chain variable region (VH) and a light chain variable region (VL), which are usually involved in antigen binding. The VH and VL regions can be further subdivided into regions of hypervariability, also known as “complementarity determining regions” (“CDR”), interspersed with regions that are more conserved, which are known as “framework regions” (“FR”). Each VH and VL is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the IMGT definition, the Chothia definition, the AbM definition, and/or (e.g., and) the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242; IMGT®, the international ImMunoGeneTics information system® http://www.imgt.org, Lefranc, M.-P. et al., Nucleic Acids Res., 27:209-212 (1999); Ruiz, M. et al., Nucleic Acids Res., 28:219-221 (2000); Lefranc, M.-P., Nucleic Acids Res., 29:207-209 (2001); Lefranc, M.-P., Nucleic Acids Res., 31:307-310 (2003); Lefranc, M.-P. et al., In Silico Biol., 5, 0006 (2004) [Epub], 5:45-60 (2005); Lefranc, M.-P. et al., Nucleic Acids Res., 33:D593-597 (2005); Lefranc, M.-P. et al., Nucleic Acids Res., 37:D1006-1012 (2009); Lefranc, M.-P. et al., Nucleic Acids Res., 43:D413-422 (2015); Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). ee also hgmp.mrc.ac.uk and bioinf.org.uk/abs. As used herein, a CDR may refer to the CDR defined by any method known in the art. Two antibodies having the same CDR means that the two antibodies have the same amino acid sequence of that CDR as determined by the same method, for example, the IMGT definition.

There are three CDRs in each of the variable regions of the heavy chain and the light chain, which are designated CDR1, CDR2 and CDR3, for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Sub-portions of CDRs may be designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262(5):732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although preferred embodiments use Kabat or Chothia defined CDRs.

CDR-grafted antibody: The term “CDR-grafted antibody” refers to antibodies which comprise heavy and light chain variable region sequences from one species but in which the sequences of one or more of the CDR regions of VH and/or (e.g., and) VL are replaced with CDR sequences of another species, such as antibodies having murine heavy and light chain variable regions in which one or more of the murine CDRs (e.g., CDR3) has been replaced with human CDR sequences.

Chimeric antibody: The term “chimeric antibody” refers to antibodies which comprise heavy and light chain variable region sequences from one species and constant region sequences from another species, such as antibodies having murine heavy and light chain variable regions linked to human constant regions.

Complementary: As used herein, the term “complementary” refers to the capacity for precise pairing between two nucleotides or two sets of nucleotides. In particular, complementary is a term that characterizes an extent of hydrogen bond pairing that brings about binding between two nucleotides or two sets of nucleotides. For example, if a base at one position of an oligonucleotide is capable of hydrogen bonding with a base at the corresponding position of a target nucleic acid (e.g., an mRNA), then the bases are considered to be complementary to each other at that position. Base pairings may include both canonical Watson-Crick base pairing and non-Watson-Crick base pairing (e.g., Wobble base pairing and Hoogsteen base pairing). For example, in some embodiments, for complementary base pairings, adenosine-type bases (A) are complementary to thymidine-type bases (T) or uracil-type bases (U), that cytosine-type bases (C) are complementary to guanosine-type bases (G), and that universal bases such as 3-nitropyrrole or 5-nitroindole can hybridize to and are considered complementary to any A, C, U, or T. Inosine (I) has also been considered in the art to be a universal base and is considered complementary to any A, C, U or T.

Conservative amino acid substitution: As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g. Molecular Cloning: A Laboratory Manual, J. Sambrook, et al., eds., Fourth Edition, Cold Spring Harbor Laboratory Press, Cold Spring Harbor, New York, 2012, or Current Protocols in Molecular Biology, F.M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

Covalently linked: As used herein, the term “covalently linked” refers to a characteristic of two or more molecules being linked together via at least one covalent bond. In some embodiments, two molecules can be covalently linked together by a single bond, e.g., a disulfide bond or disulfide bridge, that serves as a linker between the molecules. However, in some embodiments, two or more molecules can be covalently linked together via a molecule that serves as a linker that joins the two or more molecules together through multiple covalent bonds. In some embodiments, a linker may be a cleavable linker. However, in some embodiments, a linker may be a non-cleavable linker.

Cross-reactive: As used herein and in the context of a targeting agent (e.g., antibody), the term “cross-reactive,” refers to a property of the agent being capable of specifically binding to more than one antigen of a similar type or class (e.g., antigens of multiple homologs, paralogs, or orthologs) with similar affinity or avidity. For example, in some embodiments, an antibody that is cross-reactive against human and non-human primate antigens of a similar type or class (e.g., a human transferrin receptor and non-human primate transferrin receptor) is capable of binding to the human antigen and non-human primate antigens with a similar affinity or avidity. In some embodiments, an antibody is cross-reactive against a human antigen and a rodent antigen of a similar type or class. In some embodiments, an antibody is cross-reactive against a rodent antigen and a non-human primate antigen of a similar type or class. In some embodiments, an antibody is cross-reactive against a human antigen, a non-human primate antigen, and a rodent antigen of a similar type or class.

Framework: As used herein, the term “framework” or “framework sequence” refers to the remaining sequences of a variable region minus the CDRs. Because the exact definition of a CDR sequence can be determined by different systems, the meaning of a framework sequence is subject to correspondingly different interpretations. The six CDRs (CDR-L1, CDR-L2, and CDR-L3 of light chain and CDR-H1, CDR-H2, and CDR-H3 of heavy chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FRs within the variable region of a single, naturally occurring immunoglobulin chain. As used herein, a FR represents one of the four sub-regions, and FRs represents two or more of the four sub-regions constituting a framework region. Human heavy chain and light chain acceptor sequences are known in the art. In one embodiment, the acceptor sequences known in the art may be used in the antibodies disclosed herein.

Human antibody: The term “human antibody”, as used herein, is intended to include antibodies having variable and constant regions derived from human germline immunoglobulin sequences. The human antibodies of the disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

Humanized antibody: The term “humanized antibody” refers to antibodies which comprise heavy and light chain variable region sequences from a non-human species (e.g., a mouse) but in which at least a portion of the VH and/or (e.g., and) VL sequence has been altered to be more “human-like”, i.e., more similar to human germline variable sequences. One type of humanized antibody is a CDR-grafted antibody, in which human CDR sequences are introduced into non-human VH and VL sequences to replace the corresponding nonhuman CDR sequences. In one embodiment, humanized anti-transferrin receptor antibodies and antigen binding portions are provided. Such antibodies may be generated by obtaining murine anti-transferrin receptor monoclonal antibodies using traditional hybridoma technology followed by humanization using in vitro genetic engineering, such as those disclosed in Kasaian et al PCT publication No. WO 2005/123126 A2.

Internalizing cell surface receptor: As used herein, the term, “internalizing cell surface receptor” refers to a cell surface receptor that is internalized by cells, e.g., upon external stimulation, e.g., ligand binding to the receptor. In some embodiments, an internalizing cell surface receptor is internalized by endocytosis. In some embodiments, an internalizing cell surface receptor is internalized by clathrin-mediated endocytosis. However, in some embodiments, an internalizing cell surface receptor is internalized by a clathrin-independent pathway, such as, for example, phagocytosis, macropinocytosis, caveolae- and raft-mediated uptake or constitutive clathrin-independent endocytosis. In some embodiments, the internalizing cell surface receptor comprises an intracellular domain, a transmembrane domain, and/or (e.g., and) an extracellular domain, which may optionally further comprise a ligand-binding domain. In some embodiments, a cell surface receptor becomes internalized by a cell after ligand binding. In some embodiments, a ligand may be a muscle-targeting agent or a muscle-targeting antibody. In some embodiments, an internalizing cell surface receptor is a transferrin receptor.

Isolated antibody: An “isolated antibody”, as used herein, is intended to refer to an antibody that is substantially free of other antibodies having different antigenic specificities (e.g., an isolated antibody that specifically binds transferrin receptor is substantially free of antibodies that specifically bind antigens other than transferrin receptor). An isolated antibody that specifically binds transferrin receptor complex may, however, have cross-reactivity to other antigens, such as transferrin receptor molecules from other species. Moreover, an isolated antibody may be substantially free of other cellular material and/or (e.g., and) chemicals.

Kabat numbering: The terms “Kabat numbering”, “Kabat definitions and “Kabat labeling” are used interchangeably herein. These terms, which are recognized in the art, refer to a system of numbering amino acid residues which are more variable (i.e. hypervariable) than other amino acid residues in the heavy and light chain variable regions of an antibody, or an antigen binding portion thereof (Kabat et al. (1971) Ann. NY Acad, Sci. 190:382-391 and, Kabat, E. A., et al. (1991) Sequences of Proteins of Immunological Interest, Fifth Edition, U.S. Department of Health and Human Services, NIH Publication No. 91-3242). For the heavy chain variable region, the hypervariable region ranges from amino acid positions 31 to 35 for CDR1, amino acid positions 50 to 65 for CDR2, and amino acid positions 95 to 102 for CDR3. For the light chain variable region, the hypervariable region ranges from amino acid positions 24 to 34 for CDR1, amino acid positions 50 to 56 for CDR2, and amino acid positions 89 to 97 for CDR3.

Molecular payload: As used herein, the term “molecular payload” refers to a molecule or species that functions to modulate a biological outcome. In some embodiments, a molecular payload is linked to, or otherwise associated with a muscle-targeting agent. In some embodiments, the molecular payload is a small molecule, a protein, a peptide, a nucleic acid, or an oligonucleotide. In some embodiments, the molecular payload functions to modulate the transcription of a DNA sequence, to modulate the expression of a protein, or to modulate the activity of a protein. In some embodiments, the molecular payload is an oligonucleotide that comprises a strand having a region of complementarity to a target gene.

Muscle atrophy: As used herein, the term, “muscle atrophy,” refers to a condition characterized by muscle wasting. In some embodiments, muscle atrophy is a highly regulated catabolic process which occurs during periods of disuse and/or (e.g., and) in response to systemic inflammation (e.g., cachexia). In some embodiments, muscle atrophy is associated with diminishing muscle mass, reduction in muscle size, and/or (e.g., and) reduction in the number of muscle cells in a subject. Conditions, including chronic illnesses (e.g., congestive heart failure, cancer, AIDS, and renal disease), severe burns, critical care myopathy, limb denervation, stroke, limb fracture, anorexia, spinal cord injury or other conditions leading to muscle disuse may result in muscle atrophy. Muscle atrophy may also result as a natural process of aging, which may be categorized as sarcopenia.

Muscle-targeting agent: As used herein, the term, “muscle-targeting agent,” refers to a molecule that specifically binds to an antigen expressed on muscle cells. The antigen on muscle cells may be a membrane protein, for example an integral membrane protein or a peripheral membrane protein. Typically, a muscle-targeting agent specifically binds to an antigen on muscle cells that facilitates internalization of the muscle-targeting agent (and any associated molecular payload) into the muscle cells. In some embodiments, a muscle-targeting agent specifically binds to an internalizing, cell surface receptor on muscles and is capable of being internalized into muscle cells through receptor mediated internalization. In some embodiments, the muscle-targeting agent is a small molecule, a protein, a peptide, a nucleic acid (e.g., an aptamer), or an antibody. In some embodiments, the muscle-targeting agent is linked to a molecular payload.

Muscle-targeting antibody: As used herein, the term, “muscle-targeting antibody,” refers to a muscle-targeting agent that is an antibody that specifically binds to an antigen expressed on muscle cells. In some embodiments, a muscle-targeting antibody specifically binds to an antigen on muscle cells that facilitates internalization of the muscle-targeting antibody (and any associated molecular payment) into the muscle cells. In some embodiments, the muscle-targeting antibody specifically binds to an internalizing, cell surface receptor present on muscle cells. In some embodiments, the muscle-targeting antibody is an antibody that specifically binds to a transferrin receptor.

Oligonucleotide: As used herein, the term “oligonucleotide” refers to an oligomeric nucleic acid compound of up to 200 nucleotides in length. Examples of oligonucleotides include, but are not limited to, RNAi oligonucleotides (e.g., siRNAs, shRNAs), microRNAs, gapmers, mixmers, phosphorodiamidite morpholinos, peptide nucleic acids, aptamers, guide nucleic acids (e.g., Cas9 guide RNAs), etc. Oligonucleotides may be single-stranded or double-stranded. In some embodiments, an oligonucleotide may comprise one or more modified nucleotides (e.g. 2′-O-methyl sugar modifications, purine or pyrimidine modifications). In some embodiments, an oligonucleotide may comprise one or more modified internucleotide linkage. In some embodiments, an oligonucleotide may comprise one or more phosphorothioate linkages, which may be in the Rp or Sp stereochemical conformation.

Pro-atrophy gene: As used herein, the term “pro-atrophy gene” refers to a gene that encodes a product (protein or function RNA) that promotes muscle atrophy in a subject. Numerous pro-atrophy genes have been identified, including, for example, the genes identified in Table 1. Other pro-atrophy genes are disclosed, for example, in Li J. et al., miR-29b contributes to multiple types of muscle atrophy. Nature Communications volume 8, Article number: 15201 (2017); Schiaffino S. et al., Mechanisms regulating skeletal muscle growth and atrophy. FEBS Journal 280 (2013) 4294-4314; Bonaldo P, et al., Cellular and molecular mechanisms of muscle atrophy. Dis Model Mech. 2013 January; 6(1): 25-39; Otis J S, Oxidant-induced atrogin-1 and transforming growth factor-betal precede alcohol-related myopathy in rats. Muscle Nerve. 2007 December; 36(6):842-8; and Ebert S M., Stress-induced Skeletal Muscle Gadd45a Expression Reprograms Myonuclei and Causes Muscle Atrophy, Journal of Biological Chemistry Vol. 287, No. 33, pp. 27290-27301, Aug. 10, 2012, the contents of each of which are incorporated herein by reference in their entireties.

Recombinant antibody: The term “recombinant human antibody”, as used herein, is intended to include all human antibodies that are prepared, expressed, created or isolated by recombinant means, such as antibodies expressed using a recombinant expression vector transfected into a host cell (described in more details in this disclosure), antibodies isolated from a recombinant, combinatorial human antibody library (Hoogenboom H. R., (1997) TIB Tech. 15:62-70; Azzazy H., and Highsmith W. E., (2002) Clin. Biochem. 35:425-445; Gavilondo J. V., and Larrick J. W. (2002) BioTechniques 29:128-145; Hoogenboom H., and Chames P. (2000) Immunology Today 21:371-378), antibodies isolated from an animal (e.g., a mouse) that is transgenic for human immunoglobulin genes (see e.g., Taylor, L. D., et al. (1992) Nucl. Acids Res. 20:6287-6295; Kellermann S-A., and Green L. L. (2002) Current Opinion in Biotechnology 13:593-597; Little M. et al (2000) Immunology Today 21:364-370) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo. One embodiment of the disclosure provides fully human antibodies capable of binding human transferrin receptor which can be generated using techniques well known in the art, such as, but not limited to, using human Ig phage libraries such as those disclosed in Jermutus et al., PCT publication No. WO 2005/007699 A2.

Region of complementarity: As used herein, the term “region of complementarity” refers to a nucleotide sequence, e.g., of a oligonucleotide, that is sufficiently complementary to a cognate nucleotide sequence, e.g., of a target nucleic acid, such that the two nucleotide sequences are capable of annealing to one another under physiological conditions (e.g., in a cell). In some embodiments, a region of complementarity is fully complementary to a cognate nucleotide sequence of target nucleic acid. However, in some embodiments, a region of complementarity is partially complementary to a cognate nucleotide sequence of target nucleic acid (e.g., at least 80%, 90%, 95% or 99% complementarity). In some embodiments, a region of complementarity contains 1, 2, 3, or 4 mismatches compared with a cognate nucleotide sequence of a target nucleic acid.

Specifically binds: As used herein, the term “specifically binds” refers to the ability of a molecule to bind to a binding partner with a degree of affinity or avidity that enables the molecule to be used to distinguish the binding partner from an appropriate control in a binding assay or other binding context. With respect to an antibody, the term, “specifically binds”, refers to the ability of the antibody to bind to a specific antigen with a degree of affinity or avidity, compared with an appropriate reference antigen or antigens, that enables the antibody to be used to distinguish the specific antigen from others, e.g., to an extent that permits preferential targeting to certain cells, e.g., muscle cells, through binding to the antigen, as described herein. In some embodiments, an antibody specifically binds to a target if the antibody has a KD for binding the target of at least about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, or less. In some embodiments, an antibody specifically binds to the transferrin receptor, e.g., an epitope of the apical domain of transferrin receptor.

Subject: As used herein, the term “subject” refers to a mammal. In some embodiments, a subject is non-human primate, or rodent. In some embodiments, a subject is a human. In some embodiments, a subject is a patient, e.g., a human patient that has or is suspected of having a disease. In some embodiments, the subject is a human patient who has or is suspected of having muscle atrophy.

Transferrin receptor: As used herein, the term, “transferrin receptor” (also known as TFRC, CD71, p90, TFR, or TFR1) refers to an internalizing cell surface receptor that binds transferrin to facilitate iron uptake by endocytosis. In some embodiments, a transferrin receptor may be of human (NCBI Gene ID 7037), non-human primate (e.g., NCBI Gene ID 711568 or NCBI Gene ID 102136007), or rodent (e.g., NCBI Gene ID 22042) origin. In addition, multiple human transcript variants have been characterized that encoded different isoforms of the receptor (e.g., as annotated under GenBank RefSeq Accession Numbers: NP_001121620.1, NP_003225.2, NP_001300894.1, and NP_001300895.1).

2′-modified nucleoside: As used herein, the terms “2′-modified nucleoside” and “2′-modified ribonucleoside” are used interchangeably and refer to a nucleoside having a sugar moiety modified at the 2′ position. In some embodiments, the 2′-modified nucleoside is a 2′-4′ bicyclic nucleoside, where the 2′ and 4′ positions of the sugar are bridged (e.g., via a methylene, an ethylene, or a (S)-constrained ethyl bridge). In some embodiments, the 2′-modified nucleoside is a non-bicyclic 2′-modified nucleoside, e.g., where the 2′ position of the sugar moiety is substituted. Non-limiting examples of 2′-modified nucleosides include: 2′-deoxy, 2′-fluoro (2′-F), 2′-O-methyl (2′-O-Me), 2′-O-methoxyethyl (2′-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), 2′-O—N-methylacetamido (2′-O—NMA), locked nucleic acid (LNA, methylene-bridged nucleic acid), ethylene-bridged nucleic acid (ENA), and (S)-constrained ethyl-bridged nucleic acid (cEt). In some embodiments, the 2′-modified nucleosides described herein are high-affinity modified nucleotides and oligonucleotides comprising the 2′-modified nucleotides have increased affinity to a target sequences, relative to an unmodified oligonucleotide. Examples of structures of 2′-modified nucleosides are provided below:

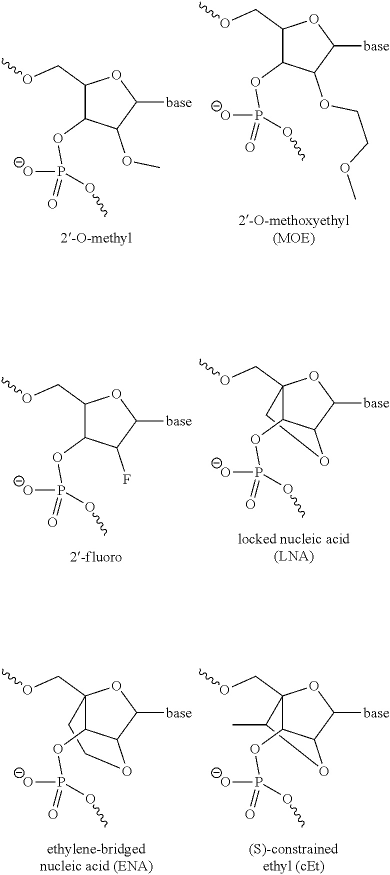

### II. Complexes

Provided herein are complexes that comprise a targeting agent, e.g. an antibody, covalently linked to a molecular payload. In some embodiments, a complex comprises a muscle-targeting antibody covalently linked to an oligonucleotide. A complex may comprise an antibody that specifically binds a single antigenic site or that binds to at least two antigenic sites that may exist on the same or different antigens.

A complex may be used to modulate the activity or function of at least one gene, protein, and/or (e.g., and) nucleic acid. In some embodiments, the molecular payload present with a complex is responsible for the modulation of a gene, protein, and/or (e.g., and) nucleic acids. A molecular payload may be a small molecule, protein, nucleic acid, oligonucleotide, or any molecular entity capable of modulating the activity or function of a gene, protein, and/or (e.g., and) nucleic acid in a cell. In some embodiments, a molecular payload is an oligonucleotide that targets a gene regulating muscle atrophy (e.g., genes listed in Table 1) in muscle cells.

In some embodiments, a complex comprises a muscle-targeting agent, e.g. an anti-transferrin receptor antibody, covalently linked to a molecular payload, e.g. an antisense oligonucleotide that targets a gene regulating muscle atrophy (e.g., genes listed in Table 1).

In some embodiments, a complex is useful for treating muscle atrophy, in which one or more molecular payloads affects the activity of one or more genes provided in Table 1. For example, a molecular payload may modulate (e.g., downregulate) transcription of the gene, modulate the expression of a protein encoded by the gene, or to modulate the activity of the encoded protein. In some embodiments, the molecular payload is an oligonucleotide that comprises a strand having a region of complementarity to a target gene provided in Table 1. In some embodiments, more than one gene in Table 1 is targeted. In some embodiments, the complex comprises molecular payloads targeting MSTN (encoding myostatin), INHBA (encoding activin A), FBXO32, TRIM63, or any combination thereof, for example MSTN and INHBA.

A composition comprising a plurality of complexes is also within the scope of the present disclosure. The molecular payloads within the plurality of complexes may be the same or different. In some embodiments, the composition comprises one or more complexes with molecular payloads targeting pro-atrophy gene or genes (e.g., MSTN (encoding myostatin) and/or (e.g., and) INHBA (encoding activin A)).

TABLE 1
List of pro-atrophy gene examples

| Gene | | |
|---|---|---|
| Symbol | GenBank Accession No. | Related Publications* |
| INHBA | NM_002192; XM_017012175.1; | Lee S J, et al., Regulation of muscle mass |
| (also known | XM_017012176.1; | by follistatin and activins., Mol |
| as EDF; | XM_017012174.1 | Endocrinol. 2010 October; 24(10): 1998- |
| FRP) | | 2008. doi: 10.1210/me.2010-0127. Epub |
| | | 2010 Sep. 1. |
| FBXO32 | NM_058229.3; | Bodine, S. C., et al., Identification of |
| (also known | NM_001242463.1; NM_148177.2 | ubiquitin ligases required for skeletal |
| as Fbx32; | | muscle atrophy. Science 294: 1704-1708, |
| MAFbx) | | 2001. |
| | | Gomes, M. D., et al., Atrogin-1, a |
| | | muscle-specific F-box protein highly |
| | | expressed during muscle atrophy. Proc. |
| | | Nat. Acad. Sci. 98: 14440-14445, 2001. |
| MSTN (also | NM_005259.2 | Saunders, M. A., et al., Human adaptive |
| known as | | evolution of myostatin (GDF8), a |
| GDF8; | | regulator of muscle growth. Am. J. Hum. |
| MSLHP) | | Genet. 79: 1089-1097, 2006. |
| | | Lin, J., et al., Myostatin knockout in |
| | | mice increases myogenesis and decreases |
| | | adipogenesis. Biochem. Biophys. Res. |
| | | Commun. 291: 701-706, 2002. |
| | | Wei Y, et al., Prevention of Muscle |
| | | Wasting by CRISPR/Cas9-mediated |
| | | Disruption of Myostatin In Vivo Volume |
| | | 24, Issue 11, p1889-1891, November |
| | | 2016 |
| TRIM63 | NM_032588.3; XM_017002559.2 | Höllriegel R, et al. Anabolic effects of |
| (also known | | exercise training in patients with |
| as IRF; | | advanced chronic heart failure (NYHA |
| SMRZ; | | IIIb): impact on ubiquitin-protein ligases |
| MURF1; | | expression and skeletal muscle size. Int J |
| MURF2; | | Cardiol, 2013 Aug. 10. |
| RNF28) | | Eddins M J, et al. Targeting the ubiquitin |
| | | E3 ligase MuRF1 to inhibit muscle |
| | | atrophy. |
| | | Cell Biochem Biophys, 2011 June. |

*The contents of the cited references are incorporated herein by reference in their entireties.

A. Muscle-Targeting Agents

Some aspects of the disclosure provide muscle-targeting agents, e.g., for delivering a molecular payload to a muscle cell. In some embodiments, such muscle-targeting agents are capable of binding to a muscle cell, e.g., via specifically binding to an antigen on the muscle cell, and delivering an associated molecular payload to the muscle cell. In some embodiments, the molecular payload is bound (e.g., covalently bound) to the muscle targeting agent and is internalized into the muscle cell upon binding of the muscle targeting agent to an antigen on the muscle cell, e.g., via endocytosis. It should be appreciated that various types of muscle-targeting agents may be used in accordance with the disclosure. For example, the muscle-targeting agent may comprise, or consist of, a nucleic acid (e.g., DNA or RNA), a peptide (e.g., an antibody), a lipid (e.g., a microvesicle), or a sugar moiety (e.g., a polysaccharide). Exemplary muscle-targeting agents are described in further detail herein, however, it should be appreciated that the exemplary muscle-targeting agents provided herein are not meant to be limiting.

Some aspects of the disclosure provide muscle-targeting agents that specifically bind to an antigen on muscle, such as skeletal muscle, smooth muscle, or cardiac muscle. In some embodiments, any of the muscle-targeting agents provided herein bind to (e.g., specifically bind to) an antigen on a skeletal muscle cell, a smooth muscle cell, and/or (e.g., and) a cardiac muscle cell.

By interacting with muscle-specific cell surface recognition elements (e.g., cell membrane proteins), both tissue localization and selective uptake into muscle cells can be achieved. In some embodiments, molecules that are substrates for muscle uptake transporters are useful for delivering a molecular payload into muscle tissue. Binding to muscle surface recognition elements followed by endocytosis can allow even large molecules such as antibodies to enter muscle cells. As another example molecular payloads conjugated to transferrin or anti-transferrin receptor antibodies can be taken up by muscle cells via binding to transferrin receptor, which may then be endocytosed, e.g., via clathrin-mediated endocytosis.

The use of muscle-targeting agents may be useful for concentrating a molecular payload (e.g., oligonucleotide) in muscle while reducing toxicity associated with effects in other tissues. In some embodiments, the muscle-targeting agent concentrates a bound molecular payload in muscle cells as compared to another cell type within a subject. In some embodiments, the muscle-targeting agent concentrates a bound molecular payload in muscle cells (e.g., skeletal, smooth, or cardiac muscle cells) in an amount that is at least 1, 2, 3, 4, 5, 6, 7, 8, 9, 10, 15, 20, 30, 40, 50, 60, 70, 80, 90, or 100 times greater than an amount in non-muscle cells (e.g., liver, neuronal, blood, or fat cells). In some embodiments, a toxicity of the molecular payload in a subject is reduced by at least 1%, 2%, 3%, 4%, 5%, 10%, 15%, 20%, 25%, 30%, 35%, 40%, 45%, 50%, 55%, 60%, 65%, 70%, 75%, 80%, 90%, or 95% when it is delivered to the subject when bound to the muscle-targeting agent.

In some embodiments, to achieve muscle selectivity, a muscle recognition element (e.g., a muscle cell antigen) may be required. As one example, a muscle-targeting agent may be a small molecule that is a substrate for a muscle-specific uptake transporter. As another example, a muscle-targeting agent may be an antibody that enters a muscle cell via transporter-mediated endocytosis. As another example, a muscle targeting agent may be a ligand that binds to cell surface receptor on a muscle cell. It should be appreciated that while transporter-based approaches provide a direct path for cellular entry, receptor-based targeting may involve stimulated endocytosis to reach the desired site of action.

Muscle cells encompassed by the present disclosure include, but are not limited to, skeletal muscle cells, smooth muscle cells, cardiac muscle cells, myoblasts and myocytes.

i. Muscle-Targeting Antibodies

In some embodiments, the muscle-targeting agent is an antibody. Generally, the high specificity of antibodies for their target antigen provides the potential for selectively targeting muscle cells (e.g., skeletal, smooth, and/or (e.g., and) cardiac muscle cells). This specificity may also limit off-target toxicity. Examples of antibodies that are capable of targeting a surface antigen of muscle cells have been reported and are within the scope of the disclosure. For example, antibodies that target the surface of muscle cells are described in Arahata K., et al. “Immunostaining of skeletal and cardiac muscle surface membrane with antibody against Duchenne muscular dystrophy peptide” Nature 1988; 333: 861-3; Song K. S., et al. “Expression of caveolin-3 in skeletal, cardiac, and smooth muscle cells. Caveolin-3 is a component of the sarcolemma and co-fractionates with dystrophin and dystrophin-associated glycoproteins” J Biol Chem 1996; 271: 15160-5; and Weisbart R. H. et al., “Cell type specific targeted intracellular delivery into muscle of a monoclonal antibody that binds myosin IIb” Mol Immunol. 2003 March, 39(13):78309; the entire contents of each of which are incorporated herein by reference.

a. Anti-Transferrin Receptor Antibodies

Some aspects of the disclosure are based on the recognition that agents binding to transferrin receptor, e.g., anti-transferrin-receptor antibodies, are capable of targeting muscle cell. Transferrin receptors are internalizing cell surface receptors that transport transferrin across the cellular membrane and participate in the regulation and homeostasis of intracellular iron levels. Some aspects of the disclosure provide transferrin receptor binding proteins, which are capable of binding to transferrin receptor. Accordingly, aspects of the disclosure provide binding proteins (e.g., antibodies) that bind to transferrin receptor. In some embodiments, binding proteins that bind to transferrin receptor are internalized, along with any bound molecular payload, into a muscle cell. As used herein, an antibody that binds to a transferrin receptor may be referred to interchangeably as an, transferrin receptor antibody, an anti-transferrin receptor antibody, or an anti-TfR antibody. Antibodies that bind, e.g. specifically bind, to a transferrin receptor may be internalized into the cell, e.g. through receptor-mediated endocytosis, upon binding to a transferrin receptor.

It should be appreciated that anti-transferrin receptor antibodies may be produced, synthesized, and/or (e.g., and) derivatized using several known methodologies, e.g. library design using phage display. Exemplary methodologies have been characterized in the art and are incorporated by reference (Díez, P. et al. “High-throughput phage-display screening in array format”, Enzyme and microbial technology, 2015, 79, 34-41.; Christoph M. H. and Stanley, J. R. “Antibody Phage Display: Technique and Applications” J Invest Dermatol. 2014, 134:2.; Engleman, Edgar (Ed.) “Human Hybridomas and Monoclonal Antibodies.” 1985, Springer.). In other embodiments, an anti-transferrin antibody has been previously characterized or disclosed. Antibodies that specifically bind to transferrin receptor are known in the art (see, e.g. U.S. Pat. No. 4,364,934, filed Dec. 4, 1979, “Monoclonal antibody to a human early thymocyte antigen and methods for preparing same”; U.S. Pat. No. 8,409,573, filed Jun. 14, 2006, “Anti-CD71 monoclonal antibodies and uses thereof for treating malignant tumor cells”; U.S. Pat. No. 9,708,406, filed May 20, 2014, “Anti-transferrin receptor antibodies and methods of use”; U.S. Pat. No. 9,611,323, filed Dec. 19, 2014, “Low affinity blood brain barrier receptor antibodies and uses therefor”; WO 2015/098989, filed Dec. 24, 2014, “Novel anti-Transferrin receptor antibody that passes through blood-brain barrier”; Schneider C. et al. “Structural features of the cell surface receptor for transferrin that is recognized by the monoclonal antibody OKT9.” J Biol Chem. 1982, 257:14, 8516-8522.; Lee et al. “Targeting Rat Anti-Mouse Transferrin Receptor Monoclonal Antibodies through Blood-Brain Barrier in Mouse” 2000, J Pharmacol. Exp. Ther., 292: 1048-1052.).

Provided herein, in some aspects, are new anti-TfR antibodies for use as the muscle targeting agents (e.g., in muscle targeting complexes). In some embodiments, the anti-TfR antibody described herein binds to transferrin receptor with high specificity and affinity. In some embodiments, the anti-TfR antibody described herein specifically binds to any extracellular epitope of a transferrin receptor or an epitope that becomes exposed to an antibody. In some embodiments, anti-TfR antibodies provided herein bind specifically to transferrin receptor from human, non-human primates, mouse, rat, etc. In some embodiments, anti-TfR antibodies provided herein bind to human transferrin receptor. In some embodiments, the anti-TfR antibody described herein binds to an amino acid segment of a human or non-human primate transferrin receptor, as provided in SEQ ID NOs: 242-245. In some embodiments, the anti-TfR antibody described herein binds to an amino acid segment corresponding to amino acids 90-96 of a human transferrin receptor as set forth in SEQ ID NO: 242, which is not in the apical domain of the transferrin receptor.

In some embodiments, an anti-TFR antibody specifically binds a TfR1 (e.g., a human or non-human primate TfR1) with binding affinity (e.g., as indicated by Kd) of at least about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, or less. In some embodiments, the anti-TfR antibodies described herein binds to TfR1 with a KD of sub-nanomolar range. In some embodiments, the anti-TfR antibodies described herein selectively binds to transferrin receptor 1 (TfR1) but do not bind to transferrin receptor 2 (TfR2). In some embodiments, the anti-TfR antibodies described herein binds to human TfR1 and cyno TfR1 (e.g., with a Kd of 10⁻⁷ M, 10⁻⁸ M, 10⁻⁹ M, 10⁻¹⁰ M, 10⁻¹¹ M, 10⁻¹² M, 10⁻¹³ M, or less), but does not bind to a mouse TfR1. The affinity and binding kinetics of the anti-TfR antibody can be tested using any suitable method including but not limited to biosensor technology (e.g., OCTET or BIACORE). In some embodiments, binding of any one of the anti-TfR antibody described herein does not complete with or inhibit transferrin binding to the TfR1. In some embodiments, binding of any one of the anti-TfR antibody described herein does not complete with or inhibit HFE-beta-2-microglobulin binding to the TfR1.

An example human transferrin receptor amino acid sequence, corresponding to NCBI sequence NP_003225.2 (transferrin receptor protein 1 isoform 1, Homo sapiens) is as follows:

(SEQ ID NO: 242)
MMDQARSAFSNLFGGEPLSYTRFSLARQVDGDNSHVEMKLAVDEEENADN
NTKANVTKPKRCSGSICYGTIAVIVFFLIGFMIGYLGYCKGVEPKTECER
LAGTESPVREEPGEDFPAARRLYWDDLKRKLSEKLDSTDFTGTIKLLNEN
SYVPREAGSQKDENLALYVENQFREFKLSKVWRDQHFVKIQVKDSAQNSV
IIVDKNGRLVYLVENPGGYVAYSKAATVTGKLVHANFGTKKDFEDLYTPV
NGSIVIVRAGKITFAEKVANAESLNAIGVLIYMDQTKFPIVNAELSFFGH
AHLGTGDPYTPGFPSFNHTQFPPSRSSGLPNIPVQTISRAAAEKLFGNME
GDCPSDWKTDSTCRMVTSESKNVKLTVSNVLKEIKILNIFGVIKGFVEPD
HYVVVGAQRDAWGPGAAKSGVGTALLLKLAQMFSDMVLKDGFQPSRSIIF
ASWSAGDFGSVGATEWLEGYLSSLHLKAFTYINLDKAVLGTSNFKVSASP
LLYTLIEKTMQNVKHPVTGQFLYQDSNWASKVEKLTLDNAAFPFLAYSGI
PAVSFCFCEDTDYPYLGTTMDTYKELIERIPELNKVARAAAEVAGQFVIK
LTHDVELNLDYERYNSQLLSFVRDLNQYRADIKEMGLSLQWLYSARGDFF
RATSRLTTDFGNAEKTDRFVMKKLNDRVMRVEYHFLSPYVSPKESPFRHV
FWGSGSHTLPALLENLKLRKQNNGAFNETLFRNQLALATWTIQGAANALS
GDVWDIDNEF.

An example non-human primate transferrin receptor amino acid sequence, corresponding to NCBI sequence NP_001244232.1(transferrin receptor protein 1, Macaca mulatta) is as follows:

(SEQ ID NO: 243)
MMDQARSAFSNLFGGEPLSYTRFSLARQVDGDNSHVEMKLGVDEEENTDN
NTKPNGTKPKRCGGNICYGTIAVIIFFLIGFMIGYLGYCKGVEPKTECER
LAGTESPAREEPEEDFPAAPRLYWDDLKRKLSEKLDTTDFTSTIKLLNEN
LYVPREAGSQKDENLALYIENQFREFKLSKVWRDQHFVKIQVKDSAQNSV
IIVDKNGGLVYLVENPGGYVAYSKAATVTGKLVHANFGTKKDFEDLDSPV
NGSIVIVRAGKITFAEKVANAESLNAIGVLIYMDQTKFPIVKADLSFFGH
AHLGTGDPYTPGFPSFNHTQFPPSQSSGLPNIPVQTISRAAAEKLFGNME
GDCPSDWKTDSTCKMVTSENKSVKLTVSNVLKETKILNIFGVIKGFVEPD
HYVVVGAQRDAWGPGAAKSSVGTALLLKLAQMFSDMVLKDGFQPSRSIIF
ASWSAGDFGSVGATEWLEGYLSSLHLKAFTYINLDKAVLGTSNFKVSASP
LLYTLIEKTMQDVKHPVTGRSLYQDSNWASKVEKLTLDNAAFPFLAYSGI
PAVSFCFCEDTDYPYLGTTMDTYKELVERIPELNKVARAAAEVAGQFVIK
LTHDTELNLDYERYNSQLLLFLRDLNQYRADVKEMGLSLQWLYSARGDFF
RATSRLTTDFRNAEKRDKFVMKKLNDRVMRVEYYFLSPYVSPKESPFRHV
FWGSGSHTLSALLESLKLRRQNNSAFNETLFRNQLALATWTIQGAANALS
GDVWDIDNEF

An example non-human primate transferrin receptor amino acid sequence, corresponding to NCBI sequence XP_005545315.1 (transferrin receptor protein 1, Macaca fascicularis) is as follows:

(SEQ ID NO: 244)
MMDQARSAFSNLFGGEPLSYTRFSLARQVDGDNSHVEMKLGVDEEENTDN
NTKANGTKPKRCGGNICYGTIAVIIFFLIGFMIGYLGYCKGVEPKTECER
LAGTESPAREEPEEDFPAAPRLYWDDLKRKLSEKLDTTDFTSTIKLLNEN
LYVPREAGSQKDENLALYIENQFREFKLSKVWRDQHFVKIQVKDSAQNSV
IIVDKNGGLVYLVENPGGYVAYSKAATVTGKLVHANFGTKKDFEDLDSPV
NGSIVIVRAGKITFAEKVANAESLNAIGVLIYMDQTKFPIVKADLSFFGH
AHLGTGDPYTPGFPSFNHTQFPPSQSSGLPNIPVQTISRAAAEKLFGNME
GDCPSDWKTDSTCKMVTSENKSVKLTVSNVLKETKILNIFGVIKGFVEPD
HYVVVGAQRDAWGPGAAKSSVGTALLLKLAQMFSDMVLKDGFQPSRSIIF
ASWSAGDFGSVGATEWLEGYLSSLHLKAFTYINLDKAVLGTSNFKVSASP
LLYTLIEKTMQDVKHPVTGRSLYQDSNWASKVEKLTLDNAAFPFLAYSGI
PAVSFCFCEDTDYPYLGTTMDTYKELVERIPELNKVARAAAEVAGQFVIK
LTHDTELNLDYERYNSQLLLFLRDLNQYRADVKEMGLSLQWLYSARGDFF
RATSRLTTDFRNAEKRDKFVMKKLNDRVMRVEYYFLSPYVSPKESPFRHV
FWGSGSHTLSALLESLKLRRQNNSAFNETLFRNQLALATWTIQGAANALS
GDVWDIDNEF.

An example mouse transferrin receptor amino acid sequence, corresponding to NCBI sequence NP_001344227.1 (transferrin receptor protein 1, mus musculus) is as follows:

(SEQ ID NO: 245)
MMDQARSAFSNLFGGEPLSYTRFSLARQVDGDNSHVEMKLAADEEENADN
NMKASVRKPKRFNGRLCFAAIALVIFFLIGFMSGYLGYCKRVEQKEECVK
LAETEETDKSETMETEDVPTSSRLYWADLKTLLSEKLNSIEFADTIKQLS
QNTYTPREAGSQKDESLAYYIENQFHEFKFSKVWRDEHYVKIQVKSSIGQ
NMVTIVQSNGNLDPVESPEGYVAFSKPTEVSGKLVHANFGTKKDFEELSY
SVNGSLVIVRAGEITFAEKVANAQSFNAIGVLIYMDKNKFPVVEADLALF
GHAHLGTGDPYTPGFPSFNHTQFPPSQSSGLPNIPVQTISRAAAEKLFGK
MEGSCPARWNIDSSCKLELSQNQNVKLIVKNVLKERRILNIFGVIKGYEE
PDRYVVVGAQRDALGAGVAAKSSVGTGLLLKLAQVFSDMISKDGFRPSRS
IIFASWTAGDFGAVGATEWLEGYLSSLHLKAFTYINLDKVVLGTSNFKVS
ASPLLYTLMGKIMQDVKHPVDGKSLYRDSNWISKVEKLSFDNAAYPFLAY
SGIPAVSFCFCEDADYPYLGTRLDTYEALTQKVPQLNQMVRTAAEVAGQL
IIKLTHDVELNLDYEMYNSKLLSFMKDLNQFKTDIRDMGLSLQWLYSARG
DYFRATSRLTTDFHNAEKTNRFVMREINDRIMKVEYHFLSPYVSPRESPF
RHIFWGSGSHTLSALVENLKLRQKNITAFNETLFRNQLALATWTIQGVAN
ALSGDIWNIDNEF

In some embodiments, an anti-transferrin receptor antibody binds to an amino acid segment of the receptor as follows: FVKIQVKDSAQNSVIIVDKNGRLVYLVENPGGYVAYSKAATVTGKLVHANFGTKKDF EDLYTPVNGSIVIVRAGKITFAEKVANAESLNAIGVLIYMDQTKFPIVNAELSFFGHAH LGTGDPYTPGFPSFNHTQFPPSRSSGLPNIPVQTISRAAAEKLFGNMEGDCPSDWKTDS TCRMVTSESKNVKLTVSNVLKE (SEQ ID NO: 246) and does not inhibit the binding interactions between transferrin receptors and transferrin and/or (e.g., and) human hemochromatosis protein (also known as HFE). In some embodiments, the anti-transferrin receptor antibody described herein does not bind an epitope in SEQ ID NO: 246.

Appropriate methodologies may be used to obtain and/or (e.g., and) produce antibodies, antibody fragments, or antigen-binding agents, e.g., through the use of recombinant DNA protocols. In some embodiments, an antibody may also be produced through the generation of hybridomas (see, e.g., Kohler, G and Milstein, C. “Continuous cultures of fused cells secreting antibody of predefined specificity” Nature, 1975, 256: 495-497). The antigen-of-interest may be used as the immunogen in any form or entity, e.g., recombinant or a naturally occurring form or entity. Hybridomas are screened using standard methods, e.g. ELISA screening, to find at least one hybridoma that produces an antibody that targets a particular antigen. Antibodies may also be produced through screening of protein expression libraries that express antibodies, e.g., phage display libraries. Phage display library design may also be used, in some embodiments, (see, e.g. U.S. Pat. No 5,223,409, filed Mar. 1, 1991, “Directed evolution of novel binding proteins”; WO 1992/18619, filed Apr. 10, 1992, “Heterodimeric receptor libraries using phagemids”; WO 1991/17271, filed May 1, 1991, “Recombinant library screening methods”; WO 1992/20791, filed May 15, 1992, “Methods for producing members of specific binding pairs”; WO 1992/15679, filed Feb. 28, 1992, and “Improved epitope displaying phage”). In some embodiments, an antigen-of-interest may be used to immunize a non-human animal, e.g., a rodent or a goat. In some embodiments, an antibody is then obtained from the non-human animal, and may be optionally modified using a number of methodologies, e.g., using recombinant DNA techniques. Additional examples of antibody production and methodologies are known in the art (see, e.g. Harlow et al. “Antibodies: A Laboratory Manual”, Cold Spring Harbor Laboratory, 1988.).

In some embodiments, an antibody is modified, e.g., modified via glycosylation, phosphorylation, sumoylation, and/or (e.g., and) methylation. In some embodiments, an antibody is a glycosylated antibody, which is conjugated to one or more sugar or carbohydrate molecules. In some embodiments, the one or more sugar or carbohydrate molecule are conjugated to the antibody via N-glycosylation, O-glycosylation, C-glycosylation, glypiation (GPI anchor attachment), and/or (e.g., and) phosphoglycosylation. In some embodiments, the one or more sugar or carbohydrate molecules are monosaccharides, disaccharides, oligosaccharides, or glycans. In some embodiments, the one or more sugar or carbohydrate molecule is a branched oligosaccharide or a branched glycan. In some embodiments, the one or more sugar or carbohydrate molecule includes a mannose unit, a glucose unit, an N-acetylglucosamine unit, an N-acetylgalactosamine unit, a galactose unit, a fucose unit, or a phospholipid unit. In some embodiments, there are about 1-10, about 1-5, about 5-10, about 1-4, about 1-3, or about 2 sugar molecules. In some embodiments, a glycosylated antibody is fully or partially glycosylated. In some embodiments, an antibody is glycosylated by chemical reactions or by enzymatic means. In some embodiments, an antibody is glycosylated in vitro or inside a cell, which may optionally be deficient in an enzyme in the N- or O-glycosylation pathway, e.g. a glycosyltransferase. In some embodiments, an antibody is functionalized with sugar or carbohydrate molecules as described in International Patent Application Publication WO2014065661, published on May 1, 2014, entitled, “Modified antibody, antibody-conjugate and process for the preparation thereof”.

In some embodiments, the anti-TfR antibody of the present disclosure comprises a VL domain and/or (e.g., and) VH domain of any one of the anti-TfR antibodies selected from Table 2, and comprises a constant region comprising the amino acid sequences of the constant regions of an IgG, IgE, IgM, IgD, IgA or IgY immunoglobulin molecule, any class (e.g., IgG1, IgG2, IgG3, IgG4, IgA1 and IgA2), or any subclass (e.g., IgG2a and IgG2b) of immunoglobulin molecule. Non-limiting examples of human constant regions are described in the art, e.g., see Kabat E A et al., (1991) supra.

The heavy chain and light chain variable domain and CDR sequences of non-limiting examples of anti-TfR antibodies are provided in Table 2.

TABLE 2
Examples of anti-TfR1 antibodies
(CDRs according to the IMGT® definition)

| Ab | CDRs | Variable domains |
|---|---|---|
| 3-A4 | CDR-H1: | VH |
| | GFNIKDDY (SEQ ID NO: 1) | EVQLQQSGAELVRPGASVKLSCTASGFNIKDDYMYWVKQ |
| | CDR-H2: | RPEQGLEWIGWIDPENGDTEYASKFQDKATVTADTSSNT |
| | IDPENGDT (SEQ ID NO: 2) | AYLQLSSLTSEDTAVYYCTLWLRRGLDYWGQGTSVTVSS |
| | CDR-H3: | (SEQ ID NO: 7) |
| | TLWLRRGLDY (SEQ ID NO: 3) | |
| | CDR-L1: | VL |
| | KSLLHSNGYTY (SEQ ID NO: 4) | DIVMTQAAPSVPVTPGESVSISCRSSKSLLHSNGYTYLF |
| | CDR-L2: | WFLQRPGQSPQLLIYRMSNLASGVPDRFSGSGSGTAFTL |
| | RMS (SEQ ID NO: 5) | RISRVEAEDVGVYYCMQHLEYPFTFGGGTKLEIK (SEQ |
| | CDR-L3: | ID NO: 8) |
| | MQHLEYPFT (SEQ ID NO: 6) | |
| 3-M12 | CDR-H1: | VH |
| | GYSITSGYY (SEQ ID NO: 9) | DVQLQESGPGLVKPSQSLSLTCSVTGYSITSGYYWNWIR |
| | CDR-H2: | QFPGNKLEWMGYITFDGANNYNPSLKNRISITRDTSKNQ |
| | ITFDGAN (SEQ ID NO: 10) | FFLKLTSVTTEDTATYYCTRSSYDYDVLDYWGQGTTLTV |
| | CDR-H3: | SS (SEQ ID NO: 15) |
| | TRSSYDYDVLDY (SEQ ID NO: 11) | |
| | CDR-L1: | VL |
| | QDISNF (SEQ ID NO: 12) | DIQMTQTTSSLSASLGDRVTISCRASQDISNFLNWYQQR |
| | CDR-L2: | PDGTVKLLIYYTSRLHSGVPSRFSGSGSGTDFSLTVSNL |
| | YTS (SEQ ID NO: 13) | EQEDIATYFCQQGHTLPYTFGGGTKLEIK (SEQ ID |
| | CDR-L3: | NO: 16) |
| | QQGHTLPYT (SEQ ID NO: 14) | |
| 5-H12 | CDR-H1: | VH |
| | GYSFTDYC (SEQ ID NO: 17) | QIQLQQSGPELVRPGASVKISCKASGYSFTDYCINWVNQ |
| | CDR-H2: | RPGQGLEWIGWIYPGSGNTRYSERFKGKATLTVDTSSNT |
| | IYPGSGNT (SEQ ID NO: 18) | AYMQLSSLTSEDSAVYFCAREDYYPYHGMDYWGQGTSVT |
| | CDR-H3: | VSS (SEQ ID NO: 23) |
| | AREDYYPYHGMDY (SEQ ID NO: 19) | |
| | CDR-L1: | VL |
| | ESVDGYDNSF (SEQ ID NO: 20) | DIVLTQSPTSLAVSLGQRATISCRASESVDGYDNSFMHW |
| | CDR-L2: | YQQKPGQPPKLLIFRASNLESGIPARFSGSGSRTDFTLT |
| | RAS (SEQ ID NO: 21) | INPVEAADVATYYCQQSSEDPWTFGGGTKLEIK (SEQ |
| | CDR-L3: | ID NO: 24) |
| | QQSSEDPWT (SEQ ID NO: 22) | |
| 8-K6 | CDR-H1: | VH |
| | GYTFTSYW (SEQ ID NO: 25) | QVHLQQPGAELVKPGASVKMSCKASGYTFTSYWITWVKQ |
| | CDR-H2: | RPGQGLEWIGDIFPNSGRTNYDEKFKSKATLTVDTSSST |
| | IFPNSGRT (SEQ ID NO: 26) | AYMQLSSLTSEDSAVYFCAREGNFGSLDYWGQGTTLTVS |
| | CDR-H3: | S (SEQ ID NO: 31) |
| | AREGNFGSLDY (SEQ ID NO: 27) | |
| | CDR-L1: | VL |
| | SNLNY (SEQ ID NO: 28) | QIVLTQSPAIMSASPGEKVTMTCSANSNLNYMNWYHQKS |
| | CDR-L2: | GTSPKRWIYDTSKLASGVPARFSASGSGTSYSLTISSME |
| | DTS (SEQ ID NO: 29) | AEDAATYYCQQWSRNPLTFGAGTRLELK (SEQ ID |
| | CDR-L3: | NO: 32) |
| | QQWSRNPLT (SEQ ID NO: 30) | |
| 9-K23 | CDR-H1: | VH |
| | GFSLNTYDVG (SEQ ID NO: 33) | QVTLKESGPGMLQPSQTLSLTCSFSGFSLNTYDVGVGWI |
| | CDR-H2: | RQPSGKGLEWLANIWWNDDKYYNSALKSRLTISKDTSNN |
| | IWWNDDK (SEQ ID NO: 34) | QVFLKISSVDTADTATYYCTLYSYDGGFAYWGQGTLVTV |
| | CDR-H3: | SA (SEQ ID NO: 39) |
| | TLYSYDGGFAY (SEQ ID NO: 35) | |
| | CDR-L1: | VL |
| | SSVSSSY (SEQ ID NO: 36) | QIVLTQSPAIMSASLGERVTMTCTASSSVSSSYLHWYQQ |
| | CDR-L2: | KPGSSPKLWIYSTSNLASGVPARFSGSGSGTSYSLTISS |
| | STS (SEQ ID NO: 37) | MEAEDAATYYCHQYHRSPYTFGGGTKLEIK (SEQ ID |
| | CDR-L3: | NO: 40) |
| | HQYHRSPYT (SEQ ID NO: 38) | |
| 3-E5 | CDR-H1: | VH |
| | GYSFTGYN (SEQ ID NO: 41) | EIQMKQSGAELVKPGASVKISCKASGYSFTGYNMNWVKQ |
| | CDR-H2: | SHGKSLEWIGNINPYYGSTGYNQKFKGKATLTVDKSSST |
| | INPYYGST (SEQ ID NO: 42) | AYMQLNSLTSEDSAVYYCARGDYGYDEGTWFAYWGQGTL |
| | CDR-H3: | VTVSA (SEQ ID NO: 47) |
| | ARGDYGYDEGTWFAY (SEQ ID | |
| | NO: 43) | |
| | CDR-L1: | VL |
| | QSLLNSRTRKNY (SEQ ID NO: 44) | DIVMSQSPSSLAVSAGEKVTMSCKSSQSLLNSRTRKNYL |
| | CDR-L2: | AWYQQKPEQSPKLLIYWASTRESGVPDRFTGSGSGTDFT |
| | WAS (SEQ ID NO: 45) | LTISSVQAEDLAVYYCKQSYNLPFTFGSGTKLEIK |
| | CDR-L3: | (SEQ ID NO: 48) |
| | KQSYNLPFT (SEQ ID NO: 46) | |
| 6-D3 | CDR-H1: | VH |
| | GYTFTRHW (SEQ ID NO: 49) | QVQLQQPGAELVKPGASVKMSCKASGYTFTRHWITWVKQ |
| | CDR-H2: | RPGQGLEWIGDIYPGSGRTNYNEKFKSTATLTVDTSSST |
| | IYPGSGRT (SEQ ID NO: 50) | AYMQLSSLTSEDSAVYYCARDGYLYINYFDYWGQGTTLT |
| | CDR-H3: | VSS (SEQ ID NO: 54) |
| | ARDGYLYINYFDY (SEQ ID NO: | |
| | 51) | |
| | CDR-L1: | VL |
| | SSVSF (SEQ ID NO: 52) | ENVLTQSPAIMSASPGEKVTMTCSASSSVSFMHWFQQKS |
| | CDR-L2: | STSPKLWIYDTSKLASGVPGRFSGSGSGSSYSLTISSMA |
| | DTS (SEQ ID NO: 29) | AEDVATYYCFQGSGYPYTFGGGTKLEIK (SEQ ID |
| | CDR-L3: | NO: 55) |
| | FQGSGYPYT (SEQ ID NO: 53) | |
| 4-O12 | CDR-H1: | VH |
| | GFNIVDDY (SEQ ID NO: 56) | EVQLQQSGAELVRPGASVKLSCTASGFNIVDDYMHWVKQ |
| | CDR-H2: | RPEQGLEWIGWIYPENADTEYASKFQGKATITADTSSNT |
| | IYPENADT (SEQ ID NO: 57) | AYLQLSSLTSEDTAVYYCTTATGTGWFAYWGQGTLVTVS |
| | CDR-H3: | A (SEQ ID NO: 62) |
| | TTATGTGWFAY (SEQ ID NO: 58) | |
| | CDR-L1: | VL |
| | QSLLDSDGKTY (SEQ ID NO: 59) | DVVMTQTPLTLSVTIGQPASISCKSSQSLLDSDGKTYLN |
| | CDR-L2: | WLFQRPGQSPKRLIYLVSKLDSGVPDRFTGSGSGTDFTL |
| | LVS (SEQ ID NO: 60) | KISRVETEDLGVYYCWQGTHFPWTFGGGAKLEIK (SEQ |
| | CDR-L3: | ID NO: 63) |
| | WQGTHFPWT (SEQ ID NO: 61) | |
| 4-C5 | CDR-H1: | VH |
| | GYTFSNYW (SEQ ID NO: 64) | QVQLQQSGAELMKPGASVKISCKATGYTFSNYWIEWVKQ |
| | CDR-H2: | RPGHGLEWIGEILPGSGSTNYNENFKGKATFTADTSSNT |
| | ILPGSGST (SEQ ID NO: 65) | AYMQLSSLTSEDSAVYYCARRGAYGNFHYWGQGTTLTVS |
| | CDR-H3: | S (SEQ ID NO: 70) |
| | ARRGAYGNFHY (SEQ ID NO: 66) | |
| | CDR-L1: | VL |
| | SSISSSN (SEQ ID NO: 67) | EIVLTQSPALMAASPGEKVTITCSVSSSISSSNLHWYQQ |
| | CDR-L2: | KSETSPKPWIYGTSNLASGVPVRFSGSGSGTSYSLTISS |
| | GTS (SEQ ID NO: 68) | MEAEDAATYYCQQWRSYPYTFGGGTKLEIK (SEQ ID |
| | CDR-L3: | NO: 71) |
| | QQWRSYPYT (SEQ ID NO: 69) | |
| 10-P5 | CDR-H1: | VH |
| | GYTFTDYN (SEQ ID NO: 72) | EVQLQQFGAELVKPGASVKISCKASGYTFTDYNMAWVKE |
| | CDR-H2: | SHGKSLEWIGDINPNYDTTSYNQKFKGKATLTVDKSSST |
| | INPNYDTT (SEQ ID NO: 73) | AHMELRSLTSEGTAVYYCARSGYYGSSYYWHFDVWGTGT |
| | CDR-H3: | TVTVSS (SEQ ID NO: 77) |
| | ARSGYYGSSYYWHFDV (SEQ ID | |
| | NO: 74) | |
| | CDR-L1: | VL |
| | QSLLYSSNQKNY (SEQ ID NO: 75) | DIVMSQSPSSLAVSVGEKVTMSCKSSQSLLYSSNQKNYL |
| | CDR-L2: | AWYQQKPGQSPKLLIYWASTRESGVPDRFTGSGSGTDFT |
| | WAS (SEQ ID NO: 45) | LTISSVKAEDLAVYYCQQYYNYPFTFGSGTKLEIK |
| | CDR-L3: | (SEQ ID NO: 78) |
| | QQYYNYPFT (SEQ ID NO: 76) | |
| 2-H19 | CDR-H1: | VH |
| | GFNIKDYY (SEQ ID NO: 79) | EVQLQQSGAELVRSGASVKLSCTASGFNIKDYYMHWVKQ |
| | CDR-H2: | RPEQGLEWIGWIDPESGDTEYAPKFQGRATMTADTSSNT |
| | IDPESGDT (SEQ ID NO: 80) | AYMQLSSLTSEDTAVYYCYGHDYRVDCWGQGTSVTVSS |
| | CDR-H3: | (SEQ ID NO: 85) |
| | YGHDYRVDC (SEQ ID NO: 81) | |
| | CDR-L1: | VL |
| | QSLVHSNGNTY (SEQ ID NO: 82) | DVVMTQTPLSLPVSLGDQASISCRSSQSLVHSNGNTYLH |
| | CDR-L2: | WYLQKPGQSPKLLIYKVSNRFSGVPDRFSGSGSGTDFTL |
| | KVS (SEQ ID NO: 83) | KISRVEAEDLGVYFCSQSTHIPWTFGGGTKLEIK (SEQ |
| | CDR-L3: | ID NO: 86) |
| | SQSTHIPWT (SEQ ID NO: 84) | |
| 3-F3 | CDR-H1: | VH |
| | GYTFTDYN (SEQ ID NO: 72) | EVQLQQFGAELVKPGASVKISCKASGYTFTDYNMGWVKQ |
| | CDR-H2: | SHGKSLEWIGDINPNYDSTSYTQKFKGKATLTVDKSSST |
| | INPNYDST (SEQ ID NO: 87) | AYMELRSLTSEDTAVYYCARSGYYGSSYYWHFDVWGTGT |
| | CDR-H3: | TVTVSS (SEQ ID NO: 89) |
| | ARSGYYGSSYYWHFDV (SEQ ID | |
| | NO: 74) | |
| | CDR-L1: | VL |
| | QSLLYSSNQKNY (SEQ ID NO: 75) | DIVMSQSPSSLAVSVGEKVTMSCKSSQSLLYSSNQKNYL |
| | CDR-L2: | AWYQQKPGQSPKLLIYWASTRESGVPDRFTGSGSGTDFT |
| | WAS (SEQ ID NO: 45) | LTISSVKAEDLAVYYCQQYYHYPFTFGSGTKLEIK |
| | CDR-L3: | (SEQ ID NO: 90) |
| | QQYYHYPFT (SEQ ID NO: 88) | |
| 8-O17 | CDR-H1: | VH |
| | GFSLTNYG (SEQ ID NO: 91) | QVQLKESGPGLVAPSQSLSITCTVSGFSLTNYGVHWVRQ |
| | CDR-H2: | PPGKGLEWLVVIWNDGSATYNSALESRLSISKDNSKSQV |
| | IWNDGSA (SEQ ID NO: 92) | FLKMNSLQTDDTAMYYCARHESSNPFAYWGQGTLVTVSA |
| | CDR-H3: | (SEQ ID NO: 97) |
| | ARHESSNPFAY (SEQ ID NO: 93) | |
| | CDR-L1: | VL |
| | QSIGTS (SEQ ID NO: 94) | DILLTQSPAILSVSPGERVSFSCRASQSIGTSIHWYQQR |
| | CDR-L2: | TNGSPRLLIKSASESIAGIPSRFSGSGSGTDFTLSINSV |
| | SAS (SEQ ID NO: 95) | ESEDIADYYCQQNNRWPYTFGGGTKLEIK (SEQ ID |
| | CDR-L3: | NO: 98) |
| | QQNNRWPYT (SEQ ID NO: 96) | |
| 3-M9 | CDR-H1: | VH |
| | DFNIKDDY (SEQ ID NO: 99) | EVQLQQSGAELVRPGASVKLSCTASDFNIKDDYIHWVKQ |
| | CDR-H2: | RPEQGLEWIGRIDPANGNTKYAPKFQDKATITADTSSNT |
| | IDPANGNT (SEQ ID NO: 100) | AYLQLSSLTSEDTAVYYCALGYTYWGQGTTLTVSS |
| | CDR-H3: | (SEQ ID NO: 104) |
| | ALGYTY (SEQ ID NO: 101) | |
| | CDR-L1: | VL |
| | QSLLHSYGKTY (SEQ ID NO: 102) | DVVMTQTPLTLSVTIGQPASISCKSSQSLLHSYGKTYLN |
| | CDR-L2: | WLLQRPGQSPKLLIYLVSKLESGVPDRFSGSGSGTDFTL |
| | LVS (SEQ ID NO: 60) | KISRVEAEDLGVYYCLQTTHFPQTFGGGTKLEIK (SEQ |
| | CDR-L3: | ID NO: 105) |
| | LQTTHFPQT (SEQ ID NO: 103) | |
| 10-H2 | CDR-H1: | VH |
| | GFTFSDYG (SEQ ID NO: 106) | EVQLVESGGDLVKPGGSLKLSCAASGFTFSDYGMHWVRQ |
| | CDR-H2: | GPEKGLEWVAYINSGSSTIYYADTVKGRFTISRDNAKNT |
| | INSGSSTI (SEQ ID NO: 107) | LFLQMTSLRSEDTAMYYCARPGDYDNYAMDYWGQGTSVT |
| | CDR-H3: | VSS (SEQ ID NO: 112) |
| | ARPGDYDNYAMDY (SEQ ID NO: | |
| | 108) | |
| | CDR-L1: | VL |
| | QDVSVA (SEQ ID NO: 109) | DIVMTQSHKFLSTSVGDRVSITCKASQDVSVAVAWYQQK |
| | CDR-L2: | PGQSPKLLIYWAYTRHTGVPDRFTGSGSGTEYTLTISSV |
| | WAY (SEQ ID NO: 110) | QAEDLALYYCQQHYNTPPWTFGGGTKLEIK (SEQ ID |
| | CDR-L3: | NO: 113) |
| | QQHYNTPPWT (SEQ ID NO: 111) | |
| 4-J22 | CDR-H1: | VH |
| | GFNIKDYY (SEQ ID NO: 79) | EVQLQQSGAELVRSGASVKLSCTASGFNIKDYYIHWVKQ |
| | CDR-H2: | RPEQGLEWIGWIDPENADTEYAPKFQGKATMTPDTSSNT |
| | IDPENADT (SEQ ID NO: 114) | AYLQLSSLTSEDTAVYYCYAWDYSMDYWGQGTSVTVSS |
| | CDR-H3: | (SEQ ID NO: 117) |
| | YAWDYSMDY (SEQ ID NO: 115) | |
| | CDR-L1: | VL |
| | QSLVHSNGNTY (SEQ ID NO: 82) | DVVMTQTPLSLSVSLGDQASISCRSSQSLVHSNGNTYLH |
| | CDR-L2: | WYLQKPGQSPKLLIYKVSNRFSGVPDRFSGSGSGTDFIL |
| | KVS (SEQ ID NO: 83) | KISRVEAEDLGVYFCSQNTHVPYTFGGGTRLEIK (SEQ |
| | CDR-L3: | ID NO: 118) |
| | SQNTHVPYT (SEQ ID NO: 116) | |
| 9-D4 | CDR-H1: | VH |
| | GFTFTDYG (SEQ ID NO: 119) | QVQLQQSGTELARPGASVKLSCKASGFTFTDYGINWVKQ |
| | CDR-H2: | RTGQGLEWIGEIYPSSGNSYYNEKFKAKATLTADKSSST |
| | IYPSSGNS (SEQ ID NO: 120) | AYMELRSLTSEDSAVYFCARSTYYGSPIDYWGQGTTLTV |
| | CDR-H3: | SS (SEQ ID NO: 124) |
| | ARSTYYGSPIDY (SEQ ID NO: 121) | |
| | CDR-L1: | VL |
| | QDVDTT (SEQ ID NO: 122) | DIVMTQSHKFMSTPVGDRVSITCKASQDVDTTVAWYQQK |
| | CDR-L2: | PGQSPKLLIYWASTRQIGVPDRFTGSGSGTDFTLTISNV |
| | WAS (SEQ ID NO: 45) | QSEDLADYFCQQYSTYPLTFGGGTKLEIK (SEQ ID |
| | CDR-L3: | NO: 125) |
| | QQYSTYPLT (SEQ ID NO: 123) | |
| 8-D15 | CDR-H1: | VH |
| | GFSLTSYA (SEQ ID NO: 126) | QVQLKESGPGLVAPSQSLSITCTVSGFSLTSYAITWVRQ |
| | CDR-H2: | SPGKGLEWLGLIWTGGGTNYNSALKSRLSISKDNSKSQV |
| | IWTGGGT (SEQ ID NO: 127) | FLKMNSLQTDDTARYYCARIYDGYYRYFDVWGTGTTVTV |
| | CDR-H3: | SS (SEQ ID NO: 132) |
| | ARIYDGYYRYFDV (SEQ ID NO: | |
| | 128) | |
| | CDR-L1: | VL |
| | QSVSND (SEQ ID NO: 129) | RIVLTQTPKFLLVSAGDRVTMTCKASQSVSNDVAWYQQK |
| | CDR-L2: | PGQSPKLLIYYASNRYTGVPDRFTGSGYGTDFTFTISTV |
| | YAS (SEQ ID NO: 130) | QAEDLAVYFCQQDYSSPWTFGGGTKLEIK (SEQ ID |
| | CDR-L3: | NO: 133) |
| | QQDYSSPWT (SEQ ID NO: 131) | |
| 4-H4 | CDR-H1: | VH |
| | GFNIKDYY (SEQ ID NO: 79) | EVQLQQSGAELVRSGASVKLSCTASGFNIKDYYMHWVKQ |
| | CDR-H2: | RPEQGLDWIGWIDPENGDTEYAPKFQGKATMTADTSSNT |
| | IDPENGDT (SEQ ID NO: 2) | AYLQLSSLTSEDTAVYYCNVLTMPTAYWGQGTLVTVSA |
| | CDR-H3: | (SEQ ID NO: 136) |
| | NVLTMPTAY (SEQ ID NO: 134) | |
| | CDR-L1: | VL |
| | QSLLYSSNQKNY (SEQ ID NO: 75) | DIVMSQSPSSLAVSVGEKVIMSCKSSQSLLYSSNQKNYL |
| | CDR-L2: | AWYQQKPGQSPKLLIYWASTRESGVPDRFTGSGSGTDFT |
| | WAS (SEQ ID NO: 45) | LTISSVKAEDLAVYYCQQYYSYPYTFGGGTKLEIK |
| | CDR-L3: | (SEQ ID NO: 137) |
| | QQYYSYPYT (SEQ ID NO: 135) | |
| 9-C4 | CDR-H1: | VH |
| | GFTFSSYG (SEQ ID NO: 138) | EVQLMESGGDLVKPGGSLKLSCAASGFTFSSYGLSWVRQ |
| | CDR-H2: | TPDKRLEWVATITSGGSYTYYPDSVKGRFTISRDNARNT |
| | ITSGGSYT (SEQ ID NO: 139) | LYLQMFSLKSEDTAMYYCALWSLDYWGQGTTLTVSS |
| | CDR-H3: | (SEQ ID NO: 143) |
| | ALWSLDY (SEQ ID NO: 140) | |
| | CDR-L1: | VL |
| | SSLSY (SEQ ID NO: 141) | QIVLTQSPAIMSASPGEKVTMTCSANSSLSYMHWYQQKP |
| | CDR-L2: | GTSPKRWIYDTSELASGVPARFSGSGSGTSYSLTISSME |
| | DTS (SEQ ID NO: 29) | AEDAATYYCHQRRSYPWTFGGGTKLEIK (SEQ ID |
| | CDR-L3: | NO: 144) |
| | HQRRSYPWT (SEQ ID NO: 142) | |

In some embodiments, the anti-TfR antibodies of the present disclosure comprises one or more of the CDR-H (e.g., CDR-H1, CDR-H2, and CDR-H3) amino acid sequences from any one of the anti-TfR antibodies selected from Table 2. In some embodiments, the anti-TfR antibodies of the present disclosure comprise the CDR-H1, CDR-H2, and CDR-H3 as provided for any one of the antibodies elected from Table 2. In some embodiments, the anti-TfR antibodies of the present disclosure comprises one or more of the CDR-L (e.g., CDR-L1, CDR-L2, and CDR-L3) amino acid sequences from any one of the anti-TfR antibodies selected from Table 2. In some embodiments, the anti-TfR antibodies of the present disclosure comprise the CDR-L1, CDR-L2, and CDR-L3 as provided for any one of the anti-TfR antibodies selected from Table 2.

In some embodiments, the anti-TfR antibodies of the present disclosure comprises the CDR-H1, CDR-H2, CDR-H3, CDR-L1, CDR-L2, and CDR-L3 as provided for any one of the anti-TfR antibodies selected from Table 2. In some embodiments, antibody heavy and light chain CDR3 domains may play a particularly important role in the binding specificity/affinity of an antibody for an antigen. Accordingly, the anti-TfR antibodies of the disclosure may include at least the heavy and/or (e.g., and) light chain CDR3s of any one of the anti-TfR antibodies selected from Table 2.

In some examples, any of the anti-TfR antibodies of the disclosure have one or more CDR (e.g., CDR-H or CDR-L) sequences substantially similar to any of the CDR-H1, CDR-H2, CDR-H3, CDR-L1, CDR-L2, and/or (e.g., and) CDR-L3 sequences from one of the anti-TfR antibodies selected from Table 2. In some embodiments, the position of one or more CDRs along the VH (e.g., CDR-H1, CDR-H2, or CDR-H3) and/or (e.g., and) VL (e.g., CDR-L1, CDR-L2, or CDR-L3) region of an antibody described herein can vary by one, two, three, four, five, or six amino acid positions so long as immunospecific binding to transferrin receptor (e.g., human transferrin receptor) is maintained (e.g., substantially maintained, for example, at least 50%, at least 60%, at least 70%, at least 80%, at least 90%, at least 95% of the binding of the original antibody from which it is derived). For example, in some embodiments, the position defining a CDR of any antibody described herein can vary by shifting the N-terminal and/or (e.g., and) C-terminal boundary of the CDR by one, two, three, four, five, or six amino acids, relative to the CDR position of any one of the antibodies described herein, so long as immunospecific binding to transferrin receptor (e.g., human transferrin receptor) is maintained (e.g., substantially maintained, for example, at least 50%, at least 60%, at least 70%, at least 80%, at least 90%, at least 95% of the binding of the original antibody from which it is derived). In another embodiment, the length of one or more CDRs along the VH (e.g., CDR-H1, CDR-H2, or CDR-H3) and/or (e.g., and) VL (e.g., CDR-L1, CDR-L2, or CDR-L3) region of an antibody described herein can vary (e.g., be shorter or longer) by one, two, three, four, five, or more amino acids, so long as immunospecific binding to transferrin receptor (e.g., human transferrin receptor) is maintained (e.g., substantially maintained, for example, at least 50%, at least 60%, at least 70%, at least 80%, at least 90%, at least 95% of the binding of the original antibody from which it is derived).

Accordingly, in some embodiments, a CDR-L1, CDR-L2, CDR-L3, CDR-H1, CDR-H2, and/or (e.g., and) CDR-H3 described herein may be one, two, three, four, five or more amino acids shorter than one or more of the CDRs described herein (e.g., CDRS from any of the anti-TfR antibodies selected from Table 2) so long as immunospecific binding to transferrin receptor (e.g., human transferrin receptor) is maintained (e.g., substantially maintained, for example, at least 50%, at least 60%, at least 70%, at least 80%, at least 90%, at least 95% relative to the binding of the original antibody from which it is derived). In some embodiments, a CDR-L1, CDR-L2, CDR-L3, CDR-H1, CDR-H2, and/or (e.g., and) CDR-H3 described herein may be one, two, three, four, five or more amino acids longer than one or more of the CDRs described herein (e.g., CDRS from any of the anti-TfR antibodies selected from Table 2) so long as immunospecific binding to transferrin receptor (e.g., human transferrin receptor) is maintained (e.g., substantially maintained, for example, at least 50%, at least 60%, at least 70%, at least 80%, at least 90%, at least 95% relative to the binding of the original antibody from which it is derived). In some embodiments, the amino portion of a CDR-L1, CDR-L2, CDR-L3, CDR-H1, CDR-H2, and/or (e.g., and) CDR-H3 described herein can be extended by one, two, three, four, five or more amino acids compared to one or more of the CDRs described herein (e.g., CDRS from any of the anti-TfR antibodies selected from Table 2) so long as immunospecific binding to transferrin receptor (e.g., human transferrin receptor is maintained (e.g., substantially maintained, for example, at least 50%, at least 60%, at least 70%, at least 80%, at least 90%, at least 95% relative to the binding of the original antibody from which it is derived). In some embodiments, the carboxy portion of a CDR-L1, CDR-L2, CDR-L3, CDR-H1, CDR-H2, and/or (e.g., and) CDR-H3 described herein can be extended by one, two, three, four, five or more amino acids compared to one or more of the CDRs described herein (e.g., CDRS from any of the anti-TfR antibodies selected from Table 2) so long as immunospecific binding to transferrin receptor (e.g., human transferrin receptor) is maintained (e.g., substantially maintained, for example, at least 50%, at least 60%, at least 70%, at least 80%, at least 90%, at least 95% relative to the binding of the original antibody from which it is derived). In some embodiments, the amino portion of a CDR-L1, CDR-L2, CDR-L3, CDR-H1, CDR-H2, and/or (e.g., and) CDR-H3 described herein can be shortened by one, two, three, four, five or more amino acids compared to one or more of the CDRs described herein (e.g., CDRS from any of the anti-TfR antibodies selected from Table 2) so long as immunospecific binding to transferrin receptor (e.g., human transferrin receptor) is maintained (e.g., substantially maintained, for example, at least 50%, at least 60%, at least 70%, at least 80%, at least 90%, at least 95% relative to the binding of the original antibody from which it is derived). In some embodiments, the carboxy portion of a CDR-L1, CDR-L2, CDR-L3, CDR-H1, CDR-H2, and/or (e.g., and) CDR-H3 described herein can be shortened by one, two, three, four, five or more amino acids compared to one or more of the CDRs described herein (e.g., CDRS from any of the anti-TfR antibodies selected from Table 2) so long as immunospecific binding to transferrin receptor (e.g., human transferrin receptor) is maintained (e.g., substantially maintained, for example, at least 50%, at least 60%, at least 70%, at least 80%, at least 90%, at least 95% relative to the binding of the original antibody from which it is derived). Any method can be used to ascertain whether immunospecific binding to transferrin receptor (e.g., human transferrin receptor) is maintained, for example, using binding assays and conditions described in the art.

In some examples, any of the anti-TfR antibodies of the disclosure have one or more CDR (e.g., CDR-H or CDR-L) sequences substantially similar to any one of the anti-TfR antibodies selected from Table 2. For example, the antibodies may include one or more CDR sequence(s) from any of the anti-TfR antibodies selected from Table 2 containing up to 5, 4, 3, 2, or 1 amino acid residue variations as compared to the corresponding CDR region in any one of the CDRs provided herein (e.g., CDRs from any of the anti-TfR antibodies selected from Table 2) so long as immunospecific binding to transferrin receptor (e.g., human transferrin receptor) is maintained (e.g., substantially maintained, for example, at least 50%, at least 60%, at least 70%, at least 80%, at least 90%, at least 95% relative to the binding of the original antibody from which it is derived). In some embodiments, any of the amino acid variations in any of the CDRs provided herein may be conservative variations. Conservative variations can be introduced into the CDRs at positions where the residues are not likely to be involved in interacting with a transferrin receptor protein (e.g., a human transferrin receptor protein), for example, as determined based on a crystal structure. Some aspects of the disclosure provide anti-TfR antibodies that comprise one or more of the heavy chain variable (VH) and/or (e.g., and) light chain variable (VL) domains provided herein. In some embodiments, any of the VH domains provided herein include one or more of the CDR-H sequences (e.g., CDR-H1, CDR-H2, and CDR-H3) provided herein, for example, any of the CDR-H sequences provided in any one of the anti-TfR selected from Table 2. In some embodiments, any of the VL domains provided herein include one or more of the CDR-L sequences (e.g., CDR-L1, CDR-L2, and CDR-L3) provided herein, for example, any of the CDR-L sequences provided in any one of the anti-TfR antibodies selected from Table 2.

In some embodiments, the anti-TfR antibodies of the disclosure include any antibody that includes a heavy chain variable domain and/or (e.g., and) a light chain variable domain of any one of the anti-TfR antibodies selected from Table 2, and variants thereof. In some embodiments, anti-TfR antibodies of the disclosure include any antibody that includes the heavy chain variable and light chain variable pairs of any anti-TfR antibodies selected from Table 2.

Aspects of the disclosure provide anti-TfR antibodies having a heavy chain variable (VH) and/or (e.g., and) a light chain variable (VL) domain amino acid sequence homologous to any of those described herein. In some embodiments, the anti-TfR antibody comprises a heavy chain variable sequence or a light chain variable sequence that is at least 75% (e.g., 80%, 85%, 90%, 95%, 98%, or 99%) identical to the heavy chain variable sequence and/ or any light chain variable sequence of any one of the anti-TfR antibodies selected from Table 2. In some embodiments, the homologous heavy chain variable and/or (e.g., and) a light chain variable amino acid sequences do not vary within any of the CDR sequences provided herein. For example, in some embodiments, the degree of sequence variation (e.g., 75%, 80%, 85%, 90%, 95%, 98%, or 99%) may occur within a heavy chain variable and/or (e.g., and) a light chain variable sequence excluding any of the CDR sequences provided herein. In some embodiments, any of the anti-TfR antibodies provided herein comprise a heavy chain variable sequence and a light chain variable sequence that comprises a framework sequence that is at least 75%, 80%, 85%, 90%, 95%, 98%, or 99% identical to the framework sequence of any anti-TfR antibodies selected from Table 2.

In some embodiments, the anti-TfR antibody of the present disclosure comprises a CDR-H1, a CDR-H2, and a CDR-H3 of a heavy chain variable domain having the amino acid sequence of SEQ ID NO: 7. Alternatively or in addition (e.g., in addition), the anti-TfR antibody of the present disclosure comprises a CDR-L1, a CDR-L2, and a CDR-L3 of a light chain variable domain having the amino acid sequence of SEQ ID NO: 8.

In some embodiments, the anti-TfR antibody of the present disclosure comprises a CDR-H1 having the amino acid sequence of SEQ ID NO: 1 (according to the IMGT definition system), a CDR-H2 having the amino acid sequence of SEQ ID NO: 2 (according to the IMGT definition system), a CDR-H3 having the amino acid sequence of SEQ ID NO: 3 (according to the IMGT definition system), a CDR-L1 having the amino acid sequence of SEQ ID NO: 4 (according to the IMGT definition system), a CDR-L2 having the amino acid sequence of SEQ ID NO: 5 (according to the IMGT definition system), and a CDR-L3 having the amino acid sequence of SEQ ID NO: 6 (according to the IMGT definition system).

In some embodiments, the anti-TfR antibody of the present disclosure comprises: a CDR-H1 having the amino acid sequence of SEQ ID NO: 1; a CDR-H2 having the amino acid sequence of SEQ ID NO: 2 with an amino acid substitution at position 5 (e.g., the asparagine at position 5 is substituted, e.g., with any one of Arg (R), Lys (K), Asp (D), Glu (E), Gln (Q), His (H), Ser (S), Thr (T), Tyr (Y), Cys (C), Trp (W), Met (M), Ala (A), Ile (I), Leu (L), Phe (F), Val (V), Pro (P), Gly (G)); and a CDR-H3 having the amino acid sequence of SEQ ID NO: 3. Alternatively or in addition (e.g., in addition), the anti-TfR antibody of the present disclosure comprises: a CDR-L1 having the amino acid sequence of SEQ ID NO: 4; a CDR-L2 having the amino acid sequence of SEQ ID NO: 5; and a CDR-L3 having the amino acid sequence of SEQ ID NO: 6. In some embodiments, the amino acid substitution at position 5 of the CDR-H2 as set forth in SEQ ID NO: 2 is N5T or N5S.

In some embodiments, the anti-TfR antibody of the present disclosure comprises: a CDR-H1 having the amino acid sequence of SEQ ID NO: 1; a CDR-H2 having the amino acid sequence of SEQ ID NO: 262 or SEQ ID NO: 80; and a CDR-H3 having the amino acid sequence of SEQ ID NO: 3. Alternatively or in addition (e.g., in addition), the anti-TfR antibody of the present disclosure comprises: a CDR-L1 having the amino acid sequence of SEQ ID NO: 4; a CDR-L2 having the amino acid sequence of SEQ ID NO: 5; and a CDR-L3 having the amino acid sequence of SEQ ID NO: 6.

In some embodiments, anti-TfR antibody of the present disclosure comprises a CDR-H1, a CDR-H2, and a CDR-H3, which collectively contains no more than 5 amino acid variations (e.g., no more than 5, 4, 3, 2, or 1 amino acid variation) as compared with the CDR-H1 having the amino acid sequence of SEQ ID NO: 1, CDR-H2 having the amino acid sequence of SEQ ID NO: 2, SEQ ID NO: 262 or SEQ ID NO: 80, and CDR-H3 having the amino acid sequence of SEQ ID NO: 3. “Collectively,” as used anywhere in the present disclosure, means that the total number of amino acid variations in all of the three heavy chain CDRs is within the defined range. Alternatively or in addition (e.g., in addition), the anti-TfR antibody of the present disclosure comprises a CDR-L1, a CDR-L2, and a CDR-L3, which collectively contains no more than 5 amino acid variations (e.g., no more than 5, 4, 3, 2 or 1 amino acid variation) as compared with the CDR-L1 having the amino acid sequence of SEQ ID NO: 4, CDR-L2 having the amino acid sequence of SEQ ID NO: 5, and CDR-L3 having the amino acid sequence of SEQ ID NO: 6.

In some embodiments, the anti-TfR antibody of the present disclosure comprises a CDR-H1, a CDR-H2, and a CDR-H3 that collectively are at least 75% (e.g., 75%, 80%, 85%, 90%, 95%, 98%, or 99%) identical to the CDR-H1 having the amino acid sequence of SEQ ID NO: 1, CDR-H2 having the amino acid sequence of SEQ ID NO: 2, SEQ ID NO: 262 or SEQ ID NO: 80, and CDR-H3 having the amino acid sequence of SEQ ID NO: 3. Alternatively or in addition (e.g., in addition), the anti-TfR antibody of the present disclosure comprises a CDR-L1, a CDR-L2, and a CDR-L3 that collectively are at least 75% (e.g., 75%, 80%, 85%, 90%, 95%, 98%, or 99%) identical to the to the CDR-L1 having the amino acid sequence of SEQ ID NO: 4, CDR-L2 having the amino acid sequence of SEQ ID NO: 5, and CDR-L3 having the amino acid sequence of SEQ ID NO: 6.

In some embodiments, the anti-TfR antibody of the present disclosure comprises: a CDR-H1 having no more than 3 amino acid variations (e.g., no more than 3, 2, or 1 amino acid variation) as compared with the CDR-H1 having the amino acid sequence of SEQ ID NO: 1; a CDR-H2 having no more than 3 amino acid variations (e.g., no more than 3, 2, or 1 amino acid variation) as compared with the CDR-H2 having the amino acid sequence of SEQ ID NO: 2, SEQ ID NO: 262 or SEQ ID NO: 80; and/or (e.g., and) a CDR-H3 having no more than 3 amino acid variations (e.g., no more than 3, 2, or 1 amino acid variation) as compared with the CDR-H3 having the amino acid sequence of SEQ ID NO: 3. Alternatively or in addition (e.g., in addition), the anti-TfR antibody of the present disclosure comprises: a CDR-L1 having no more than 3 amino acid variations (e.g., no more than 3, 2, or 1 amino acid variation) as compared with the CDR-L1 having the amino acid sequence of SEQ ID NO: 4; a CDR-L2 having no more than 3 amino acid variations (e.g., no more than 3, 2, or 1 amino acid variation) as compared with the CDR-L2 having the amino acid sequence of SEQ ID NO: 5; and/or (e.g., and) a CDR-L3 having no more than 3 amino acid variations (e.g., no more than 3, 2, or 1 amino acid variation) as compared with the CDR-L3 having the amino acid sequence of SEQ ID NO: 6.

In some embodiments, the anti-TfR antibody of the present disclosure comprises a VH comprising the amino acid sequence of SEQ ID NO: 7. Alternatively or in addition (e.g., in addition), the anti-TfR antibody of the present disclosure comprises a VL comprising the amino acid sequence of SEQ ID NO: 8.

In some embodiments, the anti-TfR antibody of the present disclosure comprises a VH containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation)no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 7. Alternatively or in addition (e.g., in addition), the anti-TfR antibody of the present disclosure comprises a VL containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VL as set forth in SEQ ID NO: 8.

In some embodiments, the anti-TfR antibody of the present disclosure comprises a VH comprising an amino acid sequence that is at least 75% (e.g., 75%, 80%, 85%, 90%, 95%, 98%, or 99%) identical to the VH as set forth in SEQ ID NO: 7. Alternatively or in addition (e.g., in addition), the anti-TfR antibody of the present disclosure comprises a VL comprising an amino acid sequence that is at least 75% (e.g., 75%, 80%, 85%, 90%, 95%, 98%, or 99%) identical to the VL as set forth in SEQ ID NO: 8.

In some embodiments, the anti-TfR antibody of the present disclosure comprises a VH as set forth in SEQ ID NO: 7 with an amino acid substitution at position 55 (e.g., the asparagine at position 55 is substituted, e.g., with any one of Arg (R), Lys (K), Asp (D), Glu (E), Gln (Q), His (H), Ser (S), Thr (T), Tyr (Y), Cys (C), Trp (W), Met (M), Ala (A), Ile (I), Leu (L), Phe (F), Val (V), Pro (P), Gly (G)). Alternatively or in addition (e.g., in addition), the anti-TfR antibody of the present disclosure comprises a VL as set forth in SEQ ID NO: 8. In some embodiments, the amino acid substitution at position 55 of the VH as set forth in SEQ ID NO: 7 is N55T or N55S. Amino acid position 55 in SEQ ID NO: 7 is assigned a number 54 when the VH set forth in SEQ ID NO: 7 is annotated using the Kabat numbering system. When N54T or N54S is referred to herein, it is referring to the mutations using the Kabat numbering system.

In some embodiments, the anti-TfR antibody of the present disclosure comprises a VH comprising an amino acid substitution at position 64 relative to SEQ ID NO: 7. In some embodiments, the anti-TfR antibody of the present disclosure comprises a VH comprising a Met at a position corresponding to position 64 of SEQ ID NO: 7. Alternatively or in addition (e.g., in addition), the anti-TfR antibody of the present disclosure comprises a VL comprising an amino acid sequence that is at least 80% (e.g., 80%, 85%, 90%, 95%, 98%, 99%, or 100%) identical to the VL as set forth in SEQ ID NO: 8.

In some embodiments, the anti-TfR antibody of the present disclosure comprises a CDR-H1, a CDR-H2, and a CDR-H3 of a heavy chain variable domain having the amino acid sequence of SEQ ID NO: 15. Alternatively or in addition (e.g., in addition), the anti-TfR antibody of the present disclosure comprises a CDR-L1, a CDR-L2, and a CDR-L3 of a light chain variable domain having the amino acid sequence of SEQ ID NO: 16.

In some embodiments, the anti-TfR antibody of the present disclosure comprises a CDR-H1 having the amino acid sequence of SEQ ID NO: 9 (according to the IMGT definition system), a CDR-H2 having the amino acid sequence of SEQ ID NO: 10 (according to the IMGT definition system), a CDR-H3 having the amino acid sequence of SEQ ID NO: 11 (according to the IMGT definition system), a CDR-L1 having the amino acid sequence of SEQ ID NO: 12 (according to the IMGT definition system), a CDR-L2 having the amino acid sequence of SEQ ID NO: 13 (according to the IMGT definition system), and a CDR-L3 having the amino acid sequence of SEQ ID NO: 14 (according to the IMGT definition system).

In some embodiments, anti-TfR antibody of the present disclosure comprises a CDR-H1, a CDR-H2, and a CDR-H3, which collectively contains no more than 5 amino acid variations (e.g., no more than 5, 4, 3, 2, or 1 amino acid variation) as compared with the CDR-H1 having the amino acid sequence of SEQ ID NO: 9, CDR-H2 having the amino acid sequence of SEQ ID NO: 10, and CDR-H3 having the amino acid sequence of SEQ ID NO: 11. Alternatively or in addition (e.g., in addition), the anti-TfR antibody of the present disclosure comprises a CDR-L1, a CDR-L2, and a CDR-L3, which collectively contains no more than 5 amino acid variations (e.g., no more than 5, 4, 3, 2 or 1 amino acid variation) as compared with the CDR-L1 having the amino acid sequence of SEQ ID NO: 12, CDR-L2 having the amino acid sequence of SEQ ID NO: 13, and CDR-L3 having the amino acid sequence of SEQ ID NO: 14.

In some embodiments, the anti-TfR antibody of the present disclosure comprises a CDR-H1, a CDR-H2, and a CDR-H3 that collectively are at least 75% (e.g., 75%, 80%, 85%, 90%, 95%, 98%, or 99%) identical to the CDR-H1 having the amino acid sequence of SEQ ID NO: 9, CDR-H2 having the amino acid sequence of SEQ ID NO: 10, and CDR-H3 having the amino acid sequence of SEQ ID NO: 11. Alternatively or in addition (e.g., in addition), the anti-TfR antibody of the present disclosure comprises a CDR-L1, a CDR-L2, and a CDR-L3 that collectively are at least 75% (e.g., 75%, 80%, 85%, 90%, 95%, 98%, or 99%) identical to the to the CDR-L1 having the amino acid sequence of SEQ ID NO: 12, CDR-L2 having the amino acid sequence of SEQ ID NO: 13, and CDR-L3 having the amino acid sequence of SEQ ID NO: 14.

In some embodiments, the anti-TfR antibody of the present disclosure comprises: a CDR-H1 having no more than 3 amino acid variations (e.g., no more than 3, 2, or 1 amino acid variation) as compared with the CDR-H1 having the amino acid sequence of SEQ ID NO: 9; a CDR-H2 having no more than 3 amino acid variations (e.g., no more than 3, 2, or 1 amino acid variation) as compared with the CDR-H2 having the amino acid sequence of SEQ ID NO: 10; and/or (e.g., and) a CDR-H3 having no more than 3 amino acid variations (e.g., no more than 3, 2, or 1 amino acid variation) as compared with the CDR-H3 having the amino acid sequence of SEQ ID NO: 11. Alternatively or in addition (e.g., in addition), the anti-TfR antibody of the present disclosure comprises: a CDR-L1 having no more than 3 amino acid variations (e.g., no more than 3, 2, or 1 amino acid variation) as compared with the CDR-L1 having the amino acid sequence of SEQ ID NO: 12; a CDR-L2 having no more than 3 amino acid variations (e.g., no more than 3, 2, or 1 amino acid variation) as compared with the CDR-L2 having the amino acid sequence of SEQ ID NO: 13; and/or (e.g., and) a CDR-L3 having no more than 3 amino acid variations (e.g., no more than 3, 2, or 1 amino acid variation) as compared with the CDR-L3 having the amino acid sequence of SEQ ID NO: 14.

In some embodiments, the anti-TfR antibody of the present disclosure comprises a VH comprising the amino acid sequence of SEQ ID NO: 15. Alternatively or in addition (e.g., in addition), the anti-TfR antibody of the present disclosure comprises a VL comprising the amino acid sequence of SEQ ID NO: 16.

In some embodiments, the anti-TfR antibody of the present disclosure comprises a VH containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 15. Alternatively or in addition (e.g., in addition), the anti-TfR antibody of the present disclosure comprises a VL containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VL as set forth in SEQ ID NO: 16.

In some embodiments, the anti-TfR antibody of the present disclosure comprises a VH comprising an amino acid sequence that is at least 75% (e.g., 75%, 80%, 85%, 90%, 95%, 98%, or 99%) identical to the VH as set forth in SEQ ID NO: 15. Alternatively or in addition (e.g., in addition), the anti-TfR antibody of the present disclosure comprises a VL comprising an amino acid sequence that is at least 75% (e.g., 75%, 80%, 85%, 90%, 95%, 98%, or 99%) identical to the VL as set forth in SEQ ID NO: 16.

In some embodiments, the anti-TfR antibody of the present disclosure comprises a CDR-H1, a CDR-H2, and a CDR-H3 of a heavy chain variable domain having the amino acid sequence of SEQ ID NO: 23. Alternatively or in addition (e.g., in addition), the anti-TfR antibody of the present disclosure comprises a CDR-L1, a CDR-L2, and a CDR-L3 of a light chain variable domain having the amino acid sequence of SEQ ID NO: 24.

In some embodiments, the anti-TfR antibody of the present disclosure comprises a CDR-H1 having the amino acid sequence of SEQ ID NO: 17 (according to the IMGT definition system), a CDR-H2 having the amino acid sequence of SEQ ID NO: 18 (according to the IMGT definition system), a CDR-H3 having the amino acid sequence of SEQ ID NO: 19 (according to the IMGT definition system), a CDR-L1 having the amino acid sequence of SEQ ID NO: 20 (according to the IMGT definition system), a CDR-L2 having the amino acid sequence of SEQ ID NO: 21 (according to the IMGT definition system), and a CDR-L3 having the amino acid sequence of SEQ ID NO: 22 (according to the IMGT definition system).

In some embodiments, the anti-TfR antibody of the present disclosure comprises: a CDR-H1 having the amino acid sequence of SEQ ID NO: 17 with an amino acid substitution at position 8 (e.g., the cysteine at position 8 is substituted, e.g., with any one of Arg (R), Lys (K), Asp (D), Glu (E), Gln (Q), His (H), Ser (S), Thr (T), Tyr (Y), Asn (N), Trp (W), Met (M), Ala (A), Ile (I), Leu (L), Phe (F), Val (V), Pro (P), Gly (G)); a CDR-H2 having the amino acid sequence of SEQ ID NO: 18; and a CDR-H3 having the amino acid sequence of SEQ ID NO: 19. Alternatively or in addition (e.g., in addition), the anti-TfR antibody of the present disclosure comprises: a CDR-L1 having the amino acid sequence of SEQ ID NO: 20; a CDR-L2 having the amino acid sequence of SEQ ID NO: 21; and a CDR-L3 having the amino acid sequence of SEQ ID NO: 22. In some embodiments, the amino acid substitution at position 8 of the CDR-H1 as set forth in SEQ ID NO: 17 is C8D or C8Y.

In some embodiments, the anti-TfR antibody of the present disclosure comprises: a CDR-H1 having the amino acid sequence of SEQ ID NO: 266 or SEQ ID NO: 268; a CDR-H2 having the amino acid sequence of SEQ ID NO: 18; and a CDR-H3 having the amino acid sequence of SEQ ID NO: 19. Alternatively or in addition (e.g., in addition), the anti-TfR antibody of the present disclosure comprises: a CDR-L1 having the amino acid sequence of SEQ ID NO: 20; a CDR-L2 having the amino acid sequence of SEQ ID NO: 21; and a CDR-L3 having the amino acid sequence of SEQ ID NO: 22.

In some embodiments, anti-TfR antibody of the present disclosure comprises a CDR-H1, a CDR-H2, and a CDR-H3, which collectively contains no more than 5 amino acid variations (e.g., no more than 5, 4, 3, 2, or 1 amino acid variation) as compared with the CDR-H1 having the amino acid sequence of SEQ ID NO: 17, SEQ ID NO: 266, or SEQ ID NO: 268, CDR-H2 having the amino acid sequence of SEQ ID NO: 18, and CDR-H3 having the amino acid sequence of SEQ ID NO: 19. Alternatively or in addition (e.g., in addition), the anti-TfR antibody of the present disclosure comprises a CDR-L1, a CDR-L2, and a CDR-L3, which collectively contains no more than 5 amino acid variations (e.g., no more than 5, 4, 3, 2 or 1 amino acid variation) as compared with the CDR-L1 having the amino acid sequence of SEQ ID NO: 20, CDR-L2 having the amino acid sequence of SEQ ID NO: 21, and CDR-L3 having the amino acid sequence of SEQ ID NO: 22.

In some embodiments, the anti-TfR antibody of the present disclosure comprises a CDR-H1, a CDR-H2, and a CDR-H3 that collectively are at least 75% (e.g., 75%, 80%, 85%, 90%, 95%, 98%, or 99%) identical to the CDR-H1 having the amino acid sequence of SEQ ID NO: 17, SEQ ID NO: 266, or SEQ ID NO: 268, CDR-H2 having the amino acid sequence of SEQ ID NO: 18, and CDR-H3 having the amino acid sequence of SEQ ID NO: 19. Alternatively or in addition (e.g., in addition), the anti-TfR antibody of the present disclosure comprises a CDR-L1, a CDR-L2, and a CDR-L3 that collectively are at least 75% (e.g., 75%, 80%, 85%, 90%, 95%, 98%, or 99%) identical to the to the CDR-L1 having the amino acid sequence of SEQ ID NO: 20, CDR-L2 having the amino acid sequence of SEQ ID NO: 21, and CDR-L3 having the amino acid sequence of SEQ ID NO: 22.

In some embodiments, the anti-TfR antibody of the present disclosure comprises: a CDR-H1 having no more than 3 amino acid variations (e.g., no more than 3, 2, or 1 amino acid variation) as compared with the CDR-H1 having the amino acid sequence of SEQ ID NO: 17, SEQ ID NO: 266, or SEQ ID NO: 268; a CDR-H2 having no more than 3 amino acid variations (e.g., no more than 3, 2, or 1 amino acid variation) as compared with the CDR-H2 having the amino acid sequence of SEQ ID NO: 18; and/or (e.g., and) a CDR-H3 having no more than 3 amino acid variations (e.g., no more than 3, 2, or 1 amino acid variation) as compared with the CDR-H3 having the amino acid sequence of SEQ ID NO: 19. Alternatively or in addition (e.g., in addition), the anti-TfR antibody of the present disclosure comprises: a CDR-L1 having no more than 3 amino acid variations (e.g., no more than 3, 2, or 1 amino acid variation) as compared with the CDR-L1 having the amino acid sequence of SEQ ID NO: 20; a CDR-L2 having no more than 3 amino acid variations (e.g., no more than 3, 2, or 1 amino acid variation) as compared with the CDR-L2 having the amino acid sequence of SEQ ID NO: 21; and/or (e.g., and) a CDR-L3 having no more than 3 amino acid variations (e.g., no more than 3, 2, or 1 amino acid variation) as compared with the CDR-L3 having the amino acid sequence of SEQ ID NO: 22.

In some embodiments, the anti-TfR antibody of the present disclosure comprises a VH comprising the amino acid sequence of SEQ ID NO: 23. Alternatively or in addition (e.g., in addition), the anti-TfR antibody of the present disclosure comprises a VL comprising the amino acid sequence of SEQ ID NO: 24.

In some embodiments, the anti-TfR antibody of the present disclosure comprises a VH containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation)no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 23. Alternatively or in addition (e.g., in addition), the anti-TfR antibody of the present disclosure comprises a VL containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VL as set forth in SEQ ID NO: 24.

In some embodiments, the anti-TfR antibody of the present disclosure comprises a VH comprising an amino acid sequence that is at least 75% (e.g., 75%, 80%, 85%, 90%, 95%, 98%, or 99%) identical to the VH as set forth in SEQ ID NO: 23. Alternatively or in addition (e.g., in addition), the anti-TfR antibody of the present disclosure comprises a VL comprising an amino acid sequence that is at least 75% (e.g., 75%, 80%, 85%, 90%, 95%, 98%, or 99%) identical to the VL as set forth in SEQ ID NO: 24.

In some embodiments, the anti-TfR antibody of the present disclosure comprises a VH as set forth in SEQ ID NO: 23 with an amino acid substitution at position 33 (e.g., the cysteine at position 33 is substituted, e.g., with any one of Arg (R), Lys (K), Asp (D), Glu (E), Gln (Q), His (H), Ser (S), Thr (T), Tyr (Y), Asn (N), Trp (W), Met (M), Ala (A), Ile (I), Leu (L), Phe (F), Val (V), Pro (P), Gly (G)). Alternatively or in addition (e.g., in addition), the anti-TfR antibody of the present disclosure comprises a VL as set forth in SEQ ID NO: 24. In some embodiments, the amino acid substitution at position 33 of the VH as set forth in SEQ ID NO: 23 is C33D or C33Y. Amino acid 33 in SEQ ID NO: 23 is assigned a number 33 when the VH set forth in SEQ ID NO: 23 is annotated with the Kabat numbering system.

In some embodiments, the anti-TfR antibody of the present disclosure comprises a CDR-H1, a CDR-H2, and a CDR-H3 of a heavy chain variable domain having the amino acid sequence of SEQ ID NO: 31. Alternatively or in addition (e.g., in addition), the anti-TfR antibody of the present disclosure comprises a CDR-L1, a CDR-L2, and a CDR-L3 of a light chain variable domain having the amino acid sequence of SEQ ID NO: 32.

In some embodiments, the anti-TfR antibody of the present disclosure comprises a CDR-H1 having the amino acid sequence of SEQ ID NO: 25 (according to the IMGT definition system), a CDR-H2 having the amino acid sequence of SEQ ID NO: 26 (according to the IMGT definition system), a CDR-H3 having the amino acid sequence of SEQ ID NO: 27 (according to the IMGT definition system), a CDR-L1 having the amino acid sequence of SEQ ID NO: 28 (according to the IMGT definition system), a CDR-L2 having the amino acid sequence of SEQ ID NO: 29 (according to the IMGT definition system), and a CDR-L3 having the amino acid sequence of SEQ ID NO: 30 (according to the IMGT definition system).

In some embodiments, anti-TfR antibody of the present disclosure comprises a CDR-H1, a CDR-H2, and a CDR-H3, which collectively contains no more than 5 amino acid variations (e.g., no more than 5, 4, 3, 2, or 1 amino acid variation) as compared with the CDR-H1 having the amino acid sequence of SEQ ID NO: 25, CDR-H2 having the amino acid sequence of SEQ ID NO: 26, and CDR-H3 having the amino acid sequence of SEQ ID NO: 27. Alternatively or in addition (e.g., in addition), the anti-TfR antibody of the present disclosure comprises a CDR-L1, a CDR-L2, and a CDR-L3, which collectively contains no more than 5 amino acid variations (e.g., no more than 5, 4, 3, 2 or 1 amino acid variation) as compared with the CDR-L1 having the amino acid sequence of SEQ ID NO: 28, CDR-L2 having the amino acid sequence of SEQ ID NO: 29, and CDR-L3 having the amino acid sequence of SEQ ID NO: 30.

In some embodiments, the anti-TfR antibody of the present disclosure comprises a CDR-H1, a CDR-H2, and a CDR-H3 that collectively are at least 75% (e.g., 75%, 80%, 85%, 90%, 95%, 98%, or 99%) identical to the CDR-H1 having the amino acid sequence of SEQ ID NO: 25, CDR-H2 having the amino acid sequence of SEQ ID NO: 26, and CDR-H3 having the amino acid sequence of SEQ ID NO: 27. Alternatively or in addition (e.g., in addition), the anti-TfR antibody of the present disclosure comprises a CDR-L1, a CDR-L2, and a CDR-L3 that collectively are at least 75% (e.g., 75%, 80%, 85%, 90%, 95%, 98%, or 99%) identical to the to the CDR-L1 having the amino acid sequence of SEQ ID NO: 28, CDR-L2 having the amino acid sequence of SEQ ID NO: 29, and CDR-L3 having the amino acid sequence of SEQ ID NO: 30.

In some embodiments, the anti-TfR antibody of the present disclosure comprises: a CDR-H1 having no more than 3 amino acid variations (e.g., no more than 3, 2, or 1 amino acid variation) as compared with the CDR-H1 having the amino acid sequence of SEQ ID NO: 25; a CDR-H2 having no more than 3 amino acid variations (e.g., no more than 3, 2, or 1 amino acid variation) as compared with the CDR-H2 having the amino acid sequence of SEQ ID NO: 26; and/or (e.g., and) a CDR-H3 having no more than 3 amino acid variations (e.g., no more than 3, 2, or 1 amino acid variation) as compared with the CDR-H3 having the amino acid sequence of SEQ ID NO: 27. Alternatively or in addition (e.g., in addition), the anti-TfR antibody of the present disclosure comprises: a CDR-L1 having no more than 3 amino acid variations (e.g., no more than 3, 2, or 1 amino acid variation) as compared with the CDR-L1 having the amino acid sequence of SEQ ID NO: 28; a CDR-L2 having no more than 3 amino acid variations (e.g., no more than 3, 2, or 1 amino acid variation) as compared with the CDR-L2 having the amino acid sequence of SEQ ID NO: 29; and/or (e.g., and) a CDR-L3 having no more than 3 amino acid variations (e.g., no more than 3, 2, or 1 amino acid variation) as compared with the CDR-L3 having the amino acid sequence of SEQ ID NO: 30.

In some embodiments, the anti-TfR antibody of the present disclosure comprises a VH comprising the amino acid sequence of SEQ ID NO: 31. Alternatively or in addition (e.g., in addition), the anti-TfR antibody of the present disclosure comprises a VL comprising the amino acid sequence of SEQ ID NO: 32.

In some embodiments, the anti-TfR antibody of the present disclosure comprises a VH containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation)no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 31. Alternatively or in addition (e.g., in addition), the anti-TfR antibody of the present disclosure comprises a VL containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VL as set forth in SEQ ID NO: 32.

In some embodiments, the anti-TfR antibody of the present disclosure comprises a VH comprising an amino acid sequence that is at least 75% (e.g., 75%, 80%, 85%, 90%, 95%, 98%, or 99%) identical to the VH as set forth in SEQ ID NO: 31. Alternatively or in addition (e.g., in addition), the anti-TfR antibody of the present disclosure comprises a VL comprising an amino acid sequence that is at least 75% (e.g., 75%, 80%, 85%, 90%, 95%, 98%, or 99%) identical to the VL as set forth in SEQ ID NO: 32.

In some embodiments, the anti-TfR antibody of the present disclosure comprises a CDR-H1, a CDR-H2, and a CDR-H3 of a heavy chain variable domain having the amino acid sequence of SEQ ID NO: 39. Alternatively or in addition (e.g., in addition), the anti-TfR antibody of the present disclosure comprises a CDR-L1, a CDR-L2, and a CDR-L3 of a light chain variable domain having the amino acid sequence of SEQ ID NO: 40.

In some embodiments, the anti-TfR antibody of the present disclosure comprises a CDR-H1 having the amino acid sequence of SEQ ID NO: 33 (according to the IMGT definition system), a CDR-H2 having the amino acid sequence of SEQ ID NO: 34 (according to the IMGT definition system), a CDR-H3 having the amino acid sequence of SEQ ID NO: 35 (according to the IMGT definition system), a CDR-L1 having the amino acid sequence of SEQ ID NO: 36 (according to the IMGT definition system), a CDR-L2 having the amino acid sequence of SEQ ID NO: 37 (according to the IMGT definition system), and a CDR-L3 having the amino acid sequence of SEQ ID NO: 38 (according to the IMGT definition system).

In some embodiments, anti-TfR antibody of the present disclosure comprises a CDR-H1, a CDR-H2, and a CDR-H3, which collectively contains no more than 5 amino acid variations (e.g., no more than 5, 4, 3, 2, or 1 amino acid variation) as compared with the CDR-H1 having the amino acid sequence of SEQ ID NO: 33, CDR-H2 having the amino acid sequence of SEQ ID NO: 34, and CDR-H3 having the amino acid sequence of SEQ ID NO: 35. Alternatively or in addition (e.g., in addition), the anti-TfR antibody of the present disclosure comprises a CDR-L1, a CDR-L2, and a CDR-L3, which collectively contains no more than 5 amino acid variations (e.g., no more than 5, 4, 3, 2 or 1 amino acid variation) as compared with the CDR-L1 having the amino acid sequence of SEQ ID NO: 36, CDR-L2 having the amino acid sequence of SEQ ID NO: 37, and CDR-L3 having the amino acid sequence of SEQ ID NO: 38.

In some embodiments, the anti-TfR antibody of the present disclosure comprises a CDR-H1, a CDR-H2, and a CDR-H3 that collectively are at least 75% (e.g., 75%, 80%, 85%, 90%, 95%, 98%, or 99%) identical to the CDR-H1 having the amino acid sequence of SEQ ID NO: 33, CDR-H2 having the amino acid sequence of SEQ ID NO: 34, and CDR-H3 having the amino acid sequence of SEQ ID NO: 35. Alternatively or in addition (e.g., in addition), the anti-TfR antibody of the present disclosure comprises a CDR-L1, a CDR-L2, and a CDR-L3 that collectively are at least 75% (e.g., 75%, 80%, 85%, 90%, 95%, 98%, or 99%) identical to the to the CDR-L1 having the amino acid sequence of SEQ ID NO: 36, CDR-L2 having the amino acid sequence of SEQ ID NO: 37, and CDR-L3 having the amino acid sequence of SEQ ID NO: 38.

In some embodiments, the anti-TfR antibody of the present disclosure comprises: a CDR-H1 having no more than 3 amino acid variations (e.g., no more than 3, 2, or 1 amino acid variation) as compared with the CDR-H1 having the amino acid sequence of SEQ ID NO: 33; a CDR-H2 having no more than 3 amino acid variations (e.g., no more than 3, 2, or 1 amino acid variation) as compared with the CDR-H2 having the amino acid sequence of SEQ ID NO: 34; and/or (e.g., and) a CDR-H3 having no more than 3 amino acid variations (e.g., no more than 3, 2, or 1 amino acid variation) as compared with the CDR-H3 having the amino acid sequence of SEQ ID NO: 35. Alternatively or in addition (e.g., in addition), the anti-TfR antibody of the present disclosure comprises: a CDR-L1 having no more than 3 amino acid variations (e.g., no more than 3, 2, or 1 amino acid variation) as compared with the CDR-L1 having the amino acid sequence of SEQ ID NO: 36; a CDR-L2 having no more than 3 amino acid variations (e.g., no more than 3, 2, or 1 amino acid variation) as compared with the CDR-L2 having the amino acid sequence of SEQ ID NO: 37; and/or (e.g., and) a CDR-L3 having no more than 3 amino acid variations (e.g., no more than 3, 2, or 1 amino acid variation) as compared with the CDR-L3 having the amino acid sequence of SEQ ID NO: 38.

In some embodiments, the anti-TfR antibody of the present disclosure comprises a VH comprising the amino acid sequence of SEQ ID NO: 39. Alternatively or in addition (e.g., in addition), the anti-TfR antibody of the present disclosure comprises a VL comprising the amino acid sequence of SEQ ID NO: 40.

In some embodiments, the anti-TfR antibody of the present disclosure comprises a VH containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 39. Alternatively or in addition (e.g., in addition), the anti-TfR antibody of the present disclosure comprises a VL containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VL as set forth in SEQ ID NO: 40.

In some embodiments, the anti-TfR antibody of the present disclosure comprises a VH comprising an amino acid sequence that is at least 75% (e.g., 75%, 80%, 85%, 90%, 95%, 98%, or 99%) identical to the VH as set forth in SEQ ID NO: 39. Alternatively or in addition (e.g., in addition), the anti-TfR antibody of the present disclosure comprises a VL comprising an amino acid sequence that is at least 75% (e.g., 75%, 80%, 85%, 90%, 95%, 98%, or 99%) identical to the VL as set forth in SEQ ID NO: 40.

In some embodiments, the anti-TfR antibody of the present disclosure comprises a CDR-H1, a CDR-H2, and a CDR-H3 of a heavy chain variable domain having the amino acid sequence of SEQ ID NO: 47. Alternatively or in addition (e.g., in addition), the anti-TfR antibody of the present disclosure comprises a CDR-L1, a CDR-L2, and a CDR-L3 of a light chain variable domain having the amino acid sequence of SEQ ID NO: 48.

In some embodiments, the anti-TfR antibody of the present disclosure comprises a CDR-H1 having the amino acid sequence of SEQ ID NO: 41 (according to the IMGT definition system), a CDR-H2 having the amino acid sequence of SEQ ID NO: 42 (according to the IMGT definition system), a CDR-H3 having the amino acid sequence of SEQ ID NO: 43 (according to the IMGT definition system), a CDR-L1 having the amino acid sequence of SEQ ID NO: 44 (according to the IMGT definition system), a CDR-L2 having the amino acid sequence of SEQ ID NO: 45 (according to the IMGT definition system), and a CDR-L3 having the amino acid sequence of SEQ ID NO: 46 (according to the IMGT definition system).

In some embodiments, anti-TfR antibody of the present disclosure comprises a CDR-H1, a CDR-H2, and a CDR-H3, which collectively contains no more than 5 amino acid variations (e.g., no more than 5, 4, 3, 2, or 1 amino acid variation) as compared with the CDR-H1 having the amino acid sequence of SEQ ID NO: 41, CDR-H2 having the amino acid sequence of SEQ ID NO: 42, and CDR-H3 having the amino acid sequence of SEQ ID NO: 43. Alternatively or in addition (e.g., in addition), the anti-TfR antibody of the present disclosure comprises a CDR-L1, a CDR-L2, and a CDR-L3, which collectively contains no more than 5 amino acid variations (e.g., no more than 5, 4, 3, 2 or 1 amino acid variation) as compared with the CDR-L1 having the amino acid sequence of SEQ ID NO: 44, CDR-L2 having the amino acid sequence of SEQ ID NO: 45, and CDR-L3 having the amino acid sequence of SEQ ID NO: 46.

In some embodiments, the anti-TfR antibody of the present disclosure comprises a CDR-H1, a CDR-H2, and a CDR-H3 that collectively are at least 75% (e.g., 75%, 80%, 85%, 90%, 95%, 98%, or 99%) identical to the CDR-H1 having the amino acid sequence of SEQ ID NO: 41, CDR-H2 having the amino acid sequence of SEQ ID NO: 42, and CDR-H3 having the amino acid sequence of SEQ ID NO: 43. Alternatively or in addition (e.g., in addition), the anti-TfR antibody of the present disclosure comprises a CDR-L1, a CDR-L2, and a CDR-L3 that collectively are at least 75% (e.g., 75%, 80%, 85%, 90%, 95%, 98%, or 99%) identical to the to the CDR-L1 having the amino acid sequence of SEQ ID NO: 44, CDR-L2 having the amino acid sequence of SEQ ID NO: 45, and CDR-L3 having the amino acid sequence of SEQ ID NO: 46.

In some embodiments, the anti-TfR antibody of the present disclosure comprises: a CDR-H1 having no more than 3 amino acid variations (e.g., no more than 3, 2, or 1 amino acid variation) as compared with the CDR-H1 having the amino acid sequence of SEQ ID NO: 41; a CDR-H2 having no more than 3 amino acid variations (e.g., no more than 3, 2, or 1 amino acid variation) as compared with the CDR-H2 having the amino acid sequence of SEQ ID NO: 42; and/or (e.g., and) a CDR-H3 having no more than 3 amino acid variations (e.g., no more than 3, 2, or 1 amino acid variation) as compared with the CDR-H3 having the amino acid sequence of SEQ ID NO: 43. Alternatively or in addition (e.g., in addition), the anti-TfR antibody of the present disclosure comprises: a CDR-L1 having no more than 3 amino acid variations (e.g., no more than 3, 2, or 1 amino acid variation) as compared with the CDR-L1 having the amino acid sequence of SEQ ID NO: 44; a CDR-L2 having no more than 3 amino acid variations (e.g., no more than 3, 2, or 1 amino acid variation) as compared with the CDR-L2 having the amino acid sequence of SEQ ID NO: 45; and/or (e.g., and) a CDR-L3 having no more than 3 amino acid variations (e.g., no more than 3, 2, or 1 amino acid variation) as compared with the CDR-L3 having the amino acid sequence of SEQ ID NO: 46.

In some embodiments, the anti-TfR antibody of the present disclosure comprises a VH comprising the amino acid sequence of SEQ ID NO: 47. Alternatively or in addition (e.g., in addition), the anti-TfR antibody of the present disclosure comprises a VL comprising the amino acid sequence of SEQ ID NO: 48.

In some embodiments, the anti-TfR antibody of the present disclosure comprises a VH containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation)no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 47. Alternatively or in addition (e.g., in addition), the anti-TfR antibody of the present disclosure comprises a VL containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VL as set forth in SEQ ID NO: 48.

In some embodiments, the anti-TfR antibody of the present disclosure comprises a VH comprising an amino acid sequence that is at least 75% (e.g., 75%, 80%, 85%, 90%, 95%, 98%, or 99%) identical to the VH as set forth in SEQ ID NO: 47. Alternatively or in addition (e.g., in addition), the anti-TfR antibody of the present disclosure comprises a VL comprising an amino acid sequence that is at least 75% (e.g., 75%, 80%, 85%, 90%, 95%, 98%, or 99%) identical to the VL as set forth in SEQ ID NO: 48.

In some embodiments, the anti-TfR antibody of the present disclosure comprises a CDR-H1, a CDR-H2, and a CDR-H3 of a heavy chain variable domain having the amino acid sequence of SEQ ID NO: 54. Alternatively or in addition (e.g., in addition), the anti-TfR antibody of the present disclosure comprises a CDR-L1, a CDR-L2, and a CDR-L3 of a light chain variable domain having the amino acid sequence of SEQ ID NO: 55.

In some embodiments, the anti-TfR antibody of the present disclosure comprises a CDR-H1 having the amino acid sequence of SEQ ID NO: 49 (according to the IMGT definition system), a CDR-H2 having the amino acid sequence of SEQ ID NO: 50 (according to the IMGT definition system), a CDR-H3 having the amino acid sequence of SEQ ID NO: 51 (according to the IMGT definition system), a CDR-L1 having the amino acid sequence of SEQ ID NO: 52 (according to the IMGT definition system), a CDR-L2 having the amino acid sequence of SEQ ID NO: 29 (according to the IMGT definition system), and a CDR-L3 having the amino acid sequence of SEQ ID NO: 53 (according to the IMGT definition system).

In some embodiments, anti-TfR antibody of the present disclosure comprises a CDR-H1, a CDR-H2, and a CDR-H3, which collectively contains no more than 5 amino acid variations (e.g., no more than 5, 4, 3, 2, or 1 amino acid variation) as compared with the CDR-H1 having the amino acid sequence of SEQ ID NO: 49, CDR-H2 having the amino acid sequence of SEQ ID NO: 50, and CDR-H3 having the amino acid sequence of SEQ ID NO: 51. Alternatively or in addition (e.g., in addition), the anti-TfR antibody of the present disclosure comprises a CDR-L1, a CDR-L2, and a CDR-L3, which collectively contains no more than 5 amino acid variations (e.g., no more than 5, 4, 3, 2 or 1 amino acid variation) as compared with the CDR-L1 having the amino acid sequence of SEQ ID NO: 52, CDR-L2 having the amino acid sequence of SEQ ID NO: 29, and CDR-L3 having the amino acid sequence of SEQ ID NO: 53.

In some embodiments, the anti-TfR antibody of the present disclosure comprises a CDR-H1, a CDR-H2, and a CDR-H3 that collectively are at least 75% (e.g., 75%, 80%, 85%, 90%, 95%, 98%, or 99%) identical to the CDR-H1 having the amino acid sequence of SEQ ID NO: 49, CDR-H2 having the amino acid sequence of SEQ ID NO: 50, and CDR-H3 having the amino acid sequence of SEQ ID NO: 51. Alternatively or in addition (e.g., in addition), the anti-TfR antibody of the present disclosure comprises a CDR-L1, a CDR-L2, and a CDR-L3 that collectively are at least 75% (e.g., 75%, 80%, 85%, 90%, 95%, 98%, or 99%) identical to the to the CDR-L1 having the amino acid sequence of SEQ ID NO: 52, CDR-L2 having the amino acid sequence of SEQ ID NO: 29, and CDR-L3 having the amino acid sequence of SEQ ID NO: 53.

In some embodiments, the anti-TfR antibody of the present disclosure comprises: a CDR-H1 having no more than 3 amino acid variations (e.g., no more than 3, 2, or 1 amino acid variation) as compared with the CDR-H1 having the amino acid sequence of SEQ ID NO: 49; a CDR-H2 having no more than 3 amino acid variations (e.g., no more than 3, 2, or 1 amino acid variation) as compared with the CDR-H2 having the amino acid sequence of SEQ ID NO: 50; and/or (e.g., and) a CDR-H3 having no more than 3 amino acid variations (e.g., no more than 3, 2, or 1 amino acid variation) as compared with the CDR-H3 having the amino acid sequence of SEQ ID NO: 51. Alternatively or in addition (e.g., in addition), the anti-TfR antibody of the present disclosure comprises: a CDR-L1 having no more than 3 amino acid variations (e.g., no more than 3, 2, or 1 amino acid variation) as compared with the CDR-L1 having the amino acid sequence of SEQ ID NO: 52; a CDR-L2 having no more than 3 amino acid variations (e.g., no more than 3, 2, or 1 amino acid variation) as compared with the CDR-L2 having the amino acid sequence of SEQ ID NO: 29; and/or (e.g., and) a CDR-L3 having no more than 3 amino acid variations (e.g., no more than 3, 2, or 1 amino acid variation) as compared with the CDR-L3 having the amino acid sequence of SEQ ID NO: 53.

In some embodiments, the anti-TfR antibody of the present disclosure comprises a VH comprising the amino acid sequence of SEQ ID NO: 54. Alternatively or in addition (e.g., in addition), the anti-TfR antibody of the present disclosure comprises a VL comprising the amino acid sequence of SEQ ID NO: 55.

In some embodiments, the anti-TfR antibody of the present disclosure comprises a VH containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation)no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 54. Alternatively or in addition (e.g., in addition), the anti-TfR antibody of the present disclosure comprises a VL containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VL as set forth in SEQ ID NO: 55.

In some embodiments, the anti-TfR antibody of the present disclosure comprises a VH comprising an amino acid sequence that is at least 75% (e.g., 75%, 80%, 85%, 90%, 95%, 98%, or 99%) identical to the VH as set forth in SEQ ID NO: 54. Alternatively or in addition (e.g., in addition), the anti-TfR antibody of the present disclosure comprises a VL comprising an amino acid sequence that is at least 75% (e.g., 75%, 80%, 85%, 90%, 95%, 98%, or 99%) identical to the VL as set forth in SEQ ID NO: 55.

In some embodiments, the anti-TfR antibody of the present disclosure comprises a CDR-H1, a CDR-H2, and a CDR-H3 of a heavy chain variable domain having the amino acid sequence of SEQ ID NO: 62. Alternatively or in addition (e.g., in addition), the anti-TfR antibody of the present disclosure comprises a CDR-L1, a CDR-L2, and a CDR-L3 of a light chain variable domain having the amino acid sequence of SEQ ID NO: 63.

In some embodiments, the anti-TfR antibody of the present disclosure comprises a CDR-H1 having the amino acid sequence of SEQ ID NO: 56 (according to the IMGT definition system), a CDR-H2 having the amino acid sequence of SEQ ID NO: 57 (according to the IMGT definition system), a CDR-H3 having the amino acid sequence of SEQ ID NO: 58 (according to the IMGT definition system), a CDR-L1 having the amino acid sequence of SEQ ID NO: 59 (according to the IMGT definition system), a CDR-L2 having the amino acid sequence of SEQ ID NO: 60 (according to the IMGT definition system), and a CDR-L3 having the amino acid sequence of SEQ ID NO: 61 (according to the IMGT definition system).

In some embodiments, anti-TfR antibody of the present disclosure comprises a CDR-H1, a CDR-H2, and a CDR-H3, which collectively contains no more than 5 amino acid variations (e.g., no more than 5, 4, 3, 2, or 1 amino acid variation) as compared with the CDR-H1 having the amino acid sequence of SEQ ID NO: 56, CDR-H2 having the amino acid sequence of SEQ ID NO: 57, and CDR-H3 having the amino acid sequence of SEQ ID NO: 58. Alternatively or in addition (e.g., in addition), the anti-TfR antibody of the present disclosure comprises a CDR-L1, a CDR-L2, and a CDR-L3, which collectively contains no more than 5 amino acid variations (e.g., no more than 5, 4, 3, 2 or 1 amino acid variation) as compared with the CDR-L1 having the amino acid sequence of SEQ ID NO: 59, CDR-L2 having the amino acid sequence of SEQ ID NO: 60, and CDR-L3 having the amino acid sequence of SEQ ID NO: 61.

In some embodiments, the anti-TfR antibody of the present disclosure comprises a CDR-H1, a CDR-H2, and a CDR-H3 that collectively are at least 75% (e.g., 75%, 80%, 85%, 90%, 95%, 98%, or 99%) identical to the CDR-H1 having the amino acid sequence of SEQ ID NO: 56, CDR-H2 having the amino acid sequence of SEQ ID NO: 57, and CDR-H3 having the amino acid sequence of SEQ ID NO: 58. Alternatively or in addition (e.g., in addition), the anti-TfR antibody of the present disclosure comprises a CDR-L1, a CDR-L2, and a CDR-L3 that collectively are at least 75% (e.g., 75%, 80%, 85%, 90%, 95%, 98%, or 99%) identical to the to the CDR-L1 having the amino acid sequence of SEQ ID NO: 59, CDR-L2 having the amino acid sequence of SEQ ID NO: 60, and CDR-L3 having the amino acid sequence of SEQ ID NO: 61.

In some embodiments, the anti-TfR antibody of the present disclosure comprises: a CDR-H1 having no more than 3 amino acid variations (e.g., no more than 3, 2, or 1 amino acid variation) as compared with the CDR-H1 having the amino acid sequence of SEQ ID NO: 56; a CDR-H2 having no more than 3 amino acid variations (e.g., no more than 3, 2, or 1 amino acid variation) as compared with the CDR-H2 having the amino acid sequence of SEQ ID NO: 57; and/or (e.g., and) a CDR-H3 having no more than 3 amino acid variations (e.g., no more than 3, 2, or 1 amino acid variation) as compared with the CDR-H3 having the amino acid sequence of SEQ ID NO: 58. Alternatively or in addition (e.g., in addition), the anti-TfR antibody of the present disclosure comprises: a CDR-L1 having no more than 3 amino acid variations (e.g., no more than 3, 2, or 1 amino acid variation) as compared with the CDR-L1 having the amino acid sequence of SEQ ID NO: 59; a CDR-L2 having no more than 3 amino acid variations (e.g., no more than 3, 2, or 1 amino acid variation) as compared with the CDR-L2 having the amino acid sequence of SEQ ID NO: 60; and/or (e.g., and) a CDR-L3 having no more than 3 amino acid variations (e.g., no more than 3, 2, or 1 amino acid variation) as compared with the CDR-L3 having the amino acid sequence of SEQ ID NO: 61.

In some embodiments, the anti-TfR antibody of the present disclosure comprises a VH comprising the amino acid sequence of SEQ ID NO: 62. Alternatively or in addition (e.g., in addition), the anti-TfR antibody of the present disclosure comprises a VL comprising the amino acid sequence of SEQ ID NO: 63.

In some embodiments, the anti-TfR antibody of the present disclosure comprises a VH containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 62. Alternatively or in addition (e.g., in addition), the anti-TfR antibody of the present disclosure comprises a VL containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VL as set forth in SEQ ID NO: 63.

In some embodiments, the anti-TfR antibody of the present disclosure comprises a VH comprising an amino acid sequence that is at least 75% (e.g., 75%, 80%, 85%, 90%, 95%, 98%, or 99%) identical to the VH as set forth in SEQ ID NO: 62. Alternatively or in addition (e.g., in addition), the anti-TfR antibody of the present disclosure comprises a VL comprising an amino acid sequence that is at least 75% (e.g., 75%, 80%, 85%, 90%, 95%, 98%, or 99%) identical to the VL as set forth in SEQ ID NO: 63.

In some embodiments, the anti-TfR antibody of the present disclosure comprises a CDR-H1, a CDR-H2, and a CDR-H3 of a heavy chain variable domain having the amino acid sequence of SEQ ID NO: 70. Alternatively or in addition (e.g., in addition), the anti-TfR antibody of the present disclosure comprises a CDR-L1, a CDR-L2, and a CDR-L3 of a light chain variable domain having the amino acid sequence of SEQ ID NO: 71.

In some embodiments, the anti-TfR antibody of the present disclosure comprises a CDR-H1 having the amino acid sequence of SEQ ID NO: 64 (according to the IMGT definition system), a CDR-H2 having the amino acid sequence of SEQ ID NO: 65 (according to the IMGT definition system), a CDR-H3 having the amino acid sequence of SEQ ID NO: 66 (according to the IMGT definition system), a CDR-L1 having the amino acid sequence of SEQ ID NO: 67 (according to the IMGT definition system), a CDR-L2 having the amino acid sequence of SEQ ID NO: 68 (according to the IMGT definition system), and a CDR-L3 having the amino acid sequence of SEQ ID NO: 69 (according to the IMGT definition system).

In some embodiments, anti-TfR antibody of the present disclosure comprises a CDR-H1, a CDR-H2, and a CDR-H3, which collectively contains no more than 5 amino acid variations (e.g., no more than 5, 4, 3, 2, or 1 amino acid variation) as compared with the CDR-H1 having the amino acid sequence of SEQ ID NO: 64, CDR-H2 having the amino acid sequence of SEQ ID NO: 65, and CDR-H3 having the amino acid sequence of SEQ ID NO: 66. Alternatively or in addition (e.g., in addition), the anti-TfR antibody of the present disclosure comprises a CDR-L1, a CDR-L2, and a CDR-L3, which collectively contains no more than 5 amino acid variations (e.g., no more than 5, 4, 3, 2 or 1 amino acid variation) as compared with the CDR-L1 having the amino acid sequence of SEQ ID NO: 67, CDR-L2 having the amino acid sequence of SEQ ID NO: 68, and CDR-L3 having the amino acid sequence of SEQ ID NO: 69.

In some embodiments, the anti-TfR antibody of the present disclosure comprises a CDR-H1, a CDR-H2, and a CDR-H3 that collectively are at least 75% (e.g., 75%, 80%, 85%, 90%, 95%, 98%, or 99%) identical to the CDR-H1 having the amino acid sequence of SEQ ID NO: 64, CDR-H2 having the amino acid sequence of SEQ ID NO: 65, and CDR-H3 having the amino acid sequence of SEQ ID NO: 66. Alternatively or in addition (e.g., in addition), the anti-TfR antibody of the present disclosure comprises a CDR-L1, a CDR-L2, and a CDR-L3 that collectively are at least 75% (e.g., 75%, 80%, 85%, 90%, 95%, 98%, or 99%) identical to the to the CDR-L1 having the amino acid sequence of SEQ ID NO: 67, CDR-L2 having the amino acid sequence of SEQ ID NO: 68, and CDR-L3 having the amino acid sequence of SEQ ID NO: 69.

In some embodiments, the anti-TfR antibody of the present disclosure comprises: a CDR-H1 having no more than 3 amino acid variations (e.g., no more than 3, 2, or 1 amino acid variation) as compared with the CDR-H1 having the amino acid sequence of SEQ ID NO: 64; a CDR-H2 having no more than 3 amino acid variations (e.g., no more than 3, 2, or 1 amino acid variation) as compared with the CDR-H2 having the amino acid sequence of SEQ ID NO: 65; and/or (e.g., and) a CDR-H3 having no more than 3 amino acid variations (e.g., no more than 3, 2, or 1 amino acid variation) as compared with the CDR-H3 having the amino acid sequence of SEQ ID NO: 66. Alternatively or in addition (e.g., in addition), the anti-TfR antibody of the present disclosure comprises: a CDR-L1 having no more than 3 amino acid variations (e.g., no more than 3, 2, or 1 amino acid variation) as compared with the CDR-L1 having the amino acid sequence of SEQ ID NO: 67; a CDR-L2 having no more than 3 amino acid variations (e.g., no more than 3, 2, or 1 amino acid variation) as compared with the CDR-L2 having the amino acid sequence of SEQ ID NO: 68; and/or (e.g., and) a CDR-L3 having no more than 3 amino acid variations (e.g., no more than 3, 2, or 1 amino acid variation) as compared with the CDR-L3 having the amino acid sequence of SEQ ID NO: 69.

In some embodiments, the anti-TfR antibody of the present disclosure comprises a VH comprising the amino acid sequence of SEQ ID NO: 70. Alternatively or in addition (e.g., in addition), the anti-TfR antibody of the present disclosure comprises a VL comprising the amino acid sequence of SEQ ID NO: 71.

In some embodiments, the anti-TfR antibody of the present disclosure comprises a VH containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation)no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 70. Alternatively or in addition (e.g., in addition), the anti-TfR antibody of the present disclosure comprises a VL containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VL as set forth in SEQ ID NO: 71.

In some embodiments, the anti-TfR antibody of the present disclosure comprises a VH comprising an amino acid sequence that is at least 75% (e.g., 75%, 80%, 85%, 90%, 95%, 98%, or 99%) identical to the VH as set forth in SEQ ID NO: 70. Alternatively or in addition (e.g., in addition), the anti-TfR antibody of the present disclosure comprises a VL comprising an amino acid sequence that is at least 75% (e.g., 75%, 80%, 85%, 90%, 95%, 98%, or 99%) identical to the VL as set forth in SEQ ID NO: 71.

In some embodiments, the anti-TfR antibody of the present disclosure comprises a CDR-H1, a CDR-H2, and a CDR-H3 of a heavy chain variable domain having the amino acid sequence of SEQ ID NO: 77. Alternatively or in addition (e.g., in addition), the anti-TfR antibody of the present disclosure comprises a CDR-L1, a CDR-L2, and a CDR-L3 of a light chain variable domain having the amino acid sequence of SEQ ID NO: 78.

In some embodiments, the anti-TfR antibody of the present disclosure comprises a CDR-H1 having the amino acid sequence of SEQ ID NO: 72 (according to the IMGT definition system), a CDR-H2 having the amino acid sequence of SEQ ID NO: 73 (according to the IMGT definition system), a CDR-H3 having the amino acid sequence of SEQ ID NO: 74 (according to the IMGT definition system), a CDR-L1 having the amino acid sequence of SEQ ID NO: 75 (according to the IMGT definition system), a CDR-L2 having the amino acid sequence of SEQ ID NO: 45 (according to the IMGT definition system), and a CDR-L3 having the amino acid sequence of SEQ ID NO: 76 (according to the IMGT definition system).

In some embodiments, anti-TfR antibody of the present disclosure comprises a CDR-H1, a CDR-H2, and a CDR-H3, which collectively contains no more than 5 amino acid variations (e.g., no more than 5, 4, 3, 2, or 1 amino acid variation) as compared with the CDR-H1 having the amino acid sequence of SEQ ID NO: 72, CDR-H2 having the amino acid sequence of SEQ ID NO: 73, and CDR-H3 having the amino acid sequence of SEQ ID NO: 74. Alternatively or in addition (e.g., in addition), the anti-TfR antibody of the present disclosure comprises a CDR-L1, a CDR-L2, and a CDR-L3, which collectively contains no more than 5 amino acid variations (e.g., no more than 5, 4, 3, 2 or 1 amino acid variation) as compared with the CDR-L1 having the amino acid sequence of SEQ ID NO: 75, CDR-L2 having the amino acid sequence of SEQ ID NO: 45, and CDR-L3 having the amino acid sequence of SEQ ID NO: 76.

In some embodiments, the anti-TfR antibody of the present disclosure comprises a CDR-H1, a CDR-H2, and a CDR-H3 that collectively are at least 75% (e.g., 75%, 80%, 85%, 90%, 95%, 98%, or 99%) identical to the CDR-H1 having the amino acid sequence of SEQ ID NO: 72, CDR-H2 having the amino acid sequence of SEQ ID NO: 73, and CDR-H3 having the amino acid sequence of SEQ ID NO: 74. Alternatively or in addition (e.g., in addition), the anti-TfR antibody of the present disclosure comprises a CDR-L1, a CDR-L2, and a CDR-L3 that collectively are at least 75% (e.g., 75%, 80%, 85%, 90%, 95%, 98%, or 99%) identical to the to the CDR-L1 having the amino acid sequence of SEQ ID NO: 75, CDR-L2 having the amino acid sequence of SEQ ID NO: 45, and CDR-L3 having the amino acid sequence of SEQ ID NO: 76.

In some embodiments, the anti-TfR antibody of the present disclosure comprises: a CDR-H1 having no more than 3 amino acid variations (e.g., no more than 3, 2, or 1 amino acid variation) as compared with the CDR-H1 having the amino acid sequence of SEQ ID NO: 72; a CDR-H2 having no more than 3 amino acid variations (e.g., no more than 3, 2, or 1 amino acid variation) as compared with the CDR-H2 having the amino acid sequence of SEQ ID NO: 73; and/or (e.g., and) a CDR-H3 having no more than 3 amino acid variations (e.g., no more than 3, 2, or 1 amino acid variation) as compared with the CDR-H3 having the amino acid sequence of SEQ ID NO: 74. Alternatively or in addition (e.g., in addition), the anti-TfR antibody of the present disclosure comprises: a CDR-L1 having no more than 3 amino acid variations (e.g., no more than 3, 2, or 1 amino acid variation) as compared with the CDR-L1 having the amino acid sequence of SEQ ID NO: 75; a CDR-L2 having no more than 3 amino acid variations (e.g., no more than 3, 2, or 1 amino acid variation) as compared with the CDR-L2 having the amino acid sequence of SEQ ID NO: 45; and/or (e.g., and) a CDR-L3 having no more than 3 amino acid variations (e.g., no more than 3, 2, or 1 amino acid variation) as compared with the CDR-L3 having the amino acid sequence of SEQ ID NO: 76.

In some embodiments, the anti-TfR antibody of the present disclosure comprises a VH comprising the amino acid sequence of SEQ ID NO: 77. Alternatively or in addition (e.g., in addition), the anti-TfR antibody of the present disclosure comprises a VL comprising the amino acid sequence of SEQ ID NO: 78.

In some embodiments, the anti-TfR antibody of the present disclosure comprises a VH containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 77. Alternatively or in addition (e.g., in addition), the anti-TfR antibody of the present disclosure comprises a VL containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VL as set forth in SEQ ID NO: 78.

In some embodiments, the anti-TfR antibody of the present disclosure comprises a VH comprising an amino acid sequence that is at least 75% (e.g., 75%, 80%, 85%, 90%, 95%, 98%, or 99%) identical to the VH as set forth in SEQ ID NO: 77. Alternatively or in addition (e.g., in addition), the anti-TfR antibody of the present disclosure comprises a VL comprising an amino acid sequence that is at least 75% (e.g., 75%, 80%, 85%, 90%, 95%, 98%, or 99%) identical to the VL as set forth in SEQ ID NO: 78.

In some embodiments, the anti-TfR antibody of the present disclosure comprises a CDR-H1, a CDR-H2, and a CDR-H3 of a heavy chain variable domain having the amino acid sequence of SEQ ID NO: 85. Alternatively or in addition (e.g., in addition), the anti-TfR antibody of the present disclosure comprises a CDR-L1, a CDR-L2, and a CDR-L3 of a light chain variable domain having the amino acid sequence of SEQ ID NO: 86.

In some embodiments, the anti-TfR antibody of the present disclosure comprises a CDR-H1 having the amino acid sequence of SEQ ID NO: 79 (according to the IMGT definition system), a CDR-H2 having the amino acid sequence of SEQ ID NO: 80 (according to the IMGT definition system), a CDR-H3 having the amino acid sequence of SEQ ID NO: 81 (according to the IMGT definition system), a CDR-L1 having the amino acid sequence of SEQ ID NO: 82 (according to the IMGT definition system), a CDR-L2 having the amino acid sequence of SEQ ID NO: 83 (according to the IMGT definition system), and a CDR-L3 having the amino acid sequence of SEQ ID NO: 84 (according to the IMGT definition system).

In some embodiments, anti-TfR antibody of the present disclosure comprises a CDR-H1, a CDR-H2, and a CDR-H3, which collectively contains no more than 5 amino acid variations (e.g., no more than 5, 4, 3, 2, or 1 amino acid variation) as compared with the CDR-H1 having the amino acid sequence of SEQ ID NO: 79, CDR-H2 having the amino acid sequence of SEQ ID NO: 80, and CDR-H3 having the amino acid sequence of SEQ ID NO: 81. Alternatively or in addition (e.g., in addition), the anti-TfR antibody of the present disclosure comprises a CDR-L1, a CDR-L2, and a CDR-L3, which collectively contains no more than 5 amino acid variations (e.g., no more than 5, 4, 3, 2 or 1 amino acid variation) as compared with the CDR-L1 having the amino acid sequence of SEQ ID NO: 82, CDR-L2 having the amino acid sequence of SEQ ID NO: 83, and CDR-L3 having the amino acid sequence of SEQ ID NO: 84.

In some embodiments, the anti-TfR antibody of the present disclosure comprises a CDR-H1, a CDR-H2, and a CDR-H3 that collectively are at least 75% (e.g., 75%, 80%, 85%, 90%, 95%, 98%, or 99%) identical to the CDR-H1 having the amino acid sequence of SEQ ID NO: 79, CDR-H2 having the amino acid sequence of SEQ ID NO: 80, and CDR-H3 having the amino acid sequence of SEQ ID NO: 81. Alternatively or in addition (e.g., in addition), the anti-TfR antibody of the present disclosure comprises a CDR-L1, a CDR-L2, and a CDR-L3 that collectively are at least 75% (e.g., 75%, 80%, 85%, 90%, 95%, 98%, or 99%) identical to the to the CDR-L1 having the amino acid sequence of SEQ ID NO: 82, CDR-L2 having the amino acid sequence of SEQ ID NO: 83, and CDR-L3 having the amino acid sequence of SEQ ID NO: 84.

In some embodiments, the anti-TfR antibody of the present disclosure comprises: a CDR-H1 having no more than 3 amino acid variations (e.g., no more than 3, 2, or 1 amino acid variation) as compared with the CDR-H1 having the amino acid sequence of SEQ ID NO: 79; a CDR-H2 having no more than 3 amino acid variations (e.g., no more than 3, 2, or 1 amino acid variation) as compared with the CDR-H2 having the amino acid sequence of SEQ ID NO: 80; and/or (e.g., and) a CDR-H3 having no more than 3 amino acid variations (e.g., no more than 3, 2, or 1 amino acid variation) as compared with the CDR-H3 having the amino acid sequence of SEQ ID NO: 81. Alternatively or in addition (e.g., in addition), the anti-TfR antibody of the present disclosure comprises: a CDR-L1 having no more than 3 amino acid variations (e.g., no more than 3, 2, or 1 amino acid variation) as compared with the CDR-L1 having the amino acid sequence of SEQ ID NO: 82; a CDR-L2 having no more than 3 amino acid variations (e.g., no more than 3, 2, or 1 amino acid variation) as compared with the CDR-L2 having the amino acid sequence of SEQ ID NO: 83; and/or (e.g., and) a CDR-L3 having no more than 3 amino acid variations (e.g., no more than 3, 2, or 1 amino acid variation) as compared with the CDR-L3 having the amino acid sequence of SEQ ID NO: 84.

In some embodiments, the anti-TfR antibody of the present disclosure comprises a VH comprising the amino acid sequence of SEQ ID NO: 85. Alternatively or in addition (e.g., in addition), the anti-TfR antibody of the present disclosure comprises a VL comprising the amino acid sequence of SEQ ID NO: 86.

In some embodiments, the anti-TfR antibody of the present disclosure comprises a VH containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation)no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 85. Alternatively or in addition (e.g., in addition), the anti-TfR antibody of the present disclosure comprises a VL containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VL as set forth in SEQ ID NO: 86.

In some embodiments, the anti-TfR antibody of the present disclosure comprises a VH comprising an amino acid sequence that is at least 75% (e.g., 75%, 80%, 85%, 90%, 95%, 98%, or 99%) identical to the VH as set forth in SEQ ID NO: 85. Alternatively or in addition (e.g., in addition), the anti-TfR antibody of the present disclosure comprises a VL comprising an amino acid sequence that is at least 75% (e.g., 75%, 80%, 85%, 90%, 95%, 98%, or 99%) identical to the VL as set forth in SEQ ID NO: 86.

In some embodiments, the anti-TfR antibody of the present disclosure comprises a CDR-H1, a CDR-H2, and a CDR-H3 of a heavy chain variable domain having the amino acid sequence of SEQ ID NO: 89. Alternatively or in addition (e.g., in addition), the anti-TfR antibody of the present disclosure comprises a CDR-L1, a CDR-L2, and a CDR-L3 of a light chain variable domain having the amino acid sequence of SEQ ID NO: 90.

In some embodiments, the anti-TfR antibody of the present disclosure comprises a CDR-H1 having the amino acid sequence of SEQ ID NO: 72 (according to the IMGT definition system), a CDR-H2 having the amino acid sequence of SEQ ID NO: 87 (according to the IMGT definition system), a CDR-H3 having the amino acid sequence of SEQ ID NO: 74 (according to the IMGT definition system), a CDR-L1 having the amino acid sequence of SEQ ID NO: 75 (according to the IMGT definition system), a CDR-L2 having the amino acid sequence of SEQ ID NO: 45 (according to the IMGT definition system), and a CDR-L3 having the amino acid sequence of SEQ ID NO: 88 (according to the IMGT definition system).

In some embodiments, anti-TfR antibody of the present disclosure comprises a CDR-H1, a CDR-H2, and a CDR-H3, which collectively contains no more than 5 amino acid variations (e.g., no more than 5, 4, 3, 2, or 1 amino acid variation) as compared with the CDR-H1 having the amino acid sequence of SEQ ID NO: 72, CDR-H2 having the amino acid sequence of SEQ ID NO: 87, and CDR-H3 having the amino acid sequence of SEQ ID NO: 74. Alternatively or in addition (e.g., in addition), the anti-TfR antibody of the present disclosure comprises a CDR-L1, a CDR-L2, and a CDR-L3, which collectively contains no more than 5 amino acid variations (e.g., no more than 5, 4, 3, 2 or 1 amino acid variation) as compared with the CDR-L1 having the amino acid sequence of SEQ ID NO: 75, CDR-L2 having the amino acid sequence of SEQ ID NO: 45, and CDR-L3 having the amino acid sequence of SEQ ID NO: 88.

In some embodiments, the anti-TfR antibody of the present disclosure comprises a CDR-H1, a CDR-H2, and a CDR-H3 that collectively are at least 75% (e.g., 75%, 80%, 85%, 90%, 95%, 98%, or 99%) identical to the CDR-H1 having the amino acid sequence of SEQ ID NO: 72, CDR-H2 having the amino acid sequence of SEQ ID NO: 87, and CDR-H3 having the amino acid sequence of SEQ ID NO: 74. Alternatively or in addition (e.g., in addition), the anti-TfR antibody of the present disclosure comprises a CDR-L1, a CDR-L2, and a CDR-L3 that collectively are at least 75% (e.g., 75%, 80%, 85%, 90%, 95%, 98%, or 99%) identical to the to the CDR-L1 having the amino acid sequence of SEQ ID NO: 75, CDR-L2 having the amino acid sequence of SEQ ID NO: 45, and CDR-L3 having the amino acid sequence of SEQ ID NO: 88.

In some embodiments, the anti-TfR antibody of the present disclosure comprises: a CDR-H1 having no more than 3 amino acid variations (e.g., no more than 3, 2, or 1 amino acid variation) as compared with the CDR-H1 having the amino acid sequence of SEQ ID NO: 72; a CDR-H2 having no more than 3 amino acid variations (e.g., no more than 3, 2, or 1 amino acid variation) as compared with the CDR-H2 having the amino acid sequence of SEQ ID NO: 87; and/or (e.g., and) a CDR-H3 having no more than 3 amino acid variations (e.g., no more than 3, 2, or 1 amino acid variation) as compared with the CDR-H3 having the amino acid sequence of SEQ ID NO: 74. Alternatively or in addition (e.g., in addition), the anti-TfR antibody of the present disclosure comprises: a CDR-L1 having no more than 3 amino acid variations (e.g., no more than 3, 2, or 1 amino acid variation) as compared with the CDR-L1 having the amino acid sequence of SEQ ID NO: 75; a CDR-L2 having no more than 3 amino acid variations (e.g., no more than 3, 2, or 1 amino acid variation) as compared with the CDR-L2 having the amino acid sequence of SEQ ID NO: 45; and/or (e.g., and) a CDR-L3 having no more than 3 amino acid variations (e.g., no more than 3, 2, or 1 amino acid variation) as compared with the CDR-L3 having the amino acid sequence of SEQ ID NO: 88.

In some embodiments, the anti-TfR antibody of the present disclosure comprises a VH comprising the amino acid sequence of SEQ ID NO: 89. Alternatively or in addition (e.g., in addition), the anti-TfR antibody of the present disclosure comprises a VL comprising the amino acid sequence of SEQ ID NO: 90.

In some embodiments, the anti-TfR antibody of the present disclosure comprises a VH containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation)no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 89. Alternatively or in addition (e.g., in addition), the anti-TfR antibody of the present disclosure comprises a VL containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VL as set forth in SEQ ID NO: 90.

In some embodiments, the anti-TfR antibody of the present disclosure comprises a VH comprising an amino acid sequence that is at least 75% (e.g., 75%, 80%, 85%, 90%, 95%, 98%, or 99%) identical to the VH as set forth in SEQ ID NO: 89. Alternatively or in addition (e.g., in addition), the anti-TfR antibody of the present disclosure comprises a VL comprising an amino acid sequence that is at least 75% (e.g., 75%, 80%, 85%, 90%, 95%, 98%, or 99%) identical to the VL as set forth in SEQ ID NO: 90.

In some embodiments, the anti-TfR antibody of the present disclosure comprises a CDR-H1, a CDR-H2, and a CDR-H3 of a heavy chain variable domain having the amino acid sequence of SEQ ID NO: 97. Alternatively or in addition (e.g., in addition), the anti-TfR antibody of the present disclosure comprises a CDR-L1, a CDR-L2, and a CDR-L3 of a light chain variable domain having the amino acid sequence of SEQ ID NO: 98.

In some embodiments, the anti-TfR antibody of the present disclosure comprises a CDR-H1 having the amino acid sequence of SEQ ID NO: 91 (according to the IMGT definition system), a CDR-H2 having the amino acid sequence of SEQ ID NO: 92 (according to the IMGT definition system), a CDR-H3 having the amino acid sequence of SEQ ID NO: 93 (according to the IMGT definition system), a CDR-L1 having the amino acid sequence of SEQ ID NO: 94 (according to the IMGT definition system), a CDR-L2 having the amino acid sequence of SEQ ID NO: 95 (according to the IMGT definition system), and a CDR-L3 having the amino acid sequence of SEQ ID NO: 96 (according to the IMGT definition system).

In some embodiments, anti-TfR antibody of the present disclosure comprises a CDR-H1, a CDR-H2, and a CDR-H3, which collectively contains no more than 5 amino acid variations (e.g., no more than 5, 4, 3, 2, or 1 amino acid variation) as compared with the CDR-H1 having the amino acid sequence of SEQ ID NO: 91, CDR-H2 having the amino acid sequence of SEQ ID NO: 92, and CDR-H3 having the amino acid sequence of SEQ ID NO: 93. Alternatively or in addition (e.g., in addition), the anti-TfR antibody of the present disclosure comprises a CDR-L1, a CDR-L2, and a CDR-L3, which collectively contains no more than 5 amino acid variations (e.g., no more than 5, 4, 3, 2 or 1 amino acid variation) as compared with the CDR-L1 having the amino acid sequence of SEQ ID NO: 94, CDR-L2 having the amino acid sequence of SEQ ID NO: 95, and CDR-L3 having the amino acid sequence of SEQ ID NO: 96.

In some embodiments, the anti-TfR antibody of the present disclosure comprises a CDR-H1, a CDR-H2, and a CDR-H3 that collectively are at least 75% (e.g., 75%, 80%, 85%, 90%, 95%, 98%, or 99%) identical to the CDR-H1 having the amino acid sequence of SEQ ID NO: 91, CDR-H2 having the amino acid sequence of SEQ ID NO: 92, and CDR-H3 having the amino acid sequence of SEQ ID NO: 93. Alternatively or in addition (e.g., in addition), the anti-TfR antibody of the present disclosure comprises a CDR-L1, a CDR-L2, and a CDR-L3 that collectively are at least 75% (e.g., 75%, 80%, 85%, 90%, 95%, 98%, or 99%) identical to the to the CDR-L1 having the amino acid sequence of SEQ ID NO: 94, CDR-L2 having the amino acid sequence of SEQ ID NO: 95, and CDR-L3 having the amino acid sequence of SEQ ID NO: 96.

In some embodiments, the anti-TfR antibody of the present disclosure comprises: a CDR-H1 having no more than 3 amino acid variations (e.g., no more than 3, 2, or 1 amino acid variation) as compared with the CDR-H1 having the amino acid sequence of SEQ ID NO: 91; a CDR-H2 having no more than 3 amino acid variations (e.g., no more than 3, 2, or 1 amino acid variation) as compared with the CDR-H2 having the amino acid sequence of SEQ ID NO: 92; and/or (e.g., and) a CDR-H3 having no more than 3 amino acid variations (e.g., no more than 3, 2, or 1 amino acid variation) as compared with the CDR-H3 having the amino acid sequence of SEQ ID NO: 93. Alternatively or in addition (e.g., in addition), the anti-TfR antibody of the present disclosure comprises: a CDR-L1 having no more than 3 amino acid variations (e.g., no more than 3, 2, or 1 amino acid variation) as compared with the CDR-L1 having the amino acid sequence of SEQ ID NO: 94; a CDR-L2 having no more than 3 amino acid variations (e.g., no more than 3, 2, or 1 amino acid variation) as compared with the CDR-L2 having the amino acid sequence of SEQ ID NO: 95; and/or (e.g., and) a CDR-L3 having no more than 3 amino acid variations (e.g., no more than 3, 2, or 1 amino acid variation) as compared with the CDR-L3 having the amino acid sequence of SEQ ID NO: 96.

In some embodiments, the anti-TfR antibody of the present disclosure comprises a VH comprising the amino acid sequence of SEQ ID NO: 97. Alternatively or in addition (e.g., in addition), the anti-TfR antibody of the present disclosure comprises a VL comprising the amino acid sequence of SEQ ID NO: 98.

In some embodiments, the anti-TfR antibody of the present disclosure comprises a VH containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 97. Alternatively or in addition (e.g., in addition), the anti-TfR antibody of the present disclosure comprises a VL containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VL as set forth in SEQ ID NO: 98.

In some embodiments, the anti-TfR antibody of the present disclosure comprises a VH comprising an amino acid sequence that is at least 75% (e.g., 75%, 80%, 85%, 90%, 95%, 98%, or 99%) identical to the VH as set forth in SEQ ID NO: 97. Alternatively or in addition (e.g., in addition), the anti-TfR antibody of the present disclosure comprises a VL comprising an amino acid sequence that is at least 75% (e.g., 75%, 80%, 85%, 90%, 95%, 98%, or 99%) identical to the VL as set forth in SEQ ID NO: 98.

In some embodiments, the anti-TfR antibody of the present disclosure comprises a CDR-H1, a CDR-H2, and a CDR-H3 of a heavy chain variable domain having the amino acid sequence of SEQ ID NO: 104. Alternatively or in addition (e.g., in addition), the anti-TfR antibody of the present disclosure comprises a CDR-L1, a CDR-L2, and a CDR-L3 of a light chain variable domain having the amino acid sequence of SEQ ID NO: 105.

In some embodiments, anti-TfR antibody of the present disclosure comprises a CDR-H1, a CDR-H2, and a CDR-H3, which collectively contains no more than 5 amino acid variations (e.g., no more than 5, 4, 3, 2, or 1 amino acid variation) as compared with the CDR-H1 having the amino acid sequence of SEQ ID NO: 99, CDR-H2 having the amino acid sequence of SEQ ID NO: 100, and CDR-H3 having the amino acid sequence of SEQ ID NO: 101. Alternatively or in addition (e.g., in addition), the anti-TfR antibody of the present disclosure comprises a CDR-L1, a CDR-L2, and a CDR-L3, which collectively contains no more than 5 amino acid variations (e.g., no more than 5, 4, 3, 2 or 1 amino acid variation) as compared with the CDR-L1 having the amino acid sequence of SEQ ID NO: 102, CDR-L2 having the amino acid sequence of SEQ ID NO: 60, and CDR-L3 having the amino acid sequence of SEQ ID NO: 103.

In some embodiments, the anti-TfR antibody of the present disclosure comprises a CDR-H1, a CDR-H2, and a CDR-H3 that collectively are at least 75% (e.g., 75%, 80%, 85%, 90%, 95%, 98%, or 99%) identical to the CDR-H1 having the amino acid sequence of SEQ ID NO: 99, CDR-H2 having the amino acid sequence of SEQ ID NO: 100, and CDR-H3 having the amino acid sequence of SEQ ID NO: 101. Alternatively or in addition (e.g., in addition), the anti-TfR antibody of the present disclosure comprises a CDR-L1, a CDR-L2, and a CDR-L3 that collectively are at least 75% (e.g., 75%, 80%, 85%, 90%, 95%, 98%, or 99%) identical to the to the CDR-L1 having the amino acid sequence of SEQ ID NO: 102, CDR-L2 having the amino acid sequence of SEQ ID NO: 60, and CDR-L3 having the amino acid sequence of SEQ ID NO: 103.

In some embodiments, the anti-TfR antibody of the present disclosure comprises: a CDR-H1 having no more than 3 amino acid variations (e.g., no more than 3, 2, or 1 amino acid variation) as compared with the CDR-H1 having the amino acid sequence of SEQ ID NO: 99; a CDR-H2 having no more than 3 amino acid variations (e.g., no more than 3, 2, or 1 amino acid variation) as compared with the CDR-H2 having the amino acid sequence of SEQ ID NO: 100; and/or (e.g., and) a CDR-H3 having no more than 3 amino acid variations (e.g., no more than 3, 2, or 1 amino acid variation) as compared with the CDR-H3 having the amino acid sequence of SEQ ID NO: 101. Alternatively or in addition (e.g., in addition), the anti-TfR antibody of the present disclosure comprises: a CDR-L1 having no more than 3 amino acid variations (e.g., no more than 3, 2, or 1 amino acid variation) as compared with the CDR-L1 having the amino acid sequence of SEQ ID NO: 102; a CDR-L2 having no more than 3 amino acid variations (e.g., no more than 3, 2, or 1 amino acid variation) as compared with the CDR-L2 having the amino acid sequence of SEQ ID NO: 60; and/or (e.g., and) a CDR-L3 having no more than 3 amino acid variations (e.g., no more than 3, 2, or 1 amino acid variation) as compared with the CDR-L3 having the amino acid sequence of SEQ ID NO: 103.

In some embodiments, the anti-TfR antibody of the present disclosure comprises a VH comprising the amino acid sequence of SEQ ID NO: 104. Alternatively or in addition (e.g., in addition), the anti-TfR antibody of the present disclosure comprises a VL comprising the amino acid sequence of SEQ ID NO: 105.

In some embodiments, the anti-TfR antibody of the present disclosure comprises a VH containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 104. Alternatively or in addition (e.g., in addition), the anti-TfR antibody of the present disclosure comprises a VL containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VL as set forth in SEQ ID NO: 105.

In some embodiments, the anti-TfR antibody of the present disclosure comprises a VH comprising an amino acid sequence that is at least 75% (e.g., 75%, 80%, 85%, 90%, 95%, 98%, or 99%) identical to the VH as set forth in SEQ ID NO: 104. Alternatively or in addition (e.g., in addition), the anti-TfR antibody of the present disclosure comprises a VL comprising an amino acid sequence that is at least 75% (e.g., 75%, 80%, 85%, 90%, 95%, 98%, or 99%) identical to the VL as set forth in SEQ ID NO: 105.

In some embodiments, the anti-TfR antibody of the present disclosure comprises a CDR-H1, a CDR-H2, and a CDR-H3 of a heavy chain variable domain having the amino acid sequence of SEQ ID NO: 112. Alternatively or in addition (e.g., in addition), the anti-TfR antibody of the present disclosure comprises a CDR-L1, a CDR-L2, and a CDR-L3 of a light chain variable domain having the amino acid sequence of SEQ ID NO: 113.

In some embodiments, the anti-TfR antibody of the present disclosure comprises a CDR-H1 having the amino acid sequence of SEQ ID NO: 106 (according to the IMGT definition system), a CDR-H2 having the amino acid sequence of SEQ ID NO: 107 (according to the IMGT definition system), a CDR-H3 having the amino acid sequence of SEQ ID NO: 108 (according to the IMGT definition system), a CDR-L1 having the amino acid sequence of SEQ ID NO: 109 (according to the IMGT definition system), a CDR-L2 having the amino acid sequence of SEQ ID NO: 110 (according to the IMGT definition system), and a CDR-L3 having the amino acid sequence of SEQ ID NO: 111 (according to the IMGT definition system).

In some embodiments, anti-TfR antibody of the present disclosure comprises a CDR-H1, a CDR-H2, and a CDR-H3, which collectively contains no more than 5 amino acid variations (e.g., no more than 5, 4, 3, 2, or 1 amino acid variation) as compared with the CDR-H1 having the amino acid sequence of SEQ ID NO: 106, CDR-H2 having the amino acid sequence of SEQ ID NO: 107, and CDR-H3 having the amino acid sequence of SEQ ID NO: 108. Alternatively or in addition (e.g., in addition), the anti-TfR antibody of the present disclosure comprises a CDR-L1, a CDR-L2, and a CDR-L3, which collectively contains no more than 5 amino acid variations (e.g., no more than 5, 4, 3, 2 or 1 amino acid variation) as compared with the CDR-L1 having the amino acid sequence of SEQ ID NO: 109, CDR-L2 having the amino acid sequence of SEQ ID NO: 110, and CDR-L3 having the amino acid sequence of SEQ ID NO: 111.

In some embodiments, the anti-TfR antibody of the present disclosure comprises a CDR-H1, a CDR-H2, and a CDR-H3 that collectively are at least 75% (e.g., 75%, 80%, 85%, 90%, 95%, 98%, or 99%) identical to the CDR-H1 having the amino acid sequence of SEQ ID NO: 106, CDR-H2 having the amino acid sequence of SEQ ID NO: 107, and CDR-H3 having the amino acid sequence of SEQ ID NO: 108. Alternatively or in addition (e.g., in addition), the anti-TfR antibody of the present disclosure comprises a CDR-L1, a CDR-L2, and a CDR-L3 that collectively are at least 75% (e.g., 75%, 80%, 85%, 90%, 95%, 98%, or 99%) identical to the to the CDR-L1 having the amino acid sequence of SEQ ID NO: 109, CDR-L2 having the amino acid sequence of SEQ ID NO: 110, and CDR-L3 having the amino acid sequence of SEQ ID NO: 111.

In some embodiments, the anti-TfR antibody of the present disclosure comprises: a CDR-H1 having no more than 3 amino acid variations (e.g., no more than 3, 2, or 1 amino acid variation) as compared with the CDR-H1 having the amino acid sequence of SEQ ID NO: 106; a CDR-H2 having no more than 3 amino acid variations (e.g., no more than 3, 2, or 1 amino acid variation) as compared with the CDR-H2 having the amino acid sequence of SEQ ID NO: 107; and/or (e.g., and) a CDR-H3 having no more than 3 amino acid variations (e.g., no more than 3, 2, or 1 amino acid variation) as compared with the CDR-H3 having the amino acid sequence of SEQ ID NO: 108. Alternatively or in addition (e.g., in addition), the anti-TfR antibody of the present disclosure comprises: a CDR-L1 having no more than 3 amino acid variations (e.g., no more than 3, 2, or 1 amino acid variation) as compared with the CDR-L1 having the amino acid sequence of SEQ ID NO: 109; a CDR-L2 having no more than 3 amino acid variations (e.g., no more than 3, 2, or 1 amino acid variation) as compared with the CDR-L2 having the amino acid sequence of SEQ ID NO: 110; and/or (e.g., and) a CDR-L3 having no more than 3 amino acid variations (e.g., no more than 3, 2, or 1 amino acid variation) as compared with the CDR-L3 having the amino acid sequence of SEQ ID NO: 111.

In some embodiments, the anti-TfR antibody of the present disclosure comprises a VH comprising the amino acid sequence of SEQ ID NO: 112. Alternatively or in addition (e.g., in addition), the anti-TfR antibody of the present disclosure comprises a VL comprising the amino acid sequence of SEQ ID NO: 113.

In some embodiments, the anti-TfR antibody of the present disclosure comprises a VH containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 112. Alternatively or in addition (e.g., in addition), the anti-TfR antibody of the present disclosure comprises a VL containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VL as set forth in SEQ ID NO: 113.

In some embodiments, the anti-TfR antibody of the present disclosure comprises a VH comprising an amino acid sequence that is at least 75% (e.g., 75%, 80%, 85%, 90%, 95%, 98%, or 99%) identical to the VH as set forth in SEQ ID NO: 112. Alternatively or in addition (e.g., in addition), the anti-TfR antibody of the present disclosure comprises a VL comprising an amino acid sequence that is at least 75% (e.g., 75%, 80%, 85%, 90%, 95%, 98%, or 99%) identical to the VL as set forth in SEQ ID NO: 113.

In some embodiments, the anti-TfR antibody of the present disclosure comprises a CDR-H1, a CDR-H2, and a CDR-H3 of a heavy chain variable domain having the amino acid sequence of SEQ ID NO: 117. Alternatively or in addition (e.g., in addition), the anti-TfR antibody of the present disclosure comprises a CDR-L1, a CDR-L2, and a CDR-L3 of a light chain variable domain having the amino acid sequence of SEQ ID NO: 118.

In some embodiments, the anti-TfR antibody of the present disclosure comprises a CDR-H1 having the amino acid sequence of SEQ ID NO: 79 (according to the IMGT definition system), a CDR-H2 having the amino acid sequence of SEQ ID NO: 114 (according to the IMGT definition system), a CDR-H3 having the amino acid sequence of SEQ ID NO: 115 (according to the IMGT definition system), a CDR-L1 having the amino acid sequence of SEQ ID NO: 82 (according to the IMGT definition system), a CDR-L2 having the amino acid sequence of SEQ ID NO: 83 (according to the IMGT definition system), and a CDR-L3 having the amino acid sequence of SEQ ID NO: 116 (according to the IMGT definition system).

In some embodiments, anti-TfR antibody of the present disclosure comprises a CDR-H1, a CDR-H2, and a CDR-H3, which collectively contains no more than 5 amino acid variations (e.g., no more than 5, 4, 3, 2, or 1 amino acid variation) as compared with the CDR-H1 having the amino acid sequence of SEQ ID NO: 79, CDR-H2 having the amino acid sequence of SEQ ID NO: 114, and CDR-H3 having the amino acid sequence of SEQ ID NO: 115. Alternatively or in addition (e.g., in addition), the anti-TfR antibody of the present disclosure comprises a CDR-L1, a CDR-L2, and a CDR-L3, which collectively contains no more than 5 amino acid variations (e.g., no more than 5, 4, 3, 2 or 1 amino acid variation) as compared with the CDR-L1 having the amino acid sequence of SEQ ID NO: 82, CDR-L2 having the amino acid sequence of SEQ ID NO: 83, and CDR-L3 having the amino acid sequence of SEQ ID NO: 116.

In some embodiments, the anti-TfR antibody of the present disclosure comprises a CDR-H1, a CDR-H2, and a CDR-H3 that collectively are at least 75% (e.g., 75%, 80%, 85%, 90%, 95%, 98%, or 99%) identical to the CDR-H1 having the amino acid sequence of SEQ ID NO: 79, CDR-H2 having the amino acid sequence of SEQ ID NO: 114, and CDR-H3 having the amino acid sequence of SEQ ID NO: 115. Alternatively or in addition (e.g., in addition), the anti-TfR antibody of the present disclosure comprises a CDR-L1, a CDR-L2, and a CDR-L3 that collectively are at least 75% (e.g., 75%, 80%, 85%, 90%, 95%, 98%, or 99%) identical to the to the CDR-L1 having the amino acid sequence of SEQ ID NO: 82, CDR-L2 having the amino acid sequence of SEQ ID NO: 83, and CDR-L3 having the amino acid sequence of SEQ ID NO: 116.

In some embodiments, the anti-TfR antibody of the present disclosure comprises: a CDR-H1 having no more than 3 amino acid variations (e.g., no more than 3, 2, or 1 amino acid variation) as compared with the CDR-H1 having the amino acid sequence of SEQ ID NO: 79; a CDR-H2 having no more than 3 amino acid variations (e.g., no more than 3, 2, or 1 amino acid variation) as compared with the CDR-H2 having the amino acid sequence of SEQ ID NO: 114; and/or (e.g., and) a CDR-H3 having no more than 3 amino acid variations (e.g., no more than 3, 2, or 1 amino acid variation) as compared with the CDR-H3 having the amino acid sequence of SEQ ID NO: 115. Alternatively or in addition (e.g., in addition), the anti-TfR antibody of the present disclosure comprises: a CDR-L1 having no more than 3 amino acid variations (e.g., no more than 3, 2, or 1 amino acid variation) as compared with the CDR-L1 having the amino acid sequence of SEQ ID NO: 82; a CDR-L2 having no more than 3 amino acid variations (e.g., no more than 3, 2, or 1 amino acid variation) as compared with the CDR-L2 having the amino acid sequence of SEQ ID NO: 83; and/or (e.g., and) a CDR-L3 having no more than 3 amino acid variations (e.g., no more than 3, 2, or 1 amino acid variation) as compared with the CDR-L3 having the amino acid sequence of SEQ ID NO: 116.

In some embodiments, the anti-TfR antibody of the present disclosure comprises a VH comprising the amino acid sequence of SEQ ID NO: 117. Alternatively or in addition (e.g., in addition), the anti-TfR antibody of the present disclosure comprises a VL comprising the amino acid sequence of SEQ ID NO: 118.

In some embodiments, the anti-TfR antibody of the present disclosure comprises a VH containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 117. Alternatively or in addition (e.g., in addition), the anti-TfR antibody of the present disclosure comprises a VL containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VL as set forth in SEQ ID NO: 118.

In some embodiments, the anti-TfR antibody of the present disclosure comprises a VH comprising an amino acid sequence that is at least 75% (e.g., 75%, 80%, 85%, 90%, 95%, 98%, or 99%) identical to the VH as set forth in SEQ ID NO: 117. Alternatively or in addition (e.g., in addition), the anti-TfR antibody of the present disclosure comprises a VL comprising an amino acid sequence that is at least 75% (e.g., 75%, 80%, 85%, 90%, 95%, 98%, or 99%) identical to the VL as set forth in SEQ ID NO: 118.

In some embodiments, the anti-TfR antibody of the present disclosure comprises a CDR-H1, a CDR-H2, and a CDR-H3 of a heavy chain variable domain having the amino acid sequence of SEQ ID NO: 124. Alternatively or in addition (e.g., in addition), the anti-TfR antibody of the present disclosure comprises a CDR-L1, a CDR-L2, and a CDR-L3 of a light chain variable domain having the amino acid sequence of SEQ ID NO: 125.

In some embodiments, the anti-TfR antibody of the present disclosure comprises a CDR-H1 having the amino acid sequence of SEQ ID NO: 119 (according to the IMGT definition system), a CDR-H2 having the amino acid sequence of SEQ ID NO: 120 (according to the IMGT definition system), a CDR-H3 having the amino acid sequence of SEQ ID NO: 121 (according to the IMGT definition system), a CDR-L1 having the amino acid sequence of SEQ ID NO: 122 (according to the IMGT definition system), a CDR-L2 having the amino acid sequence of SEQ ID NO: 45 (according to the IMGT definition system), and a CDR-L3 having the amino acid sequence of SEQ ID NO: 123 (according to the IMGT definition system).

In some embodiments, anti-TfR antibody of the present disclosure comprises a CDR-H1, a CDR-H2, and a CDR-H3, which collectively contains no more than 5 amino acid variations (e.g., no more than 5, 4, 3, 2, or 1 amino acid variation) as compared with the CDR-H1 having the amino acid sequence of SEQ ID NO: 119, CDR-H2 having the amino acid sequence of SEQ ID NO: 120, and CDR-H3 having the amino acid sequence of SEQ ID NO: 121. Alternatively or in addition (e.g., in addition), the anti-TfR antibody of the present disclosure comprises a CDR-L1, a CDR-L2, and a CDR-L3, which collectively contains no more than 5 amino acid variations (e.g., no more than 5, 4, 3, 2 or 1 amino acid variation) as compared with the CDR-L1 having the amino acid sequence of SEQ ID NO: 122, CDR-L2 having the amino acid sequence of SEQ ID NO: 45, and CDR-L3 having the amino acid sequence of SEQ ID NO: 123.

In some embodiments, the anti-TfR antibody of the present disclosure comprises a CDR-H1, a CDR-H2, and a CDR-H3 that collectively are at least 75% (e.g., 75%, 80%, 85%, 90%, 95%, 98%, or 99%) identical to the CDR-H1 having the amino acid sequence of SEQ ID NO: 119, CDR-H2 having the amino acid sequence of SEQ ID NO: 120, and CDR-H3 having the amino acid sequence of SEQ ID NO: 121. Alternatively or in addition (e.g., in addition), the anti-TfR antibody of the present disclosure comprises a CDR-L1, a CDR-L2, and a CDR-L3 that collectively are at least 75% (e.g., 75%, 80%, 85%, 90%, 95%, 98%, or 99%) identical to the to the CDR-L1 having the amino acid sequence of SEQ ID NO: 122, CDR-L2 having the amino acid sequence of SEQ ID NO: 45, and CDR-L3 having the amino acid sequence of SEQ ID NO: 123.

In some embodiments, the anti-TfR antibody of the present disclosure comprises: a CDR-H1 having no more than 3 amino acid variations (e.g., no more than 3, 2, or 1 amino acid variation) as compared with the CDR-H1 having the amino acid sequence of SEQ ID NO: 119; a CDR-H2 having no more than 3 amino acid variations (e.g., no more than 3, 2, or 1 amino acid variation) as compared with the CDR-H2 having the amino acid sequence of SEQ ID NO: 120; and/or (e.g., and) a CDR-H3 having no more than 3 amino acid variations (e.g., no more than 3, 2, or 1 amino acid variation) as compared with the CDR-H3 having the amino acid sequence of SEQ ID NO: 121. Alternatively or in addition (e.g., in addition), the anti-TfR antibody of the present disclosure comprises: a CDR-L1 having no more than 3 amino acid variations (e.g., no more than 3, 2, or 1 amino acid variation) as compared with the CDR-L1 having the amino acid sequence of SEQ ID NO: 122; a CDR-L2 having no more than 3 amino acid variations (e.g., no more than 3, 2, or 1 amino acid variation) as compared with the CDR-L2 having the amino acid sequence of SEQ ID NO: 45; and/or (e.g., and) a CDR-L3 having no more than 3 amino acid variations (e.g., no more than 3, 2, or 1 amino acid variation) as compared with the CDR-L3 having the amino acid sequence of SEQ ID NO: 123.

In some embodiments, the anti-TfR antibody of the present disclosure comprises a VH comprising the amino acid sequence of SEQ ID NO: 124. Alternatively or in addition (e.g., in addition), the anti-TfR antibody of the present disclosure comprises a VL comprising the amino acid sequence of SEQ ID NO: 125.

In some embodiments, the anti-TfR antibody of the present disclosure comprises a VH containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 124. Alternatively or in addition (e.g., in addition), the anti-TfR antibody of the present disclosure comprises a VL containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VL as set forth in SEQ ID NO: 125.

n some embodiments, the anti-TfR antibody of the present disclosure comprises a VH comprising an amino acid sequence that is at least 75% (e.g., 75%, 80%, 85%, 90%, 95%, 98%, or 99%) identical to the VH as set forth in SEQ ID NO: 124. Alternatively or in addition (e.g., in addition), the anti-TfR antibody of the present disclosure comprises a VL comprising an amino acid sequence that is at least 75% (e.g., 75%, 80%, 85%, 90%, 95%, 98%, or 99%) identical to the VL as set forth in SEQ ID NO: 125.

In some embodiments, the anti-TfR antibody of the present disclosure comprises a CDR-H1, a CDR-H2, and a CDR-H3 of a heavy chain variable domain having the amino acid sequence of SEQ ID NO: 132. Alternatively or in addition (e.g., in addition), the anti-TfR antibody of the present disclosure comprises a CDR-L1, a CDR-L2, and a CDR-L3 of a light chain variable domain having the amino acid sequence of SEQ ID NO: 133.

In some embodiments, the anti-TfR antibody of the present disclosure comprises a CDR-H1 having the amino acid sequence of SEQ ID NO: 126 (according to the IMGT definition system), a CDR-H2 having the amino acid sequence of SEQ ID NO: 127 (according to the IMGT definition system), a CDR-H3 having the amino acid sequence of SEQ ID NO: 128 (according to the IMGT definition system), a CDR-L1 having the amino acid sequence of SEQ ID NO: 129 (according to the IMGT definition system), a CDR-L2 having the amino acid sequence of SEQ ID NO: 130 (according to the IMGT definition system), and a CDR-L3 having the amino acid sequence of SEQ ID NO: 131 (according to the IMGT definition system).

In some embodiments, anti-TfR antibody of the present disclosure comprises a CDR-H1, a CDR-H2, and a CDR-H3, which collectively contains no more than 5 amino acid variations (e.g., no more than 5, 4, 3, 2, or 1 amino acid variation) as compared with the CDR-H1 having the amino acid sequence of SEQ ID NO: 126, CDR-H2 having the amino acid sequence of SEQ ID NO: 127, and CDR-H3 having the amino acid sequence of SEQ ID NO: 128. Alternatively or in addition (e.g., in addition), the anti-TfR antibody of the present disclosure comprises a CDR-L1, a CDR-L2, and a CDR-L3, which collectively contains no more than 5 amino acid variations (e.g., no more than 5, 4, 3, 2 or 1 amino acid variation) as compared with the CDR-L1 having the amino acid sequence of SEQ ID NO: 129, CDR-L2 having the amino acid sequence of SEQ ID NO: 130, and CDR-L3 having the amino acid sequence of SEQ ID NO: 131.

In some embodiments, the anti-TfR antibody of the present disclosure comprises a CDR-H1, a CDR-H2, and a CDR-H3 that collectively are at least 75% (e.g., 75%, 80%, 85%, 90%, 95%, 98%, or 99%) identical to the CDR-H1 having the amino acid sequence of SEQ ID NO: 126, CDR-H2 having the amino acid sequence of SEQ ID NO: 127, and CDR-H3 having the amino acid sequence of SEQ ID NO: 128. Alternatively or in addition (e.g., in addition), the anti-TfR antibody of the present disclosure comprises a CDR-L1, a CDR-L2, and a CDR-L3 that collectively are at least 75% (e.g., 75%, 80%, 85%, 90%, 95%, 98%, or 99%) identical to the to the CDR-L1 having the amino acid sequence of SEQ ID NO: 129, CDR-L2 having the amino acid sequence of SEQ ID NO: 130, and CDR-L3 having the amino acid sequence of SEQ ID NO: 131.

In some embodiments, the anti-TfR antibody of the present disclosure comprises: a CDR-H1 having no more than 3 amino acid variations (e.g., no more than 3, 2, or 1 amino acid variation) as compared with the CDR-H1 having the amino acid sequence of SEQ ID NO: 126; a CDR-H2 having no more than 3 amino acid variations (e.g., no more than 3, 2, or 1 amino acid variation) as compared with the CDR-H2 having the amino acid sequence of SEQ ID NO: 127; and/or (e.g., and) a CDR-H3 having no more than 3 amino acid variations (e.g., no more than 3, 2, or 1 amino acid variation) as compared with the CDR-H3 having the amino acid sequence of SEQ ID NO: 128. Alternatively or in addition (e.g., in addition), the anti-TfR antibody of the present disclosure comprises: a CDR-L1 having no more than 3 amino acid variations (e.g., no more than 3, 2, or 1 amino acid variation) as compared with the CDR-L1 having the amino acid sequence of SEQ ID NO: 129; a CDR-L2 having no more than 3 amino acid variations (e.g., no more than 3, 2, or 1 amino acid variation) as compared with the CDR-L2 having the amino acid sequence of SEQ ID NO: 130; and/or (e.g., and) a CDR-L3 having no more than 3 amino acid variations (e.g., no more than 3, 2, or 1 amino acid variation) as compared with the CDR-L3 having the amino acid sequence of SEQ ID NO: 131.

In some embodiments, the anti-TfR antibody of the present disclosure comprises a VH comprising the amino acid sequence of SEQ ID NO: 132. Alternatively or in addition (e.g., in addition), the anti-TfR antibody of the present disclosure comprises a VL comprising the amino acid sequence of SEQ ID NO: 133.

In some embodiments, the anti-TfR antibody of the present disclosure comprises a VH containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 132. Alternatively or in addition (e.g., in addition), the anti-TfR antibody of the present disclosure comprises a VL containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VL as set forth in SEQ ID NO: 133.

In some embodiments, the anti-TfR antibody of the present disclosure comprises a VH comprising an amino acid sequence that is at least 75% (e.g., 75%, 80%, 85%, 90%, 95%, 98%, or 99%) identical to the VH as set forth in SEQ ID NO: 132. Alternatively or in addition (e.g., in addition), the anti-TfR antibody of the present disclosure comprises a VL comprising an amino acid sequence that is at least 75% (e.g., 75%, 80%, 85%, 90%, 95%, 98%, or 99%) identical to the VL as set forth in SEQ ID NO: 133.

In some embodiments, the anti-TfR antibody of the present disclosure comprises a CDR-H1, a CDR-H2, and a CDR-H3 of a heavy chain variable domain having the amino acid sequence of SEQ ID NO: 136. Alternatively or in addition (e.g., in addition), the anti-TfR antibody of the present disclosure comprises a CDR-L1, a CDR-L2, and a CDR-L3 of a light chain variable domain having the amino acid sequence of SEQ ID NO: 137.

In some embodiments, the anti-TfR antibody of the present disclosure comprises a CDR-H1 having the amino acid sequence of SEQ ID NO: 79 (according to the IMGT definition system), a CDR-H2 having the amino acid sequence of SEQ ID NO: 2 (according to the IMGT definition system), a CDR-H3 having the amino acid sequence of SEQ ID NO: 134 (according to the IMGT definition system), a CDR-L1 having the amino acid sequence of SEQ ID NO: 75 (according to the IMGT definition system), a CDR-L2 having the amino acid sequence of SEQ ID NO: 45 (according to the IMGT definition system), and a CDR-L3 having the amino acid sequence of SEQ ID NO: 135 (according to the IMGT definition system).

In some embodiments, anti-TfR antibody of the present disclosure comprises a CDR-H1, a CDR-H2, and a CDR-H3, which collectively contains no more than 5 amino acid variations (e.g., no more than 5, 4, 3, 2, or 1 amino acid variation) as compared with the CDR-H1 having the amino acid sequence of SEQ ID NO: 79, CDR-H2 having the amino acid sequence of SEQ ID NO: 2, and CDR-H3 having the amino acid sequence of SEQ ID NO: 134. Alternatively or in addition (e.g., in addition), the anti-TfR antibody of the present disclosure comprises a CDR-L1, a CDR-L2, and a CDR-L3, which collectively contains no more than 5 amino acid variations (e.g., no more than 5, 4, 3, 2 or 1 amino acid variation) as compared with the CDR-L1 having the amino acid sequence of SEQ ID NO: 75, CDR-L2 having the amino acid sequence of SEQ ID NO: 45, and CDR-L3 having the amino acid sequence of SEQ ID NO: 135.

In some embodiments, the anti-TfR antibody of the present disclosure comprises a CDR-H1, a CDR-H2, and a CDR-H3 that collectively are at least 75% (e.g., 75%, 80%, 85%, 90%, 95%, 98%, or 99%) identical to the CDR-H1 having the amino acid sequence of SEQ ID NO: 79, CDR-H2 having the amino acid sequence of SEQ ID NO: 2, and CDR-H3 having the amino acid sequence of SEQ ID NO: 134. Alternatively or in addition (e.g., in addition), the anti-TfR antibody of the present disclosure comprises a CDR-L1, a CDR-L2, and a CDR-L3 that collectively are at least 75% (e.g., 75%, 80%, 85%, 90%, 95%, 98%, or 99%) identical to the to the CDR-L1 having the amino acid sequence of SEQ ID NO: 75, CDR-L2 having the amino acid sequence of SEQ ID NO: 45, and CDR-L3 having the amino acid sequence of SEQ ID NO: 135.

In some embodiments, the anti-TfR antibody of the present disclosure comprises: a CDR-H1 having no more than 3 amino acid variations (e.g., no more than 3, 2, or 1 amino acid variation) as compared with the CDR-H1 having the amino acid sequence of SEQ ID NO: 79; a CDR-H2 having no more than 3 amino acid variations (e.g., no more than 3, 2, or 1 amino acid variation) as compared with the CDR-H2 having the amino acid sequence of SEQ ID NO: 2; and/or (e.g., and) a CDR-H3 having no more than 3 amino acid variations (e.g., no more than 3, 2, or 1 amino acid variation) as compared with the CDR-H3 having the amino acid sequence of SEQ ID NO: 134. Alternatively or in addition (e.g., in addition), the anti-TfR antibody of the present disclosure comprises: a CDR-L1 having no more than 3 amino acid variations (e.g., no more than 3, 2, or 1 amino acid variation) as compared with the CDR-L1 having the amino acid sequence of SEQ ID NO: 75; a CDR-L2 having no more than 3 amino acid variations (e.g., no more than 3, 2, or 1 amino acid variation) as compared with the CDR-L2 having the amino acid sequence of SEQ ID NO: 45; and/or (e.g., and) a CDR-L3 having no more than 3 amino acid variations (e.g., no more than 3, 2, or 1 amino acid variation) as compared with the CDR-L3 having the amino acid sequence of SEQ ID NO: 135.

In some embodiments, the anti-TfR antibody of the present disclosure comprises a VH comprising the amino acid sequence of SEQ ID NO: 136. Alternatively or in addition (e.g., in addition), the anti-TfR antibody of the present disclosure comprises a VL comprising the amino acid sequence of SEQ ID NO: 137.

In some embodiments, the anti-TfR antibody of the present disclosure comprises a VH containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 136. Alternatively or in addition (e.g., in addition), the anti-TfR antibody of the present disclosure comprises a VL containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VL as set forth in SEQ ID NO: 137.

In some embodiments, the anti-TfR antibody of the present disclosure comprises a VH comprising an amino acid sequence that is at least 75% (e.g., 75%, 80%, 85%, 90%, 95%, 98%, or 99%) identical to the VH as set forth in SEQ ID NO: 136. Alternatively or in addition (e.g., in addition), the anti-TfR antibody of the present disclosure comprises a VL comprising an amino acid sequence that is at least 75% (e.g., 75%, 80%, 85%, 90%, 95%, 98%, or 99%) identical to the VL as set forth in SEQ ID NO: 137.

In some embodiments, the anti-TfR antibody of the present disclosure comprises a CDR-H1, a CDR-H2, and a CDR-H3 of a heavy chain variable domain having the amino acid sequence of SEQ ID NO: 143. Alternatively or in addition (e.g., in addition), the anti-TfR antibody of the present disclosure comprises a CDR-L1, a CDR-L2, and a CDR-L3 of a light chain variable domain having the amino acid sequence of SEQ ID NO: 144.

In some embodiments, the anti-TfR antibody of the present disclosure comprises a CDR-H1 having the amino acid sequence of SEQ ID NO: 138 (according to the IMGT definition system), a CDR-H2 having the amino acid sequence of SEQ ID NO: 139 (according to the IMGT definition system), a CDR-H3 having the amino acid sequence of SEQ ID NO: 140 (according to the IMGT definition system), a CDR-L1 having the amino acid sequence of SEQ ID NO: 141 (according to the IMGT definition system), a CDR-L2 having the amino acid sequence of SEQ ID NO: 29 (according to the IMGT definition system), and a CDR-L3 having the amino acid sequence of SEQ ID NO: 142 (according to the IMGT definition system).

In some embodiments, anti-TfR antibody of the present disclosure comprises a CDR-H1, a CDR-H2, and a CDR-H3, which collectively contains no more than 5 amino acid variations (e.g., no more than 5, 4, 3, 2, or 1 amino acid variation) as compared with the CDR-H1 having the amino acid sequence of SEQ ID NO: 138, CDR-H2 having the amino acid sequence of SEQ ID NO: 139, and CDR-H3 having the amino acid sequence of SEQ ID NO: 140. Alternatively or in addition (e.g., in addition), the anti-TfR antibody of the present disclosure comprises a CDR-L1, a CDR-L2, and a CDR-L3, which collectively contains no more than 5 amino acid variations (e.g., no more than 5, 4, 3, 2 or 1 amino acid variation) as compared with the CDR-L1 having the amino acid sequence of SEQ ID NO: 141, CDR-L2 having the amino acid sequence of SEQ ID NO: 29, and CDR-L3 having the amino acid sequence of SEQ ID NO: 142.

In some embodiments, the anti-TfR antibody of the present disclosure comprises a CDR-H1, a CDR-H2, and a CDR-H3 that collectively are at least 75% (e.g., 75%, 80%, 85%, 90%, 95%, 98%, or 99%) identical to the CDR-H1 having the amino acid sequence of SEQ ID NO: 138, CDR-H2 having the amino acid sequence of SEQ ID NO: 139, and CDR-H3 having the amino acid sequence of SEQ ID NO: 140. Alternatively or in addition (e.g., in addition), the anti-TfR antibody of the present disclosure comprises a CDR-L1, a CDR-L2, and a CDR-L3 that collectively are at least 75% (e.g., 75%, 80%, 85%, 90%, 95%, 98%, or 99%) identical to the to the CDR-L1 having the amino acid sequence of SEQ ID NO: 141, CDR-L2 having the amino acid sequence of SEQ ID NO: 29, and CDR-L3 having the amino acid sequence of SEQ ID NO: 142.

In some embodiments, the anti-TfR antibody of the present disclosure comprises: a CDR-H1 having no more than 3 amino acid variations (e.g., no more than 3, 2, or 1 amino acid variation) as compared with the CDR-H1 having the amino acid sequence of SEQ ID NO: 138; a CDR-H2 having no more than 3 amino acid variations (e.g., no more than 3, 2, or 1 amino acid variation) as compared with the CDR-H2 having the amino acid sequence of SEQ ID NO: 139; and/or (e.g., and) a CDR-H3 having no more than 3 amino acid variations (e.g., no more than 3, 2, or 1 amino acid variation) as compared with the CDR-H3 having the amino acid sequence of SEQ ID NO: 140. Alternatively or in addition (e.g., in addition), the anti-TfR antibody of the present disclosure comprises: a CDR-L1 having no more than 3 amino acid variations (e.g., no more than 3, 2, or 1 amino acid variation) as compared with the CDR-L1 having the amino acid sequence of SEQ ID NO: 141; a CDR-L2 having no more than 3 amino acid variations (e.g., no more than 3, 2, or 1 amino acid variation) as compared with the CDR-L2 having the amino acid sequence of SEQ ID NO: 29; and/or (e.g., and) a CDR-L3 having no more than 3 amino acid variations (e.g., no more than 3, 2, or 1 amino acid variation) as compared with the CDR-L3 having the amino acid sequence of SEQ ID NO: 142.

In some embodiments, the anti-TfR antibody of the present disclosure comprises a VH comprising the amino acid sequence of SEQ ID NO: 143. Alternatively or in addition (e.g., in addition), the anti-TfR antibody of the present disclosure comprises a VL comprising the amino acid sequence of SEQ ID NO: 144.

In some embodiments, the anti-TfR antibody of the present disclosure comprises a VH containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 143. Alternatively or in addition (e.g., in addition), the anti-TfR antibody of the present disclosure comprises a VL containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VL as set forth in SEQ ID NO: 144.

In some embodiments, the anti-TfR antibody of the present disclosure comprises a VH comprising an amino acid sequence that is at least 75% (e.g., 75%, 80%, 85%, 90%, 95%, 98%, or 99%) identical to the VH as set forth in SEQ ID NO: 143. Alternatively or in addition (e.g., in addition), the anti-TfR antibody of the present disclosure comprises a VL comprising an amino acid sequence that is at least 75% (e.g., 75%, 80%, 85%, 90%, 95%, 98%, or 99%) identical to the VL as set forth in SEQ ID NO: 144.

The CDRs of an antibody may have different amino acid sequences when different definition systems are used (e.g., the IMGT definition, the Kabat definition, or the Chothia definition). A definition system annotates each amino acid in a given antibody sequence (e.g., VH or VL sequence) with a number, and numbers corresponding to the heavy chain and light chain CDRs are provided in Table 3. The CDRs listed in Table 2 are defined in accordance with the IMGT definition. CDR sequences of examples of anti-TfR antibodies according to the different definition systems are provided in Table 4. One skilled in the art is able to derive the CDR sequences using the different numbering systems for the anti-TfR antibodies provided in Table 2.

TABLE 3
CDR Definitions

| | IMGT¹ | Kabat² | Chothia³ |
|---|---|---|---|
| CDR-H1 | 27-38 | 31-35 | 26-32 |
| CDR-H2 | 56-65 | 50-65 | 53-55 |
| CDR-H3 | 105-116/117 | 95-102 | 96-101 |
| CDR-L1 | 27-38 | 24-34 | 26-32 |
| CDR-L2 | 56-65 | 50-56 | 50-52 |
| CDR-L3 | 105-116/117 | 89-97 | 91-96 |

¹IMGT ®, the international ImMunoGeneTics information system ®, imgt.org, Lefranc, M.-P. et al., Nucleic Acids Res., 27: 209-212 (1999)
²Kabat et al. (1991) Sequences of Proteins of Immunological Interest, Fifth Edition, U.S. Department of Health and Human Services, NIH Publication No. 91-3242
³Chothia et al., J. Mol. Biol. 196: 901-917 (1987))

TABLE 4
CDR sequences of examples of anti-TfR antibodies
according to different definition systems

| | No. | | | |
|---|---|---|---|---|
| | system | IMGT | Kabat | Chothia |
| 3-A4 | CDR-H1 | GFNIKDDY (SEQ ID NO: 1) | DDYMY (SEQ ID NO: | GFNIKDD (SEQ ID NO: |
| | | | 145) | 150) |
| | CDR-H2 | IDPENGDT (SEQ ID NO: 2) | WIDPENGDTEYASKFQD | ENG (SEQ ID NO: 151) |
| | | | (SEQ ID NO: 146) | |
| | CDR-H3 | TLWLRRGLDY (SEQ ID | WLRRGLDY (SEQ ID | LRRGLD (SEQ ID NO: |
| | | NO: 3) | NO: 147) | 152) |
| | CDR-L1 | KSLLHSNGYTY (SEQ ID | RSSKSLLHSNGYTYLF | SKSLLHSNGYTY(SEQ |
| | | NO: 4) | (SEQ ID NO: 148) | ID NO: 153) |
| | CDR-L2 | RMS (SEQ ID NO: 5) | RMSNLAS (SEQ ID NO: | RMS(SEQ ID NO: 5) |
| | | | 149) | |
| | CDR-L3 | MQHLEYPFT (SEQ ID NO: | MQHLEYPFT (SEQ ID | HLEYPF (SEQ ID NO: |
| | | 6) | NO: 6) | 154) |
| 3-A4 | CDR-H1 | GFNIKDDY (SEQ ID NO: 1) | DDYMY (SEQ ID NO: | GFNIKDD (SEQ ID NO: |
| Variant | | | 145) | 150) |
| 1 | | | | |
| | CDR-H2 | IDPETGDT (SEQ ID NO: | WIDPETGDTEYASKFQD | ETG (SEQ ID NO: 270) |
| | | 262) | (SEQ ID NO: 263) | |
| | CDR-H3 | TLWLRRGLDY (SEQ ID | WLRRGLDY (SEQ ID | LRRGLD (SEQ ID NO: |
| | | NO: 3) | NO: 147) | 152) |
| | CDR-L1 | KSLLHSNGYTY (SEQ ID | RSSKSLLHSNGYTYLF | SKSLLHSNGYTY(SEQ |
| | | NO: 4) | (SEQ ID NO: 148) | ID NO: 153) |
| | CDR-L2 | RMS (SEQ ID NO: 5) | RMSNLAS (SEQ ID NO: | RMS(SEQ ID NO: 5) |
| | | | 149) | |
| | CDR-L3 | MQHLEYPFT (SEQ ID NO: | MQHLEYPFT (SEQ ID | HLEYPF (SEQ ID NO: |
| | | 6) | NO: 6) | 154) |
| 3-A4 | CDR-H1 | GFNIKDDY (SEQ ID NO: 1) | DDYMY (SEQ ID NO: | GFNIKDD (SEQ ID NO: |
| Variant | | | 145) | 150) |
| 2 | | | | |
| | CDR-H2 | IDPESGDT (SEQ ID NO: | WIDPESGDTEYASKFQD | ESG (SEQ ID NO: 271) |
| | | 80) | (SEQ ID NO: 265) | |
| | CDR-H3 | TLWLRRGLDY (SEQ ID | WLRRGLDY (SEQ ID | LRRGLD (SEQ ID NO: |
| | | NO: 3) | NO: 147) | 152) |
| | CDR-L1 | KSLLHSNGYTY (SEQ ID | RSSKSLLHSNGYTYLF | SKSLLHSNGYTY(SEQ |
| | | NO: 4) | (SEQ ID NO: 148) | ID NO: 153) |
| | CDR-L2 | RMS (SEQ ID NO: 5) | RMSNLAS (SEQ ID NO: | RMS(SEQ ID NO: 5) |
| | | | 149) | |
| | CDR-L3 | MQHLEYPFT (SEQ ID NO: | MQHLEYPFT (SEQ ID | HLEYPF (SEQ ID NO: |
| | | 6) | NO: 6) | 154) |
| 3-M12 | CDR-H1 | GYSITSGYY (SEQ ID NO: | SGYYWN (SEQ ID NO: | GYSITSGY (SEQ ID NO: |
| | | 9) | 155) | 160) |
| | CDR-H2 | ITFDGAN (SEQ ID NO: 10) | YITFDGANNYNPSLKN | FDG (SEQ ID NO: 161) |
| | | | (SEQ ID NO: 156) | |
| | CDR-H3 | TRSSYDYDVLDY (SEQ ID | SSYDYDVLDY (SEQ ID | SYDYDVLD (SEQ ID |
| | | NO: 11) | NO: 157) | NO: 162) |
| | CDR-L1 | QDISNF (SEQ ID NO: 12) | RASQDISNFLN (SEQ ID | SQDISNF (SEQ ID NO: |
| | | | NO: 158) | 163) |
| | CDR-L2 | YTS (SEQ ID NO: 13) | YTSRLHS (SEQ ID NO: | YTS (SEQ ID NO: 13) |
| | | | 159) | |
| | CDR-L3 | QQGHTLPYT (SEQ ID NO: | QQGHTLPYT (SEQ ID | GHTLPY (SEQ ID NO: |
| | | 14) | NO: 14) | 164) |
| 5-H12 | CDR-H1 | GYSFTDYC (SEQ ID NO: | DYCIN (SEQ ID NO: 165) | GYSFTDY (SEQ ID NO: |
| | | 17) | | 170) |
| | CDR-H2 | IYPGSGNT (SEQ ID NO: | WIYPGSGNTRYSERFKG | GSG (SEQ ID NO: 171) |
| | | 18) | (SEQ ID NO: 166) | |
| | CDR-H3 | AREDYYPYHGMDY (SEQ | EDYYPYHGMDY (SEQ | DYYPYHGMD (SEQ ID |
| | | ID NO: 19) | ID NO: 167) | NO: 172) |
| | CDR-L1 | ESVDGYDNSF (SEQ ID | RASES VDGYDNSFMH | SESVDGYDNSF (SEQ ID |
| | | NO: 20) | (SEQ ID NO: 168) | NO: 173) |
| | CDR-L2 | RAS (SEQ ID NO: 21) | RASNLES (SEQ ID NO: | RAS (SEQ ID NO: 21) |
| | | | 169) | |
| | CDR-L3 | QQSSEDPWT (SEQ ID NO: | QQSSEDPWT (SEQ ID | SSEDPW (SEQ ID NO: |
| | | 22) | NO: 22) | 174) |
| 5-H12 | CDR-H1 | GYSFTDYY (SEQ ID NO: | DYYIN (SEQ ID NO: 267) | GYSFTDY (SEQ ID NO: |
| Variant | | 266) | | 170) |
| 1 | | | | |
| | CDR-H2 | IYPGSGNT (SEQ ID NO: | WIYPGSGNTRYSERFKG | GSG (SEQ ID NO: 171) |
| | | 18) | (SEQ ID NO: 166) | |
| | CDR-H3 | AREDYYPYHGMDY (SEQ | EDYYPYHGMDY (SEQ | DYYPYHGMD (SEQ ID |
| | | ID NO: 19) | ID NO: 167) | NO: 172) |
| | CDR-L1 | ESVDGYDNSF (SEQ ID | RASES VDGYDNSFMH | SESVDGYDNSF (SEQ ID |
| | | NO: 20) | (SEQ ID NO: 168) | NO: 173) |
| | CDR-L2 | RAS (SEQ ID NO: 21) | RASNLES (SEQ ID NO: | RAS (SEQ ID NO: 21) |
| | | | 169) | |
| | CDR-L3 | QQSSEDPWT (SEQ ID NO: | QQSSEDPWT (SEQ ID | SSEDPW (SEQ ID NO: |
| | | 22) | NO: 22) | 174) |
| 5-H12 | CDR-H1 | GYSFTDYD (SEQ ID NO: | DYDIN (SEQ ID NO: 269) | GYSFTDY (SEQ ID NO: |
| Variant | | 268) | | 170) |
| 2 | | | | |
| | CDR-H2 | IYPGSGNT (SEQ ID NO: | WIYPGSGNTRYSERFKG | GSG (SEQ ID NO: 171) |
| | | 18) | (SEQ ID NO: 166) | |
| | CDR-H3 | AREDYYPYHGMDY (SEQ | EDYYPYHGMDY (SEQ | DYYPYHGMD (SEQ ID |
| | | ID NO: 19) | ID NO: 167) | NO: 172) |
| | CDR-L1 | ESVDGYDNSF (SEQ ID | RASESVDGYDNSFMH | SESVDGYDNSF (SEQ ID |
| | | NO: 20) | (SEQ ID NO: 168) | NO: 173) |
| | CDR-L2 | RAS (SEQ ID NO: 21) | RASNLES (SEQ ID NO: | RAS (SEQ ID NO: 21) |
| | | | 169) | |
| | CDR-L3 | QQSSEDPWT (SEQ ID NO: | QQSSEDPWT (SEQ ID | SSEDPW (SEQ ID NO: |
| | | 22) | NO: 22) | 174) |

In some embodiments, the anti-TfR antibody of the present disclosure comprises a CDR-H1 having the amino acid sequence of SEQ ID NO: 145 (according to the Kabat definition system), a CDR-H2 having the amino acid sequence of SEQ ID NO: 146, SEQ ID NO: 263, or SEQ ID NO: 265 (according to the Kabat definition system), a CDR-H3 having the amino acid sequence of SEQ ID NO: 147 (according to the Kabat definition system), a CDR-L1 having the amino acid sequence of SEQ ID NO: 148 (according to the Kabat definition system), a CDR-L2 having the amino acid sequence of SEQ ID NO: 149 (according to the Kabat definition system), and a CDR-L3 having the amino acid sequence of SEQ ID NO: 6 (according to the Kabat definition system).

In some embodiments, anti-TfR antibody of the present disclosure comprises a CDR-H1, a CDR-H2, and a CDR-H3, which collectively contains no more than 5 amino acid variations (e.g., no more than 5, 4, 3, 2, or 1 amino acid variation) as compared with the CDR-H1 having the amino acid sequence of SEQ ID NO: 145, CDR-H2 having the amino acid sequence of SEQ ID NO: 146, SEQ ID NO: 263, or SEQ ID NO: 265, and CDR-H3 having the amino acid sequence of SEQ ID NO: 147. Alternatively or in addition (e.g., in addition), the anti-TfR antibody of the present disclosure comprises a CDR-L1, a CDR-L2, and a CDR-L3, which collectively contains no more than 5 amino acid variations (e.g., no more than 5, 4, 3, 2 or 1 amino acid variation) as compared with the CDR-L1 having the amino acid sequence of SEQ ID NO: 148, CDR-L2 having the amino acid sequence of SEQ ID NO: 149, and CDR-L3 having the amino acid sequence of SEQ ID NO: 6.

In some embodiments, the anti-TfR antibody of the present disclosure comprises a CDR-H1, a CDR-H2, and a CDR-H3 that collectively are at least 75% (e.g., 75%, 80%, 85%, 90%, 95%, 98%, or 99%) identical to the CDR-H1 having the amino acid sequence of SEQ ID NO: 145, CDR-H2 having the amino acid sequence of SEQ ID NO: 146, SEQ ID NO: 263, or SEQ ID NO: 265, and CDR-H3 having the amino acid sequence of SEQ ID NO: 147. Alternatively or in addition (e.g., in addition), the anti-TfR antibody of the present disclosure comprises a CDR-L1, a CDR-L2, and a CDR-L3 that collectively are at least 75% (e.g., 75%, 80%, 85%, 90%, 95%, 98%, or 99%) identical to the to the CDR-L1 having the amino acid sequence of SEQ ID NO: 148, CDR-L2 having the amino acid sequence of SEQ ID NO: 149, and CDR-L3 having the amino acid sequence of SEQ ID NO: 6.

In some embodiments, the anti-TfR antibody of the present disclosure comprises: a CDR-H1 having no more than 3 amino acid variations (e.g., no more than 3, 2, or 1 amino acid variation) as compared with the CDR-H1 having the amino acid sequence of SEQ ID NO: 145; a CDR-H2 having no more than 3 amino acid variations (e.g., no more than 3, 2, or 1 amino acid variation) as compared with the CDR-H2 having the amino acid sequence of SEQ ID NO: 146, SEQ ID NO: 263, or SEQ ID NO: 265; and/or (e.g., and) a CDR-H3 having no more than 3 amino acid variations (e.g., no more than 3, 2, or 1 amino acid variation) as compared with the CDR-H3 having the amino acid sequence of SEQ ID NO: 147. Alternatively or in addition (e.g., in addition), the anti-TfR antibody of the present disclosure comprises: a CDR-L1 having no more than 3 amino acid variations (e.g., no more than 3, 2, or 1 amino acid variation) as compared with the CDR-L1 having the amino acid sequence of SEQ ID NO: 148; a CDR-L2 having no more than 3 amino acid variations (e.g., no more than 3, 2, or 1 amino acid variation) as compared with the CDR-L2 having the amino acid sequence of SEQ ID NO: 149; and/or (e.g., and) a CDR-L3 having no more than 3 amino acid variations (e.g., no more than 3, 2, or 1 amino acid variation) as compared with the CDR-L3 having the amino acid sequence of SEQ ID NO: 6.

In some embodiments, the anti-TfR antibody of the present disclosure comprises a CDR-H1 having the amino acid sequence of SEQ ID NO: 150 (according to the Chothia definition system), a CDR-H2 having the amino acid sequence of SEQ ID NO: 151, SEQ ID NO: 270, or SEQ ID NO: 271 (according to the Chothia definition system), a CDR-H3 having the amino acid sequence of SEQ ID NO: 152 (according to the Chothia definition system), a CDR-L1 having the amino acid sequence of SEQ ID NO: 153 (according to the Chothia definition system), a CDR-L2 having the amino acid sequence of SEQ ID NO: 5 according to the Chothia definition system), and a CDR-L3 having the amino acid sequence of SEQ ID NO: 154 (according to the Chothia definition system).

In some embodiments, anti-TfR antibody of the present disclosure comprises a CDR-H1, a CDR-H2, and a CDR-H3, which collectively contains no more than 5 amino acid variations (e.g., no more than 5, 4, 3, 2, or 1 amino acid variation) as compared with the CDR-H1 having the amino acid sequence of SEQ ID NO: 150, CDR-H2 having the amino acid sequence of SEQ ID NO: 151, SEQ ID NO: 270, or SEQ ID NO: 271, and CDR-H3 having the amino acid sequence of SEQ ID NO: 152. Alternatively or in addition (e.g., in addition), the anti-TfR antibody of the present disclosure comprises a CDR-L1, a CDR-L2, and a CDR-L3, which collectively contains no more than 5 amino acid variations (e.g., no more than 5, 4, 3, 2 or 1 amino acid variation) as compared with the CDR-L1 having the amino acid sequence of SEQ ID NO: 153, CDR-L2 having the amino acid sequence of SEQ ID NO: 5, and CDR-L3 having the amino acid sequence of SEQ ID NO: 154.

In some embodiments, the anti-TfR antibody of the present disclosure comprises a CDR-H1, a CDR-H2, and a CDR-H3 that collectively are at least 75% (e.g., 75%, 80%, 85%, 90%, 95%, 98%, or 99%) identical to the CDR-H1 having the amino acid sequence of SEQ ID NO: 150, CDR-H2 having the amino acid sequence of SEQ ID NO: 151, SEQ ID NO: 270, or SEQ ID NO: 271, and CDR-H3 having the amino acid sequence of SEQ ID NO: 152. Alternatively or in addition (e.g., in addition), the anti-TfR antibody of the present disclosure comprises a CDR-L1, a CDR-L2, and a CDR-L3 that collectively are at least 75% (e.g., 75%, 80%, 85%, 90%, 95%, 98%, or 99%) identical to the to the CDR-L1 having the amino acid sequence of SEQ ID NO: 153, CDR-L2 having the amino acid sequence of SEQ ID NO: 5, and CDR-L3 having the amino acid sequence of SEQ ID NO: 154.

In some embodiments, the anti-TfR antibody of the present disclosure comprises: a CDR-H1 having no more than 3 amino acid variations (e.g., no more than 3, 2, or 1 amino acid variation) as compared with the CDR-H1 having the amino acid sequence of SEQ ID NO: 150; a CDR-H2 having no more than 3 amino acid variations (e.g., no more than 3, 2, or 1 amino acid variation) as compared with the CDR-H2 having the amino acid sequence of SEQ ID NO: 151, SEQ ID NO: 270, or SEQ ID NO: 271; and/or (e.g., and) a CDR-H3 having no more than 3 amino acid variations (e.g., no more than 3, 2, or 1 amino acid variation) as compared with the CDR-H3 having the amino acid sequence of SEQ ID NO: 152. Alternatively or in addition (e.g., in addition), the anti-TfR antibody of the present disclosure comprises: a CDR-L1 having no more than 3 amino acid variations (e.g., no more than 3, 2, or 1 amino acid variation) as compared with the CDR-L1 having the amino acid sequence of SEQ ID NO: 153; a CDR-L2 having no more than 3 amino acid variations (e.g., no more than 3, 2, or 1 amino acid variation) as compared with the CDR-L2 having the amino acid sequence of SEQ ID NO: 5; and/or (e.g., and) a CDR-L3 having no more than 3 amino acid variations (e.g., no more than 3, 2, or 1 amino acid variation) as compared with the CDR-L3 having the amino acid sequence of SEQ ID NO: 154.

In some embodiments, the anti-TfR antibody of the present disclosure comprises a CDR-H1 having the amino acid sequence of SEQ ID NO: 155 (according to the Kabat definition system), a CDR-H2 having the amino acid sequence of SEQ ID NO: 156 (according to the Kabat definition system), a CDR-H3 having the amino acid sequence of SEQ ID NO: 157 (according to the Kabat definition system), a CDR-L1 having the amino acid sequence of SEQ ID NO: 158 (according to the Kabat definition system), a CDR-L2 having the amino acid sequence of SEQ ID NO: 159 (according to the Kabat definition system), and a CDR-L3 having the amino acid sequence of SEQ ID NO: 14 (according to the Kabat definition system).

In some embodiments, anti-TfR antibody of the present disclosure comprises a CDR-H1, a CDR-H2, and a CDR-H3, which collectively contains no more than 5 amino acid variations (e.g., no more than 5, 4, 3, 2, or 1 amino acid variation) as compared with the CDR-H1 having the amino acid sequence of SEQ ID NO: 155, CDR-H2 having the amino acid sequence of SEQ ID NO: 156, and CDR-H3 having the amino acid sequence of SEQ ID NO: 157. Alternatively or in addition (e.g., in addition), the anti-TfR antibody of the present disclosure comprises a CDR-L1, a CDR-L2, and a CDR-L3, which collectively contains no more than 5 amino acid variations (e.g., no more than 5, 4, 3, 2 or 1 amino acid variation) as compared with the CDR-L1 having the amino acid sequence of SEQ ID NO: 158, CDR-L2 having the amino acid sequence of SEQ ID NO: 159, and CDR-L3 having the amino acid sequence of SEQ ID NO: 14.

In some embodiments, the anti-TfR antibody of the present disclosure comprises a CDR-H1, a CDR-H2, and a CDR-H3 that collectively are at least 75% (e.g., 75%, 80%, 85%, 90%, 95%, 98%, or 99%) identical to the CDR-H1 having the amino acid sequence of SEQ ID NO: 155, CDR-H2 having the amino acid sequence of SEQ ID NO: 156, and CDR-H3 having the amino acid sequence of SEQ ID NO: 157. Alternatively or in addition (e.g., in addition), the anti-TfR antibody of the present disclosure comprises a CDR-L1, a CDR-L2, and a CDR-L3 that collectively are at least 75% (e.g., 75%, 80%, 85%, 90%, 95%, 98%, or 99%) identical to the to the CDR-L1 having the amino acid sequence of SEQ ID NO: 158, CDR-L2 having the amino acid sequence of SEQ ID NO: 159, and CDR-L3 having the amino acid sequence of SEQ ID NO: 14.

In some embodiments, the anti-TfR antibody of the present disclosure comprises: a CDR-H1 having no more than 3 amino acid variations (e.g., no more than 3, 2, or 1 amino acid variation) as compared with the CDR-H1 having the amino acid sequence of SEQ ID NO: 155; a CDR-H2 having no more than 3 amino acid variations (e.g., no more than 3, 2, or 1 amino acid variation) as compared with the CDR-H2 having the amino acid sequence of SEQ ID NO: 156; and/or (e.g., and) a CDR-H3 having no more than 3 amino acid variations (e.g., no more than 3, 2, or 1 amino acid variation) as compared with the CDR-H3 having the amino acid sequence of SEQ ID NO: 157. Alternatively or in addition (e.g., in addition), the anti-TfR antibody of the present disclosure comprises: a CDR-L1 having no more than 3 amino acid variations (e.g., no more than 3, 2, or 1 amino acid variation) as compared with the CDR-L1 having the amino acid sequence of SEQ ID NO: 158; a CDR-L2 having no more than 3 amino acid variations (e.g., no more than 3, 2, or 1 amino acid variation) as compared with the CDR-L2 having the amino acid sequence of SEQ ID NO: 159; and/or (e.g., and) a CDR-L3 having no more than 3 amino acid variations (e.g., no more than 3, 2, or 1 amino acid variation) as compared with the CDR-L3 having the amino acid sequence of SEQ ID NO: 14.

In some embodiments, the anti-TfR antibody of the present disclosure comprises a CDR-H1 having the amino acid sequence of SEQ ID NO: 160 (according to the Chothia definition system), a CDR-H2 having the amino acid sequence of SEQ ID NO: 161 (according to the Chothia definition system), a CDR-H3 having the amino acid sequence of SEQ ID NO: 162 (according to the Chothia definition system), a CDR-L1 having the amino acid sequence of SEQ ID NO: 163 (according to the Chothia definition system), a CDR-L2 having the amino acid sequence of SEQ ID NO: 13 (according to the Chothia definition system), and a CDR-L3 having the amino acid sequence of SEQ ID NO: 164 (according to the Chothia definition system).

In some embodiments, anti-TfR antibody of the present disclosure comprises a CDR-H1, a CDR-H2, and a CDR-H3, which collectively contains no more than 5 amino acid variations (e.g., no more than 5, 4, 3, 2, or 1 amino acid variation) as compared with the CDR-H1 having the amino acid sequence of SEQ ID NO: 160, CDR-H2 having the amino acid sequence of SEQ ID NO: 161, and CDR-H3 having the amino acid sequence of SEQ ID NO: 162. Alternatively or in addition (e.g., in addition), the anti-TfR antibody of the present disclosure comprises a CDR-L1, a CDR-L2, and a CDR-L3, which collectively contains no more than 5 amino acid variations (e.g., no more than 5, 4, 3, 2 or 1 amino acid variation) as compared with the CDR-L1 having the amino acid sequence of SEQ ID NO: 163, CDR-L2 having the amino acid sequence of SEQ ID NO: 13, and CDR-L3 having the amino acid sequence of SEQ ID NO: 164.

In some embodiments, the anti-TfR antibody of the present disclosure comprises a CDR-H1, a CDR-H2, and a CDR-H3 that collectively are at least 75% (e.g., 75%, 80%, 85%, 90%, 95%, 98%, or 99%) identical to the CDR-H1 having the amino acid sequence of SEQ ID NO: 160, CDR-H2 having the amino acid sequence of SEQ ID NO: 161, and CDR-H3 having the amino acid sequence of SEQ ID NO: 162. Alternatively or in addition (e.g., in addition), the anti-TfR antibody of the present disclosure comprises a CDR-L1, a CDR-L2, and a CDR-L3 that collectively are at least 75% (e.g., 75%, 80%, 85%, 90%, 95%, 98%, or 99%) identical to the to the CDR-L1 having the amino acid sequence of SEQ ID NO: 163, CDR-L2 having the amino acid sequence of SEQ ID NO: 13, and CDR-L3 having the amino acid sequence of SEQ ID NO: 164.

In some embodiments, the anti-TfR antibody of the present disclosure comprises: a CDR-H1 having no more than 3 amino acid variations (e.g., no more than 3, 2, or 1 amino acid variation) as compared with the CDR-H1 having the amino acid sequence of SEQ ID NO: 160; a CDR-H2 having no more than 3 amino acid variations (e.g., no more than 3, 2, or 1 amino acid variation) as compared with the CDR-H2 having the amino acid sequence of SEQ ID NO: 161; and/or (e.g., and) a CDR-H3 having no more than 3 amino acid variations (e.g., no more than 3, 2, or 1 amino acid variation) as compared with the CDR-H3 having the amino acid sequence of SEQ ID NO: 162. Alternatively or in addition (e.g., in addition), the anti-TfR antibody of the present disclosure comprises: a CDR-L1 having no more than 3 amino acid variations (e.g., no more than 3, 2, or 1 amino acid variation) as compared with the CDR-L1 having the amino acid sequence of SEQ ID NO: 163; a CDR-L2 having no more than 3 amino acid variations (e.g., no more than 3, 2, or 1 amino acid variation) as compared with the CDR-L2 having the amino acid sequence of SEQ ID NO: 13; and/or (e.g., and) a CDR-L3 having no more than 3 amino acid variations (e.g., no more than 3, 2, or 1 amino acid variation) as compared with the CDR-L3 having the amino acid sequence of SEQ ID NO: 164.

In some embodiments, the anti-TfR antibody of the present disclosure comprises a CDR-H1 having the amino acid sequence of SEQ ID NO: 165, SEQ ID NO: 267, or SEQ ID NO: 269 (according to the Kabat definition system), a CDR-H2 having the amino acid sequence of SEQ ID NO: 166 (according to the Kabat definition system), a CDR-H3 having the amino acid sequence of SEQ ID NO: 167 (according to the Kabat definition system), a CDR-L1 having the amino acid sequence of SEQ ID NO: 168 (according to the Kabat definition system), a CDR-L2 having the amino acid sequence of SEQ ID NO: 169 (according to the Kabat definition system), and a CDR-L3 having the amino acid sequence of SEQ ID NO: 22 (according to the Kabat definition system).

In some embodiments, anti-TfR antibody of the present disclosure comprises a CDR-H1, a CDR-H2, and a CDR-H3, which collectively contains no more than 5 amino acid variations (e.g., no more than 5, 4, 3, 2, or 1 amino acid variation) as compared with the CDR-H1 having the amino acid sequence of SEQ ID NO: 165, SEQ ID NO: 267, or SEQ ID NO: 269, CDR-H2 having the amino acid sequence of SEQ ID NO: 166, and CDR-H3 having the amino acid sequence of SEQ ID NO: 167. Alternatively or in addition (e.g., in addition), the anti-TfR antibody of the present disclosure comprises a CDR-L1, a CDR-L2, and a CDR-L3, which collectively contains no more than 5 amino acid variations (e.g., no more than 5, 4, 3, 2 or 1 amino acid variation) as compared with the CDR-L1 having the amino acid sequence of SEQ ID NO: 168, CDR-L2 having the amino acid sequence of SEQ ID NO: 169, and CDR-L3 having the amino acid sequence of SEQ ID NO: 22.

In some embodiments, the anti-TfR antibody of the present disclosure comprises a CDR-H1, a CDR-H2, and a CDR-H3 that collectively are at least 75% (e.g., 75%, 80%, 85%, 90%, 95%, 98%, or 99%) identical to the CDR-H1 having the amino acid sequence of SEQ ID NO: 165, SEQ ID NO: 267, or SEQ ID NO: 269, CDR-H2 having the amino acid sequence of SEQ ID NO: 166, and CDR-H3 having the amino acid sequence of SEQ ID NO: 167. Alternatively or in addition (e.g., in addition), the anti-TfR antibody of the present disclosure comprises a CDR-L1, a CDR-L2, and a CDR-L3 that collectively are at least 75% (e.g., 75%, 80%, 85%, 90%, 95%, 98%, or 99%) identical to the to the CDR-L1 having the amino acid sequence of SEQ ID NO: 168, CDR-L2 having the amino acid sequence of SEQ ID NO: 169, and CDR-L3 having the amino acid sequence of SEQ ID NO: 22.

In some embodiments, the anti-TfR antibody of the present disclosure comprises: a CDR-H1 having no more than 3 amino acid variations (e.g., no more than 3, 2, or 1 amino acid variation) as compared with the CDR-H1 having the amino acid sequence of SEQ ID NO: 165, SEQ ID NO: 267, or SEQ ID NO: 269; a CDR-H2 having no more than 3 amino acid variations (e.g., no more than 3, 2, or 1 amino acid variation) as compared with the CDR-H2 having the amino acid sequence of SEQ ID NO: 166; and/or (e.g., and) a CDR-H3 having no more than 3 amino acid variations (e.g., no more than 3, 2, or 1 amino acid variation) as compared with the CDR-H3 having the amino acid sequence of SEQ ID NO: 167. Alternatively or in addition (e.g., in addition), the anti-TfR antibody of the present disclosure comprises: a CDR-L1 having no more than 3 amino acid variations (e.g., no more than 3, 2, or 1 amino acid variation) as compared with the CDR-L1 having the amino acid sequence of SEQ ID NO: 168; a CDR-L2 having no more than 3 amino acid variations (e.g., no more than 3, 2, or 1 amino acid variation) as compared with the CDR-L2 having the amino acid sequence of SEQ ID NO: 169; and/or (e.g., and) a CDR-L3 having no more than 3 amino acid variations (e.g., no more than 3, 2, or 1 amino acid variation) as compared with the CDR-L3 having the amino acid sequence of SEQ ID NO: 22.

In some embodiments, the anti-TfR antibody of the present disclosure comprises a CDR-H1 having the amino acid sequence of SEQ ID NO: 170 (according to the Chothia definition system), a CDR-H2 having the amino acid sequence of SEQ ID NO: 171 (according to the Chothia definition system), a CDR-H3 having the amino acid sequence of SEQ ID NO: 172 (according to the Chothia definition system), a CDR-L1 having the amino acid sequence of SEQ ID NO: 173 (according to the Chothia definition system), a CDR-L2 having the amino acid sequence of SEQ ID NO: 21 (according to the Chothia definition system), and a CDR-L3 having the amino acid sequence of SEQ ID NO: 174 (according to the Chothia definition system).

In some embodiments, anti-TfR antibody of the present disclosure comprises a CDR-H1, a CDR-H2, and a CDR-H3, which collectively contains no more than 5 amino acid variations (e.g., no more than 5, 4, 3, 2, or 1 amino acid variation) as compared with the CDR-H1 having the amino acid sequence of SEQ ID NO: 170, CDR-H2 having the amino acid sequence of SEQ ID NO: 171, and CDR-H3 having the amino acid sequence of SEQ ID NO: 172. Alternatively or in addition (e.g., in addition), the anti-TfR antibody of the present disclosure comprises a CDR-L1, a CDR-L2, and a CDR-L3, which collectively contains no more than 5 amino acid variations (e.g., no more than 5, 4, 3, 2 or 1 amino acid variation) as compared with the CDR-L1 having the amino acid sequence of SEQ ID NO: 173, CDR-L2 having the amino acid sequence of SEQ ID NO: 21, and CDR-L3 having the amino acid sequence of SEQ ID NO: 174.

In some embodiments, the anti-TfR antibody of the present disclosure comprises a CDR-H1, a CDR-H2, and a CDR-H3 that collectively are at least 75% (e.g., 75%, 80%, 85%, 90%, 95%, 98%, or 99%) identical to the CDR-H1 having the amino acid sequence of SEQ ID NO: 170, CDR-H2 having the amino acid sequence of SEQ ID NO: 171, and CDR-H3 having the amino acid sequence of SEQ ID NO: 172. Alternatively or in addition (e.g., in addition), the anti-TfR antibody of the present disclosure comprises a CDR-L1, a CDR-L2, and a CDR-L3 that collectively are at least 75% (e.g., 75%, 80%, 85%, 90%, 95%, 98%, or 99%) identical to the to the CDR-L1 having the amino acid sequence of SEQ ID NO: 173, CDR-L2 having the amino acid sequence of SEQ ID NO: 21, and CDR-L3 having the amino acid sequence of SEQ ID NO: 174.

In some embodiments, the anti-TfR antibody of the present disclosure comprises: a CDR-H1 having no more than 3 amino acid variations (e.g., no more than 3, 2, or 1 amino acid variation) as compared with the CDR-H1 having the amino acid sequence of SEQ ID NO: 170; a CDR-H2 having no more than 3 amino acid variations (e.g., no more than 3, 2, or 1 amino acid variation) as compared with the CDR-H2 having the amino acid sequence of SEQ ID NO: 171; and/or (e.g., and) a CDR-H3 having no more than 3 amino acid variations (e.g., no more than 3, 2, or 1 amino acid variation) as compared with the CDR-H3 having the amino acid sequence of SEQ ID NO: 172. Alternatively or in addition (e.g., in addition), the anti-TfR antibody of the present disclosure comprises: a CDR-L1 having no more than 3 amino acid variations (e.g., no more than 3, 2, or 1 amino acid variation) as compared with the CDR-L1 having the amino acid sequence of SEQ ID NO: 173; a CDR-L2 having no more than 3 amino acid variations (e.g., no more than 3, 2, or 1 amino acid variation) as compared with the CDR-L2 having the amino acid sequence of SEQ ID NO: 21; and/or (e.g., and) a CDR-L3 having no more than 3 amino acid variations (e.g., no more than 3, 2, or 1 amino acid variation) as compared with the CDR-L3 having the amino acid sequence of SEQ ID NO: 174.

In some embodiments, the anti-TfR antibody of the present disclosure is a humanized antibody (e.g., a humanized variant containing one or more CDRs of Table 2 or Table 4). In some embodiments, the anti-TfR antibody of the present disclosure comprises a CDR-H1, a CDR-H2, a CDR-H3, a CDR-L1, a CDR-L2, and a CDR-L3 that are the same as the CDR-H1, CDR-H2, and CDR-H3 shown in Table 2 or Table 4, and comprises a humanized heavy chain variable region and/or (e.g., and) a humanized light chain variable region.

Humanized antibodies are human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity. In some embodiments, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in WO 99/58572. Other forms of humanized antibodies have one or more CDRs (one, two, three, four, five, six) which are altered with respect to the original antibody, which are also termed one or more CDRs derived from one or more CDRs from the original antibody. Humanized antibodies may also involve affinity maturation.

Humanized antibodies and methods of making them are known, e.g., as described in Almagro et al., Front. Biosci. 13:1619-1633 (2008); Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005); Padlan et al., Mol. Immunol. 28:489-498 (1991); Dall'Acqua et al., Methods 36:43-60 (2005); Osbourn et al., Methods 36:61-68 (2005); and Klimka et al., Br. J. Cancer, 83:252-260 (2000), the contents of all of which are incorporated herein by reference. Human framework regions that may be used for humanization are described in e.g., Sims et al. J. Immunol. 151:2296 (1993); Carter et al. Proc. Natl. Acad. Sci. USA, 89:4285 (1992); Presta et al. J. Immunol., 151:2623 (1993); Almagro et al., Front. Biosci. 13:1619-1633 (2008)); Baca et al., J. Biol. Chem. 272:10678-10684 (1997); and Rosok et al., J Biol. Chem. 271:22611-22618 (1996), the contents of all of which are incorporated herein by reference. In some embodiments, humanization is achieved by grafting the CDRs (e.g., as shown in Table 2 or Table 4) into the IGKV1-NL1*01 and IGHV1-3*01 human variable domains.

In some embodiments, a humanized VH framework or VL framework is a consensus human framework. In some embodiments, a consensus humanized framework can represent the most commonly occurring amino acid residue in a selection of human immunoglobulin VL or VH framework sequences.

In some embodiments, the consensus human VH framework regions suitable for use with heavy chain CDRs in the humanized anti-TfR antibodies described herein include (subgroup III consensus):

| | a) VH FR1: |
|---|---|
| (SEQ ID NO: 272) | |
| | EVQLVESGGGLVQPGGSLRLSCAAS; |
| | b) VH FR2: |
| (SEQ ID NO: 273) | |
| | WVRQAPGKGLEWV; |
| | c) VH FR3: |
| (SEQ ID NO: 274) | |
| | RFTISRDNSKNTLYLQMNSLRAEDTAVYYC; |
| | and |
| | d) VH FR4: |
| (SEQ ID NO: 275) | |
| | WGQGTLVTVSS. |

In some embodiments, the consensus human VH framework regions suitable for use with heavy chain CDRs in the humanized anti-TfR antibodies described herein include (subgroup I consensus):

| | a) VH FR1: |
|---|---|
| (SEQ ID NO: 276) | |
| | QVQLVQSGAEVKKPGASVKVSCKAS; |
| | b) VH FR2: |
| (SEQ ID NO: 277) | |
| | WVRQAPGQGLEWM; |
| | c) VH FR3: |
| (SEQ ID NO: 278) | |
| | RVTITADTSTSTAYMELSSLRSEDTAVYYC; |
| | and |
| | d) VH FR4: |
| (SEQ ID NO: 275) | |
| | WGQGTLVTVSS. |

In some embodiments, the consensus human VH framework regions suitable for use with heavy chain CDRs in the humanized anti-TfR antibodies described herein include (subgroup II consensus):

| | a) VH FR1: |
|---|---|
| (SEQ ID NO: 280) | |
| | QVQLQESGPGLVKPSQTLSLTCTVS; |
| | b) VH FR2: |
| (SEQ ID NO: 281) | |
| | WIRQPPGKGLEWI; |
| | c) VH FR3: |
| (SEQ ID NO: 282) | |
| | RVTISVDTSKNQFSLKLSSVTAADTAVYYC; |
| | and |
| | d) VH FR4: |
| (SEQ ID NO: 275) | |
| | WGQGTLVTVSS. |

In some embodiments, the consensus human VL framework regions suitable for use with light chain CDRs in the humanized anti-TfR antibodies described herein include (subgroup I consensus):

a) VL FR1:
(SEQ ID NO: 284)
DIQMTQSPSSLSASVGDRVTITC;
b) VL FR2:
(SEQ ID NO: 285)
WYQQKPGKAPKLLIY;
c) VL FR3:
(SEQ ID NO: 286)
GVPSRFSGSGSGTDFTLTISSLQPEDFATYYC;
and
d) VL FR4:
(SEQ ID NO: 287)
FGQGTKVEIK.

In some embodiments, the consensus human VL framework regions suitable for use with light chain CDRs in the humanized anti-TfR antibodies described herein include (subgroup II consensus):

a) VL FR1:
(SEQ ID NO: 288)
DIVMTQSPLSLPVTPGEPASISC;
b) VL FR2:
(SEQ ID NO: 289)
WYLQKPGQSPQLLIY;
c) VL FR3:
(SEQ ID NO: 290)
GVPDRFSGSGSGTDFTLKISRVEAEDVGVYYC;
and
d) VL FR4:
(SEQ ID NO: 287)
FGQGTKVEIK.

In some embodiments, the consensus human VL framework regions suitable for use with light chain CDRs in the humanized anti-TfR antibodies described herein include (subgroup III consensus):

a) VL FR1:
(SEQ ID NO: 279)
DIVMTQSPDSLAVSLGERATINC;
b) VL FR2:
(SEQ ID NO: 283)
WYQQKPGQPPKLLIY;
c) VL FR3:
(SEQ ID NO: 291)
GVPDRFSGSGSGTDFTLTISSLQAEDFAVYYC;
and
d) VL FR4:
(SEQ ID NO: 287)
FGQGTKVEIK.

In some embodiments, the consensus human VL framework regions suitable for use with light chain CDRs in the humanized anti-TfR antibodies described herein include (subgroup IV consensus):

a) VL FR1:
(SEQ ID NO: 279)
DIVMTQSPDSLAVSLGERATINC;
b) VL FR2:
(SEQ ID NO: 283)
WYQQKPGQPPKLLIY;
c) VL FR3:
(SEQ ID NO: 291)
GVPDRFSGSGSGTDFTLTISSLQAEDFAVYYC;
and
d) VL FR4:
(SEQ ID NO: 287)
FGQGTKVEIK.

In some embodiments, the humanized anti-TfR antibody of the present disclosure comprises humanized VH framework regions that collectively contain no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with any one of the consensus human VH framework region subgroups described herein. Alternatively or in addition (e.g., in addition), the humanized anti-TfR antibody of the present disclosure comprises humanized VL framework regions that collectively contain no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with any one of the consensus human VL framework region subgroups described herein.

In some embodiments, the humanized anti-TfR antibody of the present disclosure comprises humanized VH framework regions that collectively are at least 75% (e.g., 75%, 80%, 85%, 90%, 95%, 98%, or 99%) identical to any one of the consensus human VH framework region subgroups described herein. Alternatively or in addition (e.g., in addition), the humanized anti-TfR antibody of the present disclosure comprises humanized VL framework regions that are at least 75% (e.g., 75%, 80%, 85%, 90%, 95%, 98%, or 99%) identical to any one of the consensus human VL framework region subgroups described herein.

In some embodiments, the anti-TfR antibody of the present disclosure is a humanized variant comprising one or more amino acid variations (e.g., in the VH framework region) as compared with any one of the VHs listed in Table 2 or Table 4, and/or (e.g., and) one or more amino acid variations (e.g., in the VL framework region) as compared with any one of the VLs listed in Table 2 or Table 4.

In some embodiments, the anti-TfR antibody of the present disclosure is a humanized antibody comprising a VH containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VH of any of the anti-TfR antibodies listed in Table 2. Alternatively or in addition (e.g., in addition), the anti-TfR antibody of the present disclosure is a humanized antibody comprising a VL containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VL of any one of the anti-TfR antibodies listed in Table 2.

In some embodiments, the anti-TfR antibody of the present disclosure is a humanized antibody comprising a VH comprising an amino acid sequence that is at least 75% (e.g., 75%, 80%, 85%, 90%, 95%, 98%, or 99%) identical to the VH as set forth in any one of SEQ ID NOs: 7, 15, and 23. Alternatively or in addition (e.g., in addition), the anti-TfR antibody of the present disclosure is a humanized antibody comprising a VL comprising an amino acid sequence that is at least 75% (e.g., 75%, 80%, 85%, 90%, 95%, 98%, or 99%) identical to the VL as set forth in any one of SEQ ID NOs: 8, 16, and 24.

In some embodiments, the anti-TfR antibody of the present disclosure is a humanized antibody comprising a VH containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VH as set forth in any one of SEQ ID NOs: 7, 15, and 23. Alternatively or in addition (e.g., in addition), the anti-TfR antibody of the present disclosure is a humanized antibody comprising a VL containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VL as set forth in any one of SEQ ID NOs: 8, 16, and 24.

In some embodiments, the anti-TfR antibody of the present disclosure is a humanized antibody comprising a VH comprising an amino acid sequence that is at least 75% (e.g., 75%, 80%, 85%, 90%, 95%, 98%, or 99%) identical to the VH as set forth in any one of SEQ ID NOs: 7, 15, and 23. Alternatively or in addition (e.g., in addition), the anti-TfR antibody of the present disclosure is a humanized antibody comprising a VL comprising an amino acid sequence that is at least 75% (e.g., 75%, 80%, 85%, 90%, 95%, 98%, or 99%) identical to the VL as set forth in any one of SEQ ID NOs: 8, 16, and 24.

In some embodiments, the anti-TfR antibody of the present disclosure is a humanized antibody comprising a VH having one or more (e.g., 10-25) amino acid variations at positions 1, 2, 5, 9, 11, 12, 13, 17, 20, 23, 33, 38, 40, 41, 42, 43, 44, 45, 48, 49, 55, 67, 68, 70, 71, 72, 76, 77, 80, 81, 82, 84, 87, 88, 91, 95, 112, or 115 relative to the VH as set forth in any one of SEQ ID NOs: 7, 15, and 23. Alternatively or in addition (e.g., in addition), the anti-TfR antibody of the present disclosure is a humanized antibody comprising a VL having one or more (e.g., 10-20) amino acid variations at positions 4, 7, 8, 9, 11, 15, 17, 18, 19, 22, 39, 41, 42, 43, 50, 62, 64, 72, 75, 77, 79, 80, 81, 82, 83, 85, 87, 89, 100, 104, or 109 relative to the VL as set forth in any one of SEQ ID NOs: 8, 16, and 24.

In some embodiments, the anti-TfR antibody of the present disclosure comprises a humanized VH comprising a CDR-H1 having the amino acid sequence of SEQ ID NO: 1 (according to the IMGT definition system), a CDR-H2 having the amino acid sequence of SEQ ID NO: 2, SEQ ID NO: 262, or SEQ ID NO: 80 (according to the IMGT definition system), a CDR-H3 having the amino acid sequence of SEQ ID NO: 3 (according to the IMGT definition system), and containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VH as set forth in SEQ ID NO: 7. Alternatively or in addition (e.g., in addition), the anti-TfR antibody of the present disclosure comprises a humanized VL comprising a CDR-L1 having the amino acid sequence of SEQ ID NO: 4 (according to the IMGT definition system), a CDR-L2 having the amino acid sequence of SEQ ID NO: 5 (according to the IMGT definition system), and a CDR-L3 having the amino acid sequence of SEQ ID NO: 6 (according to the IMGT definition system), and containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VL as set forth in SEQ ID NO: 8.

In some embodiments, the anti-TfR antibody of the present disclosure comprises a humanized VH comprising a CDR-H1 having the amino acid sequence of SEQ ID NO: 1 (according to the IMGT definition system), a CDR-H2 having the amino acid sequence of SEQ ID NO: 2, SEQ ID NO: 262, or SEQ ID NO: 80 (according to the IMGT definition system), a CDR-H3 having the amino acid sequence of SEQ ID NO: 3 (according to the IMGT definition system), and is at least 75% (e.g., 75%, 80%, 85%, 90%, 95%, 98%, or 99%) identical in the framework regions to the VH as set forth in SEQ ID NO: 7. Alternatively or in addition (e.g., in addition), the anti-TfR antibody of the present disclosure comprises a humanized VL comprising a CDR-L1 having the amino acid sequence of SEQ ID NO: 4 (according to the IMGT definition system), a CDR-L2 having the amino acid sequence of SEQ ID NO: 5 (according to the IMGT definition system), and a CDR-L3 having the amino acid sequence of SEQ ID NO: 6 (according to the IMGT definition system), and is at least 75% (e.g., 75%, 80%, 85%, 90%, 95%, 98%, or 99%) identical in the framework regions to the VL as set forth in SEQ ID NO: 8.

In some embodiments, the anti-TfR antibody of the present disclosure comprises a humanized VH comprising a CDR-H1 having the amino acid sequence of SEQ ID NO: 145 (according to the Kabat definition system), a CDR-H2 having the amino acid sequence of SEQ ID NO: 146, SEQ ID NO: 263, or SEQ ID NO: 265 (according to the Kabat definition system), a CDR-H3 having the amino acid sequence of SEQ ID NO: 147 (according to the Kabat definition system), and containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VH as set forth in SEQ ID NO: 7. Alternatively or in addition (e.g., in addition), the anti-TfR antibody of the present disclosure comprises a humanized VL comprising a CDR-L1 having the amino acid sequence of SEQ ID NO: 148 (according to the Kabat definition system), a CDR-L2 having the amino acid sequence of SEQ ID NO: 149 (according to the Kabat definition system), and a CDR-L3 having the amino acid sequence of SEQ ID NO: 6 (according to the Kabat definition system), and containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VL as set forth in SEQ ID NO: 8.

In some embodiments, the anti-TfR antibody of the present disclosure comprises a humanized VH comprising a CDR-H1 having the amino acid sequence of SEQ ID NO: 145 (according to the Kabat definition system), a CDR-H2 having the amino acid sequence of SEQ ID NO: 146, SEQ ID NO: 263, or SEQ ID NO: 265 (according to the Kabat definition system), a CDR-H3 having the amino acid sequence of SEQ ID NO: 147 (according to the Kabat definition system), and is at least 75% (e.g., 75%, 80%, 85%, 90%, 95%, 98%, or 99%) identical in the framework regions to the VH as set forth in SEQ ID NO: 7. Alternatively or in addition (e.g., in addition), the anti-TfR antibody of the present disclosure comprises a humanized VL comprising a CDR-L1 having the amino acid sequence of SEQ ID NO: 148 (according to the Kabat definition system), a CDR-L2 having the amino acid sequence of SEQ ID NO: 149 (according to the Kabat definition system), and a CDR-L3 having the amino acid sequence of SEQ ID NO: 6 (according to the Kabat definition system), and is at least 75% (e.g., 75%, 80%, 85%, 90%, 95%, 98%, or 99%) identical in the framework regions to the VL as set forth in SEQ ID NO: 8.

In some embodiments, the anti-TfR antibody of the present disclosure comprises a humanized VH comprising a CDR-H1 having the amino acid sequence of SEQ ID NO: 150 (according to the Chothia definition system), a CDR-H2 having the amino acid sequence of SEQ ID NO: 151, SEQ ID NO: 270, or SEQ ID NO: 271 (according to the Chothia definition system), a CDR-H3 having the amino acid sequence of SEQ ID NO: 152 (according to the Chothia definition system), and containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VH as set forth in SEQ ID NO: 7. Alternatively or in addition (e.g., in addition), the anti-TfR antibody of the present disclosure comprises a humanized VL comprising a CDR-L1 having the amino acid sequence of SEQ ID NO: 153 (according to the Chothia definition system), a CDR-L2 having the amino acid sequence of SEQ ID NO: 5 (according to the Chothia definition system), and a CDR-L3 having the amino acid sequence of SEQ ID NO: 154 (according to the Chothia definition system), and containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VL as set forth in SEQ ID NO: 8.

In some embodiments, the anti-TfR antibody of the present disclosure comprises a humanized VH comprising a CDR-H1 having the amino acid sequence of SEQ ID NO: 150 (according to the Chothia definition system), a CDR-H2 having the amino acid sequence of SEQ ID NO: 151, SEQ ID NO: 270, or SEQ ID NO: 271 (according to the Chothia definition system), a CDR-H3 having the amino acid sequence of SEQ ID NO: 152 (according to the Chothia definition system), and is at least 75% (e.g., 75%, 80%, 85%, 90%, 95%, 98%, or 99%) identical in the framework regions to the VH as set forth in SEQ ID NO: 7. Alternatively or in addition (e.g., in addition), the anti-TfR antibody of the present disclosure comprises a humanized VL comprising a CDR-L1 having the amino acid sequence of SEQ ID NO: 153 (according to the Chothia definition system), a CDR-L2 having the amino acid sequence of SEQ ID NO: 5 (according to the Chothia definition system), and a CDR-L3 having the amino acid sequence of SEQ ID NO: 154 (according to the Chothia definition system), and is at least 75% (e.g., 75%, 80%, 85%, 90%, 95%, 98%, or 99%) identical in the framework regions to the VL as set forth in SEQ ID NO: 8.

In some embodiments, the anti-TfR antibody of the present disclosure comprises a humanized VH comprising a CDR-H1 having the amino acid sequence of SEQ ID NO: 9 (according to the IMGT definition system), a CDR-H2 having the amino acid sequence of SEQ ID NO: 10 (according to the IMGT definition system), a CDR-H3 having the amino acid sequence of SEQ ID NO: 11 (according to the IMGT definition system), and containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VH as set forth in SEQ ID NO: 15. Alternatively or in addition (e.g., in addition), the anti-TfR antibody of the present disclosure comprises a humanized VL comprising a CDR-L1 having the amino acid sequence of SEQ ID NO: 12 (according to the IMGT definition system), a CDR-L2 having the amino acid sequence of SEQ ID NO: 13 (according to the IMGT definition system), and a CDR-L3 having the amino acid sequence of SEQ ID NO: 14 (according to the IMGT definition system), and containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VL as set forth in SEQ ID NO: 16.

In some embodiments, the anti-TfR antibody of the present disclosure comprises a humanized VH comprising a CDR-H1 having the amino acid sequence of SEQ ID NO: 9 (according to the IMGT definition system), a CDR-H2 having the amino acid sequence of SEQ ID NO: 10 (according to the IMGT definition system), a CDR-H3 having the amino acid sequence of SEQ ID NO: 11 (according to the IMGT definition system), and is at least 75% (e.g., 75%, 80%, 85%, 90%, 95%, 98%, or 99%) identical in the framework regions to the VH as set forth in SEQ ID NO: 15. Alternatively or in addition (e.g., in addition), the anti-TfR antibody of the present disclosure comprises a humanized VL comprising a CDR-L1 having the amino acid sequence of SEQ ID NO: 12 (according to the IMGT definition system), a CDR-L2 having the amino acid sequence of SEQ ID NO: 13 (according to the IMGT definition system), and a CDR-L3 having the amino acid sequence of SEQ ID NO: 14 (according to the IMGT definition system), and is at least 75% (e.g., 75%, 80%, 85%, 90%, 95%, 98%, or 99%) identical in the framework regions to the VL as set forth SEQ ID NO: 16.

In some embodiments, the anti-TfR antibody of the present disclosure comprises a humanized VH comprising a CDR-H1 having the amino acid sequence of SEQ ID NO: 155 (according to the Kabat definition system), a CDR-H2 having the amino acid sequence of SEQ ID NO: 156 (according to the Kabat definition system), a CDR-H3 having the amino acid sequence of SEQ ID NO: 157 (according to the Kabat definition system), and containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VH as set forth in SEQ ID NO: 15. Alternatively or in addition (e.g., in addition), the anti-TfR antibody of the present disclosure comprises a humanized VL comprising a CDR-L1 having the amino acid sequence of SEQ ID NO: 158 (according to the Kabat definition system), a CDR-L2 having the amino acid sequence of SEQ ID NO: 159 (according to the Kabat definition system), and a CDR-L3 having the amino acid sequence of SEQ ID NO: 14 (according to the Kabat definition system), and containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VL as set forth in SEQ ID NO: 16.

In some embodiments, the anti-TfR antibody of the present disclosure comprises a humanized VH comprising a CDR-H1 having the amino acid sequence of SEQ ID NO: 155 (according to the Kabat definition system), a CDR-H2 having the amino acid sequence of SEQ ID NO: 156 (according to the Kabat definition system), a CDR-H3 having the amino acid sequence of SEQ ID NO: 157 (according to the Kabat definition system), and is at least 75% (e.g., 75%, 80%, 85%, 90%, 95%, 98%, or 99%) identical in the framework regions to the VH as set forth in SEQ ID NO: 15. Alternatively or in addition (e.g., in addition), the anti-TfR antibody of the present disclosure comprises a humanized VL comprising a CDR-L1 having the amino acid sequence of SEQ ID NO: 158 (according to the Kabat definition system), a CDR-L2 having the amino acid sequence of SEQ ID NO: 159 (according to the Kabat definition system), and a CDR-L3 having the amino acid sequence of SEQ ID NO: 14 (according to the Kabat definition system), and is at least 75% (e.g., 75%, 80%, 85%, 90%, 95%, 98%, or 99%) identical in the framework regions to the VL as set forth in SEQ ID NO: 16.

In some embodiments, the anti-TfR antibody of the present disclosure comprises a humanized VH comprising a CDR-H1 having the amino acid sequence of SEQ ID NO: 160 (according to the Chothia definition system), a CDR-H2 having the amino acid sequence of SEQ ID NO: 161 (according to the Chothia definition system), a CDR-H3 having the amino acid sequence of SEQ ID NO: 162 (according to the Chothia definition system), and containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VH as set forth in SEQ ID NO: 15. Alternatively or in addition (e.g., in addition), the anti-TfR antibody of the present disclosure comprises a humanized VL comprising a CDR-L1 having the amino acid sequence of SEQ ID NO: 163 (according to the Chothia definition system), a CDR-L2 having the amino acid sequence of SEQ ID NO: 13 (according to the Chothia definition system), and a CDR-L3 having the amino acid sequence of SEQ ID NO: 164 (according to the Chothia definition system), and containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VL as set forth in SEQ ID NO: 16.

In some embodiments, the anti-TfR antibody of the present disclosure comprises a humanized VH comprising a CDR-H1 having the amino acid sequence of SEQ ID NO: 160 (according to the Chothia definition system), a CDR-H2 having the amino acid sequence of SEQ ID NO: 161 (according to the Chothia definition system), a CDR-H3 having the amino acid sequence of SEQ ID NO: 162 (according to the Chothia definition system), and is at least 75% (e.g., 75%, 80%, 85%, 90%, 95%, 98%, or 99%) identical in the framework regions to the VH as set forth in SEQ ID NO: 15. Alternatively or in addition (e.g., in addition), the anti-TfR antibody of the present disclosure comprises a humanized VL comprising a CDR-L1 having the amino acid sequence of SEQ ID NO: 163 (according to the Chothia definition system), a CDR-L2 having the amino acid sequence of SEQ ID NO: 13 (according to the Chothia definition system), and a CDR-L3 having the amino acid sequence of SEQ ID NO: 164 (according to the Chothia definition system), and is at least 75% (e.g., 75%, 80%, 85%, 90%, 95%, 98%, or 99%) identical in the framework regions to the VL as set forth in SEQ ID NO: 16.

In some embodiments, the anti-TfR antibody of the present disclosure comprises a humanized VH comprising a CDR-H1 having the amino acid sequence of SEQ ID NO: 17, SEQ ID NO: 266, or SEQ ID NO: 268 (according to the IMGT definition system), a CDR-H2 having the amino acid sequence of SEQ ID NO: 18 (according to the IMGT definition system), a CDR-H3 having the amino acid sequence of SEQ ID NO: 19 (according to the IMGT definition system), and containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VH as set forth in SEQ ID NO: 23. Alternatively or in addition (e.g., in addition), the anti-TfR antibody of the present disclosure comprises a humanized VL comprising a CDR-L1 having the amino acid sequence of SEQ ID NO: 20 (according to the IMGT definition system), a CDR-L2 having the amino acid sequence of SEQ ID NO: 21 (according to the IMGT definition system), and a CDR-L3 having the amino acid sequence of SEQ ID NO: 22 (according to the IMGT definition system), and containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VL as set forth in SEQ ID NO: 24.

In some embodiments, the anti-TfR antibody of the present disclosure comprises a humanized VH comprising a CDR-H1 having the amino acid sequence of SEQ ID NO: 17, SEQ ID NO: 266, or SEQ ID NO: 268 (according to the IMGT definition system), a CDR-H2 having the amino acid sequence of SEQ ID NO: 18 (according to the IMGT definition system), a CDR-H3 having the amino acid sequence of SEQ ID NO: 19 (according to the IMGT definition system), and is at least 75% (e.g., 75%, 80%, 85%, 90%, 95%, 98%, or 99%) identical in the framework regions to the VH as set forth in SEQ ID NO: 23. Alternatively or in addition (e.g., in addition), the anti-TfR antibody of the present disclosure comprises a humanized VL comprising a CDR-L1 having the amino acid sequence of SEQ ID NO: 20 (according to the IMGT definition system), a CDR-L2 having the amino acid sequence of SEQ ID NO: 21 (according to the IMGT definition system), and a CDR-L3 having the amino acid sequence of SEQ ID NO: 22 (according to the IMGT definition system), and is at least 75% (e.g., 75%, 80%, 85%, 90%, 95%, 98%, or 99%) identical in the framework regions to the VL as set forth in SEQ ID NO: 24.

In some embodiments, the anti-TfR antibody of the present disclosure comprises a humanized VH comprising a CDR-H1 having the amino acid sequence of SEQ ID NO: 165, SEQ ID NO: 267, or SEQ ID NO: 269 (according to the Kabat definition system), a CDR-H2 having the amino acid sequence of SEQ ID NO: 166 (according to the Kabat definition system), a CDR-H3 having the amino acid sequence of SEQ ID NO: 167 (according to the Kabat definition system), and containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VH as set forth in SEQ ID NO: 23. Alternatively or in addition (e.g., in addition), the anti-TfR antibody of the present disclosure comprises a humanized VL comprising a CDR-L1 having the amino acid sequence of SEQ ID NO: 168 (according to the Kabat definition system), a CDR-L2 having the amino acid sequence of SEQ ID NO: 169 (according to the Kabat definition system), and a CDR-L3 having the amino acid sequence of SEQ ID NO: 22 (according to the Kabat definition system), and containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VL as set forth in SEQ ID NO: 24.

In some embodiments, the anti-TfR antibody of the present disclosure comprises a humanized VH comprising a CDR-H1 having the amino acid sequence of SEQ ID NO: 165, SEQ ID NO: 267, or SEQ ID NO: 269 (according to the Kabat definition system), a CDR-H2 having the amino acid sequence of SEQ ID NO: 166 (according to the Kabat definition system), a CDR-H3 having the amino acid sequence of SEQ ID NO: 167 (according to the Kabat definition system), and is at least 75% (e.g., 75%, 80%, 85%, 90%, 95%, 98%, or 99%) identical in the framework regions to the VH as set forth in SEQ ID NO: 23. Alternatively or in addition (e.g., in addition), the anti-TfR antibody of the present disclosure comprises a humanized VL comprising a CDR-L1 having the amino acid sequence of SEQ ID NO: 168 (according to the Kabat definition system), a CDR-L2 having the amino acid sequence of SEQ ID NO: 169 (according to the Kabat definition system), and a CDR-L3 having the amino acid sequence of SEQ ID NO: 22 (according to the Kabat definition system), and is at least 75% (e.g., 75%, 80%, 85%, 90%, 95%, 98%, or 99%) identical in the framework regions to the VL as set forth in SEQ ID NO: 24.

In some embodiments, the anti-TfR antibody of the present disclosure comprises a humanized VH comprising a CDR-H1 having the amino acid sequence of SEQ ID NO: 170 (according to the Chothia definition system), a CDR-H2 having the amino acid sequence of SEQ ID NO: 171 (according to the Chothia definition system), a CDR-H3 having the amino acid sequence of SEQ ID NO: 172 (according to the Chothia definition system), and containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VH as set forth in SEQ ID NO: 23. Alternatively or in addition (e.g., in addition), the anti-TfR antibody of the present disclosure comprises a humanized VL comprising a CDR-L1 having the amino acid sequence of SEQ ID NO: 173 (according to the Chothia definition system), a CDR-L2 having the amino acid sequence of SEQ ID NO: 21 (according to the Chothia definition system), and a CDR-L3 having the amino acid sequence of SEQ ID NO: 174 (according to the Chothia definition system), and containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VL as set forth in SEQ ID NO: 24.

In some embodiments, the anti-TfR antibody of the present disclosure comprises a humanized VH comprising a CDR-H1 having the amino acid sequence of SEQ ID NO: 170 (according to the Chothia definition system), a CDR-H2 having the amino acid sequence of SEQ ID NO: 171 (according to the Chothia definition system), a CDR-H3 having the amino acid sequence of SEQ ID NO: 172 (according to the Chothia definition system), and is at least 75% (e.g., 75%, 80%, 85%, 90%, 95%, 98%, or 99%) identical in the framework regions to the VH as set forth in SEQ ID NO: 23. Alternatively or in addition (e.g., in addition), the anti-TfR antibody of the present disclosure comprises a humanized VL comprising a CDR-L1 having the amino acid sequence of SEQ ID NO: 173 (according to the Chothia definition system), a CDR-L2 having the amino acid sequence of SEQ ID NO: 21 (according to the Chothia definition system), and a CDR-L3 having the amino acid sequence of SEQ ID NO: 174 (according to the Chothia definition system), and is at least 75% (e.g., 75%, 80%, 85%, 90%, 95%, 98%, or 99%) identical in the framework regions to the VL as set forth in SEQ ID NO: 24.

In some embodiments, the anti-TfR antibody of the present disclosure is a chimeric antibody, which can include a heavy constant region and a light constant region from a human antibody. Chimeric antibodies refer to antibodies having a variable region or part of variable region from a first species and a constant region from a second species. Typically, in these chimeric antibodies, the variable region of both light and heavy chains mimics the variable regions of antibodies derived from one species of mammals (e.g., a non-human mammal such as mouse, rabbit, and rat), while the constant portions are homologous to the sequences in antibodies derived from another mammal such as human. In some embodiments, amino acid modifications can be made in the variable region and/or (e.g., and) the constant region.

In some embodiments, the anti-TfR antibody described herein is a chimeric antibody, which can include a heavy constant region and a light constant region from a human antibody. Chimeric antibodies refer to antibodies having a variable region or part of variable region from a first species and a constant region from a second species. Typically, in these chimeric antibodies, the variable region of both light and heavy chains mimics the variable regions of antibodies derived from one species of mammals (e.g., a non-human mammal such as mouse, rabbit, and rat), while the constant portions are homologous to the sequences in antibodies derived from another mammal such as human. In some embodiments, amino acid modifications can be made in the variable region and/or (e.g., and) the constant region.

In some embodiments, the heavy chain of any of the anti-TfR antibodies as described herein may comprises a heavy chain constant region (CH) or a portion thereof (e.g., CH1, CH2, CH3, or a combination thereof). The heavy chain constant region can of any suitable origin, e.g., human, mouse, rat, or rabbit. In one specific example, the heavy chain constant region is from a human IgG (a gamma heavy chain), e.g., IgG1, IgG2, or IgG4. An example of a human IgG1 constant region is given below:

(SEQ ID NO: 175)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPGK

In some embodiments, the heavy chain of any of the anti-TfR antibodies described herein comprises a mutant human IgG1 constant region. For example, the introduction of LALA mutations (a mutant derived from mAb b12 that has been mutated to replace the lower hinge residues Leu234 Leu235 with Ala234 and Ala235) in the CH2 domain of human IgG1 is known to reduce Fcg receptor binding (Bruhns, P., et al . (2009) and Xu, D. et al. (2000)). The mutant human IgG1 constant region is provided below (mutations bonded and underlined):

(SEQ ID NO: 176)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPEAAGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPGK

In some embodiments, the light chain of any of the anti-TfR antibodies described herein may further comprise a light chain constant region (CL), which can be any CL known in the art. In some examples, the CL is a kappa light chain. In other examples, the CL is a lambda light chain. In some embodiments, the CL is a kappa light chain, the sequence of which is provided below:

(SEQ ID NO: 177)
RTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPV
TKSFNRGEC

Other antibody heavy and light chain constant regions are well known in the art, e.g., those provided in the IMGT database (www.imgt.org) or at www.vbase2.org/vbstat.php., both of which are incorporated by reference herein.

In some embodiments, the anti-TfR antibody described herein comprises a heavy chain comprising any one of the VH as listed in Table 2 or any variants thereof and a heavy chain constant region that is at least 80%, at least 85%, at least 90%, at least 95%, or at least 99% identical to SEQ ID NO: 175 or SEQ ID NO: 176. In some embodiments, the anti-TfR antibody described herein comprises a heavy chain comprising any one of the VH as listed in Table 2 or any variants thereof and a heavy chain constant region that contains no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with SEQ ID NO: 175 or SEQ ID NO: 176. In some embodiments, the anti-TfR antibody described herein comprises a heavy chain comprising any one of the VH as listed in Table 2 or any variants thereof and a heavy chain constant region as set forth in SEQ ID NO: 175. In some embodiments, the anti-TfR antibody described herein comprises heavy chain comprising any one of the VH as listed in Table 2 or any variants thereof and a heavy chain constant region as set forth in SEQ ID NO: 176.

In some embodiments, the anti-TfR antibody described herein comprises a light chain comprising any one of the VL as listed in Table 2 or any variants thereof and a light chain constant region that is at least 80%, at least 85%, at least 90%, at least 95%, or at least 99% identical to SEQ ID NO: 177. In some embodiments, the anti-TfR antibody described herein comprises a light chain comprising any one of the VL as listed in Table 2 or any variants thereof and a light chain constant region contains no more than 25 amino acid variations (e.g., no more than 25 ,24, 23, 22, 21, 20, 19, 18, 17, 16, 15, 14, 13, 12, 11, 10, 9, 8, 7, 6, 5, 4, 3, 2, or 1 amino acid variation) as compared with SEQ ID NO: 177. In some embodiments, the anti-TfR antibody described herein comprises a light chain comprising any one of the VL as listed in Table 2 or any variants thereof and a light chain constant region set forth in SEQ ID NO: 177.

Examples of IgG heavy chain and light chain amino acid sequences of the anti-TfR antibodies described are provided in Table 5 below.

TABLE 5
Heavy chain and light chain sequences of examples of anti-TfR IgGs

| Antibody | IgG Heavy Chain/Light Chain Sequences |
|---|---|
| 3-A4 | Heavy Chain (with wild type human IgG1 constant region) |
| | EVQLQQSGAELVRPGASVKLSCTASGFNIKDDYMYWVKQRPEQGLEWIGWIDPENGDTEYAS |
| | KFQDKATVTADTSSNTAYLQLSSLTSEDTAVYYCTLWLRRGLDYWGQGTSVTVSSASTKGPS |
| | VFPLAPSSKSTSGGTAALGCLVKDYFPEPVTVSWNSGALTSGVHTFPAVLQSSGLYSLSSVVTV |
| | PSSSLGTQTYICNVNHKPSNTKVDKKVEPKSCDKTHTCPPCPAPELLGGPSVFLFPPKPKDTLMI |
| | SRTPEVTCVVVDVSHEDPEVKFNWYVDGVEVHNAKTKPREEQYNSTYRVVSVLTVLHQDWL |
| | NGKEYKCKVSNKALPAPIEKTISKAKGQPREPQVYTLPPSRDELTKNQVSLTCLVKGFYPSDIA |
| | VEWESNGQPENNYKTTPPVLDSDGSFFLYSKLTVDKSRWQQGNVFSCSVMHEALHNHYTQKS |
| | LSLSPGK (SEQ ID NO: 178) |
| | Light Chain (with kappa light chain constant region) |
| | DIVMTQAAPSVPVTPGESVSISCRSSKSLLHSNGYTYLFWFLQRPGQSPQLLIYRMSNLASGVP |
| | DRFSGSGSGTAFTLRISRVEAEDVGVYYCMQHLEYPFTFGGGTKLEIKRTVAAPSVFIFPPSDEQ |
| | LKSGTASVVCLLNNFYPREAKVQWKVDNALQSGNSQESVTEQDSKDSTYSLSSTLTLSKADYE |
| | KHKVYACEVTHQGLSSPVTKSFNRGEC (SEQ ID NO: 179) |
| 3-A4 | Heavy Chain (with wild type human IgG1 constant region) |
| Variant 1 | EVQLQQSGAELVRPGASVKLSCTASGFNIKDDYMYWVKQRPEQGLEWIGWIDPETGDTEYAS |
| | KFQDKATVTADTSSNTAYLQLSSLTSEDTAVYYCTLWLRRGLDYWGQGTSVTVSSASTKGPS |
| | VFPLAPSSKSTSGGTAALGCLVKDYFPEPVTVSWNSGALTSGVHTFPAVLQSSGLYSLSSVVTV |
| | PSSSLGTQTYICNVNHKPSNTKVDKKVEPKSCDKTHTCPPCPAPELLGGPSVFLFPPKPKDTLMI |
| | SRTPEVTCVVVDVSHEDPEVKFNWYVDGVEVHNAKTKPREEQYNSTYRVVSVLTVLHQDWL |
| | NGKEYKCKVSNKALPAPIEKTISKAKGQPREPQVYTLPPSRDELTKNQVSLTCLVKGFYPSDIA |
| | VEWESNGQPENNYKTTPPVLDSDGSFFLYSKLTVDKSRWQQGNVFSCSVMHEALHNHYTQKS |
| | LSLSPGK (SEQ ID NO: 551) |
| | Light Chain (with kappa light chain constant region) |
| | DIVMTQAAPSVPVTPGESVSISCRSSKSLLHSNGYTYLFWFLQRPGQSPQLLIYRMSNLASGVP |
| | DRFSGSGSGTAFTLRISRVEAEDVGVYYCMQHLEYPFTFGGGTKLEIKRTVAAPSVFIFPPSDEQ |
| | LKSGTASVVCLLNNFYPREAKVQWKVDNALQSGNSQESVTEQDSKDSTYSLSSTLTLSKADYE |
| | KHKVYACEVTHQGLSSPVTKSFNRGEC (SEQ ID NO: 179) |
| 3-A4 | Heavy Chain (with wild type human IgG1 constant region) |
| Variant 2 | EVQLQQSGAELVRPGASVKLSCTASGFNIKDDYMYWVKQRPEQGLEWIGWIDPESGDTEYAS |
| | KFQDKATVTADTSSNTAYLQLSSLTSEDTAVYYCTLWLRRGLDYWGQGTSVTVSSASTKGPS |
| | VFPLAPSSKSTSGGTAALGCLVKDYFPEPVTVSWNSGALTSGVHTFPAVLQSSGLYSLSSVVTV |
| | PSSSLGTQTYICNVNHKPSNTKVDKKVEPKSCDKTHTCPPCPAPELLGGPSVFLFPPKPKDTLMI |
| | SRTPEVTCVVVDVSHEDPEVKFNWYVDGVEVHNAKTKPREEQYNSTYRVVSVLTVLHQDWL |
| | NGKEYKCKVSNKALPAPIEKTISKAKGQPREPQVYTLPPSRDELTKNQVSLTCLVKGFYPSDIA |
| | VEWESNGQPENNYKTTPPVLDSDGSFFLYSKLTVDKSRWQQGNVFSCSVMHEALHNHYTQKS |
| | LSLSPGK (SEQ ID NO: 552) |
| | Light Chain (with kappa light chain constant region) |
| | DIVMTQAAPSVPVTPGESVSISCRSSKSLLHSNGYTYLFWFLQRPGQSPQLLIYRMSNLASGVP |
| | DRFSGSGSGTAFTLRISRVEAEDVGVYYCMQHLEYPFTFGGGTKLEIKRTVAAPSVFIFPPSDEQ |
| | LKSGTASVVCLLNNFYPREAKVQWKVDNALQSGNSQESVTEQDSKDSTYSLSSTLTLSKADYE |
| | KHKVYACEVTHQGLSSPVTKSFNRGEC (SEQ ID NO: 179) |
| 3-M12 | Heavy Chain (with wild type human IgG1 constant region) |
| | DVQLQESGPGLVKPSQSLSLTCSVTGYSITSGYYWNWIRQFPGNKLEWMGYITFDGANNYNPS |
| | LKNRISITRDTSKNQFFLKLTSVTTEDTATYYCTRSSYDYDVLDYWGQGTTLTVSSASTKGPSV |
| | FPLAPSSKSTSGGTAALGCLVKDYFPEPVTVSWNSGALTSGVHTFPAVLQSSGLYSLSSVVTVP |
| | SSSLGTQTYICNVNHKPSNTKVDKKVEPKSCDKTHTCPPCPAPELLGGPSVFLFPPKPKDTLMIS |
| | RTPEVTCVVVDVSHEDPEVKFNWYVDGVEVHNAKTKPREEQYNSTYRVVSVLTVLHQDWLN |
| | GKEYKCKVSNKALPAPIEKTISKAKGQPREPQVYTLPPSRDELTKNQVSLTCLVKGFYPSDIAV |
| | EWESNGQPENNYKTTPPVLDSDGSFFLYSKLTVDKSRWQQGNVFSCSVMHEALHNHYTQKSL |
| | SLSPGK (SEQ ID NO: 180) |
| | Light Chain (with kappa light chain constant region) |
| | DIQMTQTTSSLSASLGDRVTISCRASQDISNFLNWYQQRPDGTVKLLIYYTSRLHSGVPSRFSGS |
| | GSGTDFSLTVSNLEQEDIATYFCQQGHTLPYTFGGGTKLEIKRTVAAPSVFIFPPSDEQLKSGTA |
| | SVVCLLNNFYPREAKVQWKVDNALQSGNSQESVTEQDSKDSTYSLSSTLTLSKADYEKHKVY |
| | ACEVTHQGLSSPVTKSFNRGEC (SEQ ID NO: 181) |
| 5-H12 | Heavy Chain (with wild type human IgG1 constant region) |
| | QIQLQQSGPELVRPGASVKISCKASGYSFTDYCINWVNQRPGQGLEWIGWIYPGSGNTRYSERF |
| | KGKATLTVDTSSNTAYMQLSSLTSEDSAVYFCAREDYYPYHGMDYWGQGTSVTVSSASTKGP |
| | SVFPLAPSSKSTSGGTAALGCLVKDYFPEPVTVSWNSGALTSGVHTFPAVLQSSGLYSLSSVVT |
| | VPSSSLGTQTYICNVNHKPSNTKVDKKVEPKSCDKTHTCPPCPAPELLGGPSVFLFPPKPKDTL |
| | MISRTPEVTCVVVDVSHEDPEVKFNWYVDGVEVHNAKTKPREEQYNSTYRVVSVLTVLHQD |
| | WLNGKEYKCKVSNKALPAPIEKTISKAKGQPREPQVYTLPPSRDELTKNQVSLTCLVKGFYPS |
| | DIAVEWESNGQPENNYKTTPPVLDSDGSFFLYSKLTVDKSRWQQGNVFSCSVMHEALHNHYT |
| | QKSLSLSPGK (SEQ ID NO: 182) |
| | Light Chain (with kappa light chain constant region) |
| | DIVLTQSPTSLAVSLGQRATISCRASESVDGYDNSFMHWYQQKPGQPPKLLIFRASNLESGIPAR |
| | FSGSGSRTDFTLTINPVEAADVATYYCQQSSEDPWTFGGGTKLEIKRTVAAPSVFIFPPSDEQLK |
| | SGTASVVCLLNNFYPREAKVQWKVDNALQSGNSQESVTEQDSKDSTYSLSSTLTLSKADYEK |
| | HKVYACEVTHQGLSSPVTKSFNRGEC (SEQ ID NO: 183) |
| 5-H12 | Heavy Chain (with wild type human IgG1 constant region) |
| Variant 1 | QIQLQQSGPELVRPGASVKISCKASGYSFTDYYINWVNQRPGQGLEWIGWIYPGSGNTRYSERF |
| | KGKATLTVDTSSNTAYMQLSSLTSEDSAVYFCAREDYYPYHGMDYWGQGTSVTVSSASTKGP |
| | SVFPLAPSSKSTSGGTAALGCLVKDYFPEPVTVSWNSGALTSGVHTFPAVLQSSGLYSLSSVVT |
| | VPSSSLGTQTYICNVNHKPSNTKVDKKVEPKSCDKTHTCPPCPAPELLGGPSVFLFPPKPKDTL |
| | MISRTPEVTCVVVDVSHEDPEVKFNWYVDGVEVHNAKTKPREEQYNSTYRVVSVLTVLHQD |
| | WLNGKEYKCKVSNKALPAPIEKTISKAKGQPREPQVYTLPPSRDELTKNQVSLTCLVKGFYPS |
| | DIAVEWESNGQPENNYKTTPPVLDSDGSFFLYSKLTVDKSRWQQGNVFSCSVMHEALHNHYT |
| | QKSLSLSPGK (SEQ ID NO: 553) |
| | Light Chain (with kappa light chain constant region) |
| | DIVLTQSPTSLAVSLGQRATISCRASESVDGYDNSFMHWYQQKPGQPPKLLIFRASNLESGIPAR |
| | FSGSGSRTDFTLTINPVEAADVATYYCQQSSEDPWTFGGGTKLEIKRTVAAPSVFIFPPSDEQLK |
| | SGTASVVCLLNNFYPREAKVQWKVDNALQSGNSQESVTEQDSKDSTYSLSSTLTLSKADYEK |
| | HKVYACEVTHQGLSSPVTKSFNRGEC (SEQ ID NO: 183) |
| 5-H12 | Heavy Chain (with wild type human IgG1 constant region) |
| Variant 2 | QIQLQQSGPELVRPGASVKISCKASGYSFTDYDINWVNQRPGQGLEWIGWIYPGSGNTRYSERF |
| | KGKATLTVDTSSNTAYMQLSSLTSEDSAVYFCAREDYYPYHGMDYWGQGTSVTVSSASTKGP |
| | SVFPLAPSSKSTSGGTAALGCLVKDYFPEPVTVSWNSGALTSGVHTFPAVLQSSGLYSLSSVVT |
| | VPSSSLGTQTYICNVNHKPSNTKVDKKVEPKSCDKTHTCPPCPAPELLGGPSVFLFPPKPKDTL |
| | MISRTPEVTCVVVDVSHEDPEVKFNWYVDGVEVHNAKTKPREEQYNSTYRVVSVLTVLHQD |
| | WLNGKEYKCKVSNKALPAPIEKTISKAKGQPREPQVYTLPPSRDELTKNQVSLTCLVKGFYPS |
| | DIAVEWESNGQPENNYKTTPPVLDSDGSFFLYSKLTVDKSRWQQGNVFSCSVMHEALHNHYT |
| | QKSLSLSPGK (SEQ ID NO: 554) |
| | Light Chain (with kappa light chain constant region) |
| | DIVLTQSPTSLAVSLGQRATISCRASESVDGYDNSFMHWYQQKPGQPPKLLIFRASNLESGIPAR |
| | FSGSGSRTDFTLTINPVEAADVATYYCQQSSEDPWTFGGGTKLEIKRTVAAPSVFIFPPSDEQLK |
| | SGTASVVCLLNNFYPREAKVQWKVDNALQSGNSQESVTEQDSKDSTYSLSSTLTLSKADYEK |
| | HKVYACEVTHQGLSSPVTKSFNRGEC (SEQ ID NO: 183) |

*VH/VL sequences underlined

In some embodiments, the anti-TfR antibody of the present disclosure comprises a heavy chain containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the heavy chain as set forth in SEQ ID NO: 178, SEQ ID NO: 180, SEQ ID NO: 182, SEQ ID NO: 300, SEQ ID NO: 301, SEQ ID NO: 302, or SEQ ID NO: 303. Alternatively or in addition (e.g., in addition), the anti-TfR antibody of the present disclosure comprises a light chain containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the light chain as set forth in SEQ ID NO: 179, SEQ ID NO: 181, or SEQ ID NO: 183. In some embodiments, the anti-TfR antibody described herein comprises a heavy chain comprising an amino acid sequence that is at least 75% (e.g., 75%, 80%, 85%, 90%, 95%, 98%, or 99%) identical to SEQ ID NO: 178, SEQ ID NO: 180, SEQ ID NO: 182, SEQ ID NO: 300, SEQ ID NO: 301, SEQ ID NO: 302, or SEQ ID NO: 303. Alternatively or in addition (e.g., in addition), the anti-TfR antibody described herein comprises a light chain comprising an amino acid sequence that is at least 75% (e.g., 75%, 80%, 85%, 90%, 95%, 98%, or 99%) identical to SEQ ID NO: 179, SEQ ID NO: 181, or SEQ ID NO: 183. In some embodiments, the anti-TfR antibody described herein comprises a heavy chain comprising the amino acid sequence of SEQ ID NO: 178, SEQ ID NO: 180, SEQ ID NO: 182, SEQ ID NO: 300, SEQ ID NO: 301, SEQ ID NO: 302, or SEQ ID NO: 303. Alternatively or in addition (e.g., in addition), the anti-TfR antibody described herein comprises a light chain comprising the amino acid sequence of SEQ ID NO: 179, SEQ ID NO: 181, or SEQ ID NO: 183.

In some embodiments, the anti-TfR antibody of the present disclosure comprises a heavy chain containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the heavy chain as set forth in SEQ ID NO: 178, SEQ ID NO: 300, or SEQ ID NO: 301. Alternatively or in addition (e.g., in addition), the anti-TfR antibody of the present disclosure comprises a light chain containing no more than 25 amino acid variations (e.g., no more than 25, 24, 23, 22, 21, 20, 19, 18, 17, 16, 15, 14, 13, 12, 10, 11, 9, 8, 7, 6, 5, 4, 3, 2, or 1 amino acid variation) as compared with the light chain as set forth in SEQ ID NO: 179. In some embodiments, the anti-TfR antibody described herein comprises a heavy chain comprising an amino acid sequence that is at least 75% (e.g., 75%, 80%, 85%, 90%, 95%, 98%, or 99%) identical to SEQ ID NO: 178, SEQ ID NO: 300, or SEQ ID NO: 301. Alternatively or in addition (e.g., in addition), the anti-TfR antibody described herein comprises a light chain comprising an amino acid sequence that is at least 75% (e.g., 75%, 80%, 85%, 90%, 95%, 98%, or 99%) identical to SEQ ID NO: 179. In some embodiments, the anti-TfR antibody described herein comprises a heavy chain comprising the amino acid sequence of SEQ ID NO: 178, SEQ ID NO: 300, or SEQ ID NO: 301. Alternatively or in addition (e.g., in addition), the anti-TfR antibody described herein comprises a light chain comprising the amino acid sequence of SEQ ID NO: 179.

In some embodiments, the anti-TfR antibody of the present disclosure comprises a heavy chain containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the heavy chain as set forth in SEQ ID NO: 180. Alternatively or in addition (e.g., in addition), the anti-TfR antibody of the present disclosure comprises a light chain containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the light chain as set forth in SEQ ID NO: 181. In some embodiments, the anti-TfR antibody described herein comprises a heavy chain comprising an amino acid sequence that is at least 75% (e.g., 75%, 80%, 85%, 90%, 95%, 98%, or 99%) identical to SEQ ID NO: 180. Alternatively or in addition (e.g., in addition), the anti-TfR antibody described herein comprises a light chain comprising an amino acid sequence that is at least 75% (e.g., 75%, 80%, 85%, 90%, 95%, 98%, or 99%) identical to SEQ ID NO: 181. In some embodiments, the anti-TfR antibody described herein comprises a heavy chain comprising the amino acid sequence of SEQ ID NO: 180. Alternatively or in addition (e.g., in addition), the anti-TfR antibody described herein comprises a light chain comprising the amino acid sequence of SEQ ID NO: 181.

In some embodiments, the anti-TfR antibody of the present disclosure comprises a heavy chain containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the heavy chain as set forth in SEQ ID NO: 182, SEQ ID NO: 302 or SEQ ID NO: 303. Alternatively or in addition (e.g., in addition), the anti-TfR antibody of the present disclosure comprises a light chain containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the light chain as set forth in SEQ ID NO: 183. In some embodiments, the anti-TfR antibody described herein comprises a heavy chain comprising an amino acid sequence that is at least 75% (e.g., 75%, 80%, 85%, 90%, 95%, 98%, or 99%) identical to SEQ ID NO: 182, SEQ ID NO: 302 or SEQ ID NO: 303. Alternatively or in addition (e.g., in addition), the anti-TfR antibody described herein comprises a light chain comprising an amino acid sequence that is at least 75% (e.g., 75%, 80%, 85%, 90%, 95%, 98%, or 99%) identical to SEQ ID NO: 183. In some embodiments, the anti-TfR antibody described herein comprises a heavy chain comprising the amino acid sequence of SEQ ID NO: 182, SEQ ID NO: 302 or SEQ ID NO: 303. Alternatively or in addition (e.g., in addition), the anti-TfR antibody described herein comprises a light chain comprising the amino acid sequence of SEQ ID NO: 183.

In some embodiments, the anti-TfR antibody is a FAB fragment, F(ab′) fragment, or F(ab′)₂ fragment of an intact antibody (full-length antibody). Antigen binding fragment of an intact antibody (full-length antibody) can be prepared via routine methods (e.g., recombinantly or by digesting the heavy chain constant region of a full length IgG using an enzyme such as papain). For example, F(ab′)₂ fragments can be produced by pepsin or papain digestion of an antibody molecule, and Fab fragments that can be generated by reducing the disulfide bridges of F(ab′)₂ fragments. In some embodiments, a heavy chain constant region in a F(ab′) fragment of the anti-TfR1 antibody described herein comprises the amino acid sequence of:

(SEQ ID NO: 184)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHT

In some embodiments, the anti-TfR antibody described herein comprises a heavy chain comprising any one of the VH as listed in Table 2 or any variants thereof and a heavy chain constant region that is at least 80%, at least 85%, at least 90%, at least 95%, or at least 99% identical to SEQ ID NO: 184. In some embodiments, the anti-TfR antibody described herein comprises a heavy chain comprising any one of the VH as listed in Table 2 or any variants thereof and a heavy chain constant region that contains no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with SEQ ID NO: 184. In some embodiments, the anti-TfR antibody described herein comprises a heavy chain comprising any one of the VH as listed in Table 2 or any variants thereof and a heavy chain constant region as set forth in SEQ ID NO: 184.

Examples of F(ab′) amino acid sequences of the anti-TfR antibodies described herein are provided in Table 6.

TABLE 6
Heavy chain and light chain sequences of examples of anti-TfR F(ab′)

| Antibody | F(ab′) Heavy Chain/Light Chain Sequences |
|---|---|
| 3-A4 | Heavy Chain (with partial human IgG1 constant region) |
| | EVQLQQSGAELVRPGASVKLSCTASGFNIKDDYMYWVKQRPEQGLEWIGWIDPENGDTEYAS |
| | KFQDKATVTADTSSNTAYLQLSSLTSEDTAVYYCTLWLRRGLDYWGQGTSVTVSSASTKGPS |
| | VFPLAPSSKSTSGGTAALGCLVKDYFPEPVTVSWNSGALTSGVHTFPAVLQSSGLYSLSSVVTV |
| | PSSSLGTQTYICNVNHKPSNTKVDKKVEPKSCDKTHT (SEQ ID NO: 185) |
| | Light Chain (with kappa light chain constant region) |
| | DIVMTQAAPSVPVTPGESVSISCRSSKSLLHSNGYTYLFWFLQRPGQSPQLLIYRMSNLASGVP |
| | DRFSGSGSGTAFTLRISRVEAEDVGVYYCMQHLEYPFTFGGGTKLEIKRTVAAPSVFIFPPSDEQ |
| | LKSGTASVVCLLNNFYPREAKVQWKVDNALQSGNSQESVTEQDSKDSTYSLSSTLTLSKADYE |
| | KHKVYACEVTHQGLSSPVTKSFNRGEC (SEQ ID NO: 179) |
| 3-A4 | Heavy Chain (with partial human IgG1 constant region) |
| Variant 1 | EVQLQQSGAELVRPGASVKLSCTASGFNIKDDYMYWVKQRPEQGLEWIGWIDPETGDTEYAS |
| | KFQDKATVTADTSSNTAYLQLSSLTSEDTAVYYCTLWLRRGLDYWGQGTSVTVSSASTKGPS |
| | VFPLAPSSKSTSGGTAALGCLVKDYFPEPVTVSWNSGALTSGVHTFPAVLQSSGLYSLSSVVTV |
| | PSSSLGTQTYICNVNHKPSNTKVDKKVEPKSCDKTHT (SEQ ID NO: 555) |
| | Light Chain (with kappa light chain constant region) |
| | DIVMTQAAPSVPVTPGESVSISCRSSKSLLHSNGYTYLFWFLQRPGQSPQLLIYRMSNLASGVP |
| | DRFSGSGSGTAFTLRISRVEAEDVGVYYCMQHLEYPFTFGGGTKLEIKRTVAAPSVFIFPPSDEQ |
| | LKSGTASVVCLLNNFYPREAKVQWKVDNALQSGNSQESVTEQDSKDSTYSLSSTLTLSKADYE |
| | KHKVYACEVTHQGLSSPVTKSFNRGEC (SEQ ID NO: 179) |
| 3-A4 | Heavy Chain (with partial human IgG1 constant region) |
| Variant 2 | EVQLQQSGAELVRPGASVKLSCTASGFNIKDDYMYWVKQRPEQGLEWIGWIDPESGDTEYAS |
| | KFQDKATVTADTSSNTAYLQLSSLTSEDTAVYYCTLWLRRGLDYWGQGTSVTVSSASTKGPS |
| | VFPLAPSSKSTSGGTAALGCLVKDYFPEPVTVSWNSGALTSGVHTFPAVLQSSGLYSLSSVVTV |
| | PSSSLGTQTYICNVNHKPSNTKVDKKVEPKSCDKTHT (SEQ ID NO: 556) |
| | Light Chain (with kappa light chain constant region) |
| | DIVMTQAAPSVPVTPGESVSISCRSSKSLLHSNGYTYLFWFLQRPGQSPQLLIYRMSNLASGVP |
| | DRFSGSGSGTAFTLRISRVEAEDVGVYYCMQHLEYPFTFGGGTKLEIKRTVAAPSVFIFPPSDEQ |
| | LKSGTASVVCLLNNFYPREAKVQWKVDNALQSGNSQESVTEQDSKDSTYSLSSTLTLSKADYE |
| | KHKVYACEVTHQGLSSPVTKSFNRGEC (SEQ ID NO: 179) |
| 3-M12 | Heavy Chain (with partial human IgG1 constant region) |
| | DVQLQESGPGLVKPSQSLSLTCSVTGYSITSGYYWNWIRQFPGNKLEWMGYITFDGANNYNPS |
| | LKNRISITRDTSKNQFFLKLTSVTTEDTATYYCTRSSYDYDVLDYWGQGTTLTVSSASTKGPSV |
| | FPLAPSSKSTSGGTAALGCLVKDYFPEPVTVSWNSGALTSGVHTFPAVLQSSGLYSLSSVVTVP |
| | SSSLGTQTYICNVNHKPSNTKVDKKVEPKSCDKTHT (SEQ ID NO: 186) |
| | Light Chain (with kappa light chain constant region) |
| | DIQMTQTTSSLSASLGDRVTISCRASQDISNFLNWYQQRPDGTVKLLIYYTSRLHSGVPSRFSGS |
| | GSGTDFSLTVSNLEQEDIATYFCQQGHTLPYTFGGGTKLEIKRTVAAPSVFIFPPSDEQLKSGTA |
| | SVVCLLNNFYPREAKVQWKVDNALQSGNSQESVTEQDSKDSTYSLSSTLTLSKADYEKHKVY |
| | ACEVTHQGLSSPVTKSFNRGEC (SEQ ID NO: 181) |
| 5-H12 | Heavy Chain (with partial human IgG1 constant region) |
| | QIQLQQSGPELVRPGASVKISCKASGYSFTDYCINWVNQRPGQGLEWIGWIYPGSGNTRYSERF |
| | KGKATLTVDTSSNTAYMQLSSLTSEDSAVYFCAREDYYPYHGMDYWGQGTSVTVSSASTKGP |
| | SVFPLAPSSKSTSGGTAALGCLVKDYFPEPVTVSWNSGALTSGVHTFPAVLQSSGLYSLSSVVT |
| | VPSSSLGTQTYICNVNHKPSNTKVDKKVEPKSCDKTHT (SEQ ID NO: 187) |
| | Light Chain (with kappa light chain constant region) |
| | DIVLTQSPTSLAVSLGQRATISCRASESVDGYDNSFMHWYQQKPGQPPKLLIFRASNLESGIPAR |
| | FSGSGSRTDFTLTINPVEAADVATYYCQQSSEDPWTFGGGTKLEIKRTVAAPSVFIFPPSDEQLK |
| | SGTASVVCLLNNFYPREAKVQWKVDNALQSGNSQESVTEQDSKDSTYSLSSTLTLSKADYEK |
| | HKVYACEVTHQGLSSPVTKSFNRGEC (SEQ ID NO: 183) |
| 5-H12 | Heavy Chain (with partial human IgG1 constant region) |
| Variant 1 | QIQLQQSGPELVRPGASVKISCKASGYSFTDYYINWVNQRPGQGLEWIGWIYPGSGNTRYSERF |
| | KGKATLTVDTSSNTAYMQLSSLTSEDSAVYFCAREDYYPYHGMDYWGQGTSVTVSSASTKGP |
| | SVFPLAPSSKSTSGGTAALGCLVKDYFPEPVTVSWNSGALTSGVHTFPAVLQSSGLYSLSSVVT |
| | VPSSSLGTQTYICNVNHKPSNTKVDKKVEPKSCDKTHT (SEQ ID NO: 557) |
| | Light Chain (with kappa light chain constant region) |
| | DIVLTQSPTSLAVSLGQRATISCRASESVDGYDNSFMHWYQQKPGQPPKLLIFRASNLESGIPAR |
| | FSGSGSRTDFTLTINPVEAADVATYYCQQSSEDPWTFGGGTKLEIKRTVAAPSVFIFPPSDEQLK |
| | SGTASVVCLLNNFYPREAKVQWKVDNALQSGNSQESVTEQDSKDSTYSLSSTLTLSKADYEK |
| | HKVYACEVTHQGLSSPVTKSFNRGEC (SEQ ID NO: 183) |
| 5-H12 | Heavy Chain (with partial human IgG1 constant region) |
| Variant 2 | QIQLQQSGPELVRPGASVKISCKASGYSFTDYDINWVNQRPGQGLEWIGWIYPGSGNTRYSERF |
| | KGKATLTVDTSSNTAYMQLSSLTSEDSAVYFCAREDYYPYHGMDYWGQGTSVTVSSASTKGP |
| | SVFPLAPSSKSTSGGTAALGCLVKDYFPEPVTVSWNSGALTSGVHTFPAVLQSSGLYSLSSVVT |
| | VPSSSLGTQTYICNVNHKPSNTKVDKKVEPKSCDKTHT (SEQ ID NO: 558) |
| | Light Chain (with kappa light chain constant region) |
| | DIVLTQSPTSLAVSLGQRATISCRASESVDGYDNSFMHWYQQKPGQPPKLLIFRASNLESGIPAR |
| | FSGSGSRTDFTLTINPVEAADVATYYCQQSSEDPWTFGGGTKLEIKRTVAAPSVFIFPPSDEQLK |
| | SGTASVVCLLNNFYPREAKVQWKVDNALQSGNSQESVTEQDSKDSTYSLSSTLTLSKADYEK |
| | HKVYACEVTHQGLSSPVTKSFNRGEC (SEQ ID NO: 183) |

*VH/VL sequences underlined

In some embodiments, the anti-TfR antibody of the present disclosure comprises a heavy chain containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the heavy chain as set forth in SEQ ID NO: 185, SEQ ID NO: 186, SEQ ID NO: 187, SEQ ID NO: 304, SEQ ID NO: 305. SEQ ID NO: 306, or SEQ ID NO: 307. Alternatively or in addition (e.g., in addition), the anti-TfR antibody of the present disclosure comprises a light chain containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the light chain as set forth in SEQ ID NO: 179, SEQ ID NO: 181, or SEQ ID NO: 183. In some embodiments, the anti-TfR antibody described herein comprises a heavy chain comprising an amino acid sequence that is at least 75% (e.g., 75%, 80%, 85%, 90%, 95%, 98%, or 99%) identical to SEQ ID NO: 185, SEQ ID NO: 186, SEQ ID NO: 187, SEQ ID NO: 304, SEQ ID NO: 305. SEQ ID NO: 306, or SEQ ID NO: 307. Alternatively or in addition (e.g., in addition), the anti-TfR antibody described herein comprises a light chain comprising an amino acid sequence that is at least 75% (e.g., 75%, 80%, 85%, 90%, 95%, 98%, or 99%) identical to SEQ ID NO: 179, SEQ ID NO: 181, or SEQ ID NO: 183. In some embodiments, the anti-TfR antibody described herein comprises a heavy chain comprising the amino acid sequence of SEQ ID NO: 185, SEQ ID NO: 186, SEQ ID NO: 187, SEQ ID NO: 304, SEQ ID NO: 305. SEQ ID NO: 306, or SEQ ID NO: 307. Alternatively or in addition (e.g., in addition), the anti-TfR antibody described herein comprises a light chain comprising the amino acid sequence of SEQ ID NO: 179, SEQ ID NO: 181, or SEQ ID NO: 183.

In some embodiments, the anti-TfR antibody of the present disclosure comprises a heavy chain containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the heavy chain as set forth in SEQ ID NO: 185, SEQ ID NO: 304, or SEQ ID NO: 305. Alternatively or in addition (e.g., in addition), the anti-TfR antibody of the present disclosure comprises a light chain containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the light chain as set forth in SEQ ID NO: 179. In some embodiments, the anti-TfR antibody described herein comprises a heavy chain comprising an amino acid sequence that is at least 75% (e.g., 75%, 80%, 85%, 90%, 95%, 98%, or 99%) identical to SEQ ID NO: 185, SEQ ID NO: 304, or SEQ ID NO: 305. Alternatively or in addition (e.g., in addition), the anti-TfR antibody described herein comprises a light chain comprising an amino acid sequence that is at least 75% (e.g., 75%, 80%, 85%, 90%, 95%, 98%, or 99%) identical to SEQ ID NO: 179. In some embodiments, the anti-TfR antibody described herein comprises a heavy chain comprising the amino acid sequence of SEQ ID NO: 185, SEQ ID NO: 304, or SEQ ID NO: 305. Alternatively or in addition (e.g., in addition), the anti-TfR antibody described herein comprises a light chain comprising the amino acid sequence of SEQ ID NO: 179.

In some embodiments, the anti-TfR antibody of the present disclosure comprises a heavy chain containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the heavy chain as set forth in SEQ ID NO: 186. Alternatively or in addition (e.g., in addition), the anti-TfR antibody of the present disclosure comprises a light chain containing no more than 25 amino acid variations (e.g., no more than 25, 24, 23, 22, 21, 20, 19, 18, 17, 16, 15, 14, 13, 12, 10, 11, 9, 8, 7, 6, 5, 4, 3, 2, or 1 amino acid variation) as compared with the light chain as set forth in SEQ ID NO: 181. In some embodiments, the anti-TfR antibody described herein comprises a heavy chain comprising an amino acid sequence that is at least 75% (e.g., 75%, 80%, 85%, 90%, 95%, 98%, or 99%) identical to SEQ ID NO: 186. Alternatively or in addition (e.g., in addition), the anti-TfR antibody described herein comprises a light chain comprising an amino acid sequence that is at least 75% (e.g., 75%, 80%, 85%, 90%, 95%, 98%, or 99%) identical to SEQ ID NO: 181. In some embodiments, the anti-TfR antibody described herein comprises a heavy chain comprising the amino acid sequence of SEQ ID NO: 186. Alternatively or in addition (e.g., in addition), the anti-TfR antibody described herein comprises a light chain comprising the amino acid sequence of SEQ ID NO: 181.

In some embodiments, the anti-TfR antibody of the present disclosure comprises a heavy chain containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the heavy chain as set forth in SEQ ID NO: 187, SEQ ID NO: 306, or SEQ ID NO: 307. Alternatively or in addition (e.g., in addition), the anti-TfR antibody of the present disclosure comprises a light chain containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the light chain as set forth in SEQ ID NO: 183. In some embodiments, the anti-TfR antibody described herein comprises a heavy chain comprising an amino acid sequence that is at least 75% (e.g., 75%, 80%, 85%, 90%, 95%, 98%, or 99%) identical to SEQ ID NO: 187, SEQ ID NO: 306, or SEQ ID NO: 307. Alternatively or in addition (e.g., in addition), the anti-TfR antibody described herein comprises a light chain comprising an amino acid sequence that is at least 75% (e.g., 75%, 80%, 85%, 90%, 95%, 98%, or 99%) identical to SEQ ID NO: 183. In some embodiments, the anti-TfR antibody described herein comprises a heavy chain comprising the amino acid sequence of SEQ ID NO: 187, SEQ ID NO: 306, or SEQ ID NO: 307. Alternatively or in addition (e.g., in addition), the anti-TfR antibody described herein comprises a light chain comprising the amino acid sequence of SEQ ID NO: 183.

The anti-TfR receptor antibodies described herein can be in any antibody form, including, but not limited to, intact (i.e., full-length) antibodies, antigen-binding fragments thereof (such as Fab, F(ab′), F(ab′)2, Fv), single chain antibodies, bi-specific antibodies, or nanobodies. In some embodiments, the anti-TfR antibody described herein is a scFv. In some embodiments, the anti-TfR antibody described herein is a scFv-Fab (e.g., scFv fused to a portion of a constant region). In some embodiments, the anti-TfR receptor antibody described herein is a scFv fused to a constant region (e.g., human IgG1 constant region as set forth in SEQ ID NO: 175 or SEQ ID NO: 176, or a portion thereof such as the Fc portion) at either the N-terminus of C-terminus.

In some embodiments, any one of the anti-TfR1 antibodies described herein may comprise a signal peptide in the heavy and/or (e.g., and) light chain sequence (e.g., a N-terminal signal peptide). In some embodiments, the anti-TfR1 antibody described herein comprises any one of the VH and VL sequences, any one of the IgG heavy chain and light chain sequences, or any one of the F(ab′) heavy chain and light chain sequences described herein, and further comprises a signal peptide (e.g., a N-terminal signal peptide). In some embodiments, the signal peptide comprises the amino acid sequence of MGWSCIILFLVATATGVHS (SEQ ID NO: 214).

The present disclosure, in some aspects, provide another new anti-TfR antibody that can be used as a muscle-targeting agent (e.g., in a muscle-targeting complex). The CDR sequences and variable domain sequences of the antibody are provided in Table 7.

TABLE 7
CDR sequences of an anti-TfR antibody according to different definition systems and
variable domain sequences

| No. | | | |
|---|---|---|---|
| system | IMGT | Kabat | Chothia |
| CDR-H1 | GYSFTSYW (SEQ ID NO: 188) | SYWIG (SEQ ID NO: 194) | GYSFTSY (SEQ ID NO: 199) |
| CDR-H2 | IYPGDSDT (SEQ ID NO: 189) | IIYPGDSDTRYSPSFQGQ | GDS (SEQ ID NO: 200) |
| | | (SEQ ID NO: 195) | |
| CDR-H3 | ARFPYDSSGYYSFDY (SEQ ID | FPYDSSGYYSFDY (SEQ | PYDSSGYYSFD (SEQ ID |
| | NQ: 190) | ID NQ: 196) | NQ: 201) |
| CDR-L1 | QSISSY (SEQ ID NO: 191) | RASQSISSYLN (SEQ ID | SQSISSY (SEQ ID NO: 202) |
| | | NQ: 197) | |
| CDR-L2 | AAS (SEQ ID NO: 192) | AASSLQS (SEQ ID NO: | AAS (SEQ ID NO: 192) |
| | | 198) | |
| CDR-L3 | QQSYSTPLT (SEQ ID NO: 193) | QQSYSTPLT (SEQ ID NO: | SYSTPL (SEQ ID NO: 203) |
| | | 193) | |

| | |
|---|---|
| VH | QVQLVQSGAEVKKPGESLKISCKGSGYSFTSYWIGWVRQMPGKGLEWMGIIYPGDSDTRY |
| | SPSFQGQVTISADKSISTAYLQWSSLKASDTAMYYCARFPYDSSGYYSFDYWGQGTLVTVS |
| | S (SEQ ID NO: 204) |
| VL | DIQMTQSPSSLSASVGDRVTITCRASQSISSYLNWYQQKPGKAPKLLIYAASSLQSGVPSRFS |
| | GSGSGTDFTLTISSLQPEDFATYYCQQSYSTPLTFGGGTKVEIK (SEQ ID NO: 205) |

In some embodiments, the anti-TfR antibodies of the present disclosure comprises one or more of the CDR-H (e.g., CDR-H1, CDR-H2, and CDR-H3) amino acid sequences from any one of the anti-TfR antibodies selected from Table 7. In some embodiments, the anti-TfR antibodies of the present disclosure comprise the CDR-H1, CDR-H2, and CDR-H3 as provided for each numbering system provided in Table 7. In some embodiments, the anti-TfR antibodies of the present disclosure comprises one or more of the CDR-L (e.g., CDR-L1, CDR-L2, and CDR-L3) amino acid sequences from any one of the anti-TfR antibodies selected from Table 7. In some embodiments, the anti-TfR antibodies of the present disclosure comprise the CDR-L1, CDR-L2, and CDR-L3 as provided for teach numbering system provided in Table 7.

In some embodiments, the anti-TfR antibodies of the present disclosure comprises the CDR-H1, CDR-H2, CDR-H3, CDR-L1, CDR-L2, and CDR-L3 as provided for each numbering system provided in Table 7. In some embodiments, antibody heavy and light chain CDR3 domains may play a particularly important role in the binding specificity/affinity of an antibody for an antigen. Accordingly, the anti-TfR antibodies of the disclosure may include at least the heavy and/or (e.g., and) light chain CDR3s of any one of the anti-TfR antibody provided in Table 7.

In some examples, any of the anti-TfR antibodies of the disclosure have one or more CDR (e.g., CDR-H or CDR-L) sequences substantially similar to any of the CDR-H1, CDR-H2, CDR-H3, CDR-L1, CDR-L2, and/or (e.g., and) CDR-L3 sequences provided in Table 7. In some embodiments, the position of one or more CDRs along the VH (e.g., CDR-H1, CDR-H2, or CDR-H3) and/or (e.g., and) VL (e.g., CDR-L1, CDR-L2, or CDR-L3) region of an antibody described herein can vary by one, two, three, four, five, or six amino acid positions so long as immunospecific binding to transferrin receptor (e.g., human transferrin receptor) is maintained (e.g., substantially maintained, for example, at least 50%, at least 60%, at least 70%, at least 80%, at least 90%, at least 95% of the binding of the original antibody from which it is derived). For example, in some embodiments, the position defining a CDR of any antibody described herein can vary by shifting the N-terminal and/or (e.g., and) C-terminal boundary of the CDR by one, two, three, four, five, or six amino acids, relative to the CDR position of any one of the antibodies described herein, so long as immunospecific binding to transferrin receptor (e.g., human transferrin receptor) is maintained (e.g., substantially maintained, for example, at least 50%, at least 60%, at least 70%, at least 80%, at least 90%, at least 95% of the binding of the original antibody from which it is derived). In another embodiment, the length of one or more CDRs along the VH (e.g., CDR-H1, CDR-H2, or CDR-H3) and/or (e.g., and) VL (e.g., CDR-L1, CDR-L2, or CDR-L3) region of an antibody described herein can vary (e.g., be shorter or longer) by one, two, three, four, five, or more amino acids, so long as immunospecific binding to transferrin receptor (e.g., human transferrin receptor) is maintained (e.g., substantially maintained, for example, at least 50%, at least 60%, at least 70%, at least 80%, at least 90%, at least 95% of the binding of the original antibody from which it is derived).

Accordingly, in some embodiments, a CDR-L1, CDR-L2, CDR-L3, CDR-H1, CDR-H2, and/or (e.g., and) CDR-H3 described herein may be one, two, three, four, five or more amino acids shorter than one or more of the CDRs described herein (e.g., provided in Table 7) so long as immunospecific binding to transferrin receptor (e.g., human transferrin receptor) is maintained (e.g., substantially maintained, for example, at least 50%, at least 60%, at least 70%, at least 80%, at least 90%, at least 95% relative to the binding of the original antibody from which it is derived). In some embodiments, a CDR-L1, CDR-L2, CDR-L3, CDR-H1, CDR-H2, and/or (e.g., and) CDR-H3 described herein may be one, two, three, four, five or more amino acids longer than one or more of the CDRs described herein (e.g., CDRS from the anti-TfR antibody provided in Table 7) so long as immunospecific binding to transferrin receptor (e.g., human transferrin receptor) is maintained (e.g., substantially maintained, for example, at least 50%, at least 60%, at least 70%, at least 80%, at least 90%, at least 95% relative to the binding of the original antibody from which it is derived). In some embodiments, the amino portion of a CDR-L1, CDR-L2, CDR-L3, CDR-H1, CDR-H2, and/or (e.g., and) CDR-H3 described herein can be extended by one, two, three, four, five or more amino acids compared to one or more of the CDRs described herein (e.g., CDRs from the anti-TfR antibody provided in Table 7) so long as immunospecific binding to transferrin receptor (e.g., human transferrin receptor is maintained (e.g., substantially maintained, for example, at least 50%, at least 60%, at least 70%, at least 80%, at least 90%, at least 95% relative to the binding of the original antibody from which it is derived). In some embodiments, the carboxy portion of a CDR-L1, CDR-L2, CDR-L3, CDR-H1, CDR-H2, and/or (e.g., and) CDR-H3 described herein can be extended by one, two, three, four, five or more amino acids compared to one or more of the CDRs described herein (e.g., CDRS from the anti-TfR antibody provided in Table 7) so long as immunospecific binding to transferrin receptor (e.g., human transferrin receptor) is maintained (e.g., substantially maintained, for example, at least 50%, at least 60%, at least 70%, at least 80%, at least 90%, at least 95% relative to the binding of the original antibody from which it is derived). In some embodiments, the amino portion of a CDR-L1, CDR-L2, CDR-L3, CDR-H1, CDR-H2, and/or (e.g., and) CDR-H3 described herein can be shortened by one, two, three, four, five or more amino acids compared to one or more of the CDRs described herein (e.g., CDRS from the anti-TfR antibody provided in Table 7) so long as immunospecific binding to transferrin receptor (e.g., human transferrin receptor) is maintained (e.g., substantially maintained, for example, at least 50%, at least 60%, at least 70%, at least 80%, at least 90%, at least 95% relative to the binding of the original antibody from which it is derived). In some embodiments, the carboxy portion of a CDR-L1, CDR-L2, CDR-L3, CDR-H1, CDR-H2, and/or (e.g., and) CDR-H3 described herein can be shortened by one, two, three, four, five or more amino acids compared to one or more of the CDRs described herein (e.g., CDRS from the anti-TfR antibody provided in Table 7) so long as immunospecific binding to transferrin receptor (e.g., human transferrin receptor) is maintained (e.g., substantially maintained, for example, at least 50%, at least 60%, at least 70%, at least 80%, at least 90%, at least 95% relative to the binding of the original antibody from which it is derived). Any method can be used to ascertain whether immunospecific binding to transferrin receptor (e.g., human transferrin receptor) is maintained, for example, using binding assays and conditions described in the art.

In some examples, any of the anti-TfR antibodies of the disclosure have one or more CDR (e.g., CDR-H or CDR-L) sequences substantially similar to any one of the anti-TfR antibody provided in Table 7. For example, the antibodies may include one or more CDR sequence(s) from the anti-TfR antibody provided in Table 7 and containing up to 5, 4, 3, 2, or 1 amino acid residue variations as compared to the corresponding CDR region in any one of the CDRs provided herein (e.g., CDRs from the anti-TfR antibody provided in Table 7) so long as immunospecific binding to transferrin receptor (e.g., human transferrin receptor) is maintained (e.g., substantially maintained, for example, at least 50%, at least 60%, at least 70%, at least 80%, at least 90%, at least 95% relative to the binding of the original antibody from which it is derived). In some embodiments, any of the amino acid variations in any of the CDRs provided herein may be conservative variations. Conservative variations can be introduced into the CDRs at positions where the residues are not likely to be involved in interacting with a transferrin receptor protein (e.g., a human transferrin receptor protein), for example, as determined based on a crystal structure.

Some aspects of the disclosure provide anti-TfR antibodies that comprise one or more of the heavy chain variable (VH) and/or (e.g., and) light chain variable (VL) domains provided herein. In some embodiments, the anti-TfR antibodies of the disclosure include any antibody that includes a heavy chain variable domain and/or (e.g., and) a light chain variable domain of the anti-TfR1 antibody provided in Table 7.

Aspects of the disclosure provide anti-TfR antibodies having a heavy chain variable (VH) and/or (e.g., and) a light chain variable (VL) domain amino acid sequence homologous to any of those described herein. In some embodiments, the anti-TfR antibody comprises a heavy chain variable sequence or a light chain variable sequence that is at least 75% (e.g., 80%, 85%, 90%, 95%, 98%, or 99%) identical to the heavy chain variable sequence and/or any light chain variable sequence provided in Table 7. In some embodiments, the homologous heavy chain variable and/or (e.g., and) a light chain variable amino acid sequences do not vary within any of the CDR sequences provided herein. For example, in some embodiments, the degree of sequence variation (e.g., 75%, 80%, 85%, 90%, 95%, 98%, or 99%) may occur within a heavy chain variable and/or (e.g., and) a light chain variable sequence excluding any of the CDR sequences provided herein. In some embodiments, any of the anti-TfR antibodies provided herein comprise a heavy chain variable sequence and a light chain variable sequence that comprises a framework sequence that is at least 75%, 80%, 85%, 90%, 95%, 98%, or 99% identical to the framework sequence of any anti-TfR antibody provided in Table 7.

In some embodiments, the anti-TfR antibody of the present disclosure comprises a CDR-H1, a CDR-H2, and a CDR-H3 of a heavy chain variable domain having the amino acid sequence of SEQ ID NO: 204. Alternatively or in addition (e.g., in addition), the anti-TfR antibody of the present disclosure comprises a CDR-L1, a CDR-L2, and a CDR-L3 of a light chain variable domain having the amino acid sequence of SEQ ID NO: 205.

In some embodiments, the anti-TfR antibody of the present disclosure comprises a CDR-H1 having the amino acid sequence of SEQ ID NO: 188 (according to the IMGT definition system), a CDR-H2 having the amino acid sequence of SEQ ID NO: 189 (according to the IMGT definition system), a CDR-H3 having the amino acid sequence of SEQ ID NO: 190 (according to the IMGT definition system), a CDR-L1 having the amino acid sequence of SEQ ID NO: 191 (according to the IMGT definition system), a CDR-L2 having the amino acid sequence of SEQ ID NO: 192 (according to the IMGT definition system), and a CDR-L3 having the amino acid sequence of SEQ ID NO: 193 (according to the IMGT definition system).

In some embodiments, anti-TfR antibody of the present disclosure comprises a CDR-H1, a CDR-H2, and a CDR-H3, which collectively contains no more than 5 amino acid variations (e.g., no more than 5, 4, 3, 2, or 1 amino acid variation) as compared with the CDR-H1 having the amino acid sequence of SEQ ID NO: 188, CDR-H2 having the amino acid sequence of SEQ ID NO: 189, and CDR-H3 having the amino acid sequence of SEQ ID NO: 190. Alternatively or in addition (e.g., in addition), the anti-TfR antibody of the present disclosure comprises a CDR-L1, a CDR-L2, and a CDR-L3, which collectively contains no more than 5 amino acid variations (e.g., no more than 5, 4, 3, 2 or 1 amino acid variation) as compared with the CDR-L1 having the amino acid sequence of SEQ ID NO: 191, CDR-L2 having the amino acid sequence of SEQ ID NO: 192, and CDR-L3 having the amino acid sequence of SEQ ID NO: 193.

In some embodiments, the anti-TfR antibody of the present disclosure comprises a CDR-H1, a CDR-H2, and a CDR-H3 that collectively are at least 75% (e.g., 75%, 80%, 85%, 90%, 95%, 98%, or 99%) identical to the CDR-H1 having the amino acid sequence of SEQ ID NO: 188, CDR-H2 having the amino acid sequence of SEQ ID NO: 189, and CDR-H3 having the amino acid sequence of SEQ ID NO: 190. Alternatively or in addition (e.g., in addition), the anti-TfR antibody of the present disclosure comprises a CDR-L1, a CDR-L2, and a CDR-L3 that collectively are at least 75% (e.g., 75%, 80%, 85%, 90%, 95%, 98%, or 99%) identical to the to the CDR-L1 having the amino acid sequence of SEQ ID NO: 191, CDR-L2 having the amino acid sequence of SEQ ID NO: 192, and CDR-L3 having the amino acid sequence of SEQ ID NO: 193.

In some embodiments, the anti-TfR antibody of the present disclosure comprises: a CDR-H1 having no more than 3 amino acid variations (e.g., no more than 3, 2, or 1 amino acid variation) as compared with the CDR-H1 having the amino acid sequence of SEQ ID NO: 188; a CDR-H2 having no more than 3 amino acid variations (e.g., no more than 3, 2, or 1 amino acid variation) as compared with the CDR-H2 having the amino acid sequence of SEQ ID NO: 189; and/or (e.g., and) a CDR-H3 having no more than 3 amino acid variations (e.g., no more than 3, 2, or 1 amino acid variation) as compared with the CDR-H3 having the amino acid sequence of SEQ ID NO: 190. Alternatively or in addition (e.g., in addition), the anti-TfR antibody of the present disclosure comprises: a CDR-L1 having no more than 3 amino acid variations (e.g., no more than 3, 2, or 1 amino acid variation) as compared with the CDR-L1 having the amino acid sequence of SEQ ID NO: 191; a CDR-L2 having no more than 3 amino acid variations (e.g., no more than 3, 2, or 1 amino acid variation) as compared with the CDR-L2 having the amino acid sequence of SEQ ID NO: 192; and/or (e.g., and) a CDR-L3 having no more than 3 amino acid variations (e.g., no more than 3, 2, or 1 amino acid variation) as compared with the CDR-L3 having the amino acid sequence of SEQ ID NO: 193.

In some embodiments, the anti-TfR antibody of the present disclosure comprises a CDR-H1 having the amino acid sequence of SEQ ID NO: 194 (according to the Kabat definition system), a CDR-H2 having the amino acid sequence of SEQ ID NO: 195 (according to the Kabat definition system), a CDR-H3 having the amino acid sequence of SEQ ID NO: 196 (according to the Kabat definition system), a CDR-L1 having the amino acid sequence of SEQ ID NO: 197 (according to the Kabat definition system), a CDR-L2 having the amino acid sequence of SEQ ID NO: 198 (according to the Kabat definition system), and a CDR-L3 having the amino acid sequence of SEQ ID NO: 193 (according to the Kabat definition system).

In some embodiments, anti-TfR antibody of the present disclosure comprises a CDR-H1, a CDR-H2, and a CDR-H3, which collectively contains no more than 5 amino acid variations (e.g., no more than 5, 4, 3, 2, or 1 amino acid variation) as compared with the CDR-H1 having the amino acid sequence of SEQ ID NO: 194 , CDR-H2 having the amino acid sequence of SEQ ID NO: 195, and CDR-H3 having the amino acid sequence of SEQ ID NO: 196. “Collectively” means that the total number of amino acid variations in all of the three heavy chain CDRs is within the defined range. Alternatively or in addition (e.g., in addition), the anti-TfR antibody of the present disclosure comprises a CDR-L1, a CDR-L2, and a CDR-L3, which collectively contains no more than 5 amino acid variations (e.g., no more than 5, 4, 3, 2 or 1 amino acid variation) as compared with the CDR-L1 having the amino acid sequence of SEQ ID NO: 197, CDR-L2 having the amino acid sequence of SEQ ID NO: 198, and CDR-L3 having the amino acid sequence of SEQ ID NO: 193.

In some embodiments, the anti-TfR antibody of the present disclosure comprises a CDR-H1, a CDR-H2, and a CDR-H3 that collectively are at least 75% (e.g., 75%, 80%, 85%, 90%, 95%, 98%, or 99%) identical to the CDR-H1 having the amino acid sequence of SEQ ID NO: 194 , CDR-H2 having the amino acid sequence of SEQ ID NO: 195, and CDR-H3 having the amino acid sequence of SEQ ID NO: 196. Alternatively or in addition (e.g., in addition), the anti-TfR antibody of the present disclosure comprises a CDR-L1, a CDR-L2, and a CDR-L3 that collectively are at least 75% (e.g., 75%, 80%, 85%, 90%, 95%, 98%, or 99%) identical to the to the CDR-L1 having the amino acid sequence of SEQ ID NO: 197, CDR-L2 having the amino acid sequence of SEQ ID NO: 198, and CDR-L3 having the amino acid sequence of SEQ ID NO: 193.

In some embodiments, the anti-TfR antibody of the present disclosure comprises: a CDR-H1 having no more than 3 amino acid variations (e.g., no more than 3, 2, or 1 amino acid variation) as compared with the CDR-H1 having the amino acid sequence of SEQ ID NO: 194 ; a CDR-H2 having no more than 3 amino acid variations (e.g., no more than 3, 2, or 1 amino acid variation) as compared with the CDR-H2 having the amino acid sequence of SEQ ID NO: 195; and/or (e.g., and) a CDR-H3 having no more than 3 amino acid variations (e.g., no more than 3, 2, or 1 amino acid variation) as compared with the CDR-H3 having the amino acid sequence of SEQ ID NO: 196. Alternatively or in addition (e.g., in addition), the anti-TfR antibody of the present disclosure comprises: a CDR-L1 having no more than 3 amino acid variations (e.g., no more than 3, 2, or 1 amino acid variation) as compared with the CDR-L1 having the amino acid sequence of SEQ ID NO: 197; a CDR-L2 having no more than 3 amino acid variations (e.g., no more than 3, 2, or 1 amino acid variation) as compared with the CDR-L2 having the amino acid sequence of SEQ ID NO: 198; and/or (e.g., and) a CDR-L3 having no more than 3 amino acid variations (e.g., no more than 3, 2, or 1 amino acid variation) as compared with the CDR-L3 having the amino acid sequence of SEQ ID NO: 193.

In some embodiments, the anti-TfR antibody of the present disclosure comprises a CDR-H1 having the amino acid sequence of SEQ ID NO: 199 (according to the Chothia definition system), a CDR-H2 having the amino acid sequence of SEQ ID NO: 200 (according to the Chothia definition system), a CDR-H3 having the amino acid sequence of SEQ ID NO: 201 (according to the Chothia definition system), a CDR-L1 having the amino acid sequence of SEQ ID NO: 202 (according to the Chothia definition system), a CDR-L2 having the amino acid sequence of SEQ ID NO: 192 (according to the Chothia definition system), and a CDR-L3 having the amino acid sequence of SEQ ID NO: 203 (according to the Chothia definition system).

In some embodiments, anti-TfR antibody of the present disclosure comprises a CDR-H1, a CDR-H2, and a CDR-H3, which collectively contains no more than 5 amino acid variations (e.g., no more than 5, 4, 3, 2, or 1 amino acid variation) as compared with the CDR-H1 having the amino acid sequence of SEQ ID NO: 199, CDR-H2 having the amino acid sequence of SEQ ID NO: 200, and CDR-H3 having the amino acid sequence of SEQ ID NO: 201. “Collectively” means that the total number of amino acid variations in all of the three heavy chain CDRs is within the defined range. Alternatively or in addition (e.g., in addition), the anti-TfR antibody of the present disclosure comprises a CDR-L1, a CDR-L2, and a CDR-L3, which collectively contains no more than 5 amino acid variations (e.g., no more than 5, 4, 3, 2 or 1 amino acid variation) as compared with the CDR-L1 having the amino acid sequence of SEQ ID NO: 202, CDR-L2 having the amino acid sequence of SEQ ID NO: 192, and CDR-L3 having the amino acid sequence of SEQ ID NO: 203.

In some embodiments, the anti-TfR antibody of the present disclosure comprises a CDR-H1, a CDR-H2, and a CDR-H3 that collectively are at least 75% (e.g., 75%, 80%, 85%, 90%, 95%, 98%, or 99%) identical to the CDR-H1 having the amino acid sequence of SEQ ID NO: 199, CDR-H2 having the amino acid sequence of SEQ ID NO: 200, and CDR-H3 having the amino acid sequence of SEQ ID NO: 201. Alternatively or in addition (e.g., in addition), the anti-TfR antibody of the present disclosure comprises a CDR-L1, a CDR-L2, and a CDR-L3 that collectively are at least 75% (e.g., 75%, 80%, 85%, 90%, 95%, 98%, or 99%) identical to the to the CDR-L1 having the amino acid sequence of SEQ ID NO: 202, CDR-L2 having the amino acid sequence of SEQ ID NO: 192, and CDR-L3 having the amino acid sequence of SEQ ID NO: 203.

In some embodiments, the anti-TfR antibody of the present disclosure comprises: a CDR-H1 having no more than 3 amino acid variations (e.g., no more than 3, 2, or 1 amino acid variation) as compared with the CDR-H1 having the amino acid sequence of SEQ ID NO: 199; a CDR-H2 having no more than 3 amino acid variations (e.g., no more than 3, 2, or 1 amino acid variation) as compared with the CDR-H2 having the amino acid sequence of SEQ ID NO: 200; and/or (e.g., and) a CDR-H3 having no more than 3 amino acid variations (e.g., no more than 3, 2, or 1 amino acid variation) as compared with the CDR-H3 having the amino acid sequence of SEQ ID NO: 201. Alternatively or in addition (e.g., in addition), the anti-TfR antibody of the present disclosure comprises: a CDR-L1 having no more than 3 amino acid variations (e.g., no more than 3, 2, or 1 amino acid variation) as compared with the CDR-L1 having the amino acid sequence of SEQ ID NO: 202; a CDR-L2 having no more than 3 amino acid variations (e.g., no more than 3, 2, or 1 amino acid variation) as compared with the CDR-L2 having the amino acid sequence of SEQ ID NO: 192; and/or (e.g., and) a CDR-L3 having no more than 3 amino acid variations (e.g., no more than 3, 2, or 1 amino acid variation) as compared with the CDR-L3 having the amino acid sequence of SEQ ID NO: 203.

In some embodiments, the In some embodiments, the anti-TfR antibody of the present disclosure comprises a CDR-H1 comprising the amino acid sequence of SEQ ID NO: 194, a CDR-H2 comprising the amino acid sequence of SEQ ID NO: 189, a CDR-H3 comprising the amino acid sequence of SEQ ID NO: 196, a CDR-L1 comprising the amino acid sequence of SEQ ID NO: 197, a CDR-L2 comprising the amino acid sequence of SEQ ID NO: 198, and a CDR-L3 comprising the amino acid sequence of SEQ ID NO: 193.

In some embodiments, the anti-TfR antibody of the present disclosure is a human antibody comprising a VH comprising the amino acid sequence of SEQ ID NO: 204. Alternatively or in addition (e.g., in addition), the anti-TfR antibody of the present disclosure is a human antibody comprising a VL comprising the amino acid sequence of SEQ ID NO: 205. In some embodiments, the present disclosure contemplate other humanized/human antibodies comprising the CDR-H1, CDR-H1, CDR-H3 of the VH comprising SEQ ID NO: 204 and the CDR-L1, CDR-L1, and CDR-L3 of the VL comprising SEQ ID NO: 205 with human framework regions.

In some embodiments, the anti-TfR antibody of the present disclosure comprises a VH containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 204. Alternatively or in addition (e.g., in addition), the anti-TfR antibody of the present disclosure comprises a VL containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VL as set forth in SEQ ID NO: 205.

In some embodiments, the anti-TfR antibody of the present disclosure comprises a VH comprising an amino acid sequence that is at least 75% (e.g., 75%, 80%, 85%, 90%, 95%, 98%, or 99%) identical to the VH as set forth in SEQ ID NO: 204. Alternatively or in addition (e.g., in addition), the anti-TfR antibody of the present disclosure comprises a VL comprising an amino acid sequence that is at least 75% (e.g., 75%, 80%, 85%, 90%, 95%, 98%, or 99%) identical to the VL as set forth in SEQ ID NO: 205.

In some embodiments, the anti-TfR antibody of the present disclosure is a humanized antibody. In some embodiments, the humanized anti-TfR antibody comprises a humanized VH comprising a CDR-H1 having the amino acid sequence of SEQ ID NO: 188 (according to the IMGT definition system), a CDR-H2 having the amino acid sequence of SEQ ID NO: 189 (according to the IMGT definition system), a CDR-H3 having the amino acid sequence of SEQ ID NO: 190 (according to the IMGT definition system); and a humanized VL comprising a CDR-L1 having the amino acid sequence of SEQ ID NO: 191 (according to the IMGT definition system), a CDR-L2 having the amino acid sequence of SEQ ID NO: 192 (according to the IMGT definition system), and a CDR-L3 having the amino acid sequence of SEQ ID NO: 193 (according to the IMGT definition system), wherein the humanized VH comprises an amino acid sequence that is at least 75% (e.g., 75%, 80%, 85%, 90%, 95%, 98%, or 99%) identical to the VH as set forth in SEQ ID NO: 204, and the humanized VL comprises an amino acid sequence that is at least 75% (e.g., 75%, 80%, 85%, 90%, 95%, 98%, or 99%) identical to the VL as set forth in SEQ ID NO: 205.

In some embodiments, the humanized anti-TfR antibody comprises a humanized VH comprising a CDR-H1 having the amino acid sequence of SEQ ID NO: 188 (according to the IMGT definition system), a CDR-H2 having the amino acid sequence of SEQ ID NO: 189 (according to the IMGT definition system), a CDR-H3 having the amino acid sequence of SEQ ID NO: 190 (according to the IMGT definition system); and a humanized VL comprising a CDR-L1 having the amino acid sequence of SEQ ID NO: 191 (according to the IMGT definition system), a CDR-L2 having the amino acid sequence of SEQ ID NO: 192 (according to the IMGT definition system), and a CDR-L3 having the amino acid sequence of SEQ ID NO: 193 (according to the IMGT definition system), wherein the humanized VH contains no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 204, and the humanized VL contains no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VL as set forth in SEQ ID NO: 205.

In some embodiments, the humanized anti-TfR antibody comprises a humanized VH comprising a CDR-H1 having the amino acid sequence of SEQ ID NO: 194 (according to the Kabat definition system), a CDR-H2 having the amino acid sequence of SEQ ID NO: 195 (according to the Kabat definition system), a CDR-H3 having the amino acid sequence of SEQ ID NO: 196 (according to the Kabat definition system), a CDR-L1 having the amino acid sequence of SEQ ID NO: 197 (according to the Kabat definition system), a CDR-L2 having the amino acid sequence of SEQ ID NO: 198 (according to the Kabat definition system), and a CDR-L3 having the amino acid sequence of SEQ ID NO: 193 (according to the Kabat definition system), wherein the humanized VH comprises an amino acid sequence that is at least 75% (e.g., 75%, 80%, 85%, 90%, 95%, 98%, or 99%) identical to the VH as set forth in SEQ ID NO: 204, and the humanized VL comprises an amino acid sequence that is at least 75% (e.g., 75%, 80%, 85%, 90%, 95%, 98%, or 99%) identical to the VL as set forth in SEQ ID NO: 205.

In some embodiments, the humanized anti-TfR antibody comprises a CDR-H1 having the amino acid sequence of SEQ ID NO: 194 (according to the Kabat definition system), a CDR-H2 having the amino acid sequence of SEQ ID NO: 195 (according to the Kabat definition system), a CDR-H3 having the amino acid sequence of SEQ ID NO: 196 (according to the Kabat definition system), a CDR-L1 having the amino acid sequence of SEQ ID NO: 197 (according to the Kabat definition system), a CDR-L2 having the amino acid sequence of SEQ ID NO: 198 (according to the Kabat definition system), and a CDR-L3 having the amino acid sequence of SEQ ID NO: 193 (according to the Kabat definition system), wherein the humanized VH contains no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 204, and the humanized VL contains no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VL as set forth in SEQ ID NO: 205.

In some embodiments, the humanized anti-TfR antibody comprises a humanized VH comprising a CDR-H1 having the amino acid sequence of SEQ ID NO: 199 (according to the Chothia definition system), a CDR-H2 having the amino acid sequence of SEQ ID NO: 200 (according to the Chothia definition system), a CDR-H3 having the amino acid sequence of SEQ ID NO: 201 (according to the Chothia definition system), a CDR-L1 having the amino acid sequence of SEQ ID NO: 202 (according to the Chothia definition system), a CDR-L2 having the amino acid sequence of SEQ ID NO: 192 (according to the Chothia definition system), and a CDR-L3 having the amino acid sequence of SEQ ID NO: 203 (according to the Chothia definition system), wherein the humanized VH comprises an amino acid sequence that is at least 75% (e.g., 75%, 80%, 85%, 90%, 95%, 98%, or 99%) identical to the VH as set forth in SEQ ID NO: 204, and the humanized VL comprises an amino acid sequence that is at least 75% (e.g., 75%, 80%, 85%, 90%, 95%, 98%, or 99%) identical to the VL as set forth in SEQ ID NO: 205.

In some embodiments, the humanized anti-TfR antibody comprises a CDR-H1 having the amino acid sequence of SEQ ID NO: 199 (according to the Chothia definition system), a CDR-H2 having the amino acid sequence of SEQ ID NO: 200 (according to the Chothia definition system), a CDR-H3 having the amino acid sequence of SEQ ID NO: 201 (according to the Chothia definition system), a CDR-L1 having the amino acid sequence of SEQ ID NO: 202 (according to the Chothia definition system), a CDR-L2 having the amino acid sequence of SEQ ID NO: 192 (according to the Chothia definition system), and a CDR-L3 having the amino acid sequence of SEQ ID NO: 203 (according to the Chothia definition system), wherein the humanized VH contains no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 204, and the humanized VL contains no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VL as set forth in SEQ ID NO: 205.

In some embodiments, the anti-TfR antibody is an IgG, a Fab fragment, a F(ab′) fragment, a F(ab′)₂ fragment, a scFv, or an scFv fused to a constant region (e.g., N- or C-terminal fusion). Non-limiting examples of anti-TfR antibodies in different formats are provided herein.

In some embodiments, the anti-TfR1 antibody is a single-chain fragment variable (scFv) comprising the VH and VL in a single polypeptide chain. In some embodiments, the scFv comprises any one of the heavy chain CDRs, light chain CDRs, VHs, and/or (e.g., and) VLs described herein on a single polypeptide chain. In some embodiments, the scFv comprises the VH linked at the N-terminus of the VL. In some embodiments, the scFv comprises the VL linked at the N-terminus of the VH. In some embodiments, the VH and VL are linked via a linker (e.g., a polypeptide linker). Any polypeptide linker can be used for linking the VH and VL in the scFv. Selection of a linker sequence is within the abilities of those skilled in the art.

In some embodiments, the scFv comprises a VH (e.g., a humanized VH) comprising a CDR-H1 having the amino acid sequence of SEQ ID NO: 188 (according to the IMGT definition system), a CDR-H2 having the amino acid sequence of SEQ ID NO: 189 (according to the IMGT definition system), a CDR-H3 having the amino acid sequence of SEQ ID NO: 190 (according to the IMGT definition system); and a VL (e.g., a humanized VL) comprising a CDR-L1 having the amino acid sequence of SEQ ID NO: 191 (according to the IMGT definition system), a CDR-L2 having the amino acid sequence of SEQ ID NO: 192 (according to the IMGT definition system), and a CDR-L3 having the amino acid sequence of SEQ ID NO: 193 (according to the IMGT definition system), wherein the VH and VL are on a single polypeptide chain (e.g., linked via an amide bond or linked via a linker such as a peptide linker), and wherein the VH is linked to the N-terminus or the C-terminus of the VL. In some embodiments, the VH and VL are linked via a linker comprising the amino acid sequence of EGKSSGSGSESKAS (SEQ ID NO: 215).

In some embodiments, the scFv comprises a VH (e.g., a humanized VH) comprising a CDR-H1 having the amino acid sequence of SEQ ID NO: 194 (according to the Kabat definition system), a CDR-H2 having the amino acid sequence of SEQ ID NO: 195 (according to the Kabat definition system), a CDR-H3 having the amino acid sequence of SEQ ID NO: 196 (according to the Kabat definition system); and a VL (e.g., a humanized VL) comprising a CDR-L1 having the amino acid sequence of SEQ ID NO: 197 (according to the Kabat definition system), a CDR-L2 having the amino acid sequence of SEQ ID NO: 198 (according to the Kabat definition system), and a CDR-L3 having the amino acid sequence of SEQ ID NO: 193 (according to the Kabat definition system), wherein the VH and VL are on a single polypeptide chain (e.g., linked via an amide bond or linked via a linker such as a peptide linker), and wherein the VH is linked to the N-terminus or the C-terminus of the VL. In some embodiments, the VH and VL are linked via a linker comprising the amino acid sequence of EGKSSGSGSESKAS (SEQ ID NO: 215).

In some embodiments, the scFv comprises a VH (e.g., a humanized VH) comprising a CDR-H1 having the amino acid sequence of SEQ ID NO: 199 (according to the Chothia definition system), a CDR-H2 having the amino acid sequence of SEQ ID NO: 200 (according to the Chothia definition system), a CDR-H3 having the amino acid sequence of SEQ ID NO: 201 (according to the Chothia definition system); and a VL (e.g., a humanized VL) comprising a CDR-L1 having the amino acid sequence of SEQ ID NO: 202 (according to the Chothia definition system), a CDR-L2 having the amino acid sequence of SEQ ID NO: 192 (according to the Chothia definition system), and a CDR-L3 having the amino acid sequence of SEQ ID NO: 203 (according to the Chothia definition system), wherein the VH and VL are on a single polypeptide chain (e.g., linked via an amide bond or linked via a linker such as a peptide linker), and wherein the VH is linked to the N-terminus or the C-terminus of the VL. In some embodiments, the VH and VL are linked via a linker comprising the amino acid sequence of EGKSSGSGSESKAS (SEQ ID NO: 215).

In some embodiments, the scFV comprises a VH (e.g., a humanized VH) comprising an amino acid sequence that is at least 75% (e.g., 75%, 80%, 85%, 90%, 95%, 98%, or 99%) identical to the VH as set forth in SEQ ID NO: 204 and a VL (e.g., a humanized VL) comprising an amino acid sequence that is at least 75% (e.g., 75%, 80%, 85%, 90%, 95%, 98%, or 99%) identical to the VL as set forth in SEQ ID NO: 205, wherein the VH and VL are in a single polypeptide chain (e.g., linked via an amide bond or linked via a linker such as a peptide linker), and wherein the VH is linked to the N-terminus or the C-terminus of the VL. In some embodiments, the VH and VL are linked via a linker comprising the amino acid sequence of EGKSSGSGSESKAS (SEQ ID NO: 215).

In some embodiments, the scFV comprises a VH (e.g., a humanized VH) that contains no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 204, and a humanized VL (e.g., a humanized VL) that contains no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VL as set forth in SEQ ID NO: 205, wherein the VH and VL are in a single polypeptide chain (e.g., linked via an amide bond or linked via a linker such as a peptide linker), and wherein the VH is linked to the N-terminus or the C-terminus of the VL. In some embodiments, the VH and VL are linked via a linker comprising the amino acid sequence of EGKSSGSGSESKAS (SEQ ID NO: 215).

In some embodiments, the scFV comprises a VH comprising the amino acid sequence of SEQ ID NO: 204 and a VL comprising the amino acid sequence of SEQ ID NO: 205, wherein the VH and VL are in a single polypeptide chain (e.g., linked via an amide bond or linked via a linker such as a peptide linker), and wherein the VH is linked to the N-terminus or the C-terminus of the VL. In some embodiments, the VH and VL are linked via a linker comprising the amino acid sequence of EGKSSGSGSESKAS (SEQ ID NO: 215).

In some embodiments, the scFv comprises a VH comprising the amino acid sequence of SEQ ID NO: 204 linked to the N-terminus of a VL comprising the amino acid sequence of SEQ ID NO: 205. In some embodiments, the VH and VL are linked via a linker comprising the amino acid sequence of EGKSSGSGSESKAS (SEQ ID NO: 215).

In some embodiments, the scFv comprises a VH comprising the amino acid sequence of SEQ ID NO: 204 linked to the C-terminus of a VL comprising the amino acid sequence of SEQ ID NO: 205. In some embodiments, the VH and VL are linked via a linker comprising the amino acid sequence of EGKSSGSGSESKAS (SEQ ID NO: 215).

The amino acid sequence of an example of a scFV is provided below (VL-linker-VH):

(SEQ ID NO: 206)
DIQMTQSPSSLSASVGDRVTITCRASQSISSYLNWYQQKPGKAPKLLIY
AASSLQSGVPSRFSGSGSGTDFTLTISSLQPEDFATYYCQQSYSTPLTF
GGGTKVEIKEGKSSGSGSESKASQVQLVQSGAEVKKPGESLKISCKGSG
YSFTSYWIGWVRQMPGKGLEWMGIIYPGDSDTRYSPSFQGQVTISADKS
ISTAYLQWSSLKASDTAMYYCARFPYDSSGYYSFDYWGQGTLVTVSS

In some embodiments, the scFv described herein comprises an amino acid sequence that is at least 75% (e.g., 75%, 80%, 85%, 90%, 95%, 98%, or 99%) identical to the VH as set forth in SEQ ID NO: 206. In some embodiments, the scFv described herein comprises an amino acid sequence that contains no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with SEQ ID NO: 206. In some embodiments, the scFv comprises the amino acid sequence of SEQ ID NO: 206.

In some embodiments, the anti-TfR antibody described herein comprises an scFv (e.g., any one of the scFv described herein) linked to a constant region. In some embodiments, the Fc region is a fragment crystallizable region (Fc region). The Fc region is a fragment of a heavy chain constant region that interacts with cell surface receptors called Fc receptors. Any known Fc regions may be used in accordance with the present disclosure and be fused to any one of the scFv described herein. The amino acid sequence of an example of a Fc region is provided below:

(SEQ ID NO: 207)
PKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWL
NGKEYKCKVSNKALPAPIEKTISKAKGQPREPQVYTLPPSRDELTKNQV
SLTCLVKGFYPSDIAVEWESNGQPENNYKTTPPVLDSDGSFFLYSKLTV
DKSRWQQGNVFSCSVMHEALHNHYTQKSLSLSPGK

In some embodiments, the anti-TfR antibody described herein comprises an scFv (e.g., any one of the scFv described herein or variants thereof) linked (e.g., via an amide bond or a linker such as a peptide linker) at the C-terminus to a Fc region that is at least 75% (e.g., 75%, 80%, 85%, 90%, 95%, 98%, or 99%) identical to the Fc region as set forth in SEQ ID NO: 207. In some embodiments, the anti-TfR antibody described herein comprises an scFv (e.g., any one of the scFv described herein or variants thereof) linked (e.g., via an amide bond or a linker such as a peptide linker) at the C-terminus to a Fc region that contains no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with SEQ ID NO: 207. In some embodiments, the anti-TfR antibody described herein comprises an scFv (e.g., any one of the scFv described herein or variants thereof) linked (e.g., via an amide bond or a linker such as a peptide linker) at the C-terminus to a Fc region set forth in SEQ ID NO: 207. In some embodiments, the scFV and the Fc are linked via a linker comprising the amino acid sequence of DIEGRMD (SEQ ID NO: 247).

The amino acid sequence of an example of an anti-TfR antibody comprising an scFv (e.g., any one of the scFv described herein) linked at the C-terminus to a Fc region is provided below (VL-linker1-VH-linker2-Fc):

(SEQ ID NO: 208)
DIQMTQSPSSLSASVGDRVTITCRASQSISSYLNWYQQKPGKAPKLLIY
AASSLQSGVPSRFSGSGSGTDFTLTISSLQPEDFATYYCQQSYSTPLTF
GGGTKVEIKEGKSSGSGSESKASQVQLVQSGAEVKKPGESLKISCKGSG
YSFTSYWIGWVRQMPGKGLEWMGIIYPGDSDTRYSPSFQGQVTISADKS
ISTAYLQWSSLKASDTAMYYCARFPYDSSGYYSFDYWGQGTLVTVSSDI
EGRMDPKSCDKTHTCPPCPAPELLGGPSVFLFPPKPKDTLMISRTPEVT
CVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSRDEL
TKNQVSLTCLVKGFYPSDIAVEWESNGQPENNYKTTPPVLDSDGSFFLY
SKLTVDKSRWQQGNVFSCSVMHEALHNHYTQKSLSLSPGK

In some embodiments, the anti-TfR antibody described herein comprises an amino acid sequence that is at least 75% (e.g., 75%, 80%, 85%, 90%, 95%, 98%, or 99%) identical to SEQ ID NO: 208. In some embodiments, the anti-TfR antibody described herein comprises an amino acid sequence that contains no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with SEQ ID NO: 208. In some embodiments, the anti-TfR antibody comprises the amino acid sequence of SEQ ID NO: 208.

In some embodiments, the anti-TfR antibody described herein comprises an scFv (e.g., any one of the scFv described herein) linked (e.g., via an amide bond or a linker such as a peptide linker) at the N-terminus to a Fc region that is at least 75% (e.g., 75%, 80%, 85%, 90%, 95%, 98%, or 99%) identical to the Fc region as set forth in SEQ ID NO: 207. In some embodiments, the anti-TfR antibody described herein comprises an scFv (e.g., any one of the scFv described herein) linked (e.g., via an amide bond or a linker such as a peptide linker) at the N-terminus to a Fc region that contains no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with SEQ ID NO: 207. In some embodiments, the anti-TfR antibody described herein comprises an scFv (e.g., any one of the scFv described herein) linked (e.g., via an amide bond or a linker such as a peptide linker) at the N-terminus to a Fc region set forth in SEQ ID NO: 207. In some embodiments, the scFV and the Fc are linked via a linker comprising the amino acid sequence of DIEGRMD (SEQ ID NO: 247).

The amino acid sequence of an example of an anti-TfR antibody comprising an scFv (e.g., any one of the scFv described herein) linked at the N-terminus to a Fc region is provided below (Fc-linker2-VL-linker1-VH):

(SEQ ID NO: 209)
PKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWL
NGKEYKCKVSNKALPAPIEKTISKAKGQPREPQVYTLPPSRDELTKNQV
SLTCLVKGFYPSDIAVEWESNGQPENNYKTTPPVLDSDGSFFLYSKLTV
DKSRWQQGNVFSCSVMHEALHNHYTQKSLSLSPGKDIEGRMDDIQMTQS
PSSLSASVGDRVTITCRASQSISSYLNWYQQKPGKAPKLLIYAASSLQS
GVPSRFSGSGSGTDFTLTISSLQPEDFATYYCQQSYSTPLTFGGGTKVE
IKEGKSSGSGSESKASQVQLVQSGAEVKKPGESLKISCKGSGYSFTSYW
IGWVRQMPGKGLEWMGIIYPGDSDTRYSPSFQGQVTISADKSISTAYLQ
WSSLKASDTAMYYCARFPYDSSGYYSFDYWGQGTLVTVSS

In some embodiments, the anti-TfR antibody described herein comprises an amino acid sequence that is at least 75% (e.g., 75%, 80%, 85%, 90%, 95%, 98%, or 99%) identical to SEQ ID NO: 209. In some embodiments, the anti-TfR antibody described herein comprises an amino acid sequence that contains no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with SEQ ID NO: 209. In some embodiments, the anti-TfR antibody comprises the amino acid sequence of SEQ ID NO: 209.

In some embodiments, the anti-TfR antibody described herein is an IgG. In some embodiments, the IgG comprises a heavy chain and a light chain, wherein the heavy chain comprises the CDR-H1, CDRH2, and CDR-H3 of any one of the anti-TfR antibodies described herein, and further comprises a heavy chain constant region or a portion thereof (e.g., CH1, CH2, CH3, or a combination thereof); and wherein the light chain comprises the CDR-L1, CDRL2, and CDR-L3 of any one of the anti-TfR antibodies described herein, and further comprises a light chain constant region. In some embodiments, the IgG comprises a heavy chain and a light chain, wherein the heavy chain comprises the VH of any one of the anti-TfR antibodies described herein, and further comprises a heavy chain constant region or a portion thereof (e.g., CH1, CH2, CH3, or a combination thereof); and wherein the light chain comprises the VL of any one of the anti-TfR antibodies described herein, and further comprises a light chain constant region.

The heavy chain constant region can of any suitable origin, e.g., human, mouse, rat, or rabbit. In one specific example, the heavy chain constant region is from a human IgG (a gamma heavy chain), e.g., IgG1, IgG2, or IgG4. An example of a human IgG1 constant region is given below:

(SEQ ID NO: 175)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK

In some embodiments, the heavy chain of any of the anti-TfR antibodies described herein comprises a mutant human IgG1 constant region. For example, the introduction of LALA mutations (a mutant derived from mAb b12 that has been mutated to replace the lower hinge residues Leu234 Leu235 with Ala234 and Ala235) in the CH2 domain of human IgG1 is known to reduce Fcg receptor binding (Bruhns, P., et al . (2009) and Xu, D. et al. (2000)). The mutant human IgG1 constant region is provided below (mutations bonded and underlined):

(SEQ ID NO: 176)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK

In some embodiments, the light chain constant region of any of the anti-TfR antibodies described herein can be any light chain constant region known in the art. In some examples, a kappa light chain or a lambda light chain. In some embodiments, the light chain constant region is a kappa light chain, the sequence of which is provided below:

(SEQ ID NO: 177)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC

Other antibody heavy and light chain constant regions are well known in the art, e.g., those provided in the IMGT database (www.imgt.org) or at www.vbase2.org/vbstat.php., both of which are incorporated by reference herein.

In some embodiments, the anti-TfR antibody described herein comprises a heavy chain comprising the a VH comprising the amino acid sequence of SEQ ID NO: 204 or any variants thereof and a heavy chain constant region that at least 75% (e.g., 75%, 80%, 85%, 90%, 95%, 98%, or 99%) identical to SEQ ID NO: 175 or SEQ ID NO: 176. In some embodiments, the anti-TfR antibody described herein comprises a heavy chain comprising the a VH comprising the amino acid sequence of SEQ ID NO: 204 or any variants thereof and a heavy chain constant region that contains no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the heavy chain as set forth in SEQ ID NO: 175 or SEQ ID NO: 176.

In some embodiments, the anti-TfR antibody described herein comprises a heavy chain comprising a VH set forth in SEQ ID NO: 204 and a heavy chain constant region set forth in SEQ ID NO: 175. In some embodiments, the anti-TfR antibody described herein comprises a heavy chain comprising a VH set forth in SEQ ID NO: 204 and a heavy chain constant region as set forth in SEQ ID NO: 176.

In some embodiments, the anti-TfR antibody described herein comprises a light chain comprising a VL comprising the amino acid sequence of SEQ ID NO: 205 or any variants thereof and a light chain constant region that is at least 75% (e.g., 75%, 80%, 85%, 90%, 95%, 98%, or 99%) identical to SEQ ID NO: 177. In some embodiments, the anti-TfR antibody described herein comprises a light chain comprising a VL comprising the amino acid sequence of SEQ ID NO: 205 or any variants thereof and a light constant region that contains no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the heavy chain as set forth in SEQ ID NO: 177.

In some embodiments, the anti-TfR antibody described herein comprises a light chain comprising a VL set forth in SEQ ID NO: 205 and a light chain constant region as set forth in SEQ ID NO: 177.

Examples of IgG heavy chain and light chain amino acid sequences of the anti-TfR antibodies described are provided below.

anti-TfR IgG heavy chain (with wild type human IgGI constant
region, VH underlined)
(SEQ ID NO: 210)
QVQLVQSGAEVKKPGESLKISCKGSGYSFTSYWIGWVRQMPGKGLEWMGIIYPGDSD
TRYSPSFQGQVTISADKSISTAYLQWSSLKASDTAMYYCARFPYDSSGYYSFDYWGQ
GTLVTVSSASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEPKSCDKTHT
CPPCPAPELLLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKA
KGQPREPQVYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGK
anti-TfR IgG heavy chain (with human IgGI constant region
mutant L234A/L235A, VH underlined)
(SEQ ID NO: 211)
QVQLVQSGAEVKKPGESLKISCKGSGYSFTSYWIGWVRQMPGKGLEWMGIIYPGDSD
TRYSPSFQGQVTISADKSISTAYLQWSSLKASDTAMYYCARFPYDSSGYYSFDYWGQ
GTLVTVSSASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEPKSCDKTHT
CPPCPAPEAAGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEKTISKAK
GQPREPQVYTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPGK
anti-TfR IgG light chain (kappa, VL underlined)
(SEQ ID NO: 212)
DIQMTQSPSSLSASVGDRVTITCRASQSISSYLNWYQQKPGKAPKLLIYAASSLQSGVP
SRFSGSGSGTDFTLTISSLQPEDFATYYCQQSYSTPLTFGGGTKVEIKRTVAAPSVFIFPP
SDEQLKSGTASVVCLLNNFYPREAKVQWKVDNALQSGNSQESVTEQDSKDSTYSLSS
TLTLSKADYEKHKVYACEVTHQGLSSPVTKSFNRGEC

In some embodiments, the anti-TfR antibody described herein comprises a heavy chain comprising an amino acid sequence that is at least 75% (e.g., 75%, 80%, 85%, 90%, 95%, 98%, or 99%) identical to SEQ ID NO: 210 or SEQ ID NO: 211. Alternatively or in addition (e.g., in addition), the anti-TfR antibody described herein comprises a light chain comprising an amino acid sequence that is at least 75% (e.g., 75%, 80%, 85%, 90%, 95%, 98%, or 99%) identical to any one of SEQ ID NOs: 212.

In some embodiments, the anti-TfR antibody of the present disclosure comprises a heavy chain containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the heavy chain as set forth in SEQ ID NO: 210 or SEQ ID NO: 211. Alternatively or in addition (e.g., in addition), the anti-TfR antibody of the present disclosure comprises a light chain containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the light chain as set forth in SEQ ID NO: 212.

In some embodiments, the anti-TfR antibody described herein comprises a heavy chain comprising the amino acid sequence of SEQ ID NO: 210 or SEQ ID NO: 211. Alternatively or in addition (e.g., in addition), the anti-TfR antibody described herein comprises a light chain comprising the amino acid sequence of any one of SEQ ID NO: 212.

In some embodiments, the anti-TfR antibody is a FAB fragment, a F(ab′) fragment, or F(ab′)₂ fragment of an intact antibody (full-length antibody). Antigen binding fragment of an intact antibody (full-length antibody) can be prepared via routine methods (e.g., recombinantly or by digesting the heavy chain constant region of a full length IgG using an enzyme such as papain). For example, F(ab′)₂ fragments can be produced by pepsin or papain digestion of an antibody molecule, and Fab fragments that can be generated by reducing the disulfide bridges of F(ab′)₂ fragments. In some embodiments, a heavy chain constant region in a F(ab′) fragment of the anti-TfR1 antibody described herein comprises the amino acid sequence of:

(SEQ ID NO: 184)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHT

In some embodiments, the anti-TfR antibody described herein comprises a heavy chain comprising the a VH comprising the amino acid sequence of SEQ ID NO: 204 or any variants thereof and a heavy chain constant region that at least 75% (e.g., 75%, 80%, 85%, 90%, 95%, 98%, or 99%) identical to SEQ ID NO: 184. In some embodiments, the anti-TfR antibody described herein comprises a heavy chain comprising the a VH comprising the amino acid sequence of SEQ ID NO: 204 or any variants thereof and a heavy chain constant region that contains no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the heavy chain as set forth in SEQ ID NO: 184.

In some embodiments, the anti-TfR antibody described herein comprises a heavy chain comprising a VH set forth in SEQ ID NO: 204 and a heavy chain constant region as set forth in SEQ ID NO: 184.

Examples of F(ab′) amino acid sequences of an anti-TfR antibody described herein are provided below.

anti-TfR Fab' heavy chain (with human IgGl constant region
fragment, VH underlined)
(SEQ ID NO: 213)
QVQLVQSGAEVKKPGESLKISCKGSGYSFTSYWIGWVRQMPGKGLEWMGIIYPGDSD
TRYSPSFQGQVTISADKSISTAYLQWSSLKASDTAMYYCARFPYDSSGYYSFDYWGQ
GTLVTVSSASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEPKSCDKTHT
CP
or
(SEQ ID NO: 308)
QVQLVQSGAEVKKPGESLKISCKGSGYSFTSYWIGWVRQMPGKGLEWMGIIYPGDSD
TRYSPSFQGQVTISADKSISTAYLQWSSLKASDTAMYYCARFPYDSSGYYSFDYWGQ
GTLVTVSSASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEPKSCDKTHT
anti-TfR Fab' light chain (kappa, VL underlined)
(SEQ ID NO: 212)
DIQMTQSPSSLSASVGDRVTITCRASQSISSYLNWYQQKPGKAPKLLIYAASSLQSGVP
SRFSGSGSGTDFTLTISSLQPEDFATYYCQQSYSTPLTFGGGTKVEIKRTVAAPSVFIFPP
SDEQLKSGTASVVCLLNNFYPREAKVQWKVDNALQSGNSQESVTEQDSKDSTYSLSS
TLTLSKADYEKHKVYACEVTHQGLSSPVTKSFNRGEC

In some embodiments, the anti-TfR antibody described herein comprises a heavy chain comprising an amino acid sequence that is at least 75% (e.g., 75%, 80%, 85%, 90%, 95%, 98%, or 99%) identical to SEQ ID NO: 213 or SEQ ID NO: 308. Alternatively or in addition (e.g., in addition), the anti-TfR antibody described herein comprises a light chain comprising an amino acid sequence that is at least 75% (e.g., 75%, 80%, 85%, 90%, 95%, 98%, or 99%) identical to SEQ ID NO: 212. In some embodiments, the anti-TfR antibody of the present disclosure comprises a heavy chain containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the heavy chain as set forth in SEQ ID NO: 213 or SEQ ID NO: 308. Alternatively or in addition (e.g., in addition), the anti-TfR antibody of the present disclosure comprises a light chain containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the light chain as set forth in SEQ ID NO: 212. In some embodiments, the anti-TfR antibody described herein comprises a heavy chain comprising the amino acid sequence of SEQ ID NO: 213 or SEQ ID NO: 308. Alternatively or in addition (e.g., in addition), the anti-TfR antibody described herein comprises a light chain comprising the amino acid sequence of SEQ ID NO: 212.

In some embodiments, any one of the anti-TfR1 antibodies described herein may comprise a signal peptide in the heavy and/or (e.g., and) light chain sequence (e.g., a N-terminal signal peptide). In some embodiments, the anti-TfR1 antibody described herein comprises any one of the VH and VL sequences, any one of the IgG heavy chain and light chain sequences listed, or any one of the F(ab′) heavy chain and light chain sequences described herein, and further comprises a signal peptide (e.g., a N-terminal signal peptide). In some embodiments, the signal peptide comprises the amino acid sequence of

(SEQ ID NO: 214)
MGWSCIILFLVATATGVHS

### Other Known Anti-Transferrin Receptor Antibodies

Any other appropriate anti-transferrin receptor antibodies known in the art may be used as the muscle-targeting agent in the complexes disclosed herein. Examples of known anti-transferrin receptor antibodies, including associated references and binding epitopes, are listed in Table 8. In some embodiments, the anti-transferrin receptor antibody comprises the complementarity determining regions (CDR-H1, CDR-H2, CDR-H3, CDR-L1, CDR-L2, and CDR-L3) of any of the anti-transferrin receptor antibodies provided herein, e.g., anti-transferrin receptor antibodies listed in Table 8.

TABLE 8
List of anti-transferrin receptor antibody clones, including associated
references and binding epitope information.

| Antibody Clone Name | Reference(s) | Epitope/Notes |
|---|---|---|
| OKT9 | U.S. Pat No. | Apical domain |
| | 4,364,934, filed | of TfR (residues |
| | Dec. 4, 1979, entitled | 305-366 of |
| | “MONOCLONAL | human TfR |
| | ANTIBODY TO A | sequence |
| | HUMAN EARLY | XM_052730.3, |
| | THYMOCYTE | available in |
| | ANTIGEN AND | GenBank) |
| | METHODS FOR | |
| | PREPARING | |
| | SAME” | |
| | Schneider C. et al. | |
| | “Structural features | |
| | of the cell surface | |
| | receptor for | |
| | transferrin that is | |
| | recognized by the | |
| | monoclonal antibody | |
| | OKT9.” J Biol Chem. | |
| | 1982, 257: 14, 8516- | |
| | 8522. | |
| (From JCR) | WO | Apical domain |
| Clone M11 | 2015/098989, filed | (residues 230- |
| Clone M23 | Dec. 24, 2014, “Novel | 244 and 326- |
| Clone M27 | anti-Transferrin | 347 of TfR) and |
| Clone B84 | receptor antibody that | protease-like |
| | passes through blood- | domain |
| | brain barrier” | (residues 461- |
| | U.S. Pat. No. | 473) |
| | 9,994,641, filed | |
| | Dec. 24, 2014, | |
| | “Novel anti- | |
| | Transferrin | |
| | receptor antibody | |
| | that passes | |
| | through blood- | |
| | brain barrier” | |
| (From Genentech) | WO | Apical domain |
| 7A4, 8A2, 15D2, 10D11, 7B10, 15G11*, | 2016/081643, filed | and non-apical |
| 16G5, 13C3, 16G4, 16F6, 7G7, 4C2, 1B12, | May 26, 2016, entitled | regions |
| and 13D4 | “ANTI- | |
| *15G11 CDRs listed in Table 9 | TRANSFERRIN | |
| VH (SEQ ID NO: 230) | RECEPTOR | |
| VL (SEQ ID NO: 231) | ANTIBODIES AND | |
| Fab′ HC (SEQ ID NO: 240) | METHODS OF | |
| Fab′ LC (SEQ ID NO: 237) | USE” | |
| | U.S. Pat. No. | |
| | 9,708,406, filed | |
| | May 20, 2014, “Anti- | |
| | transferrin receptor | |
| | antibodies and | |
| | methods of use” | |
| (From Armagen) | Lee et al. | |
| 8D3 | “Targeting Rat Anti- | |
| | Mouse Transferrin | |
| | Receptor Monoclonal | |
| | Antibodies through | |
| | Blood-Brain Barrier | |
| | in Mouse” 2000, J | |
| | Pharmacol. Exp. | |
| | Ther., 292: 1048- | |
| | 1052. | |
| | US Patent | |
| | App. 2010/077498, | |
| | filed Sep. 11, 2008, | |
| | entitled | |
| | “COMPOSITIONS | |
| | AND METHODS | |
| | FOR BLOOD- | |
| | BRAIN BARRIER | |
| | DELIVERY IN THE | |
| | MOUSE” | |
| OX26 | Haobam, B. et | |
| | al. 2014. Rab17- | |
| | mediated recycling | |
| | endosomes contribute | |
| | to autophagosome | |
| | formation in response | |
| | to Group A | |
| | Streptococcus | |
| | invasion. Cellular | |
| | microbiology. 16: | |
| | 1806-21. | |
| DF1513 | Ortiz-Zapater | |
| | E et al. Trafficking | |
| | of the human | |
| | transferrin receptor in | |
| | plant cells: effects of | |
| | tyrphostin A23 and | |
| | brefeldin A. Plant J | |
| | 48: 757-70 (2006). | |
| 1A1B2, 661G10, MEM-189, JF0956, | Commercially | Novus |
| 29806, 1A1B2, TFRC/1818, 1E6, 66Ig10, | available anti- | Biologicals |
| TFRC/1059, Q1/71, 23D10, 13E4, | transferrin receptor | 8100 Southpark |
| TFRC/1149, ER-MP21, YTA74.4, BU54, | antibodies. | Way, A-8 |
| 2B6, RI7 217 | | Littleton CO |
| | | 80120 |
| (From INSERM) | US Patent | Does not |
| BA120g | App. | compete with |
| | 2011/0311544A1, | OKT9 |
| | filed Jun. 15, 2005, | |
| | entitled “ANTI-CD71 | |
| | MONOCLONAL | |
| | ANTIBODIES AND | |
| | USES THEREOF | |
| | FOR TREATING | |
| | MALIGNANT | |
| | TUMOR CELLS” | |
| LUCA31 | U.S. Pat. No. | “LUCA31 |
| | 7,572,895, filed | epitope” |
| | Jun. 7, 2004, entitled | |
| | “TRANSFERRIN | |
| | RECEPTOR | |
| | ANTIBODIES” | |
| (Salk Institute) | Trowbridge, I. S. | |
| B3/25 | et al. “Anti- | |
| T58/30 | transferrin | |
| | receptor | |
| | monoclonal | |
| | antibody and | |
| | toxin-antibody | |
| | conjugates affect | |
| | growth of human | |
| | tumour cells.” | |
| | Nature, 1981, | |
| | volume 294, | |
| | pages 171-173 | |
| R17 217.1.3, | Commercially | BioXcell |
| 5E9C11, | available anti- | 10 Technology |
| OKT9 (BE0023 clone) | transferrin receptor | Dr., Suite 2B |
| | antibodies. | West Lebanon, |
| | | NH 03784-1671 |
| | | USA |
| BK19.9, B3/25, T56/14 and T58/1 | Gatter, K. C. et al. | |
| | “Transferrin | |
| | receptors in | |
| | human tissues: | |
| | their distribution | |
| | and possible | |
| | clinical | |
| | relevance.” J Clin | |
| | Pathol. 1983 | |
| | May; 36(5): 539- | |
| | 45. | |

Anti-TfR antibody 15G11
CDRs listed in Table 9
VH (SEQ ID NO: 230)
VL (SEQ ID NO: 231)
Fab′ HC (SEQ ID NO: 240)
Fab′ LC (SEQ ID NO: 237)
Anti-TfR antibody
CDRH1 (SEQ ID NO: 309)
CDRH2 (SEQ ID NO: 310)
CDRH3 (SEQ ID NO: 311)
CDRL1 (SEQ ID NO: 312)
CDRL2 (SEQ ID NO: 313)
CDRL3 (SEQ ID NO: 314)
VH (SEQ ID NO: 315)
VL(SEQ ID NO: 316)

Other anti-TfR antibody SEQ ID NOs

| | VH/ | | | |
|---|---|---|---|---|
| | VL | CDR1 | CDR2 | CDR3 |
| VH1 | 325 | 317 | 318 | 311 |
| VH2 | 326 | 317 | 319 | 311 |
| VH3 | 327 | 317 | 320 | 311 |
| VH4 | 328 | 317 | 319 | 311 |
| VL1 | 329 | 312 | 313 | 321 |
| VL2 | 330 | 312 | 313 | 321 |
| VL3 | 331 | 312 | 322 | 314 |
| VL4 | 332 | 323 | 324 | 314 |

In some embodiments, transferrin receptor antibodies of the present disclosure include one or more of the CDR-H (e.g., CDR-H1, CDR-H2, and CDR-H3) amino acid sequences from any one of the anti-transferrin receptor antibodies selected from Table 8. In some embodiments, transferrin receptor antibodies include the CDR-H1, CDR-H2, and CDR-H3 as provided for any one of the anti-transferrin receptor antibodies selected from Table 8. In some embodiments, anti-transferrin receptor antibodies include the CDR-L1, CDR-L2, and CDR-L3 as provided for any one of the anti-transferrin receptor antibodies selected from Table 8. In some embodiments, anti-transferrin antibodies include the CDR-H1, CDR-H2, CDR-H3, CDR-L1, CDR-L2, and CDR-L3 as provided for any one of the anti-transferrin receptor antibodies selected from Table 8. The disclosure also includes any nucleic acid sequence that encodes a molecule comprising a CDR-H1, CDR-H2, CDR-H3, CDR-L1, CDR-L2, or CDR-L3 as provided for any one of the anti-transferrin receptor antibodies selected from Table 8. In some embodiments, antibody heavy and light chain CDR3 domains may play a particularly important role in the binding specificity/affinity of an antibody for an antigen. Accordingly, anti-transferrin receptor antibodies of the disclosure may include at least the heavy and/or (e.g., and) light chain CDR3s of any one of the anti-transferrin receptor antibodies selected from Table 8.

In some examples, any of the anti-transferrin receptor antibodies of the disclosure have one or more CDR (e.g., CDR-H or CDR-L) sequences substantially similar to any of the CDR-H1, CDR-H2, CDR-H3, CDR-L1, CDR-L2, and/or (e.g., and) CDR-L3 sequences from one of the anti-transferrin receptor antibodies selected from Table 8. In some embodiments, the position of one or more CDRs along the VH (e.g., CDR-H1, CDR-H2, or CDR-H3) and/or (e.g., and) VL (e.g., CDR-L1, CDR-L2, or CDR-L3) region of an antibody described herein can vary by one, two, three, four, five, or six amino acid positions so long as immunospecific binding to transferrin receptor (e.g., human transferrin receptor) is maintained (e.g., substantially maintained, for example, at least 50%, at least 60%, at least 70%, at least 80%, at least 90%, at least 95% of the binding of the original antibody from which it is derived). For example, in some embodiments, the position defining a CDR of any antibody described herein can vary by shifting the N-terminal and/or (e.g., and) C-terminal boundary of the CDR by one, two, three, four, five, or six amino acids, relative to the CDR position of any one of the antibodies described herein, so long as immunospecific binding to transferrin receptor (e.g., human transferrin receptor) is maintained (e.g., substantially maintained, for example, at least 50%, at least 60%, at least 70%, at least 80%, at least 90%, at least 95% of the binding of the original antibody from which it is derived). In another embodiment, the length of one or more CDRs along the VH (e.g., CDR-H1, CDR-H2, or CDR-H3) and/or (e.g., and) VL (e.g., CDR-L1, CDR-L2, or CDR-L3) region of an antibody described herein can vary (e.g., be shorter or longer) by one, two, three, four, five, or more amino acids, so long as immunospecific binding to transferrin receptor (e.g., human transferrin receptor) is maintained (e.g., substantially maintained, for example, at least 50%, at least 60%, at least 70%, at least 80%, at least 90%, at least 95% of the binding of the original antibody from which it is derived).

Accordingly, in some embodiments, a CDR-L1, CDR-L2, CDR-L3, CDR-H1, CDR-H2, and/or (e.g., and) CDR-H3 described herein may be one, two, three, four, five or more amino acids shorter than one or more of the CDRs described herein (e.g., CDRS from any of the anti-transferrin receptor antibodies selected from Table 8) so long as immunospecific binding to transferrin receptor (e.g., human transferrin receptor) is maintained (e.g., substantially maintained, for example, at least 50%, at least 60%, at least 70%, at least 80%, at least 90%, at least 95% relative to the binding of the original antibody from which it is derived). In some embodiments, a CDR-L1, CDR-L2, CDR-L3, CDR-H1, CDR-H2, and/or (e.g., and) CDR-H3 described herein may be one, two, three, four, five or more amino acids longer than one or more of the CDRs described herein (e.g., CDRS from any of the anti-transferrin receptor antibodies selected from Table 8) so long as immunospecific binding to transferrin receptor (e.g., human transferrin receptor) is maintained (e.g., substantially maintained, for example, at least 50%, at least 60%, at least 70%, at least 80%, at least 90%, at least 95% relative to the binding of the original antibody from which it is derived). In some embodiments, the amino portion of a CDR-L1, CDR-L2, CDR-L3, CDR-H1, CDR-H2, and/or (e.g., and) CDR-H3 described herein can be extended by one, two, three, four, five or more amino acids compared to one or more of the CDRs described herein (e.g., CDRS from any of the anti-transferrin receptor antibodies selected from Table 8) so long as immunospecific binding to transferrin receptor (e.g., human transferrin receptor is maintained (e.g., substantially maintained, for example, at least 50%, at least 60%, at least 70%, at least 80%, at least 90%, at least 95% relative to the binding of the original antibody from which it is derived). In some embodiments, the carboxy portion of a CDR-L1, CDR-L2, CDR-L3, CDR-H1, CDR-H2, and/or (e.g., and) CDR-H3 described herein can be extended by one, two, three, four, five or more amino acids compared to one or more of the CDRs described herein (e.g., CDRS from any of the anti-transferrin receptor antibodies selected from Table 8) so long as immunospecific binding to transferrin receptor (e.g., human transferrin receptor) is maintained (e.g., substantially maintained, for example, at least 50%, at least 60%, at least 70%, at least 80%, at least 90%, at least 95% relative to the binding of the original antibody from which it is derived). In some embodiments, the amino portion of a CDR-L1, CDR-L2, CDR-L3, CDR-H1, CDR-H2, and/or (e.g., and) CDR-H3 described herein can be shortened by one, two, three, four, five or more amino acids compared to one or more of the CDRs described herein (e.g., CDRS from any of the anti-transferrin receptor antibodies selected from Table 8) so long as immunospecific binding to transferrin receptor (e.g., human transferrin receptor) is maintained (e.g., substantially maintained, for example, at least 50%, at least 60%, at least 70%, at least 80%, at least 90%, at least 95% relative to the binding of the original antibody from which it is derived). In some embodiments, the carboxy portion of a CDR-L1, CDR-L2, CDR-L3, CDR-H1, CDR-H2, and/or (e.g., and) CDR-H3 described herein can be shortened by one, two, three, four, five or more amino acids compared to one or more of the CDRs described herein (e.g., CDRS from any of the anti-transferrin receptor antibodies selected from Table 8) so long as immunospecific binding to transferrin receptor (e.g., human transferrin receptor) is maintained (e.g., substantially maintained, for example, at least 50%, at least 60%, at least 70%, at least 80%, at least 90%, at least 95% relative to the binding of the original antibody from which it is derived). Any method can be used to ascertain whether immunospecific binding to transferrin receptor (e.g., human transferrin receptor) is maintained, for example, using binding assays and conditions described in the art.

In some examples, any of the anti-transferrin receptor antibodies of the disclosure have one or more CDR (e.g., CDR-H or CDR-L) sequences substantially similar to any one of the anti-transferrin receptor antibodies selected from Table 8. For example, the antibodies may include one or more CDR sequence(s) from any of the anti-transferrin receptor antibodies selected from Table 8 containing up to 5, 4, 3, 2, or 1 amino acid residue variations as compared to the corresponding CDR region in any one of the CDRs provided herein (e.g., CDRs from any of the anti-transferrin receptor antibodies selected from Table 8) so long as immunospecific binding to transferrin receptor (e.g., human transferrin receptor) is maintained (e.g., substantially maintained, for example, at least 50%, at least 60%, at least 70%, at least 80%, at least 90%, at least 95% relative to the binding of the original antibody from which it is derived). In some embodiments, any of the amino acid variations in any of the CDRs provided herein may be conservative variations. Conservative variations can be introduced into the CDRs at positions where the residues are not likely to be involved in interacting with a transferrin receptor protein (e.g., a human transferrin receptor protein), for example, as determined based on a crystal structure. Some aspects of the disclosure provide transferrin receptor antibodies that comprise one or more of the heavy chain variable (VH) and/or (e.g., and) light chain variable (VL) domains provided herein. In some embodiments, any of the VH domains provided herein include one or more of the CDR-H sequences (e.g., CDR-H1, CDR-H2, and CDR-H3) provided herein, for example, any of the CDR-H sequences provided in any one of the anti-transferrin receptor antibodies selected from Table 8. In some embodiments, any of the VL domains provided herein include one or more of the CDR-L sequences (e.g., CDR-L1, CDR-L2, and CDR-L3) provided herein, for example, any of the CDR-L sequences provided in any one of the anti-transferrin receptor antibodies selected from Table 8.

In some embodiments, anti-transferrin receptor antibodies of the disclosure include any antibody that includes a heavy chain variable domain and/or (e.g., and) a light chain variable domain of any anti-transferrin receptor antibody, such as any one of the anti-transferrin receptor antibodies selected from Table 8. In some embodiments, anti-transferrin receptor antibodies of the disclosure include any antibody that includes the heavy chain variable and light chain variable pairs of any anti-transferrin receptor antibody, such as any one of the anti-transferrin receptor antibodies selected from Table 8.

Aspects of the disclosure provide anti-transferrin receptor antibodies having a heavy chain variable (VH) and/or (e.g., and) a light chain variable (VL) domain amino acid sequence homologous to any of those described herein. In some embodiments, the anti-transferrin receptor antibody comprises a heavy chain variable sequence or a light chain variable sequence that is at least 75% (e.g., 80%, 85%, 90%, 95%, 98%, or 99%) identical to the heavy chain variable sequence and/ or any light chain variable sequence of any anti-transferrin receptor antibody, such as any one of the anti-transferrin receptor antibodies selected from Table 8. In some embodiments, the homologous heavy chain variable and/or (e.g., and) a light chain variable amino acid sequences do not vary within any of the CDR sequences provided herein. For example, in some embodiments, the degree of sequence variation (e.g., 75%, 80%, 85%, 90%, 95%, 98%, or 99%) may occur within a heavy chain variable and/or (e.g., and) a light chain variable sequence excluding any of the CDR sequences provided herein. In some embodiments, any of the anti-transferrin receptor antibodies provided herein comprise a heavy chain variable sequence and a light chain variable sequence that comprises a framework sequence that is at least 75%, 80%, 85%, 90%, 95%, 98%, or 99% identical to the framework sequence of any anti-transferrin receptor antibody, such as any one of the anti-transferrin receptor antibodies selected from Table 8.

In some embodiments, an anti-transferrin receptor antibody, which specifically binds to transferrin receptor (e.g., human transferrin receptor), comprises a light chain variable VL domain comprising any of the CDR-L domains (CDR-L1, CDR-L2, and CDR-L3), or CDR-L domain variants provided herein, of any of the anti-transferrin receptor antibodies selected from Table 8. In some embodiments, an anti-transferrin receptor antibody, which specifically binds to transferrin receptor (e.g., human transferrin receptor), comprises a light chain variable VL domain comprising the CDR-L1, the CDR-L2, and the CDR-L3 of any anti-transferrin receptor antibody, such as any one of the anti-transferrin receptor antibodies selected from Table 8. In some embodiments, the anti-transferrin receptor antibody comprises a light chain variable (VL) region sequence comprising one, two, three or four of the framework regions of the light chain variable region sequence of any anti-transferrin receptor antibody, such as any one of the anti-transferrin receptor antibodies selected from Table 8. In some embodiments, the anti-transferrin receptor antibody comprises one, two, three or four of the framework regions of a light chain variable region sequence which is at least 75%, 80%, 85%, 90%, 95%, or 100% identical to one, two, three or four of the framework regions of the light chain variable region sequence of any anti-transferrin receptor antibody, such as any one of the anti-transferrin receptor antibodies selected from Table 8. In some embodiments, the light chain variable framework region that is derived from said amino acid sequence consists of said amino acid sequence but for the presence of up to 10 amino acid substitutions, deletions, and/or (e.g., and) insertions, preferably up to 10 amino acid substitutions. In some embodiments, the light chain variable framework region that is derived from said amino acid sequence consists of said amino acid sequence with 1, 2, 3, 4, 5, 6, 7, 8, 9 or 10 amino acid residues being substituted for an amino acid found in an analogous position in a corresponding non-human, primate, or human light chain variable framework region.

In some embodiments, an anti-transferrin receptor antibody that specifically binds to transferrin receptor comprises the CDR-L1, the CDR-L2, and the CDR-L3 of any anti-transferrin receptor antibody, such as any one of the anti-transferrin receptor antibodies selected from Table 8. In some embodiments, the antibody further comprises one, two, three or all four VL framework regions derived from the VL of a human or primate antibody. The primate or human light chain framework region of the antibody selected for use with the light chain CDR sequences described herein, can have, for example, at least 70% (e.g., at least 75%, 80%, 85%, 90%, 95%, 98%, or at least 99%) identity with a light chain framework region of a non-human parent antibody. The primate or human antibody selected can have the same or substantially the same number of amino acids in its light chain complementarity determining regions to that of the light chain complementarity determining regions of any of the antibodies provided herein, e.g., any of the anti-transferrin receptor antibodies selected from Table 8. In some embodiments, the primate or human light chain framework region amino acid residues are from a natural primate or human antibody light chain framework region having at least 75% identity, at least 80% identity, at least 85% identity, at least 90% identity, at least 95% identity, at least 98% identity, at least 99% (or more) identity with the light chain framework regions of any anti-transferrin receptor antibody, such as any one of the anti-transferrin receptor antibodies selected from Table 8. In some embodiments, an anti-transferrin receptor antibody further comprises one, two, three or all four VL framework regions derived from a human light chain variable kappa subfamily. In some embodiments, an anti-transferrin receptor antibody further comprises one, two, three or all four VL framework regions derived from a human light chain variable lambda subfamily.

In some embodiments, any of the anti-transferrin receptor antibodies provided herein comprise a light chain variable domain that further comprises a light chain constant region. In some embodiments, the light chain constant region is a kappa, or a lambda light chain constant region. In some embodiments, the kappa or lambda light chain constant region is from a mammal, e.g., from a human, monkey, rat, or mouse. In some embodiments, the light chain constant region is a human kappa light chain constant region. In some embodiments, the light chain constant region is a human lambda light chain constant region. It should be appreciated that any of the light chain constant regions provided herein may be variants of any of the light chain constant regions provided herein. In some embodiments, the light chain constant region comprises an amino acid sequence that is at least 75%, 80%, 85%, 90%, 95%, 98%, or 99% identical to any of the light chain constant regions of any anti-transferrin receptor antibody, such as any one of the anti-transferrin receptor antibodies selected from Table 8.

In some embodiments, the anti-transferrin receptor antibody is any anti-transferrin receptor antibody, such as any one of the anti-transferrin receptor antibodies selected from Table 8.

In some embodiments, an anti-transferrin receptor antibody comprises a VL domain comprising the amino acid sequence of any anti-transferrin receptor antibody, such as any one of the anti-transferrin receptor antibodies selected from Table 8, and wherein the constant regions comprise the amino acid sequences of the constant regions of an IgG, IgE, IgM, IgD, IgA or IgY immunoglobulin molecule, or a human IgG, IgE, IgM, IgD, IgA or IgY immunoglobulin molecule. In some embodiments, an anti-transferrin receptor antibody comprises any of the VL domains, or VL domain variants, and any of the VH domains, or VH domain variants, wherein the VL and VH domains, or variants thereof, are from the same antibody clone, and wherein the constant regions comprise the amino acid sequences of the constant regions of an IgG, IgE, IgM, IgD, IgA or IgY immunoglobulin molecule, any class (e.g., IgG1, IgG2, IgG3, IgG4, IgAl and IgA2), or any subclass (e.g., IgG2a and IgG2b) of immunoglobulin molecule. Non-limiting examples of human constant regions are described in the art, e.g., see Kabat E A et al., (1991) supra.

In some embodiments, the muscle-targeting agent is a transferrin receptor antibody (e.g., the antibody and variants thereof as described in International Application Publication WO 2016/081643, incorporated herein by reference).

The heavy chain and light chain CDRs of the antibody according to different definition systems are provided in Table 9. The different definition systems, e.g., the Kabat definition, the Chothia definition, and/or (e.g., and) the contact definition have been described. See, e.g., (e.g., Kabat, E. A., et al. (1991) Sequences of Proteins of Immunological Interest, Fifth Edition, U.S. Department of Health and Human Services, NIH Publication No. 91-3242, Chothia et al., (1989) Nature 342:877; Chothia, C. et al. (1987) J. Mol. Biol. 196:901-917, Allazikani et al (1997) J. Molec. Biol. 273:927-948; and Almagro, J. Mol. Recognit. 17:132-143 (2004). See also hgmp.mrc.ac.uk and bioinf.org.uk/abs).

TABLE 9
Heavy chain and light chain CDRs of a mouse transferrin
receptor antibody

| CDRs | Kabat | Chothia | Contact |
|---|---|---|---|
| CDR-H1 | SYWMH (SEQ ID NO: | GYTFTSY (SEQ ID NO: | TSYWMH (SEQ ID NO: |
| | 216) | 222) | 224) |
| CDR-H2 | EINPTNGRTNYIEKFKS | NPTNGR (SEQ ID NO: | WIGEINPTNGRTN |
| | (SEQ ID NO: 217) | 223) | (SEQ ID NO: 225) |
| CDR-H3 | GTRAYHY (SEQ ID | GTRAYHY (SEQ ID | ARGTRA (SEQ ID NO: |
| | NO: 218) | NO: 218) | 226) |
| CDR-L1 | RASDNLYSNLA (SEQ | RASDNLYSNLA (SEQ | YSNLAWY (SEQ ID |
| | ID NO: 219) | ID NO: 219) | NO: 227) |
| CDR-L2 | DATNLAD (SEQ ID | DATNLAD (SEQ ID NO: | LLVYDATNLA (SEQ ID |
| | NO: 220) | 220) | NO: 228) |
| CDR-L3 | QHFWGTPLT (SEQ ID | QHFWGTPLT (SEQ ID | QHFWGTPL (SEQ ID |
| | NO: 221) | NO: 221) | NO: 229) |

The heavy chain variable domain (VH) and light chain variable domain sequences are also provided:

VH
(SEQ ID NO: 230)
QVQLQQPGAELVKPGASVKLSCKASGYTFTSYWMHWVKQRPGQGLEWIG
EINPTNGRTNYIEKFKSKATLTVDKSSSTAYMQLSSLTSEDSAVYYCAR
GTRAYHYWGQGTSVTVSS
VL
(SEQ ID NO: 231)
DIQMTQSPASLSVSVGETVTITCRASDNLYSNLAWYQQKQGKSPQLLVY
DATNLADGVPSRFSGSGSGTQYSLKINSLQSEDFGTYYCQHFWGTPLTF
GAGTKLELK

In some embodiments, the transferrin receptor antibody of the present disclosure comprises a CDR-H1, a CDR-H2, and a CDR-H3 that are the same as the CDR-H1, CDR-H2, and CDR-H3 shown in Table 9. Alternatively or in addition (e.g., in addition), the transferrin receptor antibody of the present disclosure comprises a CDR-L1, a CDR-L2, and a CDR-L3 that are the same as the CDR-L1, CDR-L2, and CDR-L3 shown in Table 9.

In some embodiments, the transferrin receptor antibody of the present disclosure comprises a CDR-H1, a CDR-H2, and a CDR-H3, which collectively contains no more than 5 amino acid variations (e.g., no more than 5, 4, 3, 2, or 1 amino acid variation) as compared with the CDR-H1, CDR-H2, and CDR-H3 as shown in Table 9. “Collectively” means that the total number of amino acid variations in all of the three heavy chain CDRs is within the defined range. Alternatively or in addition (e.g., in addition), the transferrin receptor antibody of the present disclosure may comprise a CDR-L1, a CDR-L2, and a CDR-L3, which collectively contains no more than 5 amino acid variations (e.g., no more than 5, 4, 3, 2 or 1 amino acid variation) as compared with the CDR-L1, CDR-L2, and CDR-L3 as shown in Table 9.

In some embodiments, the transferrin receptor antibody of the present disclosure comprises a CDR-H1, a CDR-H2, and a CDR-H3, at least one of which contains no more than 3 amino acid variations (e.g., no more than 3, 2, or 1 amino acid variation) as compared with the counterpart heavy chain CDR as shown in Table 9. Alternatively or in addition (e.g., in addition), the transferrin receptor antibody of the present disclosure may comprise CDR-L1, a CDR-L2, and a CDR-L3, at least one of which contains no more than 3 amino acid variations (e.g., no more than 3, 2, or 1 amino acid variation) as compared with the counterpart light chain CDR as shown in Table 9.

In some embodiments, the transferrin receptor antibody of the present disclosure comprises a CDR-L3, which contains no more than 3 amino acid variations (e.g., no more than 3, 2, or 1 amino acid variation) as compared with the CDR-L3 as shown in Table 9. In some embodiments, the transferrin receptor antibody of the present disclosure comprises a CDR-L3 containing one amino acid variation as compared with the CDR-L3 as shown in Table 9. In some embodiments, the transferrin receptor antibody of the present disclosure comprises a CDR-L3 of QHFAGTPLT (SEQ ID NO: 232) according to the Kabat and Chothia definition system) or QHFAGTPL (SEQ ID NO: 233) according to the Contact definition system). In some embodiments, the transferrin receptor antibody of the present disclosure comprises a CDR-H1, a CDR-H2, a CDR-H3, a CDR-L1 and a CDR-L2 that are the same as the CDR-H1, CDR-H2, and CDR-H3 shown in Table 9, and comprises a CDR-L3 of QHFAGTPLT (SEQ ID NO: 232) according to the Kabat and Chothia definition system) or QHFAGTPL (SEQ ID NO: 233) according to the Contact definition system).

In some embodiments, the transferrin receptor antibody of the present disclosure comprises heavy chain CDRs that collectively are at least 80% (e.g., 80%, 85%, 90%, 95%, or 98%) identical to the heavy chain CDRs as shown in Table 9. Alternatively or in addition (e.g., in addition), the transferrin receptor antibody of the present disclosure comprises light chain CDRs that collectively are at least 80% (e.g., 80%, 85%, 90%, 95%, or 98%) identical to the light chain CDRs as shown in Table 9.

In some embodiments, the transferrin receptor antibody of the present disclosure comprises a VH comprising the amino acid sequence of SEQ ID NO: 230. Alternatively or in addition (e.g., in addition), the transferrin receptor antibody of the present disclosure comprises a VL comprising the amino acid sequence of SEQ ID NO: 231.

In some embodiments, the transferrin receptor antibody of the present disclosure comprises a VH containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 230. Alternatively or in addition (e.g., in addition), the transferrin receptor antibody of the present disclosure comprises a VL containing no more than 15 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 231.

In some embodiments, the transferrin receptor antibody of the present disclosure comprises a VH comprising an amino acid sequence that is at least 80% (e.g., 80%, 85%, 90%, 95%, or 98%) identical to the VH as set forth in SEQ ID NO: 230. Alternatively or in addition (e.g., in addition), the transferrin receptor antibody of the present disclosure comprises a VL comprising an amino acid sequence that is at least 80% (e.g., 80%, 85%, 90%, 95%, or 98%) identical to the VL as set forth in SEQ ID NO: 231.

In some embodiments, the transferrin receptor antibody of the present disclosure is a humanized antibody (e.g., a humanized variant of an antibody). In some embodiments, the transferrin receptor antibody of the present disclosure comprises a CDR-H1, a CDR-H2, a CDR-H3, a CDR-L1, a CDR-L2, and a CDR-L3 that are the same as the CDR-H1, CDR-H2, and CDR-H3 shown in Table 9, and comprises a humanized heavy chain variable region and/or (e.g., and) a humanized light chain variable region.

Humanized antibodies are human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity. In some embodiments, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in WO 99/58572. Other forms of humanized antibodies have one or more CDRs (one, two, three, four, five, six) which are altered with respect to the original antibody, which are also termed one or more CDRs derived from one or more CDRs from the original antibody. Humanized antibodies may also involve affinity maturation.

In some embodiments, humanization is achieved by grafting the CDRs (e.g., as shown in Table 9) into the IGKV1-NL1*01 and IGHV1-3*01 human variable domains. In some embodiments, the transferrin receptor antibody of the present disclosure is a humanized variant comprising one or more amino acid substitutions at positions 9, 13, 17, 18, 40, 45, and 70 as compared with the VL as set forth in SEQ ID NO: 231, and/or (e.g., and) one or more amino acid substitutions at positions 1, 5, 7, 11, 12, 20, 38, 40, 44, 66, 75, 81, 83, 87, and 108 as compared with the VH as set forth in SEQ ID NO: 230. In some embodiments, the transferrin receptor antibody of the present disclosure is a humanized variant comprising amino acid substitutions at all of positions 9, 13, 17, 18, 40, 45, and 70 as compared with the VL as set forth in SEQ ID NO: 231, and/or (e.g., and) amino acid substitutions at all of positions 1, 5, 7, 11, 12, 20, 38, 40, 44, 66, 75, 81, 83, 87, and 108 as compared with the VH as set forth in SEQ ID NO: 230.

In some embodiments, the transferrin receptor antibody of the present disclosure is a humanized antibody and contains the residues at positions 43 and 48 of the VL as set forth in SEQ ID NO: 231. Alternatively or in addition (e.g., in addition), the transferrin receptor antibody of the present disclosure is a humanized antibody and contains the residues at positions 48, 67, 69, 71, and 73 of the VH as set forth in SEQ ID NO: 230.

The VH and VL amino acid sequences of an example humanized antibody that may be used in accordance with the present disclosure are provided:

Humanized VH
(SEQ ID NO: 234)
EVQLVQSGAEVKKPGASVKVSCKASGYTFTSYWMHWVRQAPGQRLEWIG
EINPTNGRTNYIEKFKSRATLTVDKSASTAYMELSSLRSEDTAVYYCAR
GTRAYHYWGQGTMVTVSS
Humanized VL
(SEQ ID NO: 235)
DIQMTQSPSSLSASVGDRVTITCRASDNLYSNLAWYQQKPGKSPKLLVY
DATNLADGVPSRFSGSGSGTDYTLTISSLQPEDFATYYCQHFWGTPLTF
GQGTKVEIK

In some embodiments, the transferrin receptor antibody of the present disclosure comprises a VH comprising the amino acid sequence of SEQ ID NO: 234. Alternatively or in addition (e.g., in addition), the transferrin receptor antibody of the present disclosure comprises a VL comprising the amino acid sequence of SEQ ID NO: 235.

In some embodiments, the transferrin receptor antibody of the present disclosure comprises a VH containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 234. Alternatively or in addition (e.g., in addition), the transferrin receptor antibody of the present disclosure comprises a VL containing no more than 15 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 235.

In some embodiments, the transferrin receptor antibody of the present disclosure comprises a VH comprising an amino acid sequence that is at least 80% (e.g., 80%, 85%, 90%, 95%, or 98%) identical to the VH as set forth in SEQ ID NO: 234. Alternatively or in addition (e.g., in addition), the transferrin receptor antibody of the present disclosure comprises a VL comprising an amino acid sequence that is at least 80% (e.g., 80%, 85%, 90%, 95%, or 98%) identical to the VL as set forth in SEQ ID NO: 235.

In some embodiments, the transferrin receptor antibody of the present disclosure is a humanized variant comprising amino acid substitutions at one or more of positions 43 and 48 as compared with the VL as set forth in SEQ ID NO: 231, and/or (e.g., and) amino acid substitutions at one or more of positions 48, 67, 69, 71, and 73 as compared with the VH as set forth in SEQ ID NO: 230. In some embodiments, the transferrin receptor antibody of the present disclosure is a humanized variant comprising a S43A and/or (e.g., and) a V48L mutation as compared with the VL as set forth in SEQ ID NO: 231, and/or (e.g., and) one or more of A67V, L69I, V71R, and K73T mutations as compared with the VH as set forth in SEQ ID NO: 230

In some embodiments, the transferrin receptor antibody of the present disclosure is a humanized variant comprising amino acid substitutions at one or more of positions 9, 13, 17, 18, 40, 43, 48, 45, and 70 as compared with the VL as set forth in SEQ ID NO: 231, and/or (e.g., and) amino acid substitutions at one or more of positions 1, 5, 7, 11, 12, 20, 38, 40, 44, 48, 66, 67, 69, 71, 73, 75, 81, 83, 87, and 108 as compared with the VH as set forth in SEQ ID NO: 230.

In some embodiments, the transferrin receptor antibody of the present disclosure is a chimeric antibody, which can include a heavy constant region and a light constant region from a human antibody. Chimeric antibodies refer to antibodies having a variable region or part of variable region from a first species and a constant region from a second species. Typically, in these chimeric antibodies, the variable region of both light and heavy chains mimics the variable regions of antibodies derived from one species of mammals (e.g., a non-human mammal such as mouse, rabbit, and rat), while the constant portions are homologous to the sequences in antibodies derived from another mammal such as human. In some embodiments, amino acid modifications can be made in the variable region and/or (e.g., and) the constant region.

In some embodiments, the transferrin receptor antibody described herein is a chimeric antibody, which can include a heavy constant region and a light constant region from a human antibody. Chimeric antibodies refer to antibodies having a variable region or part of variable region from a first species and a constant region from a second species. Typically, in these chimeric antibodies, the variable region of both light and heavy chains mimics the variable regions of antibodies derived from one species of mammals (e.g., a non-human mammal such as mouse, rabbit, and rat), while the constant portions are homologous to the sequences in antibodies derived from another mammal such as human. In some embodiments, amino acid modifications can be made in the variable region and/or (e.g., and) the constant region.

In some embodiments, the heavy chain of any of the transferrin receptor antibodies as described herein may comprises a heavy chain constant region (CH) or a portion thereof (e.g., CH1, CH2, CH3, or a combination thereof). The heavy chain constant region can of any suitable origin, e.g., human, mouse, rat, or rabbit. In one specific example, the heavy chain constant region is from a human IgG (a gamma heavy chain), e.g., IgG1, IgG2, or IgG4. An example of a human IgG1 constant region is given below:

(SEQ ID NO: 175)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK

In some embodiments, the light chain of any of the transferrin receptor antibodies described herein may further comprise a light chain constant region (CL), which can be any CL known in the art. In some examples, the CL is a kappa light chain. In other examples, the CL is a lambda light chain. In some embodiments, the CL is a kappa light chain, the sequence of which is provided below:

(SEQ ID NO: 177)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC

Other antibody heavy and light chain constant regions are well known in the art, e.g., those provided in the IMGT database (www.imgt.org) or at www.vbase2.org/vbstat.php., both of which are incorporated by reference herein.

Examples of heavy chain and light chain amino acid sequences of the transferrin receptor antibodies described are provided below:

Heavy Chain (VH + human IgG1 constant region)
(SEQ ID NO: 236)
QVQLQQPGAELVKPGASVKLSCKASGYTFTSYWMHWVKQRPGQGLEWIGEINPTNG
RTNYIEKFKSKATLTVDKSSSTAYMQLSSLTSEDSAVYYCARGTRAYHYWGQGTSVT
VSSASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKKVEPKSCDKTHTCPPCPA
PELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPPVLDSDGSF
FLYSKLTVDKSRWQQGNVFSCSVMHEALHNHYTQKSLSLSPGK
Light Chain (VL + kappa light chain)
(SEQ ID NO: 237)
DIQMTQSPASLSVSVGETVTITCRASDNLYSNLAWYQQKQGKSPQLLVYDATNLADG
VPSRFSGSGSGTQYSLKINSLQSEDFGTYYCQHFWGTPLTFGAGTKLELKRTVAAPSVF
IFPPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC
Heavy Chain (humanized VH + human IgG1 constant region)
(SEQ ID NO: 238)
EVQLVQSGAEVKKPGASVKVSCKASGYTFTSYWMHWVRQAPGQRLEWIGEINPTNG
RTNYIEKFKSRATLTVDKSASTAYMELSSLRSEDTAVYYCARGTRAYHYWGQGTMV
TVSSASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKKVEPKSCDKTHTCPPCPA
PELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPPVLDSDGSF
FLYSKLTVDKSRWQQGNVFSCSVMHEALHNHYTQKSLSLSPGK
Light Chain (humanized VL + kappa light chain)
(SEQ ID NO: 239)
DIQMTQSPSSLSASVGDRVTITCRASDNLYSNLAWYQQKPGKSPKLLVYDATNLADG
VPSRFSGSGSGTDYTLTISSLQPEDFATYYCQHFWGTPLTFGQGTKVEIKRTVAAPSVFI
FPPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC

In some embodiments, the transferrin receptor antibody described herein comprises a heavy chain comprising an amino acid sequence that is at least 80% (e.g., 80%, 85%, 90%, 95%, or 98%) identical to SEQ ID NO: 236. Alternatively or in addition (e.g., in addition), the transferrin receptor antibody described herein comprises a light chain comprising an amino acid sequence that is at least 80% (e.g., 80%, 85%, 90%, 95%, or 98%) identical to SEQ ID NO: 237. In some embodiments, the transferrin receptor antibody described herein comprises a heavy chain comprising the amino acid sequence of SEQ ID NO: 236. Alternatively or in addition (e.g., in addition), the transferrin receptor antibody described herein comprises a light chain comprising the amino acid sequence of SEQ ID NO: 237.

In some embodiments, the transferrin receptor antibody of the present disclosure comprises a heavy chain containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the heavy chain as set forth in SEQ ID NO: 236. Alternatively or in addition (e.g., in addition), the transferrin receptor antibody of the present disclosure comprises a light chain containing no more than 15 amino acid variations (e.g., no more than 20, 19, 18, 17, 16, 15, 14, 13, 12, 11, 9, 8, 7, 6, 5, 4, 3, 2, or 1 amino acid variation) as compared with the light chain as set forth in SEQ ID NO: 237.

In some embodiments, the transferrin receptor antibody described herein comprises a heavy chain comprising an amino acid sequence that is at least 80% (e.g., 80%, 85%, 90%, 95%, or 98%) identical to SEQ ID NO: 238. Alternatively or in addition (e.g., in addition), the transferrin receptor antibody described herein comprises a light chain comprising an amino acid sequence that is at least 80% (e.g., 80%, 85%, 90%, 95%, or 98%) identical to SEQ ID NO: 239. In some embodiments, the transferrin receptor antibody described herein comprises a heavy chain comprising the amino acid sequence of SEQ ID NO: 238. Alternatively or in addition (e.g., in addition), the transferrin receptor antibody described herein comprises a light chain comprising the amino acid sequence of SEQ ID NO: 239.

In some embodiments, the transferrin receptor antibody of the present disclosure comprises a heavy chain containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the heavy chain of humanized antibody as set forth in SEQ ID NO: 238. Alternatively or in addition (e.g., in addition), the transferrin receptor antibody of the present disclosure comprises a light chain containing no more than 15 amino acid variations (e.g., no more than 20, 19, 18, 17, 16, 15, 14, 13, 12, 11, 9, 8, 7, 6, 5, 4, 3, 2, or 1 amino acid variation) as compared with the light chain of humanized antibody as set forth in SEQ ID NO: 239.

In some embodiments, the transferrin receptor antibody is an antigen binding fragment (FAB) of an intact antibody (full-length antibody). Antigen binding fragment of an intact antibody (full-length antibody) can be prepared via routine methods. For example, F(ab′)2 fragments can be produced by pepsin digestion of an antibody molecule, and Fab fragments that can be generated by reducing the disulfide bridges of F(ab′)2 fragments. Examples of FABs amino acid sequences of the transferrin receptor antibodies described herein are provided below:

Heavy Chain FAB (VH + a portion of human IgG1
constant region)
(SEQ ID NO: 240)
QVQLQQPGAELVKPGASVKLSCKASGYTFTSYWMHWVKQRPGQGLEWIG
EINPTNGRTNYIEKFKSKATLTVDKSSSTAYMQLSSLTSEDSAVYYCAR
GTRAYHYWGQGTSVTVSSASTKGPSVFPLAPSSKSTSGGTAALGCLVKD
YFPEPVTVSWNSGALTSGVHTFPAVLQSSGLYSLSSVVTVPSSSLGTQT
YICNVNHKPSNTKVDKKVEPKSCDKTHTCP
Heavy Chain FAB (humanized VH + a portion of
human IgG1 constant region)
(SEQ ID NO: 241)
EVQLVQSGAEVKKPGASVKVSCKASGYTFTSYWMHWVRQAPGQRLEWIG
EINPTNGRTNYIEKFKSRATLTVDKSASTAYMELSSLRSEDTAVYYCAR
GTRAYHYWGQGTMVTVSSASTKGPSVFPLAPSSKSTSGGTAALGCLVKD
YFPEPVTVSWNSGALTSGVHTFPAVLQSSGLYSLSSVVTVPSSSLGTQT
YICNVNHKPSNTKVDKKVEPKSCDKTHTCP

In some embodiments, the transferrin receptor antibody described herein comprises a heavy chain comprising the amino acid sequence of SEQ ID NO: 240. Alternatively or in addition (e.g., in addition), the transferrin receptor antibody described herein comprises a light chain comprising the amino acid sequence of SEQ ID NO: 237.

In some embodiments, the transferrin receptor antibody described herein comprises a heavy chain comprising the amino acid sequence of SEQ ID NO: 241. Alternatively or in addition (e.g., in addition), the transferrin receptor antibody described herein comprises a light chain comprising the amino acid sequence of SEQ ID NO: 239.

The transferrin receptor antibodies described herein can be in any antibody form, including, but not limited to, intact (i.e., full-length) antibodies, antigen-binding fragments thereof (such as Fab, Fab′, F(ab′)2, Fv), single chain antibodies, bi-specific antibodies, or nanobodies. In some embodiments, the transferrin receptor antibody described herein is a scFv. In some embodiments, the transferrin receptor antibody described herein is a scFv-Fab (e.g., scFv fused to a portion of a constant region). In some embodiments, the transferrin receptor antibody described herein is a scFv fused to a constant region (e.g., human IgG1 constant region as set forth in SEQ ID NO: 175).

b. Other Muscle-Targeting Antibodies

In some embodiments, the muscle-targeting antibody is an antibody that specifically binds hemojuvelin, caveolin-3, Duchenne muscular dystrophy peptide, myosin Iib or CD63. In some embodiments, the muscle-targeting antibody is an antibody that specifically binds a myogenic precursor protein. Exemplary myogenic precursor proteins include, without limitation, ABCG2, M-Cadherin/Cadherin-15, Caveolin-1, CD34, FoxK1, Integrin alpha 7, Integrin alpha 7 beta 1, MYF-5, MyoD, Myogenin, NCAM-1/CD56, Pax3, Pax7, and Pax9. In some embodiments, the muscle-targeting antibody is an antibody that specifically binds a skeletal muscle protein. Exemplary skeletal muscle proteins include, without limitation, alpha-Sarcoglycan, beta-Sarcoglycan, Calpain Inhibitors, Creatine Kinase MM/CKMM, eIF5A, Enolase 2/Neuron-specific Enolase, epsilon-Sarcoglycan, FABP3/H-FABP, GDF-8/Myostatin, GDF-11/GDF-8, Integrin alpha 7, Integrin alpha 7 beta 1, Integrin beta 1/CD29, MCAM/CD146, MyoD, Myogenin, Myosin Light Chain Kinase Inhibitors, NCAM-1/CD56, and Troponin I. In some embodiments, the muscle-targeting antibody is an antibody that specifically binds a smooth muscle protein. Exemplary smooth muscle proteins include, without limitation, alpha-Smooth Muscle Actin, VE-Cadherin, Caldesmon/CALD1, Calponin 1, Desmin, Histamine H2 R, Motilin R/GPR38, Transgelin/TAGLN, and Vimentin. However, it should be appreciated that antibodies to additional targets are within the scope of this disclosure and the exemplary lists of targets provided herein are not meant to be limiting.

c. Antibody Features/Alterations

In some embodiments, conservative mutations can be introduced into antibody sequences (e.g., CDRs or framework sequences) at positions where the residues are not likely to be involved in interacting with a target antigen (e.g., transferrin receptor), for example, as determined based on a crystal structure. In some embodiments, one, two or more mutations (e.g., amino acid substitutions) are introduced into the Fc region of a muscle-targeting antibody described herein (e.g., in a CH2 domain (residues 231-340 of human IgG1) and/or (e.g., and) CH3 domain (residues 341-447 of human IgG1) and/or (e.g., and) the hinge region, with numbering according to the Kabat numbering system (e.g., the EU index in Kabat)) to alter one or more functional properties of the antibody, such as serum half-life, complement fixation, Fc receptor binding and/or (e.g., and) antigen-dependent cellular cytotoxicity.

In some embodiments, one, two or more mutations (e.g., amino acid substitutions) are introduced into the hinge region of the Fc region (CH1 domain) such that the number of cysteine residues in the hinge region are altered (e.g., increased or decreased) as described in, e.g., U.S. Pat. No. 5,677,425. The number of cysteine residues in the hinge region of the CH1 domain can be altered to, e.g., facilitate assembly of the light and heavy chains, or to alter (e.g., increase or decrease) the stability of the antibody or to facilitate linker conjugation.

In some embodiments, one, two or more mutations (e.g., amino acid substitutions) are introduced into the Fc region of a muscle-targeting antibody described herein (e.g., in a CH2 domain (residues 231-340 of human IgG1) and/or (e.g., and) CH3 domain (residues 341-447 of human IgG1) and/or (e.g., and) the hinge region, with numbering according to the Kabat numbering system (e.g., the EU index in Kabat)) to increase or decrease the affinity of the antibody for an Fc receptor (e.g., an activated Fc receptor) on the surface of an effector cell. Mutations in the Fc region of an antibody that decrease or increase the affinity of an antibody for an Fc receptor and techniques for introducing such mutations into the Fc receptor or fragment thereof are known to one of skill in the art. Examples of mutations in the Fc receptor of an antibody that can be made to alter the affinity of the antibody for an Fc receptor are described in, e.g., Smith P et al., (2012) PNAS 109: 6181-6186, U.S. Pat. No. 6,737,056, and International Publication Nos. WO 02/060919; WO 98/23289; and WO 97/34631, which are incorporated herein by reference.

In some embodiments, one, two or more amino acid mutations (i.e., substitutions, insertions or deletions) are introduced into an IgG constant domain, or FcRn-binding fragment thereof (preferably an Fc or hinge-Fc domain fragment) to alter (e.g., decrease or increase) half-life of the antibody in vivo. See, e.g., International Publication Nos. WO 02/060919; WO 98/23289; and WO 97/34631; and U.S. Pat. Nos. 5,869,046, 6,121,022, 6,277,375 and 6,165,745 for examples of mutations that will alter (e.g., decrease or increase) the half-life of an antibody in vivo.

In some embodiments, one, two or more amino acid mutations (i.e., substitutions, insertions or deletions) are introduced into an IgG constant domain, or FcRn-binding fragment thereof (preferably an Fc or hinge-Fc domain fragment) to decrease the half-life of the anti-transferrin receptor antibody in vivo. In some embodiments, one, two or more amino acid mutations (i.e., substitutions, insertions or deletions) are introduced into an IgG constant domain, or FcRn-binding fragment thereof (preferably an Fc or hinge-Fc domain fragment) to increase the half-life of the antibody in vivo. In some embodiments, the antibodies can have one or more amino acid mutations (e.g., substitutions) in the second constant (CH2) domain (residues 231-340 of human IgG1) and/or (e.g., and) the third constant (CH3) domain (residues 341-447 of human IgG1), with numbering according to the EU index in Kabat (Kabat E A et al., (1991) supra). In some embodiments, the constant region of the IgG1 of an antibody described herein comprises a methionine (M) to tyrosine (Y) substitution in position 252, a serine (S) to threonine (T) substitution in position 254, and a threonine (T) to glutamic acid (E) substitution in position 256, numbered according to the EU index as in Kabat. See U.S. Pat. No. 7,658,921, which is incorporated herein by reference. This type of mutant IgG, referred to as “YTE mutant” has been shown to display fourfold increased half-life as compared to wild-type versions of the same antibody (see Dall'Acqua W F et al., (2006) J Biol Chem 281: 23514-24). In some embodiments, an antibody comprises an IgG constant domain comprising one, two, three or more amino acid substitutions of amino acid residues at positions 251-257, 285-290, 308-314, 385-389, and 428-436, numbered according to the EU index as in Kabat.

In some embodiments, one, two or more amino acid substitutions are introduced into an IgG constant domain Fc region to alter the effector function(s) of the anti-transferrin receptor antibody. The effector ligand to which affinity is altered can be, for example, an Fc receptor or the C1 component of complement. This approach is described in further detail in U.S. Pat. Nos. 5,624,821 and 5,648,260. In some embodiments, the deletion or inactivation (through point mutations or other means) of a constant region domain can reduce Fc receptor binding of the circulating antibody thereby increasing tumor localization. See, e.g., U.S. Pat. Nos. 5,585,097 and 8,591,886 for a description of mutations that delete or inactivate the constant domain and thereby increase tumor localization. In some embodiments, one or more amino acid substitutions may be introduced into the Fc region of an antibody described herein to remove potential glycosylation sites on Fc region, which may reduce Fc receptor binding (see, e.g., Shields R L et al., (2001) J Biol Chem 276: 6591-604).

In some embodiments, one or more amino in the constant region of a muscle-targeting antibody described herein can be replaced with a different amino acid residue such that the antibody has altered C1q binding and/or (e.g., and) reduced or abolished complement dependent cytotoxicity (CDC). This approach is described in further detail in U.S. Pat. No. 6,194,551 (Idusogie et al). In some embodiments, one or more amino acid residues in the N-terminal region of the CH2 domain of an antibody described herein are altered to thereby alter the ability of the antibody to fix complement. This approach is described further in International Publication No. WO 94/29351. In some embodiments, the Fc region of an antibody described herein is modified to increase the ability of the antibody to mediate antibody dependent cellular cytotoxicity (ADCC) and/or (e.g., and) to increase the affinity of the antibody for an Fey receptor. This approach is described further in International Publication No. WO 00/42072.

In some embodiments, the heavy and/or (e.g., and) light chain variable domain(s) sequence(s) of the antibodies provided herein can be used to generate, for example, CDR-grafted, chimeric, humanized, or composite human antibodies or antigen-binding fragments, as described elsewhere herein. As understood by one of ordinary skill in the art, any variant, CDR-grafted, chimeric, humanized, or composite antibodies derived from any of the antibodies provided herein may be useful in the compositions and methods described herein and will maintain the ability to specifically bind transferrin receptor, such that the variant, CDR-grafted, chimeric, humanized, or composite antibody has at least 50%, at least 60%, at least 70%, at least 80%, at least 90%, at least 95% or more binding to transferrin receptor relative to the original antibody from which it is derived.

In some embodiments, the antibodies provided herein comprise mutations that confer desirable properties to the antibodies. For example, to avoid potential complications due to Fab-arm exchange, which is known to occur with native IgG4 mAbs, the antibodies provided herein may comprise a stabilizing ‘Adair’ mutation (Angal S., et al., “A single amino acid substitution abolishes the heterogeneity of chimeric mouse/human (IgG4) antibody,” Mol Immunol 30, 105-108; 1993), where serine 228 (EU numbering; residue 241 Kabat numbering) is converted to proline resulting in an IgG1-like hinge sequence. Accordingly, any of the antibodies may include a stabilizing ‘Adair’ mutation.

As provided herein, antibodies of this disclosure may optionally comprise constant regions or parts thereof. For example, a VL domain may be attached at its C-terminal end to a light chain constant domain like C_{κ} or C_{λ}. Similarly, a VH domain or portion thereof may be attached to all or part of a heavy chain like IgA, IgD, IgE, IgG, and IgM, and any isotype subclass. Antibodies may include suitable constant regions (see, for example, Kabat et al., Sequences of Proteins of Immunological Interest, No. 91-3242, National Institutes of Health Publications, Bethesda, Md. (1991)). Therefore, antibodies within the scope of this may disclosure include VH and VL domains, or an antigen binding portion thereof, combined with any suitable constant regions.

In some embodiments, the anti-TfR antibody of the present disclosure is a humanized antibody comprising human framework regions with the CDRs of a murine antibody listed in Table 2 or Table 4 (e.g., 3A4, 3M12, or 5H12). In some embodiments, the anti-TfR antibody of the present disclosure is an IgG1 kappa that comprises human framework regions with the CDRs of a murine antibody listed in Table 2 or Table 4 (e.g., 3A4, 3M12, or 5H12). In some embodiments, the anti-TfR antibody of the present disclosure is a Fab′ fragment of an IgG1 kappa that comprises human framework regions with the CDRs of a murine antibody listed in Table 1 or Table 3 (e.g., 3A4, 3M12, or 5H12). In some embodiments, the anti-TfR antibody of the present disclose comprises the CDRs of the antibody provided in Table 7. In some embodiments, the anti-TfR antibody of the present disclosure is an IgG1 kappa that comprises the variable regions of the antibody provided in Table 7. In some embodiments, the anti-TfR antibody of the present disclosure is a Fab′ fragment of an IgG1 kappa that comprises the variable regions of the antibody provided in Table 7.

In some embodiments, any one of the anti-TfR antibodies described herein is produced by recombinant DNA technology in Chinese hamster ovary (CHO) cell suspension culture, optionally in CHO-K1 cell (e.g., CHO-K1 cells derived from European Collection of Animal Cell Culture, Cat. No. 85051005) suspension culture.

In some embodiments, an antibody provided herein may have one or more post-translational modifications. In some embodiments, N-terminal cyclization, also called pyroglutamate formation (pyro-Glu), may occur in the antibody at N-terminal Glutamate (Glu) and/or Glutamine (Gln) residues during production. In some embodiments, pyroglutamate formation occurs in a heavy chain sequence. In some embodiments, pyroglutamate formation occurs in a light chain sequence.

ii. Muscle-Targeting Peptides

Some aspects of the disclosure provide muscle-targeting peptides as muscle-targeting agents. Short peptide sequences (e.g., peptide sequences of 5-20 amino acids in length) that bind to specific cell types have been described. For example, cell-targeting peptides have been described in Vines e., et al., A. “Cell-penetrating and cell-targeting peptides in drug delivery” Biochim Biophys Acta 2008, 1786: 126-38; Jarver P., et al., “In vivo biodistribution and efficacy of peptide mediated delivery” Trends Pharmacol Sci 2010; 31: 528-35; Samoylova T. I., et al., “Elucidation of muscle-binding peptides by phage display screening” Muscle Nerve 1999; 22: 460-6; U.S. Pat. No. 6,329,501, issued on Dec. 11, 2001, entitled “METHODS AND COMPOSITIONS FOR TARGETING COMPOUNDS TO MUSCLE”; and Samoylov A.M., et al., “Recognition of cell-specific binding of phage display derived peptides using an acoustic wave sensor.” Biomol Eng 2002; 18: 269-72; the entire contents of each of which are incorporated herein by reference. By designing peptides to interact with specific cell surface antigens (e.g., receptors), selectivity for a desired tissue, e.g., muscle, can be achieved. Skeletal muscle-targeting has been investigated and a range of molecular payloads are able to be delivered. These approaches may have high selectivity for muscle tissue without many of the practical disadvantages of a large antibody or viral particle. Accordingly, in some embodiments, the muscle-targeting agent is a muscle-targeting peptide that is from 4 to 50 amino acids in length. In some embodiments, the muscle-targeting peptide is 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length. Muscle-targeting peptides can be generated using any of several methods, such as phage display.

In some embodiments, a muscle-targeting peptide may bind to an internalizing cell surface receptor that is overexpressed or relatively highly expressed in muscle cells, e.g. a transferrin receptor, compared with certain other cells. In some embodiments, a muscle-targeting peptide may target, e.g., bind to, a transferrin receptor. In some embodiments, a peptide that targets a transferrin receptor may comprise a segment of a naturally occurring ligand, e.g., transferrin. In some embodiments, a peptide that targets a transferrin receptor is as described in U.S. Pat. No. 6,743,893, filed Nov. 30, 2000, “RECEPTOR-MEDIATED UPTAKE OF PEPTIDES THAT BIND THE HUMAN TRANSFERRIN RECEPTOR”. In some embodiments, a peptide that targets a transferrin receptor is as described in Kawamoto, M. et al, “A novel transferrin receptor-targeted hybrid peptide disintegrates cancer cell membrane to induce rapid killing of cancer cells.” BMC Cancer. 2011 Aug. 18; 11:359. In some embodiments, a peptide that targets a transferrin receptor is as described in U.S. Pat. No. 8,399,653, filed May 20, 2011, “TRANSFERRIN/TRANSFERRIN RECEPTOR-MEDIATED SIRNA DELIVERY”.

As discussed above, examples of muscle targeting peptides have been reported. For example, muscle-specific peptides were identified using phage display library presenting surface heptapeptides. As one example a peptide having the amino acid sequence ASSLNIA (SEQ ID NO: 248) bound to C2C12 murine myotubes in vitro, and bound to mouse muscle tissue in vivo. Accordingly, in some embodiments, the muscle-targeting agent comprises the amino acid sequence ASSLNIA (SEQ ID NO: 248). This peptide displayed improved specificity for binding to heart and skeletal muscle tissue after intravenous injection in mice with reduced binding to liver, kidney, and brain. Additional muscle-specific peptides have been identified using phage display. For example, a 12 amino acid peptide was identified by phage display library for muscle targeting in the context of treatment for DMD. See, Yoshida D., et al., “Targeting of salicylate to skin and muscle following topical injections in rats.” Int J Pharm 2002; 231: 177-84; the entire contents of which are hereby incorporated by reference. Here, a 12 amino acid peptide having the sequence SKTFNTHPQSTP (SEQ ID NO: 249) was identified and this muscle-targeting peptide showed improved binding to C2C12 cells relative to the ASSLNIA (SEQ ID NO: 248) peptide.

An additional method for identifying peptides selective for muscle (e.g., skeletal muscle) over other cell types includes in vitro selection, which has been described in Ghosh D., et al., “Selection of muscle-binding peptides from context-specific peptide-presenting phage libraries for adenoviral vector targeting” J Virol 2005; 79: 13667-72; the entire contents of which are incorporated herein by reference. By pre-incubating a random 12-mer peptide phage display library with a mixture of non-muscle cell types, non-specific cell binders were selected out. Following rounds of selection the 12 amino acid peptide TARGEHKEEELI (SEQ ID NO: 250) appeared most frequently. Accordingly, in some embodiments, the muscle-targeting agent comprises the amino acid sequence TARGEHKEEELI (SEQ ID NO: 250).

A muscle-targeting agent may an amino acid-containing molecule or peptide. A muscle-targeting peptide may correspond to a sequence of a protein that preferentially binds to a protein receptor found in muscle cells. In some embodiments, a muscle-targeting peptide contains a high propensity of hydrophobic amino acids, e.g. valine, such that the peptide preferentially targets muscle cells. In some embodiments, a muscle-targeting peptide has not been previously characterized or disclosed. These peptides may be conceived of, produced, synthesized, and/or (e.g., and) derivatized using any of several methodologies, e.g. phage displayed peptide libraries, one-bead one-compound peptide libraries, or positional scanning synthetic peptide combinatorial libraries. Exemplary methodologies have been characterized in the art and are incorporated by reference (Gray, B. P. and Brown, K. C. “Combinatorial Peptide Libraries: Mining for Cell-Binding Peptides” Chem Rev. 2014, 114:2, 1020-1081.; Samoylova, T. I. and Smith, B. F. “Elucidation of muscle-binding peptides by phage display screening.” Muscle Nerve, 1999, 22:4. 460-6.). In some embodiments, a muscle-targeting peptide has been previously disclosed (see, e.g. Writer M. J. et al. “Targeted gene delivery to human airway epithelial cells with synthetic vectors incorporating novel targeting peptides selected by phage display.” J. Drug Targeting. 2004; 12:185; Cal, D. “BDNF-mediated enhancement of inflammation and injury in the aging heart.” Physiol Genomics. 2006, 24:3, 191-7.; Zhang, L. “Molecular profiling of heart endothelial cells.” Circulation, 2005, 112:11, 1601-11.; McGuire, M. J. et al. “In vitro selection of a peptide with high selectivity for cardiomyocytes in vivo.” J Mol Biol. 2004, 342:1, 171-82.). Exemplary muscle-targeting peptides comprise an amino acid sequence of the following group: CQAQGQLVC (SEQ ID NO: 251), CSERSMNFC (SEQ ID NO: 252), CPKTRRVPC (SEQ ID NO: 253), WLSEAGPVVTVRALRGTGSW (SEQ ID NO: 254), ASSLNIA (SEQ ID NO: 248), CMQHSMRVC (SEQ ID NO: 255), and DDTRHWG (SEQ ID NO: 256). In some embodiments, a muscle-targeting peptide may comprise about 2-25 amino acids, about 2-20 amino acids, about 2-15 amino acids, about 2-10 amino acids, or about 2-5 amino acids. Muscle-targeting peptides may comprise naturally-occurring amino acids, e.g. cysteine, alanine, or non-naturally-occurring or modified amino acids. Non-naturally occurring amino acids include (3-amino acids, homo-amino acids, proline derivatives, 3-substituted alanine derivatives, linear core amino acids, N-methyl amino acids, and others known in the art. In some embodiments, a muscle-targeting peptide may be linear; in other embodiments, a muscle-targeting peptide may be cyclic, e.g. bicyclic (see, e.g. Silvana, M. G. et al. Mol. Therapy, 2018, 26:1, 132-147.).

iii. Muscle-Targeting Receptor Ligands

A muscle-targeting agent may be a ligand, e.g. a ligand that binds to a receptor protein. A muscle-targeting ligand may be a protein, e.g. transferrin, which binds to an internalizing cell surface receptor expressed by a muscle cell. Accordingly, in some embodiments, the muscle-targeting agent is transferrin, or a derivative thereof that binds to a transferrin receptor. A muscle-targeting ligand may alternatively be a small molecule, e.g. a lipophilic small molecule that preferentially targets muscle cells relative to other cell types. Exemplary lipophilic small molecules that may target muscle cells include compounds comprising cholesterol, cholesteryl, stearic acid, palmitic acid, oleic acid, oleyl, linolene, linoleic acid, myristic acid, sterols, dihydrotestosterone, testosterone derivatives, glycerine, alkyl chains, trityl groups, and alkoxy acids.

iv. Muscle-Targeting Aptamers

A muscle-targeting agent may be an aptamer, e.g. an RNA aptamer, which preferentially targets muscle cells relative to other cell types. In some embodiments, a muscle-targeting aptamer has not been previously characterized or disclosed. These aptamers may be conceived of, produced, synthesized, and/or (e.g., and) derivatized using any of several methodologies, e.g. Systematic Evolution of Ligands by Exponential Enrichment. Exemplary methodologies have been characterized in the art and are incorporated by reference (Yan, A.C. and Levy, M. “Aptamers and aptamer targeted delivery” RNA biology, 2009, 6:3, 316-20.; Germer, K. et al. “RNA aptamers and their therapeutic and diagnostic applications.” Int. J. Biochem. Mol. Biol. 2013; 4: 27-40.). In some embodiments, a muscle-targeting aptamer has been previously disclosed (see, e.g. Phillippou, S. et al. “Selection and Identification of Skeletal-Muscle-Targeted RNA Aptamers.” Mol Ther Nucleic Acids. 2018, 10:199-214.; Thiel, W.H. et al. “Smooth Muscle Cell-targeted RNA Aptamer Inhibits Neointimal Formation.” Mol Ther. 2016, 24:4, 779-87.). Exemplary muscle-targeting aptamers include the A01B RNA aptamer and RNA Apt 14. In some embodiments, an aptamer is a nucleic acid-based aptamer, an oligonucleotide aptamer or a peptide aptamer. In some embodiments, an aptamer may be about 5-15 kDa, about 5-10 kDa, about 10-15 kDa, about 1-5 Da, about 1-3 kDa, or smaller.

v. Other Muscle-Targeting Agents

One strategy for targeting a muscle cell (e.g., a skeletal muscle cell) is to use a substrate of a muscle transporter protein, such as a transporter protein expressed on the sarcolemma. In some embodiments, the muscle-targeting agent is a substrate of an influx transporter that is specific to muscle tissue. In some embodiments, the influx transporter is specific to skeletal muscle tissue. Two main classes of transporters are expressed on the skeletal muscle sarcolemma, (1) the adenosine triphosphate (ATP) binding cassette (ABC) superfamily, which facilitate efflux from skeletal muscle tissue and (2) the solute carrier (SLC) superfamily, which can facilitate the influx of substrates into skeletal muscle. In some embodiments, the muscle-targeting agent is a substrate that binds to an ABC superfamily or an SLC superfamily of transporters. In some embodiments, the substrate that binds to the ABC or SLC superfamily of transporters is a naturally-occurring substrate. In some embodiments, the substrate that binds to the ABC or SLC superfamily of transporters is a non-naturally occurring substrate, for example, a synthetic derivative thereof that binds to the ABC or SLC superfamily of transporters.

In some embodiments, the muscle-targeting agent is a substrate of an SLC superfamily of transporters. SLC transporters are either equilibrative or use proton or sodium ion gradients created across the membrane to drive transport of substrates. Exemplary SLC transporters that have high skeletal muscle expression include, without limitation, the SATT transporter (ASCT1; SLC1A4), GLUT4 transporter (SLC2A4), GLUT7 transporter (GLUT7; SLC2A7), ATRC2 transporter (CAT-2; SLC7A2), LAT3 transporter (KIAA0245; SLC7A6), PHT1 transporter (PTR4; SLC15A4), OATP-J transporter (OATP5A1; SLC21A15), OCT3 transporter (EMT; SLC22A3), OCTN2 transporter (FLJ46769; SLC22A5), ENT transporters (ENT1; SLC29A1 and ENT2; SLC29A2), PAT2 transporter (SLC36A2), and SAT2 transporter (KIAA1382; SLC38A2). These transporters can facilitate the influx of substrates into skeletal muscle, providing opportunities for muscle targeting.

In some embodiments, the muscle-targeting agent is a substrate of an equilibrative nucleoside transporter 2 (ENT2) transporter. Relative to other transporters, ENT2 has one of the highest mRNA expressions in skeletal muscle. While human ENT2 (hENT2) is expressed in most body organs such as brain, heart, placenta, thymus, pancreas, prostate, and kidney, it is especially abundant in skeletal muscle. Human ENT2 facilitates the uptake of its substrates depending on their concentration gradient. ENT2 plays a role in maintaining nucleoside homeostasis by transporting a wide range of purine and pyrimidine nucleobases. The hENT2 transporter has a low affinity for all nucleosides (adenosine, guanosine, uridine, thymidine, and cytidine) except for inosine. Accordingly, in some embodiments, the muscle-targeting agent is an ENT2 substrate. Exemplary ENT2 substrates include, without limitation, inosine, 2′,3′-dideoxyinosine, and calofarabine. In some embodiments, any of the muscle-targeting agents provided herein are associated with a molecular payload (e.g., oligonucleotide payload). In some embodiments, the muscle-targeting agent is covalently linked to the molecular payload. In some embodiments, the muscle-targeting agent is non-covalently linked to the molecular payload.

In some embodiments, the muscle-targeting agent is a substrate of an organic cation/carnitine transporter (OCTN2), which is a sodium ion-dependent, high affinity carnitine transporter. In some embodiments, the muscle-targeting agent is carnitine, mildronate, acetylcarnitine, or any derivative thereof that binds to OCTN2. In some embodiments, the carnitine, mildronate, acetylcarnitine, or derivative thereof is covalently linked to the molecular payload (e.g., oligonucleotide payload).

A muscle-targeting agent may be a protein that is protein that exists in at least one soluble form that targets muscle cells. In some embodiments, a muscle-targeting protein may be hemojuvelin (also known as repulsive guidance molecule C or hemochromatosis type 2 protein), a protein involved in iron overload and homeostasis. In some embodiments, hemojuvelin may be full length or a fragment, or a mutant with at least 75%, at least 80%, at least 85%, at least 90%, at least 95%, at least 98% or at least 99% sequence identity to a functional hemojuvelin protein. In some embodiments, a hemojuvelin mutant may be a soluble fragment, may lack a N-terminal signaling, and/or (e.g., and) lack a C-terminal anchoring domain. In some embodiments, hemojuvelin may be annotated under GenBank RefSeq Accession Numbers NM_001316767.1, NM_145277.4, NM_202004.3, NM_213652.3, or NM_213653.3. It should be appreciated that a hemojuvelin may be of human, non-human primate, or rodent origin.

B. Molecular Payloads

Some aspects of the disclosure provide molecular payloads, e.g., for modulating a biological outcome, e.g., the transcription of a DNA sequence, the expression of a protein, or the activity of a protein. In some embodiments, a molecular payload is linked to, or otherwise associated with a muscle-targeting agent. In some embodiments, such molecular payloads are capable of targeting to a muscle cell, e.g., via specifically binding to a nucleic acid or protein in the muscle cell following delivery to the muscle cell by an associated muscle-targeting agent. It should be appreciated that various types of muscle-targeting agents may be used in accordance with the disclosure. For example, the molecular payload may comprise, or consist of, an oligonucleotide (e.g., antisense oligonucleotide), a peptide (e.g., a peptide that binds a nucleic acid or protein associated with disease in a muscle cell), a protein (e.g., a protein that binds a nucleic acid or protein associated with disease in a muscle cell), or a small molecule (e.g., a small molecule that modulates the function of a nucleic acid or protein associated with disease in a muscle cell). In some embodiments, the molecular payload is an oligonucleotide that comprises a strand having a region of complementarity to a gene provided in Table 1. Exemplary molecular payloads are described in further detail herein, however, it should be appreciated that the exemplary molecular payloads provided herein are not meant to be limiting.

In some embodiments at least one (e.g., at least 2, at least 3, at least 4) molecular payload (e.g., oligonucleotide) is linked to a muscle-targeting agent. In some embodiments, all molecular payloads linked to a muscle-targeting agent are the same. In some embodiments, all molecular payloads linked to a muscle-targeting agent are different, for example the molecular payloads may target different portions of the same target gene, or the molecular payloads may target at least two different target genes. In some embodiments, a muscle-targeting agent may be attached to some molecular payloads that are the same and some molecular payloads that are different.

The present disclosure also provides a composition comprising a plurality of complexes, for which at least 80% (e.g., at least 85%, at least 90%, at least 91%, at least 92%, at least 93%, at least 94%, at least 95%, at least 96%, at least 97%, at least 98%, or at least 99%) of the complexes comprise a muscle-targeting agent linked to the same number of molecular payloads (e.g., oligonucleotides).

i. Oligonucleotides

Any suitable oligonucleotide may be used as a molecular payload, as described herein. In some embodiments, the oligonucleotide may be designed to cause degradation of an mRNA (e.g., the oligonucleotide may be a gapmer, an siRNA, a ribozyme or an aptamer that causes degradation). In some embodiments, the oligonucleotide may be designed to block translation of an mRNA (e.g., the oligonucleotide may be a mixmer, an siRNA or an aptamer that blocks translation). In some embodiments, an oligonucleotide may be designed to caused degradation and block translation of an mRNA. In some embodiments, an oligonucleotide may be a guide nucleic acid (e.g., guide RNA) for directing activity of an enzyme (e.g., a gene editing enzyme). Other examples of oligonucleotides are provided herein. It should be appreciated that, in some embodiments, oligonucleotides in one format (e.g., antisense oligonucleotides) may be suitably adapted to another format (e.g., siRNA oligonucleotides) by incorporating functional sequences (e.g., antisense strand sequences) from one format to the other format.

In some embodiments, an oligonucleotide may comprise a region of complementarity to a pro-atrophy gene provided in Table 1. In some embodiments, the oligonucleotide is configured to inhibit expression of the pro-atrophy gene, e.g., through RNAse H or RNAi mediated mRNA degradation. However, in some embodiments, the oligonucleotide is configured to block translation of the mRNA or promote alternative splicing and/or (e.g., and) exon skipping that leads to unstable mRNAs and decreased protein expression. Other examples of oligonucleotides to inhibit pro-atrophy gene expression are provided herein.

In other embodiments, the oligonucleotide encode guide nucleic acids that direct nuclease activity, e.g., as disclosed in Wei Y, et al., Prevention of Muscle Wasting by CRISPR/Cas9-mediated Disruption of Myostatin In Vivo Volume 24, Issue 11, p 1889-1891, November 2016, the contents of which are incorporated herein by reference.

In some embodiments, any one of the oligonucleotides can be in salt form, e.g., as sodium, potassium, or magnesium salts.

In some embodiments, the 5′ or 3′ nucleoside (e.g., terminal nucleoside) of any one of the oligonucleotides described herein is conjugated to an amine group, optionally via a spacer. In some embodiments, the spacer comprises an aliphatic moiety. In some embodiments, the spacer comprises a polyethylene glycol moiety. In some embodiments, a phosphodiester linkage is present between the spacer and the 5′ or 3′ nucleoside of the oligonucleotide. In some embodiments, the 5′ or 3′ nucleoside (e.g., terminal nucleoside) of any of the oligonucleotides described herein is conjugated to a spacer that is a substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof; each R^{A} is independently hydrogen or substituted or unsubstituted alkyl. In certain embodiments, the spacer is a substituted or unsubstituted alkylene, substituted or unsubstituted heterocyclylene, substituted or unsubstituted heteroarylene, —O—, —N(R^{A})—, or —C(═O)N(R^{A})₂, or a combination thereof.

In some embodiments, the 5′ or 3′ nucleoside of any one of the oligonucleotides described herein is conjugated to a compound of the formula —NH₂—(CH₂)ₙ—, wherein n is an integer from 1 to 12. In some embodiments, n is 6, 7, 8, 9, 10, 11, or 12. In some embodiments, a phosphodiester linkage is present between the compound of the formula NH₂—(CH₂)ₙ— and the 5′ or 3′ nucleoside of the oligonucleotide. In some embodiments, a compound of the formula NH₂—(CH₂)₆— is conjugated to the oligonucleotide via a reaction between 6-amino-1-hexanol (NH₂—(CH₂)₆—OH) and the 5′ phosphate of the oligonucleotide.

In some embodiments, the oligonucleotide is conjugated to a targeting agent, e.g., a muscle targeting agent such as an anti-TfR antibody, e.g., via the amine group.

a. Oligonucleotide Size/Sequence

Oligonucleotides may be of a variety of different lengths, e.g., depending on the format. In some embodiments, an oligonucleotide is 7, 8, 9, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In a some embodiments, the oligonucleotide is 8 to 50 nucleotides in length, 8 to 40 nucleotides in length, 8 to 30 nucleotides in length, 10 to 15 nucleotides in length, 10 to 20 nucleotides in length, 15 to 25 nucleotides in length, 21 to 23 nucleotides in lengths, etc.

In some embodiments, a complementary nucleic acid sequence of an oligonucleotide for purposes of the present disclosure is specifically hybridizable or specific for the target nucleic acid when binding of the sequence to the target molecule (e.g., mRNA) interferes with the normal function of the target (e.g., mRNA) to cause a loss of activity (e.g., inhibiting translation) or expression (e.g., degrading a target mRNA) and there is a sufficient degree of complementarity to avoid non-specific binding of the sequence to non-target sequences under conditions in which avoidance of non-specific binding is desired, e.g., under physiological conditions in the case of in vivo assays or therapeutic treatment, and in the case of in vitro assays, under conditions in which the assays are performed under suitable conditions of stringency. Thus, in some embodiments, an oligonucleotide may be at least 80%, at least 85%, at least 90%, at least 91%, at least 92%, at least 93%, at least 94%, at least 95%, at least 96%, at least 97%, at least 98%, at least 99% or 100% complementary to the consecutive nucleotides of an target nucleic acid. In some embodiments a complementary nucleotide sequence need not be 100% complementary to that of its target to be specifically hybridizable or specific for a target nucleic acid.

In some embodiments, an oligonucleotide comprises region of complementarity to a target nucleic acid that is in the range of 8 to 15, 8 to 30, 8 to 40, or 10 to 50, or 5 to 50, or 5 to 40 nucleotides in length. In some embodiments, a region of complementarity of an oligonucleotide to a target nucleic acid is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the region of complementarity is complementary with at least 8 consecutive nucleotides of a target nucleic acid. In some embodiments, an oligonucleotide may contain 1, 2 or 3 base mismatches compared to the portion of the consecutive nucleotides of target nucleic acid. In some embodiments the oligonucleotide may have up to 3 mismatches over 15 bases, or up to 2 mismatches over 10 bases.

In some embodiments, the oligonucleotide is complementary (e.g., at least 85% at least 90%, at least 95%, or 100%) to a target sequence of any one of the oligonucleotides provided herein. In some embodiments, such target sequence is 100% complementary to the oligonucleotide described herein.

In some embodiments, any one or more of the thymine bases (T's) in any one of the oligonucleotides provided herein may optionally be uracil bases (U's), and/or any one or more of the U's may optionally be T's.

b. Oligonucleotide Modifications:

The oligonucleotides described herein may be modified, e.g., comprise a modified sugar moiety, a modified internucleoside linkage, a modified nucleotide and/or (e.g., and) combinations thereof. In addition, in some embodiments, oligonucleotides may exhibit one or more of the following properties: do not mediate alternative splicing; are not immune stimulatory; are nuclease resistant; have improved cell uptake compared to unmodified oligonucleotides; are not toxic to cells or mammals; have improved endosomal exit internally in a cell; minimizes TLR stimulation; or avoid pattern recognition receptors. Any of the modified chemistries or formats of oligonucleotides described herein can be combined with each other. For example, one, two, three, four, five, or more different types of modifications can be included within the same oligonucleotide.

In some embodiments, certain nucleotide modifications may be used that make an oligonucleotide into which they are incorporated more resistant to nuclease digestion than the native oligodeoxynucleotide or oligoribonucleotide molecules; these modified oligonucleotides survive intact for a longer time than unmodified oligonucleotides. Specific examples of modified oligonucleotides include those comprising modified backbones, for example, modified internucleoside linkages such as phosphorothioates, phosphotriesters, methyl phosphonates, short chain alkyl or cycloalkyl intersugar linkages or short chain heteroatomic or heterocyclic intersugar linkages. Accordingly, oligonucleotides of the disclosure can be stabilized against nucleolytic degradation such as by the incorporation of a modification, e.g., a nucleotide modification.

In some embodiments, an oligonucleotide may be of up to 50 or up to 100 nucleotides in length in which 2 to 10, 2 to 15, 2 to 16, 2 to 17, 2 to 18, 2 to 19, 2 to 20, 2 to 25, 2 to 30, 2 to 40, 2 to 45, or more nucleotides of the oligonucleotide are modified nucleotides. The oligonucleotide may be of 8 to 30 nucleotides in length in which 2 to 10, 2 to 15, 2 to 16, 2 to 17, 2 to 18, 2 to 19, 2 to 20, 2 to 25, 2 to 30 nucleotides of the oligonucleotide are modified nucleotides. The oligonucleotide may be of 8 to 15 nucleotides in length in which 2 to 4, 2 to 5, 2 to 6, 2 to 7, 2 to 8, 2 to 9, 2 to 10, 2 to 11, 2 to 12, 2 to 13, 2 to 14 nucleotides of the oligonucleotide are modified nucleotides. Optionally, the oligonucleotides may have every nucleotide except 1, 2, 3, 4, 5, 6, 7, 8, 9, or 10 nucleotides modified. Oligonucleotide modifications are described further herein.

c. Modified Nucleosides

In some embodiments, the oligonucleotide described herein comprises at least one nucleoside modified at the 2′ position of the sugar. In some embodiments, an oligonucleotide comprises at least one 2′-modified nucleoside. In some embodiments, all of the nucleosides in the oligonucleotide are 2′-modified nucleosides.

In some embodiments, the oligonucleotide described herein comprises one or more non-bicyclic 2′-modified nucleosides, e.g., 2′-deoxy, 2′-fluoro (2′-F), 2′-O-methyl (2′-O-Me), 2′-O-methoxyethyl (2′-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O—NMA) modified nucleoside.

In some embodiments, the oligonucleotide described herein comprises one or more 2′-4′ bicyclic nucleosides in which the ribose ring comprises a bridge moiety connecting two atoms in the ring, e.g., connecting the 2′-O atom to the 4′-C atom via a methylene (LNA) bridge, an ethylene (ENA) bridge, or a (S)-constrained ethyl (cEt) bridge. Examples of LNAs are described in International Patent Application Publication WO/2008/043753, published on Apr. 17, 2008, and entitled “RNA Antagonist Compounds For The Modulation Of PCSK9”, the contents of which are incorporated herein by reference in its entirety. Examples of ENAs are provided in International Patent Publication No. WO 2005/042777, published on May 12, 2005, and entitled “APP/ENA Antisense”; Morita et al., Nucleic Acid Res., Suppl 1:241-242, 2001; Surono et al., Hum. Gene Ther., 15:749-757, 2004; Koizumi, Curr. Opin. Mol. Ther., 8:144-149, 2006 and Horie et al., Nucleic Acids Symp. Ser (Oxf), 49:171-172, 2005; the disclosures of which are incorporated herein by reference in their entireties. Examples of cEt are provided in U.S. Pat. Nos. 7,101,993; 7,399,845 and 7,569,686, each of which is herein incorporated by reference in its entirety.

In some embodiments, the oligonucleotide comprises a modified nucleoside disclosed in one of the following United States Patent or Patent Application Publications: U.S. Pat. Nos. 7,399,845, issued on Jul. 15, 2008, and entitled “6-Modified Bicyclic Nucleic Acid Analogs”; U.S. Pat. Nos. 7,741,457, issued on Jun. 22, 2010, and entitled “6-Modified Bicyclic Nucleic Acid Analogs”; U.S. Pat. No. 8,022,193, issued on Sep. 20, 2011, and entitled “6-Modified Bicyclic Nucleic Acid Analogs”; U.S. Pat. No. 7,569,686, issued on Aug. 4, 2009, and entitled “Compounds And Methods For Synthesis Of Bicyclic Nucleic Acid Analogs”; U.S. Pat. No. 7,335,765, issued on Feb. 26, 2008, and entitled “Novel Nucleoside And Oligonucleotide Analogues”; U.S. Pat. No. s7,314,923, issued on Jan. 1, 2008, and entitled “Novel Nucleoside And Oligonucleotide Analogues”; U.S. Pat. No. 7,816,333, issued on Oct. 19, 2010, and entitled “Oligonucleotide Analogues And Methods Utilizing The Same” and US Publication Number 2011/0009471 now U.S. Pat. No. 8,957,201, issued on Feb. 17, 2015, and entitled “Oligonucleotide Analogues And Methods Utilizing The Same”, the entire contents of each of which are incorporated herein by reference for all purposes.

In some embodiments, the oligonucleotide comprises at least one modified nucleoside that results in an increase in Tm of the oligonucleotide in a range of 1° C., 2° C., 3° C., 4° C., or 5° C. compared with an oligonucleotide that does not have the at least one modified nucleoside . The oligonucleotide may have a plurality of modified nucleosides that result in a total increase in Tm of the oligonucleotide in a range of 2° C., 3° C., 4° C., 5° C., 6° C., 7° C., 8° C., 9° C., 10° C., 15° C., 20° C., 25° C., 30° C., 35° C., 40° C., 45° C. or more compared with an oligonucleotide that does not have the modified nucleoside.

The oligonucleotide may comprise a mix of nucleosides of different kinds. For example, an oligonucleotide may comprise a mix of 2′-deoxyribonucleosides or ribonucleosides and 2′-fluoro modified nucleosides. An oligonucleotide may comprise a mix of deoxyribonucleosides or ribonucleosides and 2′-O-Me modified nucleosides. An oligonucleotide may comprise a mix of 2′-fluoro modified nucleosides and 2′-O-Me modified nucleosides. An oligonucleotide may comprise a mix of 2′-4′ bicyclic nucleosides and 2′-MOE, 2′-fluoro, or 2′-O-Me modified nucleosides. An oligonucleotide may comprise a mix of non-bicyclic 2′-modified nucleosides (e.g., 2′-MOE, 2′-fluoro, or 2′-O-Me) and 2′-4′ bicyclic nucleosides (e.g., LNA, ENA, cEt).

The oligonucleotide may comprise alternating nucleosides of different kinds. For example, an oligonucleotide may comprise alternating 2′-deoxyribonucleosides or ribonucleosides and 2′-fluoro modified nucleosides. An oligonucleotide may comprise alternating deoxyribonucleosides or ribonucleosides and 2′-O-Me modified nucleosides. An oligonucleotide may comprise alternating 2′-fluoro modified nucleosides and 2′-O-Me modified nucleosides. An oligonucleotide may comprise alternating 2′-4′ bicyclic nucleosides and 2′-MOE, 2′-fluoro, or 2′-O-Me modified nucleosides. An oligonucleotide may comprise alternating non-bicyclic 2′-modified nucleosides (e.g., 2′-MOE, 2′-fluoro, or 2′-O-Me) and 2′-4′ bicyclic nucleosides (e.g., LNA, ENA, cEt).

In some embodiments, an oligonucleotide described herein comprises a 5′-vinylphosphonate modification, one or more abasic residues, and/or one or more inverted abasic residues.

d. Internucleoside Linkages/Backbones

In some embodiments, oligonucleotide may contain a phosphorothioate or other modified internucleoside linkage. In some embodiments, the oligonucleotide comprises phosphorothioate internucleoside linkages. In some embodiments, the oligonucleotide comprises phosphorothioate internucleoside linkages between at least two nucleotides. In some embodiments, the oligonucleotide comprises phosphorothioate internucleoside linkages between all nucleotides. For example, in some embodiments, oligonucleotides comprise modified internucleoside linkages at the first, second, and/or (e.g., and) third internucleoside linkage at the 5′ or 3′ end of the nucleotide sequence.

Phosphorus-containing linkages that may be used include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5, 177,196; 5,188,897; 5,264,423; 5,276,019; 5,278,302; 5,286,717; 5,321,131; 5,399,676; 5,405,939; 5,453,496; 5,455, 233; 5,466,677; 5,476,925; 5,519,126; 5,536,821; 5,541,306; 5,550,111; 5,563, 253; 5,571,799; 5,587,361; and 5,625,050.

In some embodiments, oligonucleotides may have heteroatom backbones, such as methylene(methylimino) or MMI backbones; amide backbones (see De Mesmaeker et al. Ace. Chem. Res. 1995, 28:366-374); morpholino backbones (see Summerton and Weller, U.S. Pat. No. 5,034,506); or peptide nucleic acid (PNA) backbones (wherein the phosphodiester backbone of the oligonucleotide is replaced with a polyamide backbone, the nucleotides being bound directly or indirectly to the aza nitrogen atoms of the polyamide backbone, see Nielsen et al., Science 1991, 254, 1497).

e. Stereospecific Oligonucleotides

In some embodiments, internucleotidic phosphorus atoms of oligonucleotides are chiral, and the properties of the oligonucleotides are adjusted based on the configuration of the chiral phosphorus atoms. In some embodiments, appropriate methods may be used to synthesize P-chiral oligonucleotide analogs in a stereocontrolled manner (e.g., as described in Oka N, Wada T, Stereocontrolled synthesis of oligonucleotide analogs containing chiral internucleotidic phosphorus atoms. Chem Soc Rev. 2011 December; 40(12):5829-43.) In some embodiments, phosphorothioate containing oligonucleotides are provided that comprise nucleoside units that are joined together by either substantially all Sp or substantially all Rp phosphorothioate intersugar linkages. In some embodiments, such phosphorothioate oligonucleotides having substantially chirally pure intersugar linkages are prepared by enzymatic or chemical synthesis, as described, for example, in U.S. Pat. No. 5,587,261, issued on Dec. 12, 1996, the contents of which are incorporated herein by reference in their entirety. In some embodiments, chirally controlled oligonucleotides provide selective cleavage patterns of a target nucleic acid. For example, in some embodiments, a chirally controlled oligonucleotide provides single site cleavage within a complementary sequence of a nucleic acid, as described, for example, in US Patent Application Publication 20170037399 A1, published on Feb. 2, 2017, entitled “CHIRAL DESIGN”, the contents of which are incorporated herein by reference in their entirety.

f. Morpholinos

In some embodiments, the oligonucleotide may be a morpholino-based compounds. Morpholino-based oligomeric compounds are described in Dwaine A. Braasch and David R. Corey, Biochemistry, 2002, 41(14), 4503-4510); Genesis, volume 30, issue 3, 2001; Heasman, J., Dev. Biol., 2002, 243, 209-214; Nasevicius et al., Nat. Genet., 2000, 26, 216-220; Lacerra et al., Proc. Natl. Acad. Sci., 2000, 97, 9591-9596; and U.S. Pat. No. 5,034,506, issued Jul. 23, 1991. In some embodiments, the morpholino-based oligomeric compound is a phosphorodiamidate morpholino oligomer (PMO) (e.g., as described in Iverson, Curr. Opin. Mol. Ther., 3:235-238, 2001; and Wang et al., J. Gene Med., 12:354-364, 2010; the disclosures of which are incorporated herein by reference in their entireties).

g. Peptide Nucleic Acids (PNAs)

In some embodiments, both a sugar and an internucleoside linkage (the backbone) of the nucleotide units of an oligonucleotide are replaced with novel groups. In some embodiments, the base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, for example, an aminoethylglycine backbone. The nucleobases are retained and are bound directly or indirectly to aza nitrogen atoms of the amide portion of the backbone. Representative publication that report the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

h. Gapmers

In some embodiments, an oligonucleotide described herein is a gapmer. A gapmer oligonucleotide generally has the formula 5′-X-Y-Z-3′, with X and Z as flanking regions around a gap region Y. In some embodiments, flanking region X of formula 5′-X-Y-Z-3′ is also referred to as X region, flanking sequence X, 5′ wing region X, or 5′ wing segment. In some embodiments, flanking region Z of formula 5′-X-Y-Z-3′ is also referred to as Z region, flanking sequence Z, 3′ wing region Z, or 3′ wing segment. In some embodiments, gap region Y of formula 5′-X-Y-Z-3′ is also referred to as Y region, Y segment, or gap-segment Y. In some embodiments, each nucleoside in the gap region Y is a 2′-deoxyribonucleoside, and neither the 5′ wing region X or the 3′ wing region Z contains any 2′-deoxyribonucleosides.

In some embodiments, the Y region is a contiguous stretch of nucleotides, e.g., a region of 6 or more DNA nucleotides, which are capable of recruiting an RNAse, such as RNAse H. In some embodiments, the gapmer binds to the target nucleic acid, at which point an RNAse is recruited and can then cleave the target nucleic acid. In some embodiments, the Y region is flanked both 5′ and 3′ by regions X and Z comprising high-affinity modified nucleosides, e.g., one to six high-affinity modified nucleosides. Examples of high affinity modified nucleosides include, but are not limited to, 2′-modified nucleosides (e.g., 2′-MOE, 2′O-Me, 2′-F) or 2′-4′ bicyclic nucleosides (e.g., LNA, cEt, ENA). In some embodiments, the flanking sequences X and Z may be of 1-20 nucleotides, 1-8 nucleotides, or 1-5 nucleotides in length. The flanking sequences X and Z may be of similar length or of dissimilar lengths. In some embodiments, the gap-segment Y may be a nucleotide sequence of 5-20 nucleotides, 5-15 twelve nucleotides, or 6-10 nucleotides in length.

In some embodiments, the gap region of the gapmer oligonucleotides may contain modified nucleotides known to be acceptable for efficient RNase H action in addition to DNA nucleotides, such as C4′-substituted nucleotides, acyclic nucleotides, and arabino-configured nucleotides. In some embodiments, the gap region comprises one or more unmodified internucleosides. In some embodiments, one or both flanking regions each independently comprise one or more phosphorothioate internucleoside linkages (e.g., phosphorothioate internucleoside linkages or other linkages) between at least two, at least three, at least four, at least five or more nucleotides. In some embodiments, the gap region and two flanking regions each independently comprise modified internucleoside linkages (e.g., phosphorothioate internucleoside linkages or other linkages) between at least two, at least three, at least four, at least five or more nucleotides.

A gapmer may be produced using appropriate methods. Representative U.S. patents, U.S. patent publications, and PCT publications that teach the preparation of gapmers include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; 5,700,922; 5,898,031; 7,015,315; 7,101,993; 7,399,845; 7,432,250; 7,569,686; 7,683,036; 7,750,131; 8,580,756; 9,045,754; 9,428,534; 9,695,418; 10,017,764; 10,260,069; 9,428,534; 8,580,756; U.S. patent publication Nos. US20050074801, US20090221685; US20090286969, US20100197762, and US20110112170; PCT publication Nos. W02004069991; WO2005023825; WO2008049085 and WO2009090182; and EP Patent No. EP2,149,605, each of which is herein incorporated by reference in its entirety.

In some embodiments, a gapmer is 10-40 nucleosides in length. For example, a gapmer may be 10-40, 10-35, 10-30, 10-25, 10-20, 10-15, 15-40, 15-35, 15-30, 15-25, 15-20, 20-40, 20-35, 20-30, 20-25, 25-40, 25-35, 25-30, 30-40, 30-35, or 35-40 nucleosides in length. In some embodiments, a gapmer is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleosides in length.

In some embodiments, the gap region Y in a gapmer is 5-20 nucleosides in length. For example, the gap region Y may be 5-20, 5-15, 5-10, 10-20, 10-15, or 15-20 nucleosides in length. In some embodiments, the gap region Y is 5, 6, 7, 8, 9, 10, 11, 12, 13, 14, 15, 16, 17, 18, 19, or 20 nucleosides in length. In some embodiments, each nucleoside in the gap region Y is a 2′-deoxyribonucleoside. In some embodiments, all nucleosides in the gap region Y are 2′-deoxyribonucleosides. In some embodiments, one or more of the nucleosides in the gap region Y is a modified nucleoside (e.g., a 2′ modified nucleoside such as those described herein). In some embodiments, one or more cytosines in the gap region Y are optionally 5-methyl-cytosines. In some embodiments, each cytosine in the gap region Y is a 5-methyl-cytosines.

In some embodiments, the 5′wing region of a gapmer (X in the 5′-X-Y-Z-3′ formula) and the 3′wing region of a gapmer (Z in the 5′-X-Y-Z-3′ formula) are independently 1-20 nucleosides long. For example, the 5′wing region of a gapmer (X in the 5′-X-Y-Z-3′ formula) and the 3′wing region of the gapmer (Z in the 5′-X-Y-Z-3′ formula) may be independently 1-20, 1-15, 1-10, 1-7, 1-5, 1-3, 1-2, 2-5, 2-7, 3-5, 3-7, 5-20, 5-15, 5-10, 10-20, 10-15, or 15-20 nucleosides long. In some embodiments, the 5′wing region of the gapmer (X in the 5′-X-Y-Z-3′ formula) and the 3′wing region of the gapmer (Z in the 5′-X-Y-Z-3′ formula) are independently 1, 2, 3, 4, 5, 6, 7, 8, 9, 10, 11, 12, 13, 14, 15, 16, 17, 18, 19, or 20 nucleosides long. In some embodiments, the 5′wing region of the gapmer (X in the 5′-X-Y-Z-3′ formula) and the 3′wing region of the gapmer (Z in the 5′-X-Y-Z-3′ formula) are of the same length. In some embodiments, the 5′wing region of the gapmer (X in the 5′-X-Y-Z-3′ formula) and the 3′wing region of the gapmer (Z in the 5′-X-Y-Z-3′ formula) are of different lengths. In some embodiments, the 5′wing region of the gapmer (X in the 5′-X-Y-Z-3′ formula) is longer than the 3′wing region of the gapmer (Z in the 5′-X-Y-Z-3′ formula). In some embodiments, the 5′wing region of the gapmer (X in the 5′-X-Y-Z-3′ formula) is shorter than the 3′wing region of the gapmer (Z in the 5′-X-Y-Z-3′ formula).

In some embodiments, a gapmer comprises a 5′-X-Y-Z-3′ of 5-10-5, 4-12-4, 3-14-3, 2-16-2, 1-18-1, 3-10-3, 2-10-2, 1-10-1, 2-8-2, 4-6-4, 3-6-3, 2-6-2, 4-7-4, 3-7-3, 2-7-2, 4-8-4, 3-8-3, 2-8-2, 1-8-1, 2-9-2, 1-9-1, 2-10-2, 1-10-1, 1-12-1, 1-16-1, 2-15-1, 1-15-2, 1-14-3, 3-14-1, 2-14-2, 1-13-4, 4-13-1, 2-13-3, 3-13-2, 1-12-5, 5-12-1, 2-12-4, 4-12-2, 3-12-3, 1-11-6, 6-11-1, 2-11-5, 5-11-2, 3-11-4, 4-11-3, 1-17-1, 2-16-1, 1-16-2, 1-15-3, 3-15-1, 2-15-2, 1-14-4, 4-14-1, 2-14-3, 3-14-2, 1-13-5, 5-13-1, 2-13-4, 4-13-2, 3-13-3, 1-12-6, 6-12-1, 2-12-5, 5-12-2, 3-12-4, 4-12-3, 1-11-7, 7-11-1, 2-11-6, 6-11-2, 3-11-5, 5-11-3, 4-11-4, 1-18-1, 1-17-2, 2-17-1, 1-16-3, 1-16-3, 2-16-2, 1-15-4, 4-15-1, 2-15-3, 3-15-2, 1-14-5, 5-14-1, 2-14-4, 4-14-2, 3-14-3, 1-13-6, 6-13-1, 2-13-5, 5-13-2, 3-13-4, 4-13-3, 1-12-7, 7-12-1, 2-12-6, 6-12-2, 3-12-5, 5-12-3, 1-11-8, 8-11-1, 2-11-7, 7-11-2, 3-11-6, 6-11-3, 4-11-5, 5-11-4, 1-18-1, 1-17-2, 2-17-1, 1-16-3, 3-16-1, 2-16-2, 1-15-4, 4-15-1, 2-15-3, 3-15-2, 1-14-5, 2-14-4, 4-14-2, 3-14-3, 1-13-6, 6-13-1, 2-13-5, 5-13-2, 3-13-4, 4-13-3, 1-12-7, 7-12-1, 2-12-6, 6-12-2, 3-12-5, 5-12-3, 1-11-8, 8-11-1, 2-11-7, 7-11-2, 3-11-6, 6-11-3, 4-11-5, 5-11-4, 1-19-1, 1-18-2, 2-18-1, 1-17-3, 3-17-1, 2-17-2, 1-16-4, 4-16-1, 2-16-3, 3-16-2, 1-15-5, 2-15-4, 4-15-2, 3-15-3, 1-14-6, 6-14-1, 2-14-5, 5-14-2, 3-14-4, 4-14-3, 1-13-7, 7-13-1, 2-13-6, 6-13-2, 3-13-5, 5-13-3, 4-13-4, 1-12-8, 8-12-1, 2-12-7, 7-12-2, 3-12-6, 6-12-3, 4-12-5, 5-12-4, 2-11-8, 8-11-2, 3-11-7, 7-11-3, 4-11-6, 6-11-4, 5-11-5, 1-20-1, 1-19-2, 2-19-1, 1-18-3, 3-18-1, 2-18-2, 1-17-4, 4-17-1, 2-17-3, 3-17-2, 1-16-5, 2-16-4, 4-16-2, 3-16-3, 1-15-6, 6-15-1, 2-15-5, 5-15-2, 3-15-4, 4-15-3, 1-14-7, 7-14-1, 2-14-6, 6-14-2, 3-14-5, 5-14-3, 4-14-4, 1-13-8, 8-13-1, 2-13-7, 7-13-2, 3-13-6, 6-13-3, 4-13-5, 5-13-4, 2-12-8, 8-12-2, 3-12-7, 7-12-3, 4-12-6, 6-12-4, 5-12-5, 3-11-8, 8-11-3, 4-11-7, 7-11-4, 5-11-6, 6-11-5, 1-21-1, 1-20-2, 2-20-1, 1-20-3, 3-19-1, 2-19-2, 1-18-4, 4-18-1, 2-18-3, 3-18-2, 1-17-5, 2-17-4, 4-17-2, 3-17-3, 1-16-6, 6-16-1, 2-16-5, 5-16-2, 3-16-4, 4-16-3, 1-15-7, 7-15-1, 2-15-6, 6-15-2, 3-15-5, 5-15-3, 4-15-4, 1-14-8, 8-14-1, 2-14-7, 7-14-2, 3-14-6, 6-14-3, 4-14-5, 5-14-4, 2-13-8, 8-13-2, 3-13-7, 7-13-3, 4-13-6, 6-13-4, 5-13-5, 1-12-10, 10-12-1, 2-12-9, 9-12-2, 3-12-8, 8-12- 3, 4-12-7, 7-12-4, 5-12-6, 6-12-5, 4-11-8, 8-11-4, 5-11-7, 7-11-5, 6-11-6, 1-22-1, 1-21-2, 2-21-1, 1-21-3, 3-20-1, 2-20-2, 1-19-4, 4-19-1, 2-19-3, 3-19-2, 1-18-5, 2-18-4, 4-18-2, 3-18-3, 1-17-6, 6-17-1, 2-17-5, 5-17-2, 3-17-4, 4-17-3, 1-16-7, 7-16-1, 2-16-6, 6-16-2, 3-16-5, 5-16-3, 4-16-4, 1-15-8, 8-15-1, 2-15-7, 7-15-2, 3-15-6, 6-15-3, 4-15-5, 5-15-4, 2-14-8, 8-14-2, 3-14-7, 7-14-3, 4-14-6, 6-14-4, 5-14-5, 3-13-8, 8-13-3, 4-13-7, 7-13-4, 5-13-6, 6-13-5, 4-12-8, 8-12-4, 5-12-7, 7-12-5, 6-12-6, 5-11-8, 8-11-5, 6-11-7, or 7-11-6. The numbers indicate the number of nucleosides in X, Y, and Z regions in the 5′-X-Y-Z-3′ gapmer.

In some embodiments, one or more nucleosides in the 5′wing region of a gapmer (X in the 5′-X-Y-Z-3′ formula) or the 3′wing region of a gapmer (Z in the 5′-X-Y-Z-3′ formula) are modified nucleotides (e.g., high-affinity modified nucleosides). In some embodiments, the modified nuclsoside (e.g., high-affinity modified nucleosides) is a 2′-modifeid nucleoside. In some embodiments, the 2′-modified nucleoside is a 2′-4′ bicyclic nucleoside or a non-bicyclic 2′-modified nucleoside. In some embodiments, the high-affinity modified nucleoside is a 2′-4′ bicyclic nucleoside (e.g., LNA, cEt, or ENA) or a non-bicyclic 2′-modified nucleoside (e.g., 2′-fluoro (2′-F), 2′-O-methyl (2′-O-Me), 2′-0-methoxyethyl (2′-MOE), 2′-0-aminopropyl (2′-O-AP), 2′-O-dimethylaminoethyl (2′-0-DMAOE), 2′-O-dimethylaminopropyl (2′-O-DMAP), 2′-O-dimethylaminoethyloxyethyl (2′-O-DMAEOE), or 2′-O—N-methylacetamido (2′-O—NMA)).

In some embodiments, one or more nucleosides in the 5′wing region of a gapmer (X in the 5′-X-Y-Z-3′ formula) are high-affinity modified nucleosides. In some embodiments, each nucleoside in the 5′wing region of the gapmer (X in the 5′-X-Y-Z-3′ formula) is a high-affinity modified nucleoside. In some embodiments, one or more nucleosides in the 3′wing region of a gapmer (Z in the 5′-X-Y-Z-3′ formula) are high-affinity modified nucleosides. In some embodiments, each nucleoside in the 3′wing region of the gapmer (Z in the 5′-X-Y-Z-3′ formula) is a high-affinity modified nucleoside. In some embodiments, one or more nucleosides in the 5′wing region of the gapmer (X in the 5′-X-Y-Z-3′ formula) are high-affinity modified nucleosides and one or more nucleosides in the 3′wing region of the gapmer (Z in the 5′-X-Y-Z-3′ formula) are high-affinity modified nucleosides. In some embodiments, each nucleoside in the 5′wing region of the gapmer (X in the 5′-X-Y-Z-3′ formula) is a high-affinity modified nucleoside and each nucleoside in the 3′wing region of the gapmer (Z in the 5′-X-Y-Z-3′ formula) is high-affinity modified nucleoside.

In some embodiments, the 5′wing region of a gapmer (X in the 5′-X-Y-Z-3′ formula) comprises the same high affinity nucleosides as the 3′wing region of the gapmer (Z in the 5′-X-Y-Z-3′ formula). For example, the 5′wing region of the gapmer (X in the 5′-X-Y-Z-3′ formula) and the 3′wing region of the gapmer (Z in the 5′-X-Y-Z-3′ formula) may comprise one or more non-bicyclic 2′-modified nucleosides (e.g., 2′-MOE or 2′-O-Me). In another example, the 5′wing region of the gapmer (X in the 5′-X-Y-Z-3′ formula) and the 3′wing region of the gapmer (Z in the 5′-X-Y-Z-3′ formula) may comprise one or more 2′-4′ bicyclic nucleosides (e.g., LNA or cEt). In some embodiments, each nucleoside in the 5′wing region of the gapmer (X in the 5′-X-Y-Z-3′ formula) and the 3′wing region of the gapmer (Z in the 5′-X-Y-Z-3′ formula) is a non-bicyclic 2′-modified nucleosides (e.g., 2′-MOE or 2′-O-Me). In some embodiments, each nucleoside in the 5′wing region of the gapmer (X in the 5′-X-Y-Z-3′ formula) and the 3′wing region of the gapmer (Z in the 5′-X-Y-Z-3′ formula) is a 2′-4′ bicyclic nucleosides (e.g., LNA or cEt).

In some embodiments, a gapmer comprises a 5′-X-Y-Z-3′ configuration, wherein X and Z is independently 1-7 (e.g., 1, 2, 3, 4, 5, 6, or 7) nucleosides in length and Y is 6-10 (e.g., 6, 7, 8, 9, or 10) nucleosides in length, wherein each nucleoside in X and Z is a non-bicyclic 2′-modified nucleosides (e.g., 2′-MOE or 2′-O-Me) and each nucleoside in Y is a 2′-deoxyribonucleoside. In some embodiments, the gapmer comprises a 5′-X-Y-Z-3′ configuration, wherein X and Z is independently 1-7 (e.g., 1, 2, 3, 4, 5, 6, or 7) nucleosides in length and Y is 6-10 (e.g., 6, 7, 8, 9, or 10) nucleosides in length, wherein each nucleoside in X and Z is a 2′-4′ bicyclic nucleosides (e.g., LNA or cEt) and each nucleoside in Y is a 2′-deoxyribonucleoside. In some embodiments, the 5′wing region of the gapmer (X in the 5′-X-Y-Z-3′ formula) comprises different high affinity nucleosides as the 3′wing region of the gapmer (Z in the 5′-X-Y-Z-3′ formula). For example, the 5′wing region of the gapmer (X in the 5′-X-Y-Z-3′ formula) may comprise one or more non-bicyclic 2′-modified nucleosides (e.g., 2′-MOE or 2′-O-Me) and the 3′wing region of the gapmer (Z in the 5′-X-Y-Z-3′ formula) may comprise one or more 2′-4′ bicyclic nucleosides (e.g., LNA or cEt). In another example, the 3′wing region of the gapmer (Z in the 5′-X-Y-Z-3′ formula) may comprise one or more non-bicyclic 2′-modified nucleosides (e.g., 2′-MOE or 2′-O-Me) and the 5′wing region of the gapmer (X in the 5′-X-Y-Z-3′ formula) may comprise one or more 2′-4′ bicyclic nucleosides (e.g., LNA or cEt).

In some embodiments, a gapmer comprises a 5′-X-Y-Z-3′ configuration, wherein X and Z is independently 1-7 (e.g., 1, 2, 3, 4, 5, 6, or 7) nucleosides in length and Y is 6-10 (e.g., 6, 7, 8, 9, or 10) nucleosides in length, wherein each nucleoside in X is a non-bicyclic 2′-modified nucleosides (e.g., 2′-MOE or 2′-O-Me), each nucleoside in Z is a 2′-4′ bicyclic nucleosides (e.g., LNA or cEt), and each nucleoside in Y is a 2′-deoxyribonucleoside. In some embodiments, the gapmer comprises a 5′-X-Y-Z-3′ configuration, wherein X and Z is independently 1-7 (e.g., 1, 2, 3, 4, 5, 6, or 7) nucleosides in length and Y is 6-10 (e.g., 6, 7, 8, 9, or 10) nucleosides in length, wherein each nucleoside in X is a 2′-4′ bicyclic nucleosides (e.g., LNA or cEt), each nucleoside in Z is a non-bicyclic 2′-modified nucleosides (e.g., 2′-MOE or 2′-O-Me) and each nucleoside in Y is a 2′-deoxyribonucleoside.

In some embodiments, the 5′wing region of a gapmer (X in the 5′-X-Y-Z-3′ formula) comprises one or more non-bicyclic 2′-modified nucleosides (e.g., 2′-MOE or 2′-O-Me) and one or more 2′-4′ bicyclic nucleosides (e.g., LNA or cEt). In some embodiments, the 3′wing region of the gapmer (Z in the 5′-X-Y-Z-3′ formula) comprises one or more non-bicyclic 2′-modified nucleosides (e.g., 2′-MOE or 2′-O-Me) and one or more 2′-4′ bicyclic nucleosides (e.g., LNA or cEt). In some embodiments, both the 5′wing region of the gapmer (X in the 5′-X-Y-Z-3′ formula) and the 3′wing region of the gapmer (Z in the 5′-X-Y-Z-3′ formula) comprise one or more non-bicyclic 2′-modified nucleosides (e.g., 2′-MOE or 2′-O-Me) and one or more 2′-4′ bicyclic nucleosides (e.g., LNA or cEt).

In some embodiments, a gapmer comprises a 5′-X-Y-Z-3′ configuration, wherein X and Z is independently 2-7 (e.g., 2, 3, 4, 5, 6, or 7) nucleosides in length and Y is 6-10 (e.g., 6, 7, 8, 9, or 10) nucleosides in length, wherein at least one but not all (e.g., 1, 2, 3, 4, 5, or 6) of positions 1, 2, 3, 4, 5, 6, or 7 in X (the 5′ most position is position 1) is a non-bicyclic 2′-modified nucleoside (e.g., 2′-MOE or 2′-O-Me), wherein the rest of the nucleosides in both X and Z are 2′-4′ bicyclic nucleosides (e.g., LNA or cEt), and wherein each nucleoside in Y is a 2′deoxyribonucleoside. In some embodiments, the gapmer comprises a 5′-X-Y-Z-3′ configuration, wherein X and Z is independently 2-7 (e.g., 2, 3, 4, 5, 6, or 7) nucleosides in length and Y is 6-10 (e.g., 6, 7, 8, 9, or 10) nucleosides in length, wherein at least one but not all (e.g., 1, 2, 3, 4, 5, or 6) of positions 1, 2, 3, 4, 5, 6, or 7 in Z (the 5′ most position is position 1) is a non-bicyclic 2′-modified nucleoside (e.g., 2′-MOE or 2′-O-Me), wherein the rest of the nucleosides in both X and Z are 2′-4′ bicyclic nucleosides (e.g., LNA or cEt), and wherein each nucleoside in Y is a 2′deoxyribonucleoside. In some embodiments, the gapmer comprises a 5′-X-Y-Z-3′ configuration, wherein X and Z is independently 2-7 (e.g., 2, 3, 4, 5, 6, or 7) nucleosides in length and Y is 6-10 (e.g., 6, 7, 8, 9, or 10) nucleosides in length, wherein at least one but not all (e.g., 1, 2, 3, 4, 5, or 6) of positions 1, 2, 3, 4, 5, 6, or 7 in X and at least one of positions but not all (e.g., 1, 2, 3, 4, 5, or 6) 1, 2, 3, 4, 5, 6, or 7 in Z (the 5′ most position is position 1) is a non-bicyclic 2′-modified nucleoside (e.g., 2′-MOE or 2′-O-Me), wherein the rest of the nucleosides in both X and Z are 2′-4′ bicyclic nucleosides (e.g., LNA or cEt), and wherein each nucleoside in Y is a 2′deoxyribonucleoside.

Non-limiting examples of gapmers configurations with a mix of non-bicyclic 2′-modified nucleoside (e.g., 2′-MOE or 2′-O-Me) and 2′-4′ bicyclic nucleosides (e.g., LNA or cEt) in the 5′wing region of the gapmer (X in the 5′-X-Y-Z-3′ formula) and/or the 3′wing region of the gapmer (Z in the 5′-X-Y-Z-3′ formula) include: BBB-(D)n-BBBAA; KKK-(D)n-KKKAA; LLL-(D)n-LLLAA; BBB-(D)n-BBBEE; KKK-(D)n-KKKEE; LLL-(D)n-LLLEE; BBB-(D)n-BBBAA; KKK-(D)n-KKKAA; LLL-(D)n-LLLAA; BBB-(D)n-BBBEE; KKK-(D)n-KKKEE; LLL-(D)n-LLLEE; BBB-(D)n-BBBAAA; KKK-(D)n-KKKAAA; LLL-(D)n-LLLAAA; BBB-(D)n-BBBEEE; KKK-(D)n-KKKEEE; LLL-(D)n-LLLEEE; BBB-(D)n-BBBAAA; KKK-(D)n-KKKAAA; LLL-(D)n-LLLAAA; BBB-(D)n-BBBEEE; KKK-(D)n-KKKEEE; LLL-(D)n-LLLEEE; BABA-(D)n-ABAB; KAKA-(D)n-AKAK; LALA-(D)n-ALAL; BEBE-(D)n-EBEB; KEKE-(D)n-EKEK; LELE-(D)n-ELEL; BABA-(D)n-ABAB; KAKA-(D)n-AKAK; LALA-(D)n-ALAL; BEBE-(D)n-EBEB; KEKE-(D)n-EKEK; LELE-(D)n-ELEL; ABAB-(D)n-ABAB; AKAK-(D)n-AKAK; ALAL-(D)n-ALAL; EBEB-(D)n-EBEB; EKEK-(D)n-EKEK; ELEL-(D)n-ELEL; ABAB-(D)n-ABAB; AKAK-(D)n-AKAK; ALAL-(D)n-ALAL; EBEB-(D)n-EBEB; EKEK-(D)n-EKEK; ELEL-(D)n-ELEL; AABB-(D)n-BBAA; BBAA-(D)n-AABB; AAKK-(D)n-KKAA; AALL-(D)n-LLAA; EEBB-(D)n-BBEE; EEKK-(D)n-KKEE; EELL-(D)n-LLEE; AABB-(D)n-BBAA; AAKK-(D)n-KKAA; AALL-(D)n-LLAA; EEBB-(D)n-BBEE; EEKK-(D)n-KKEE; EELL-(D)n-LLEE; BBB-(D)n-BBA; KKK-(D)n-KKA; LLL-(D)n-LLA; BBB-(D)n-BBE; KKK-(D)n-KKE; LLL-(D)n-LLE; BBB-(D)n-BBA; KKK-(D)n-KKA; LLL-(D)n-LLA; BBB-(D)n-BBE; KKK-(D)n-KKE; LLL-(D)n-LLE; BBB-(D)n-BBA; KKK-(D)n-KKA; LLL-(D)n-LLA; BBB-(D)n-BBE; KKK-(D)n-KKE; LLL-(D)n-LLE; ABBB-(D)n-BBBA; AKKK-(D)n-KKKA; ALLL-(D)n-LLLA; EBBB-(D)n-BBBE; EKKK-(D)n-KKKE; ELLL-(D)n-LLLE; ABBB-(D)n-BBBA; AKKK-(D)n-KKKA; ALLL-(D)n-LLLA; EBBB-(D)n-BBBE; EKKK-(D)n-KKKE; ELLL-(D)n-LLLE; ABBB-(D)n-BBBAA; AKKK-(D)n-KKKAA; ALLL-(D)n-LLLAA; EBBB-(D)n-BBBEE; EKKK-(D)n-KKKEE; ELLL-(D)n-LLLEE; ABBB-(D)n-BBBAA; AKKK-(D)n-KKKAA; ALLL-(D)n-LLLAA; EBBB-(D)n-BBBEE; EKKK-(D)n-KKKEE; ELLL-(D)n-LLLEE; AABBB-(D)n-BBB; AAKKK-(D)n-KKK; AALLL-(D)n-LLL; EEBBB-(D)n-BBB; EEKKK-(D)n-KKK; EELLL-(D)n-LLL; AABBB-(D)n-BBB; AAKKK-(D)n-KKK; AALLL-(D)n-LLL; EEBBB-(D)n-BBB; EEKKK-(D)n-KKK; EELLL-(D)n-LLL; AABBB-(D)n-BBBA; AAKKK-(D)n-KKKA; AALLL-(D)n-LLLA; EEBBB-(D)n-BBBE; EEKKK-(D)n-KKKE; EELLL-(D)n-LLLE; AABBB-(D)n-BBBA; AAKKK-(D)n-KKKA; AALLL-(D)n-LLLA; EEBBB-(D)n-BBBE; EEKKK-(D)n-KKKE; EELLL-(D)n-LLLE; ABBAABB-(D)n-BB; AKKAAKK-(D)n-KK; ALLAALLL-(D)n-LL; EBBEEBB-(D)n-BB; EKKEEKK-(D)n-KK; ELLEELL-(D)n-LL; ABBAABB-(D)n-BB; AKKAAKK-(D)n-KK; ALLAALL-(D)n-LL; EBBEEBB-(D)n-BB; EKKEEKK-(D)n-KK; ELLEELL-(D)n-LL; ABBABB-(D)n-BBB; AKKAKK-(D)n-KKK; ALLALLL-(D)n-LLL; EBBEBB-(D)n-BBB; EKKEKK-(D)n-KKK; ELLELL-(D)n-LLL; ABBABB-(D)n-BBB; AKKAKK-(D)n-KKK; ALLALL-(D)n-LLL; EBBEBB-(D)n-BBB; EKKEKK-(D)n-KKK; ELLELL-(D)n-LLL; EEEK-(D)n-EEEEEEEE; EEK-(D)n-EEEEEEEEE; EK-(D)n-EEEEEEEEEE; EK-(D)n-EEEKK; K-(D)n-EEEKEKE; K-(D)n-EEEKEKEE; K-(D)n-EEKEK; EK-(D)n-EEEEKEKE; EK-(D)n-EEEKEK; EEK-(D)n-KEEKE; EK-(D)n-EEKEK; EK-(D)n-KEEK; EEK-(D)n-EEEKEK; EK-(D)n-KEEEKEE; EK-(D)n-EEKEKE; EK-(D)n-EEEKEKE; and EK-(D)n-EEEEKEK;. “A” nucleosides comprise a 2′-modified nucleoside; “B” represents a 2′-4′ bicyclic nucleoside; “K” represents a constrained ethyl nucleoside (cEt); “L” represents an LNA nucleoside; and “E” represents a 2′-MOE modified ribonucleoside; “D” represents a 2′-deoxyribonucleoside; “n” represents the length of the gap segment (Y in the 5′-X-Y-Z-3′ configuration) and is an integer between 1-20.

In some embodiments, any one of the gapmers described herein comprises one or more modified nucleoside linkages (e.g., a phosphorothioate linkage) in each of the X, Y, and Z regions. In some embodiments, each internucleoside linkage in the any one of the gapmers described herein is a phosphorothioate linkage. In some embodiments, each of the X, Y, and Z regions independently comprises a mix of phosphorothioate linkages and phosphodiester linkages. In some embodiments, each internucleoside linkage in the gap region Y is a phosphorothioate linkage, the 5′wing region X comprises a mix of phosphorothioate linkages and phosphodiester linkages, and the 3′wing region Z comprises a mix of phosphorothioate linkages and phosphodiester linkages.

i. Mixmers

In some embodiments, an oligonucleotide described herein may be a mixmer or comprise a mixmer sequence pattern. In general, mixmers are oligonucleotides that comprise both naturally and non-naturally occurring nucleosides or comprise two different types of non-naturally occurring nucleosides typically in an alternating pattern. Mixmers generally have higher binding affinity than unmodified oligonucleotides and may be used to specifically bind a target molecule, e.g., to block a binding site on the target molecule. Generally, mixmers do not recruit an RNase to the target molecule and thus do not promote cleavage of the target molecule. Such oligonucleotides that are incapable of recruiting RNase H have been described, for example, see WO2007/112754 or WO2007/112753.

In some embodiments, the mixmer comprises or consists of a repeating pattern of nucleoside analogues and naturally occurring nucleosides, or one type of nucleoside analogue and a second type of nucleoside analogue. However, a mixmer need not comprise a repeating pattern and may instead comprise any arrangement of modified nucleoside s and naturally occurring nucleoside s or any arrangement of one type of modified nucleoside and a second type of modified nucleoside. The repeating pattern, may, for instance be every second or every third nucleoside is a modified nucleoside, such as LNA, and the remaining nucleoside s are naturally occurring nucleosides, such as DNA, or are a 2′ substituted nucleoside analogue such as 2′-MOE or 2′ fluoro analogues, or any other modified nucleoside described herein. It is recognized that the repeating pattern of modified nucleoside, such as LNA units, may be combined with modified nucleoside at fixed positions—e.g. at the 5′ or 3′ termini.

In some embodiments, a mixmer does not comprise a region of more than 5, more than 4, more than 3, or more than 2 consecutive naturally occurring nucleosides, such as DNA nucleosides. In some embodiments, the mixmer comprises at least a region consisting of at least two consecutive modified nucleoside, such as at least two consecutive LNAs. In some embodiments, the mixmer comprises at least a region consisting of at least three consecutive modified nucleoside units, such as at least three consecutive LNAs.

In some embodiments, the mixmer does not comprise a region of more than 7, more than 6, more than 5, more than 4, more than 3, or more than 2 consecutive nucleoside analogues, such as LNAs. In some embodiments, LNA units may be replaced with other nucleoside analogues, such as those referred to herein.

Mixmers may be designed to comprise a mixture of affinity enhancing modified nucleosides, such as in non-limiting example LNA nucleosides and 2′-O-Me nucleosides. In some embodiments, a mixmer comprises modified internucleoside linkages (e.g., phosphorothioate internucleoside linkages or other linkages) between at least two, at least three, at least four, at least five or more nucleosides.

A mixmer may be produced using any suitable method. Representative U.S. patents, U.S. patent publications, and PCT publications that teach the preparation of mixmers include U.S. patent publication Nos. US20060128646, US20090209748, US20090298916, US20110077288, and US20120322851, and U.S. Pat. No. 7687617.

In some embodiments, a mixmer comprises one or more morpholino nucleosides. For example, in some embodiments, a mixmer may comprise morpholino nucleosides mixed (e.g., in an alternating manner) with one or more other nucleosides (e.g., DNA, RNA nucleosides) or modified nucleosides (e.g., LNA, 2′-O-Me nucleosides).

In some embodiments, mixmers are useful for splice correcting or exon skipping, for example, as reported in Touznik A., et al., LNA/DNA mixmer-based antisense oligonucleotides correct alternative splicing of the SMN2 gene and restore SMN protein expression in type 1 SMA fibroblasts Scientific Reports, volume 7, Article number: 3672 (2017), Chen S. et al., Synthesis of a Morpholino Nucleic Acid (MNA)-Uridine Phosphoramidite, and Exon Skipping Using MNA/2′-O-Methyl Mixmer Antisense Oligonucleotide, Molecules 2016, 21, 1582, the contents of each which are incorporated herein by reference.

j. RNA Interference (RNAi)

In some embodiments, oligonucleotides provided herein may be in the form of small interfering RNAs (siRNA), also known as short interfering RNA or silencing RNA. SiRNA, is a class of double-stranded RNA molecules, typically about 20-25 base pairs in length that target nucleic acids (e.g., mRNAs) for degradation via the RNA interference (RNAi) pathway in cells. Specificity of siRNA molecules may be determined by the binding of the antisense strand of the molecule to its target RNA. Effective siRNA molecules are generally less than 30 to 35 base pairs in length to prevent the triggering of non-specific RNA interference pathways in the cell via the interferon response, although longer siRNA can also be effective. In some embodiments, the siRNA molecules are 7, 8, 9, 10, 11, 12, 13, 14, 15, 16, 17, 18, 19, 20, 21, 22, 23, 24, 25, 26, 27, 28, 29, 30, 35, 40, 45, 50, or more base pairs in length. In some embodiments, the siRNA molecules are 8 to 30 base pairs in length, 10 to 15 base pairs in length, 10 to 20 base pairs in length, 15 to 25 base pairs in length, 19 to 21 base pairs in length, 21 to 23 base pairs in length.

Following selection of an appropriate target RNA sequence, siRNA molecules that comprise a nucleotide sequence complementary to all or a portion of the target sequence, i.e. an antisense sequence, can be designed and prepared using appropriate methods (see, e.g., PCT Publication Number WO 2004/016735; and U.S. Patent Publication Nos. 2004/0077574 and 2008/0081791). The siRNA molecule can be double stranded (i.e. a dsRNA molecule comprising an antisense strand and a complementary sense strand that hybridizes to form the dsRNA) or single-stranded (i.e. a ssRNA molecule comprising just an antisense strand). The siRNA molecules can comprise a duplex, asymmetric duplex, hairpin or asymmetric hairpin secondary structure, having self-complementary sense and antisense strands.

In some embodiments, the antisense strand of the siRNA molecule is 7, 8, 9, 10, 11, 12, 13, 14, 15, 16, 17, 18, 19, 20, 21, 22, 23, 24, 25, 26, 27, 28, 29, 30, 35, 40, 45, 50, or more nucleotides in length. In some embodiments, the antisense strand is 8 to 50 nucleotides in length, 8 to 40 nucleotides in length, 8 to 30 nucleotides in length, 10 to 15 nucleotides in length, 10 to 20 nucleotides in length, 15 to 25 nucleotides in length, 19 to 21 nucleotides in length, 21 to 23 nucleotides in lengths.

In some embodiments, the sense strand of the siRNA molecule is 7, 8, 9, 10, 11, 12, 13, 14, 15, 16, 17, 18, 19, 20, 21, 22, 23, 24, 25, 26, 27, 28, 29, 30, 35, 40, 45, 50, or more nucleotides in length. In some embodiments, the sense strand is 8 to 50 nucleotides in length, 8 to 40 nucleotides in length, 8 to 30 nucleotides in length, 10 to 15 nucleotides in length, 10 to 20 nucleotides in length, 15 to 25 nucleotides in length, 19 to 21 nucleotides in length, 21 to 23 nucleotides in lengths.

In some embodiments, siRNA molecules comprise an antisense strand comprising a region of complementarity to a target region in a target mRNA. In some embodiments, the region of complementarity is at least 80%, at least 85%, at least 90%, at least 91%, at least 92%, at least 93%, at least 94%, at least 95%, at least 96%, at least 97%, at least 98%, at least 99% or 100% complementary to a target region in a target mRNA. In some embodiments, the target region is a region of consecutive nucleotides in the target mRNA. In some embodiments, a complementary nucleotide sequence need not be 100% complementary to that of its target to be specifically hybridizable or specific for a target RNA sequence.

In some embodiments, siRNA molecules comprise an antisense strand that comprises a region of complementarity to a target RNA sequence and the region of complementarity is in the range of 8 to 15, 8 to 30, 8 to 40, or 10 to 50, or 5 to 50, or 5 to 40 nucleotides in length. In some embodiments, a region of complementarity is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the region of complementarity is complementary with at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25 or more consecutive nucleotides of a target RNA sequence. In some embodiments, siRNA molecules comprise a nucleotide sequence that contains no more than 1, 2, 3, 4, or 5 base mismatches compared to the portion of the consecutive nucleotides of target RNA sequence. In some embodiments, siRNA molecules comprise a nucleotide sequence that has up to 3 mismatches over 15 bases, or up to 2 mismatches over 10 bases.

In some embodiments, siRNA molecules comprise an antisense strand comprising a nucleotide sequence that is complementary (e.g., at least 85%, at least 90%, at least 95%, or 100%) to the target RNA sequence of the oligonucleotides provided herein. In some embodiments, siRNA molecules comprise an antisense strand comprising a nucleotide sequence that is at least 85%, at least 90%, at least 95%, or 100% identical to the oligonucleotides provided herein. In some embodiments, siRNA molecules comprise an antisense strand comprising at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25 or more consecutive nucleotides of the oligonucleotides provided herein.

Double-stranded siRNA may comprise sense and anti-sense RNA strands that are the same length or different lengths. Double-stranded siRNA molecules can also be assembled from a single oligonucleotide in a stem-loop structure, wherein self-complementary sense and antisense regions of the siRNA molecule are linked by means of a nucleic acid based or non-nucleic acid-based linker(s), as well as circular single-stranded RNA having two or more loop structures and a stem comprising self-complementary sense and antisense strands, wherein the circular RNA can be processed either in vivo or in vitro to generate an active siRNA molecule capable of mediating RNAi. Small hairpin RNA (shRNA) molecules thus are also contemplated herein. These molecules comprise a specific antisense sequence in addition to the reverse complement (sense) sequence, typically separated by a spacer or loop sequence. Cleavage of the spacer or loop provides a single-stranded RNA molecule and its reverse complement, such that they may anneal to form a dsRNA molecule (optionally with additional processing steps that may result in addition or removal of one, two, three or more nucleotides from the 3′ end and/or (e.g., and) the 5′ end of either or both strands). A spacer can be of a sufficient length to permit the antisense and sense sequences to anneal and form a double-stranded structure (or stem) prior to cleavage of the spacer (and, optionally, subsequent processing steps that may result in addition or removal of one, two, three, four, or more nucleotides from the 3′ end and/or (e.g., and) the 5′ end of either or both strands). A spacer sequence is may be an unrelated nucleotide sequence that is situated between two complementary nucleotide sequence regions which, when annealed into a double-stranded nucleic acid, comprise a shRNA.

The overall length of the siRNA molecules can vary from about 14 to about 100 nucleotides depending on the type of siRNA molecule being designed. Generally between about 14 and about 50 of these nucleotides are complementary to the RNA target sequence, i.e. constitute the specific antisense sequence of the siRNA molecule. For example, when the siRNA is a double- or single-stranded siRNA, the length can vary from about 14 to about 50 nucleotides, whereas when the siRNA is a shRNA or circular molecule, the length can vary from about 40 nucleotides to about 100 nucleotides.

An siRNA molecule may comprise a 3′ overhang at one end of the molecule, The other end may be blunt-ended or have also an overhang (5′ or 3′). When the siRNA molecule comprises an overhang at both ends of the molecule, the length of the overhangs may be the same or different. In one embodiment, the siRNA molecule of the present disclosure comprises 3′ overhangs of about 1 to about 3 nucleotides on both ends of the molecule. In some embodiments, the siRNA molecule comprises 3′ overhangs of about 1 to about 3 nucleotides on the sense strand. In some embodiments, the siRNA molecule comprises 3′ overhangs of about 1 to about 3 nucleotides on the antisense strand. In some embodiments, the siRNA molecule comprises 3′ overhangs of about 1 to about 3 nucleotides on both the sense strand and the antisense strand.

In some embodiments, the siRNA molecule comprises one or more modified nucleotides (e.g. 1, 2, 3, 4, 5, 6, 7, 8, 9, 10 or more). In some embodiments, the siRNA molecule comprises one or more modified nucleotides and/or (e.g., and) one or more modified internucleotide linkages. In some embodiments, the modified nucleotide is a modified sugar moiety (e.g. a 2′ modified nucleotide). In some embodiments, the siRNA molecule comprises one or more 2′ modified nucleotides, e.g., a 2′-deoxy, 2′-fluoro (2′-F), 2′-O-methyl (2′-O-Me), 2′-O-methoxyethyl (2′-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O--N-methylacetamido (2′-O--NMA). In some embodiments, each nucleotide of the siRNA molecule is a modified nucleotide (e.g., a 2′-modified nucleotide). In some embodiments, the siRNA molecule comprises one or more phosphorodiamidate morpholinos. In some embodiments, each nucleotide of the siRNA molecule is a phosphorodiamidate morpholino.

In some embodiments, the siRNA molecule contains a phosphorothioate or other modified internucleotide linkage. In some embodiments, the siRNA molecule comprises phosphorothioate internucleoside linkages. In some embodiments, the siRNA molecule comprises phosphorothioate internucleoside linkages between at least two nucleotides. In some embodiments, the siRNA molecule comprises phosphorothioate internucleoside linkages between all nucleotides. For example, in some embodiments, the siRNA molecule comprises modified internucleotide linkages at the first, second, and/or (e.g., and) third internucleoside linkage at the 5′ or 3′ end of the siRNA molecule.

In some embodiments, the modified internucleotide linkages are phosphorus-containing linkages. In some embodiments, phosphorus-containing linkages that may be used include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5, 177,196; 5,188,897; 5,264,423; 5,276,019; 5,278,302; 5,286,717; 5,321,131; 5,399,676; 5,405,939; 5,453,496; 5,455, 233; 5,466,677; 5,476,925; 5,519,126; 5,536,821; 5,541,306; 5,550,111; 5,563, 253; 5,571,799; 5,587,361; and 5,625,050.

Any of the modified chemistries or formats of siRNA molecules described herein can be combined with each other. For example, one, two, three, four, five, or more different types of modifications can be included within the same siRNA molecule.

In some embodiments, the antisense strand comprises one or more modified nucleotides (e.g. 1, 2, 3, 4, 5, 6, 7, 8, 9, 10 or more). In some embodiments, the antisense strand comprises one or more modified nucleotides and/or (e.g., and) one or more modified internucleotide linkages. In some embodiments, the modified nucleotide comprises a modified sugar moiety (e.g. a 2′ modified nucleotide). In some embodiments, the antisense strand comprises one or more 2′ modified nucleotides, e.g., a 2′-deoxy, 2′-fluoro (2′-F), 2′-O-methyl (2′-O-Me), 2′-O-methoxyethyl (2′-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O—NMA). In some embodiments, each nucleotide of the antisense strand is a modified nucleotide (e.g., a 2′-modified nucleotide). In some embodiments, the antisense strand comprises one or more phosphorodiamidate morpholinos. In some embodiments, the antisense strand is a phosphorodiamidate morpholino oligomer (PMO).

In some embodiments, antisense strand contains a phosphorothioate or other modified internucleotide linkage. In some embodiments, the antisense strand comprises phosphorothioate internucleoside linkages. In some embodiments, the antisense strand comprises phosphorothioate internucleoside linkages between at least two nucleotides. In some embodiments, the antisense strand comprises phosphorothioate internucleoside linkages between all nucleotides. For example, in some embodiments, the antisense strand comprises modified internucleotide linkages at the first, second, and/or (e.g., and) third internucleoside linkage at the 5′ or 3′ end of the siRNA molecule. In some embodiments, the modified internucleotide linkages are phosphorus-containing linkages. In some embodiments, phosphorus-containing linkages that may be used include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5, 177,196; 5,188,897; 5,264,423; 5,276,019; 5,278,302; 5,286,717; 5,321,131; 5,399,676; 5,405,939; 5,453,496; 5,455, 233; 5,466,677; 5,476,925; 5,519,126; 5,536,821; 5,541,306; 5,550,111; 5,563, 253; 5,571,799; 5,587,361; and 5,625,050.

Any of the modified chemistries or formats of the antisense strand described herein can be combined with each other. For example, one, two, three, four, five, or more different types of modifications can be included within the same antisense strand.

In some embodiments, the sense strand comprises one or more modified nucleotides (e.g. 1, 2, 3, 4, 5, 6, 7, 8, 9, 10 or more). In some embodiments, the sense strand comprises one or more modified nucleotides and/or (e.g., and) one or more modified internucleotide linkages. In some embodiments, the modified nucleotide is a modified sugar moiety (e.g. a 2′ modified nucleotide). In some embodiments, the sense strand comprises one or more 2′ modified nucleotides, e.g., a 2′-deoxy, 2′-fluoro (2′-F), 2′-O-methyl (2′-O-Me), 2′-O-methoxyethyl (2′-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O—NMA). In some embodiments, each nucleotide of the sense strand is a modified nucleotide (e.g., a 2′-modified nucleotide). In some embodiments, the sense strand comprises one or more phosphorodiamidate morpholinos. In some embodiments, the antisense strand is a phosphorodiamidate morpholino oligomer (PMO). In some embodiments, the sense strand contains a phosphorothioate or other modified internucleotide linkage. In some embodiments, the sense strand comprises phosphorothioate internucleoside linkages. In some embodiments, the sense strand comprises phosphorothioate internucleoside linkages between at least two nucleotides. In some embodiments, the sense strand comprises phosphorothioate internucleoside linkages between all nucleotides. For example, in some embodiments, the sense strand comprises modified internucleotide linkages at the first, second, and/or (e.g., and) third internucleoside linkage at the 5′ or 3′ end of the sense strand.

In some embodiments, the modified internucleotide linkages are phosphorus-containing linkages. In some embodiments, phosphorus-containing linkages that may be used include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5, 177,196; 5,188,897; 5,264,423; 5,276,019; 5,278,302; 5,286,717; 5,321,131; 5,399,676; 5,405,939; 5,453,496; 5,455, 233; 5,466,677; 5,476,925; 5,519,126; 5,536,821; 5,541,306; 5,550,111; 5,563, 253; 5,571,799; 5,587,361; and 5,625,050.

Any of the modified chemistries or formats of the sense strand described herein can be combined with each other. For example, one, two, three, four, five, or more different types of modifications can be included within the same sense strand.

In some embodiments, the antisense or sense strand of the siRNA molecule comprises modifications that enhance or reduce RNA-induced silencing complex (RISC) loading. In some embodiments, the antisense strand of the siRNA molecule comprises modifications that enhance RISC loading. In some embodiments, the sense strand of the siRNA molecule comprises modifications that reduce RISC loading and reduce off-target effects. In some embodiments, the antisense strand of the siRNA molecule comprises a 2′-O-methoxyethyl (2′-MOE) modification. The addition of the 2′-O-methoxyethyl (2′-MOE) group at the cleavage site improves both the specificity and silencing activity of siRNAs by facilitating the oriented RNA-induced silencing complex (RISC) loading of the modified strand, as described in Song et al., (2017) Mol Ther Nucleic Acids 9:242-250, incorporated herein by reference in its entirety. In some embodiments, the antisense strand of the siRNA molecule comprises a 2′-OMe-phosphorodithioate modification, which increases RISC loading as described in Wu et al., (2014) Nat Commun 5:3459, incorporated herein by reference in its entirety.

In some embodiments, the sense strand of the siRNA molecule comprises a 5′-morpholino, which reduces RISC loading of the sense strand and improves antisense strand selection and RNAi activity, as described in Kumar et al., (2019) Chem Commun (Camb) 55(35):5139-5142, incorporated herein by reference in its entirety. In some embodiments, the sense strand of the siRNA molecule is modified with a synthetic RNA-like high affinity nucleotide analogue, Locked Nucleic Acid (LNA), which reduces RISC loading of the sense strand and further enhances antisense strand incorporation into RISC, as described in Elman et al., (2005) Nucleic Acids Res. 33(1): 439-447, incorporated herein by reference in its entirety. In some embodiments, the sense strand of the siRNA molecule comprises a 5′ unlocked nucleic acic (UNA) modification, which reduce RISC loading of the sense strand and improve silencing potentcy of the antisense strand, as described in Snead et al., (2013) Mol Ther Nucleic Acids 2(7):e103, incorporated herein by reference in its entirety. In some embodiments, the sense strand of the siRNA molecule comprises a 5-nitroindole modification, which descresed the RNAi potency of the sense strand and reduces off-targent effects as described in Zhang et al., (2012) Chembiochem 13(13):1940-1945, incorporated herein by reference in its entirety. In some embodiments, the sense strand comprises a 2′-O′methyl (2′-O-Me) modification, which reduces RISC loading and the off-target effects of the sense strand, as described in Zheng et al., FASEB (2013) 27(10): 4017-4026, incorporated herein by reference in its entirety. In some embodiments, the sense strand of the siRNA molecule is fully substituted with morpholino, 2′-MOE or 2′-O-Me residues, and are not recognized by RISC as described in Kole et al., (2012) Nature reviews. Drug Discovery 11(2):125-140, incorporated herein by reference in its entirety. In some embodiments the antisense strand of the siRNA molecule comprises a 2′-MOE modification and the sense strand comprises an 2′-O-Me modification (see e.g., Song et al., (2017) Mol Ther Nucleic Acids 9:242-250),In some embodiments at least one (e.g., at least 2, at least 3, at least 4, at least 5, at least 10) siRNA molecule is linked (e.g., covalently) to a muscle-targeting agent. In some embodiments, the muscle-targeting agent may comprise, or consist of, a nucleic acid (e.g., DNA or RNA), a peptide (e.g., an antibody), a lipid (e.g., a microvesicle), or a sugar moiety (e.g., a polysaccharide). In some embodiments, the muscle-targeting agent is an antibody. In some embodiments, the muscle-targeting agent is an anti-transferrin receptor antibody (e.g., any one of the anti-TfR antibodies provided herein). In some embodiments, the muscle-targeting agent may be linked to the 5′ end of the sense strand of the siRNA molecule. In some embodiments, the muscle-targeting agent may be linked to the 3′ end of the sense strand of the siRNA molecule. In some embodiments, the muscle-targeting agent may be linked internally to the sense strand of the siRNA molecule. In some embodiments, the muscle-targeting agent may be linked to the 5′ end of the antisense strand of the siRNA molecule. In some embodiments, the muscle-targeting agent may be linked to the 3′ end of the antisense strand of the siRNA molecule. In some embodiments, the muscle-targeting agent may be linked internally to the antisense strand of the siRNA molecule.

k. microRNA (miRNAs)

In some embodiments, an oligonucleotide may be a microRNA (miRNA). MicroRNAs (referred to as “miRNAs”) are small non-coding RNAs, belonging to a class of regulatory molecules that control gene expression by binding to complementary sites on a target RNA transcript. Typically, miRNAs are generated from large RNA precursors (termed pri-miRNAs) that are processed in the nucleus into approximately 70 nucleotide pre-miRNAs, which fold into imperfect stem-loop structures. These pre-miRNAs typically undergo an additional processing step within the cytoplasm where mature miRNAs of 18-25 nucleotides in length are excised from one side of the pre-miRNA hairpin by an RNase III enzyme, Dicer.

As used herein, miRNAs including pri-miRNA, pre-miRNA, mature miRNA or fragments of variants thereof that retain the biological activity of mature miRNA. In one embodiment, the size range of the miRNA can be from 21 nucleotides to 170 nucleotides. In one embodiment the size range of the miRNA is from 70 to 170 nucleotides in length. In another embodiment, mature miRNAs of from 21 to 25 nucleotides in length can be used.

1. Aptamers

In some embodiments, oligonucleotides provided herein may be in the form of aptamers. Generally, in the context of molecular payloads, aptamer is any nucleic acid that binds specifically to a target, such as a small molecule, protein, nucleic acid in a cell. In some embodiments, the aptamer is a DNA aptamer or an RNA aptamer. In some embodiments, a nucleic acid aptamer is a single-stranded DNA or RNA (ssDNA or ssRNA). It is to be understood that a single-stranded nucleic acid aptamer may form helices and/or (e.g., and) loop structures. The nucleic acid that forms the nucleic acid aptamer may comprise naturally occurring nucleotides, modified nucleotides, naturally occurring nucleotides with hydrocarbon linkers (e.g., an alkylene) or a polyether linker (e.g., a PEG linker) inserted between one or more nucleotides, modified nucleotides with hydrocarbon or PEG linkers inserted between one or more nucleotides, or a combination of thereof. Exemplary publications and patents describing aptamers and method of producing aptamers include, e.g., Lorsch and Szostak, 1996; Jayasena, 1999; U.S. Pat. Nos. 5,270,163; 5,567,588; 5,650,275; 5,670,637; 5,683,867; 5,696,249; 5,789,157; 5,843,653; 5,864,026; 5,989,823; 6,569,630; 8,318,438 and PCT application WO 99/31275, each incorporated herein by reference.

m. Ribozymes

In some embodiments, oligonucleotides provided herein may be in the form of a ribozyme. A ribozyme (ribonucleic acid enzyme) is a molecule, typically an RNA molecule, that is capable of performing specific biochemical reactions, similar to the action of protein enzymes. Ribozymes are molecules with catalytic activities including the ability to cleave at specific phosphodiester linkages in RNA molecules to which they have hybridized, such as mRNAs, RNA-containing substrates, lncRNAs, and ribozymes, themselves.

Ribozymes may assume one of several physical structures, one of which is called a “hammerhead.” A hammerhead ribozyme is composed of a catalytic core containing nine conserved bases, a double-stranded stem and loop structure (stem-loop II), and two regions complementary to the target RNA flanking regions the catalytic core. The flanking regions enable the ribozyme to bind to the target RNA specifically by forming double-stranded stems I and III. Cleavage occurs in cis (i.e., cleavage of the same RNA molecule that contains the hammerhead motif) or in trans (cleavage of an RNA substrate other than that containing the ribozyme) next to a specific ribonucleotide triplet by a transesterification reaction from a 3′,5′-phosphate diester to a 2′,3′-cyclic phosphate diester. Without wishing to be bound by theory, it is believed that this catalytic activity requires the presence of specific, highly conserved sequences in the catalytic region of the ribozyme.

Modifications in ribozyme structure have also included the substitution or replacement of various non-core portions of the molecule with non-nucleotidic molecules. For example, Benseler et al. (J. Am. Chem. Soc. (1993) 115:8483-8484) disclosed hammerhead-like molecules in which two of the base pairs of stem II, and all four of the nucleotides of loop II were replaced with non-nucleoside linkers based on hexaethylene glycol, propanediol, bis(triethylene glycol) phosphate, tris(propanediol)bisphosphate, or bis(propanediol) phosphate. Ma et al. (Biochem. (1993) 32:1751-1758; Nucleic Acids Res. (1993) 21:2585-2589) replaced the six nucleotide loop of the TAR ribozyme hairpin with non-nucleotidic, ethylene glycol-related linkers. Thomson et al. (Nucleic Acids Res. (1993) 21:5600-5603) replaced loop II with linear, non-nucleotidic linkers of 13, 17, and 19 atoms in length.

Ribozyme oligonucleotides can be prepared using well known methods (see, e.g., PCT Publications WO9118624; WO9413688; WO9201806; and WO 92/07065; and U.S. Pat. Nos. 5,436,143 and 5,650,502) or can be purchased from commercial sources (e.g., US Biochemicals) and, if desired, can incorporate nucleotide analogs to increase the resistance of the oligonucleotide to degradation by nucleases in a cell. The ribozyme may be synthesized in any known manner, e.g., by use of a commercially available synthesizer produced, e.g., by Applied Biosystems, Inc. or Milligen. The ribozyme may also be produced in recombinant vectors by conventional means. See, Molecular Cloning: A Laboratory Manual, Cold Spring Harbor Laboratory (Current edition). The ribozyme RNA sequences maybe synthesized conventionally, for example, by using RNA polymerases such as T7 or SP6.

n. Guide Nucleic Acids

In some embodiments, oligonucleotides are guide nucleic acid, e.g., guide RNA (gRNA) molecules. Generally, a guide RNA is a short synthetic RNA composed of (1) a scaffold sequence that binds to a nucleic acid programmable DNA binding protein (napDNAbp), such as Cas9, and (2) a nucleotide spacer portion that defines the DNA target sequence (e.g., genomic DNA target) to which the gRNA binds in order to bring the nucleic acid programmable DNA binding protein in proximity to the DNA target sequence. In some embodiments, the napDNAbp is a nucleic acid-programmable protein that forms a complex with (e.g., binds or associates with) one or more RNA(s) that targets the nucleic acid-programmable protein to a target DNA sequence (e.g., a target genomic DNA sequence). In some embodiments, a nucleic acid -programmable nuclease, when in a complex with an RNA, may be referred to as a nuclease:RNA complex. Guide RNAs can exist as a complex of two or more RNAs, or as a single RNA molecule.

Guide RNAs (gRNAs) that exist as a single RNA molecule may be referred to as single-guide RNAs (sgRNAs), though gRNA is also used to refer to guide RNAs that exist as either single molecules or as a complex of two or more molecules. Typically, gRNAs that exist as a single RNA species comprise two domains: (1) a domain that shares homology to a target nucleic acid (i.e., directs binding of a Cas9 complex to the target); and (2) a domain that binds a Cas9 protein. In some embodiments, domain (2) corresponds to a sequence known as a tracrRNA and comprises a stem-loop structure. In some embodiments, domain (2) is identical or homologous to a tracrRNA as provided in Jinek et al., Science 337:816-821 (2012), the entire contents of which is incorporated herein by reference.

In some embodiments, a gRNA comprises two or more of domains (1) and (2), and may be referred to as an extended gRNA. For example, an extended gRNA will bind two or more Cas9 proteins and bind a target nucleic acid at two or more distinct regions, as described herein. The gRNA comprises a nucleotide sequence that complements a target site, which mediates binding of the nuclease/RNA complex to said target site, providing the sequence specificity of the nuclease:RNA complex. In some embodiments, the RNA-programmable nuclease is the (CRISPR-associated system) Cas9 endonuclease, for example, Cas9 (Csnl) from Streptococcus pyogenes (see, e.g., “Complete genome sequence of an M1 strain of Streptococcus pyogenes.” Ferretti J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R.E., Proc. Natl. Acad. Sci. U.S.A. 98:4658-4663 (2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607 (2011); and “A programmable dual-RNA-guided DNA endonuclease in adaptive bacterial immunity.” Jinek M., Chylinski K., Fonfara I., Hauer M., Doudna J. A., Charpentier E. Science 337:816-821 (2012), the entire contents of each of which are incorporated herein by reference.

o. Multimers

In some embodiments, molecular payloads may comprise multimers (e.g., concatemers) of 2 or more oligonucleotides connected by a linker. In this way, in some embodiments, the oligonucleotide loading of a complex/conjugate can be increased beyond the available linking sites on a targeting agent (e.g., available thiol sites on an antibody) or otherwise tuned to achieve a particular payload loading content. Oligonucleotides in a multimer can be the same or different (e.g., targeting different genes or different sites on the same gene or products thereof).

In some embodiments, multimers comprise 2 or more oligonucleotides linked together by a cleavable linker. However, in some embodiments, multimers comprise 2 or more oligonucleotides linked together by a non-cleavable linker. In some embodiments, a multimer comprises 2, 3, 4, 5, 6, 7, 8, 9, 10 or more oligonucleotides linked together. In some embodiments, a multimer comprises 2 to 5, 2 to 10 or 4 to 20 oligonucleotides linked together.

In some embodiments, a multimer comprises 2 or more oligonucleotides linked end-to-end (in a linear arrangement). In some embodiments, a multimer comprises 2 or more oligonucleotides linked end-to-end via a oligonucleotide based linker (e.g., poly-dT linker, an abasic linker). In some embodiments, a multimer comprises a 5′ end of one oligonucleotide linked to a 3′ end of another oligonucleotide. In some embodiments, a multimer comprises a 3′ end of one oligonucleotide linked to a 3′ end of another oligonucleotide. In some embodiments, a multimer comprises a 5′ end of one oligonucleotide linked to a 5′ end of another oligonucleotide. Still, in some embodiments, multimers can comprise a branched structure comprising multiple oligonucleotides linked together by a branching linker.

Further examples of multimers that may be used in the complexes provided herein are disclosed, for example, in US Patent Application Number 2015/0315588 A1, entitled Methods of delivering multiple targeting oligonucleotides to a cell using cleavable linkers, which was published on Nov. 5, 2015; US Patent Application Number 2015/0247141 A1, entitled Multimeric Oligonucleotide Compounds, which was published on Sep. 3, 2015, US Patent Application Number US 2011/0158937 A1, entitled Immunostimulatory Oligonucleotide Multimers, which was published on Jun. 30, 2011; and U.S. Pat. No. 5,693,773, entitled Triplex-Forming Antisense Oligonucleotides Having Abasic Linkers Targeting Nucleic Acids Comprising Mixed Sequences Of Purines And Pyrimidines, which issued on Dec. 2, 1997, the contents of each of which are incorporated herein by reference in their entireties.

ii. Small Molecules:

Any suitable small molecule may be used as a molecular payload, as described herein. For example, in some embodiments, the small molecule is a ubiquitin ligase inhibitor, such as a MuRF1 inhibitor. In some embodiments, the small molecule is a MuRF1 inhibitor as disclosed in Eddins M J, et al., Targeting the ubiquitin E3 ligase MuRF1 to inhibit muscle atrophy. Cell Biochem Biophys. 2011 June; 60(1-2):113-8 or Brown T S, et al., Small-molecule inhibition of MuRF1 attenuates skeletal muscle atrophy and dysfunction in cardiac cachexia J Cachexia Sarcopenia Muscle. 2017 December; 8(6): 939-953, the contents of each of which are incorporated herein by reference in their entireties.

iii. Peptides/Proteins

Any suitable peptide or protein may be used as a molecular payload, as described herein. In some embodiments, a protein is an enzyme. In some embodiments, a protein payload may be an intrabody (intracellular antibody) engineered to inhibit activity of a ubiquitin ligase, e.g., ubiquitin E3 ligase MuRF1. In some embodiments, a payload is a peptide inhibitor that inhibits activity of a ubiquitin ligase, e.g., ubiquitin E3 ligase. These peptides or proteins may be produced, synthesized, and/or (e.g., and) derivatized using several methodologies, e.g. phage displayed peptide libraries, one-bead one-compound peptide libraries, or positional scanning synthetic peptide combinatorial libraries.

iv. Nucleic Acid Constructs

Any suitable gene expression construct may be used as a molecular payload, as described herein. In some embodiments, a gene expression construct may be a vector or a cDNA fragment. In some embodiments, a gene expression construct may be messenger RNA (mRNA). In some embodiments, a mRNA used herein may be a modified mRNA, e.g., as described in U.S. Pat. No. 8,710,200, issued on Apr. 24, 2014, entitled “Engineered nucleic acids encoding a modified erythropoietin and their expression”. In some embodiments, a mRNA may comprise a 5′ methyl cap. In some embodiments, a mRNA may comprise a polyA tail, optionally of up to 160 nucleotides in length. A gene expression construct may encode a sequence of a protein that leads to reduced expression or activity of a pro-atrophy gene listed in Table 1. In some embodiments the gene expression construct encodes an oligonucleotide (e.g., an shRNA or miRNA) that inhibits expression of a target gene in Table 1.

In some embodiments, the gene expression construct encodes a gene editing enzyme, e.g., Cas9.

Examples of nucleic acid constructs that may be used as molecular payloads are provided in International Patent Application Publication WO2017152149A1, published on Sep. 19, 2017, entitled, “CLOSED-ENDED LINEAR DUPLEX DNA FOR NON-VIRAL GENE TRANSFER”; U.S. Pat. No. 8,853,377B2, issued on Oct. 7, 2014, entitled, “MRNA FOR USE IN TREATMENT OF HUMAN GENETIC DISEASES”; and U.S. Pat. No. 8,822,663B2, issued on Sep. 2, 2014, ENGINEERED NUCLEIC ACIDS AND METHODS OF USE THEREOF,” the contents of each of which are incorporated herein by reference in their entireties.

Further examples of complexes and molecular payloads (e.g., oligonucleotides useful for targeting muscle genes) are provided in International Patent Application Publication WO2020/028836, published on Feb. 6, 2020, entitled, “MUSCLE-TARGETING COMPLEXES AND USES THEREOF IN TREATING MUSCLE ATROPHY”, the contents of which are incorporated herein by reference.

C. Linkers

Complexes described herein generally comprise a linker that connects a muscle-targeting agent to a molecular payload. A linker comprises at least one covalent bond. In some embodiments, a linker may be a single bond, e.g., a disulfide bond or disulfide bridge, that connects a muscle-targeting agent to a molecular payload. However, in some embodiments, a linker may connect a muscle-targeting agent to a molecular payload through multiple covalent bonds. In some embodiments, a linker may be a cleavable linker. However, in some embodiments, a linker may be a non-cleavable linker. A linker is generally stable in vitro and in vivo, and may be stable in certain cellular environments. Additionally, generally a linker does not negatively impact the functional properties of either the muscle-targeting agent or the molecular payload. Examples and methods of synthesis of linkers are known in the art (see, e.g. Kline, T. et al. “Methods to Make Homogenous Antibody Drug Conjugates.” Pharmaceutical Research, 2015, 32:11, 3480-3493.; Jain, N. et al. “Current ADC Linker Chemistry” Pharm Res. 2015, 32:11, 3526-3540.; McCombs, J. R. and Owen, S. C. “Antibody Drug Conjugates: Design and Selection of Linker, Payload and Conjugation Chemistry” AAPS J. 2015, 17:2, 339-351.).

A precursor to a linker typically will contain two different reactive species that allow for attachment to both the muscle-targeting agent and a molecular payload. In some embodiments, the two different reactive species may be a nucleophile and/or (e.g., and) an electrophile. In some embodiments, a linker is connected to a muscle-targeting agent via conjugation to a lysine residue or a cysteine residue of the muscle-targeting agent. In some embodiments, a linker is connected to a cysteine residue of a muscle-targeting agent via a maleimide-containing linker, wherein optionally the maleimide-containing linker comprises a maleimidocaproyl or maleimidomethyl cyclohexane-l-carboxylate group. In some embodiments, a linker is connected to a cysteine residue of a muscle-targeting agent or thiol functionalized molecular payload via a 3-arylpropionitrile functional group. In some embodiments, a linker is connected to a lysine residue of an anti-TfR antibody. In some embodiments, a linker is connected to a muscle-targeting agent and/or (e.g., and) a molecular payload via an amide bond, a carbamate bond, a hydrazide, a triazole, a thioether or a disulfide bond.

i. Cleavable Linkers

A cleavable linker may be a protease-sensitive linker, a pH-sensitive linker, or a glutathione-sensitive linker. These linkers are generally cleavable only intracellularly and are preferably stable in extracellular environments, e.g. extracellular to a muscle cell.

Protease-sensitive linkers are cleavable by protease enzymatic activity. These linkers typically comprise peptide sequences and may be 2-10 amino acids, about 2-5 amino acids, about 5-10 amino acids, about 10 amino acids, about 5 amino acids, about 3 amino acids, or about 2 amino acids in length. In some embodiments, a peptide sequence may comprise naturally-occurring amino acids, e.g. cysteine, alanine, or non-naturally-occurring or modified amino acids. Non-naturally occurring amino acids include (3-amino acids, homo-amino acids, proline derivatives, 3-substituted alanine derivatives, linear core amino acids, N-methyl amino acids, and others known in the art. In some embodiments, a protease-sensitive linker comprises a valine-citrulline or alanine-citrulline dipeptide sequence. In some embodiments, a protease-sensitive linker can be cleaved by a lysosomal protease, e.g. cathepsin B, and/or (e.g., and) an endosomal protease.

A pH-sensitive linker is a covalent linkage that readily degrades in high or low pH environments. In some embodiments, a pH-sensitive linker may be cleaved at a pH in a range of 4 to 6. In some embodiments, a pH-sensitive linker comprises a hydrazone or cyclic acetal. In some embodiments, a pH-sensitive linker is cleaved within an endosome or a lysosome.

In some embodiments, a glutathione-sensitive linker comprises a disulfide moiety. In some embodiments, a glutathione-sensitive linker is cleaved by an disulfide exchange reaction with a glutathione species inside a cell. In some embodiments, the disulfide moiety further comprises at least one amino acid, e.g. a cysteine residue.

In some embodiments, the linker is a Val-cit linker (e.g., as described in U.S. Pat. No. 6,214,345, incorporated herein by reference). In some embodiments, before conjugation, the val-cit linker has a structure of:

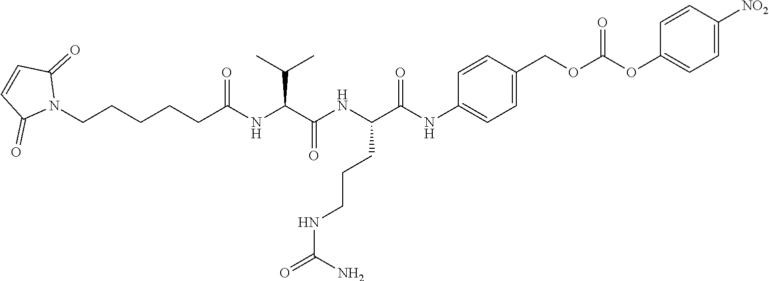 [SMILES: CC(C)[C@H](NC(=O)CCCCCN1C(=O)C=CC1=O)C(=O)N[C@@H](CCCNC(N)=O)C(=O)Nc1ccc(COC(=O)Oc2ccc([N+](=O)[O-])cc2)cc1]

In some embodiments, after conjugation, the val-cit linker has a structure of:

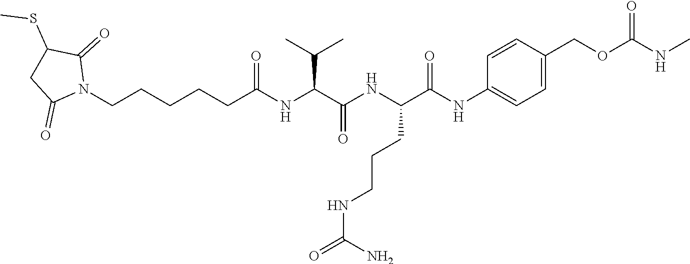

In some embodiments, the Val-cit linker is attached to a reactive chemical moiety (e.g., SPAAC for click chemistry conjugation). In some embodiments, before click chemistry conjugation, the val-cit linker attached to a reactive chemical moiety (e.g., SPAAC for click chemistry conjugation) has the structure of:

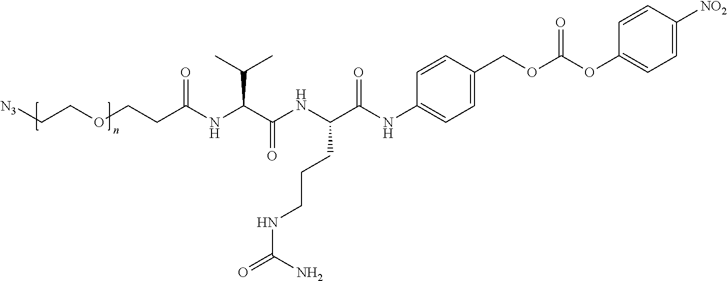

wherein n is any number from 0-10. In some embodiments, n is 3.

In some embodiments, the val-cit linker attached to a reactive chemical moiety (e.g., SPAAC for click chemistry conjugation) is conjugated (e.g., via a different chemical moiety) to a molecular payload (e.g., an oligonucleotide). In some embodiments, the val-cit linker attached to a reactive chemical moiety (e.g., SPAAC for click chemistry conjugation) and is conjugated to a molecular payload (e.g., an oligonucleotide) has the structure of (before click chemistry conjugation):

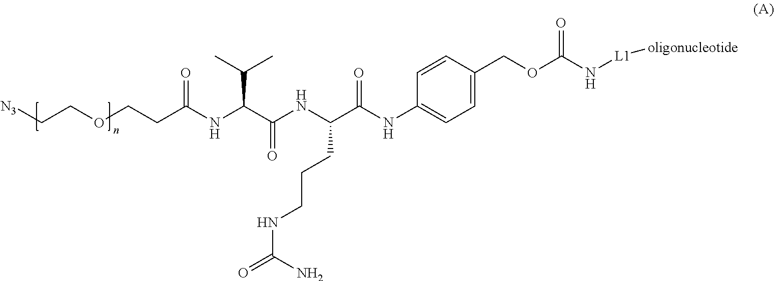

wherein n is any number from 0-10. In some embodiments, n is 3.

In some embodiments, after conjugation to a molecular payload (e.g., an oligonucleotide) and, the val-cit linker has a structure of:

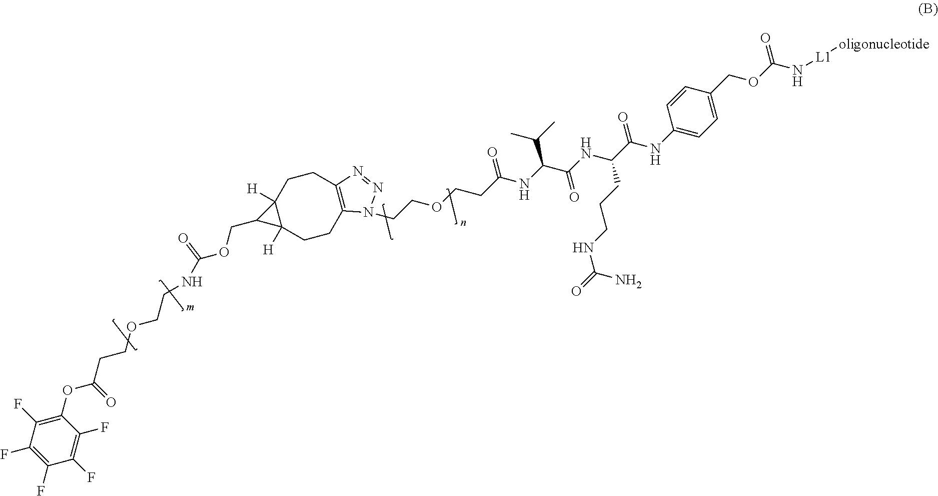

wherein n is any number from 0-10, and wherein m is any number from 0-10. In some embodiments, n is 3 and m is 4.

ii. Non-Cleavable Linkers

In some embodiments, non-cleavable linkers may be used. Generally, a non-cleavable linker cannot be readily degraded in a cellular or physiological environment. In some embodiments, a non-cleavable linker comprises an optionally substituted alkyl group, wherein the substitutions may include halogens, hydroxyl groups, oxygen species, and other common substitutions. In some embodiments, a linker may comprise an optionally substituted alkyl, an optionally substituted alkylene, an optionally substituted arylene, a heteroarylene, a peptide sequence comprising at least one non-natural amino acid, a truncated glycan, a sugar or sugars that cannot be enzymatically degraded, an azide, an alkyne-azide, a peptide sequence comprising a LPXTG sequence (SEQ ID NO: 257), a thioether, a biotin, a biphenyl, repeating units of polyethylene glycol or equivalent compounds, acid esters, acid amides, sulfamides, and/or (e.g., and) an alkoxy-amine linker. In some embodiments, sortase-mediated ligation will be utilized to covalently link a muscle-targeting agent comprising a LPXTG sequence (SEQ ID NO: 257) to a molecular payload comprising a (G)ₙ sequence (see, e.g. Proft T. Sortase-mediated protein ligation: an emerging biotechnology tool for protein modification and immobilization. Biotechnol Lett. 2010, 32(1):1-10.). In some embodiments, a linker comprises a LPXTG sequence (SEQ ID NO: 257), where X is any amino acid.

In some embodiments, a linker may comprise a substituted alkylene, an optionally substituted alkenylene, an optionally substituted alkynylene, an optionally substituted cycloalkylene, an optionally substituted cycloalkenylene, an optionally substituted arylene, an optionally substituted heteroarylene further comprising at least one heteroatom selected from N, O, and S,; an optionally substituted heterocyclylene further comprising at least one heteroatom selected from N, O, and S; an imino, an optionally substituted nitrogen species, an optionally substituted oxygen species 0, an optionally substituted sulfur species, or a poly(alkylene oxide), e.g. polyethylene oxide or polypropylene oxide.

iii. Linker conjugation

In some embodiments, a linker is connected to a muscle-targeting agent and/or (e.g., and) molecular payload via a phosphate, thioether, ether, carbon-carbon, a carbamate, or amide bond. In some embodiments, a linker is connected to an oligonucleotide through a phosphate or phosphorothioate group, e.g. a terminal phosphate of an oligonucleotide backbone. In some embodiments, a linker is connected to an muscle-targeting agent, e.g. an antibody, through a lysine or cysteine residue present on the muscle-targeting agent

In some embodiments, a linker is connected to a muscle-targeting agent and/or (e.g., and) molecular payload by a cycloaddition reaction between an azide and an alkyne to form a triazole, wherein the azide and the alkyne may be located on the muscle-targeting agent, molecular payload, or the linker. In some embodiments, an alkyne may be a cyclic alkyne, e.g., a cyclooctyne. In some embodiments, an alkyne may be bicyclononyne (also known as bicyclo[6.1.0]nonyne or BCN) or substituted bicyclononyne. In some embodiments, a cyclooctane is as described in International Patent Application Publication WO2011136645, published on Nov. 3, 2011, entitled, “Fused Cyclooctyne Compounds And Their Use In Metal free Click Reactions”. In some embodiments, an azide may be a sugar or carbohydrate molecule that comprises an azide. In some embodiments, an azide may be 6-azido-6-deoxygalactose or 6-azido-N-acetylgalactosamine. In some embodiments, a sugar or carbohydrate molecule that comprises an azide is as described in International Patent Application Publication WO2016170186, published on Oct. 27, 2016, entitled, “Process For The Modification Of A Glycoprotein Using A Glycosyltransferase That Is Or Is Derived From A β(1,4)-N-Acetylgalactosaminyltransferase”. In some embodiments, a cycloaddition reaction between an azide and an alkyne to form a triazole, wherein the azide and the alkyne may be located on the muscle-targeting agent, molecular payload, or the linker is as described in International Patent Application Publication WO2014065661, published on May 1, 2014, entitled, “Modified antibody, antibody-conjugate and process for the preparation thereof”; or International Patent Application Publication WO2016170186, published on Oct. 27, 2016, entitled, “Process For The Modification Of A Glycoprotein Using A Glycosyltransferase That Is Or Is Derived From A β(1,4)-N-Acetylgalactosaminyltransferase”.

In some embodiments, a linker further comprises a spacer, e.g., a polyethylene glycol spacer or an acyl/carbomoyl sulfamide spacer, e.g., a HydraSpace™ spacer. In some embodiments, a spacer is as described in Verkade, J. M. M. et al., “A Polar Sulfamide Spacer Significantly Enhances the Manufacturability, Stability, and Therapeutic Index of Antibody-Drug Conjugates”, Antibodies, 2018, 7, 12.

In some embodiments, a linker is connected to a muscle-targeting agent and/or (e.g., and) molecular payload by the Diels-Alder reaction between a dienophile and a diene/hetero-diene, wherein the dienophile and the diene/hetero-diene may be located on the muscle-targeting agent, molecular payload, or the linker. In some embodiments a linker is connected to a muscle-targeting agent and/or (e.g., and) molecular payload by other pericyclic reactions, e.g. ene reaction. In some embodiments, a linker is connected to a muscle-targeting agent and/or (e.g., and) molecular payload by an amide, thioamide, or sulfonamide bond reaction. In some embodiments, a linker is connected to a muscle-targeting agent and/or (e.g., and) molecular payload by a condensation reaction to form an oxime, hydrazone, or semicarbazide group existing between the linker and the muscle-targeting agent and/or (e.g., and) molecular payload.

In some embodiments, a linker is connected to a muscle-targeting agent and/or (e.g., and) molecular payload by a conjugate addition reactions between a nucleophile, e.g. an amine or a hydroxyl group, and an electrophile, e.g. a carboxylic acid, carbonate, or an aldehyde. In some embodiments, a nucleophile may exist on a linker and an electrophile may exist on a muscle-targeting agent or molecular payload prior to a reaction between a linker and a muscle-targeting agent or molecular payload. In some embodiments, an electrophile may exist on a linker and a nucleophile may exist on a muscle-targeting agent or molecular payload prior to a reaction between a linker and a muscle-targeting agent or molecular payload. In some embodiments, an electrophile may be an azide, a pentafluorophenyl, a silicon centers, a carbonyl, a carboxylic acid, an anhydride, an isocyanate, a thioisocyanate, a succinimidyl ester, a sulfosuccinimidyl ester, a maleimide, an alkyl halide, an alkyl pseudohalide, an epoxide, an episulfide, an aziridine, an aryl, an activated phosphorus center, and/or (e.g., and) an activated sulfur center. In some embodiments, a nucleophile may be an optionally substituted alkene, an optionally substituted alkyne, an optionally substituted aryl, an optionally substituted heterocyclyl, a hydroxyl group, an amino group, an alkylamino group, an anilido group, or a thiol group.

In some embodiments, the val-cit linker attached to a reactive chemical moiety (e.g., SPAAC for click chemistry conjugation) is conjugated to the anti-TfR antibody by a structure of:

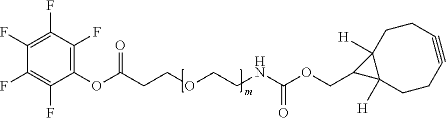

wherein m is any number from 0-10. In some embodiments, m is 4.

In some embodiments, the val-cit linker attached to a reactive chemical moiety (e.g., SPAAC for click chemistry conjugation) is conjugated to an anti-TfR antibody having a structure of:

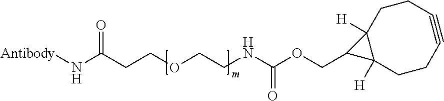

wherein m is any number from 0-10. In some embodiments, m is 4.

In some embodiments, the val-cit linker attached to a reactive chemical moiety (e.g., SPAAC for click chemistry conjugation) and is conjugated to an anti-TfR antibody has a structure of:

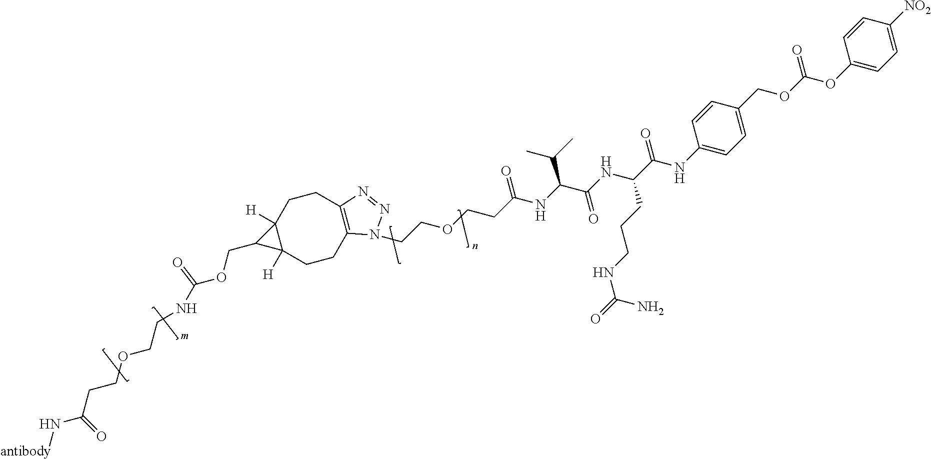

wherein n is any number from 0-10, wherein m is any number from 0-10. In some embodiments, n is 3 and/or (e.g., and) m is 4.

In some embodiments, an anti-TfR antibody and a molecular payload (e.g., an oligonucleotide) is linked via a structure of:

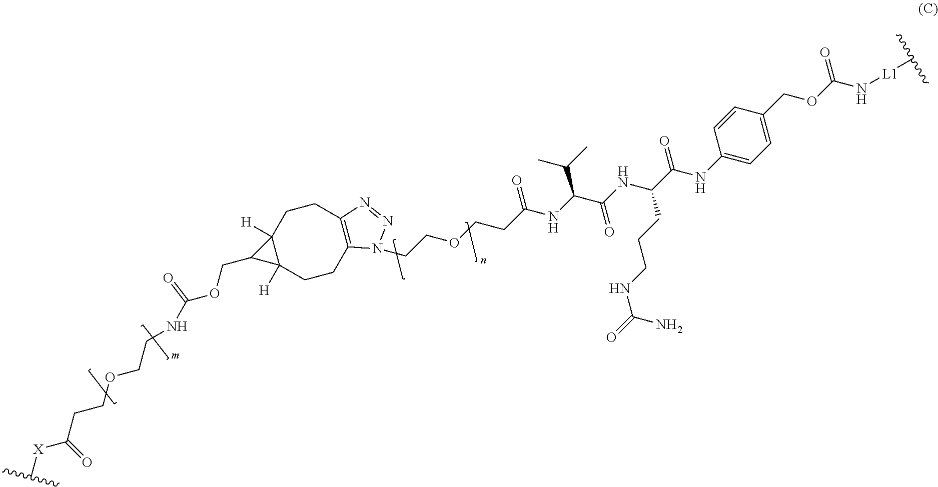

wherein n is any number from 0-10, wherein m is any number from 0-10. In some embodiments, n is 3 and/or (e.g., and) m is 4. In some embodiments, X is NH (e.g., NH from an amine group of a lysine). In some embodiments, X is S and the antibody is linked via conjugation to a cysteine of the antibody. In some embodiments, X is 0 and the antibody is linked via conjugation to a hydroxyl group of a serine, threonine, or tyrosine of the antibody.

In some embodiments, the complex described herein has a structure of:

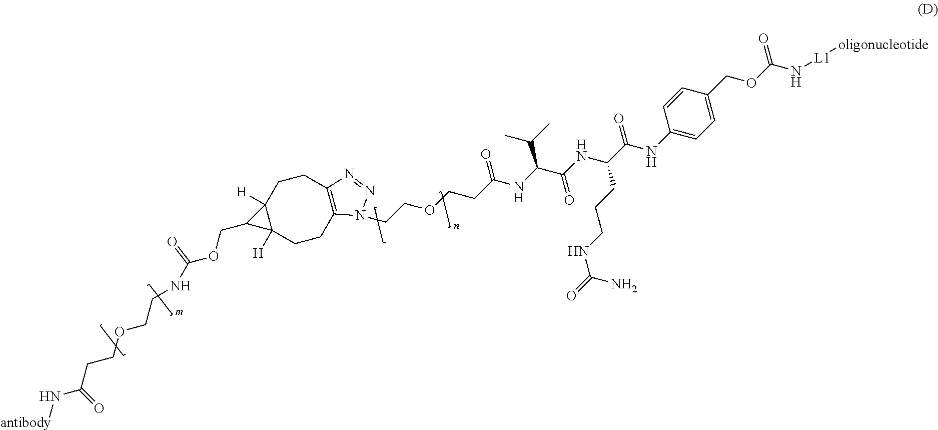

wherein n is any number from 0-10, wherein m is any number from 0-10. In some embodiments, n is 3 and/or (e.g., and) m is 4.

In structures formula (A), (B), (C), and (D), L1 is, in some embodiments, is a spacer that is substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof, wherein each RA is independently hydrogen or substituted or unsubstituted alkyl. In some embodiments, L1 is

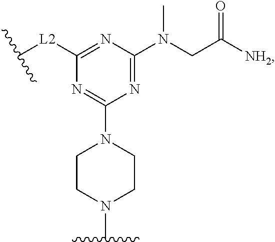

wherein the piperazine moiety links to the oligonucleotide, wherein L2 is

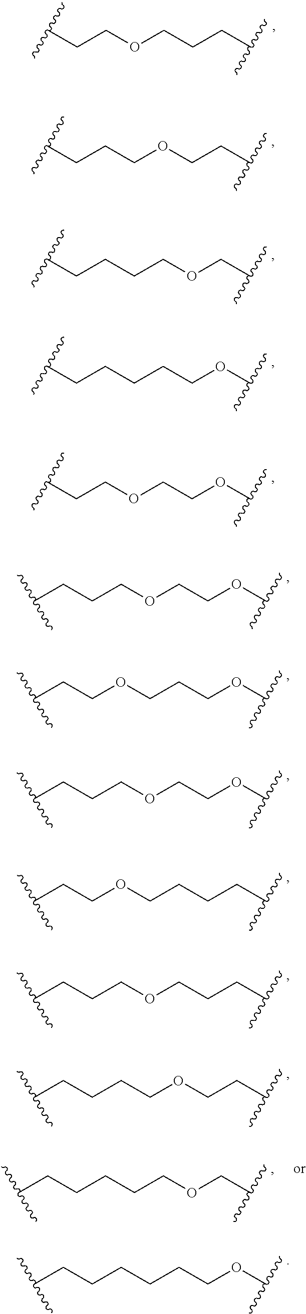

In some embodiments, L1 is:

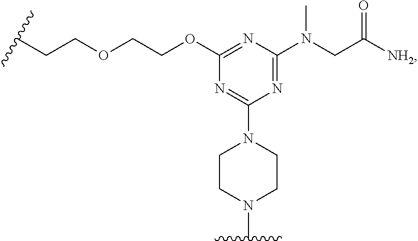 [SMILES: *CCOCCOc1nc(N(C)CC(N)=O)nc(N2CCN(*)CC2)n1]

wherein the piperazine moiety links to the oligonucleotide.

In some embodiments, L1 is

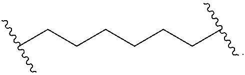 [SMILES: *CCCCCC*]

In some embodiments, L1 is linked to the 5′ phosphate of the oligonucleotide. In some embodiments, L1 is linked to the 5′ phosphorothioate of the oligonucleotide. In some embodiments, L1 is linked to the 5′ phosphonoamidate of the oligonucleotide.

In some embodiments, L1 is optional (e.g., need not be present).

D. Examples of Antibody-Molecular Payload Complexes

Other aspects of the present disclosure provide complexes comprising any one the muscle targeting agent (e.g., a transferrin receptor antibodies) described herein covalently linked to any of the molecular payloads (e.g., an oligonucleotide) described herein. In some embodiments, the muscle targeting agent (e.g., a transferrin receptor antibody) is covalently linked to a molecular payload (e.g., an oligonucleotide) via a linker. Any of the linkers described herein may be used. In some embodiments, the linker is linked to the 5′ end, the 3′ end, or internally of the oligonucleotide. In some embodiments, the linker is linked to the antibody via a thiol-reactive linkage (e.g., via a cysteine in the antibody). In some embodiments, the linker (e.g., a Val-cit linker) is linked to the antibody (e.g., an anti-TfR antibody described herein) via a n amine group (e.g., via a lysine in the antibody).

An example of a structure of a complex comprising a transferrin receptor antibody covalently linked to an oligonucleotide via a Val-cit linker is provided below:

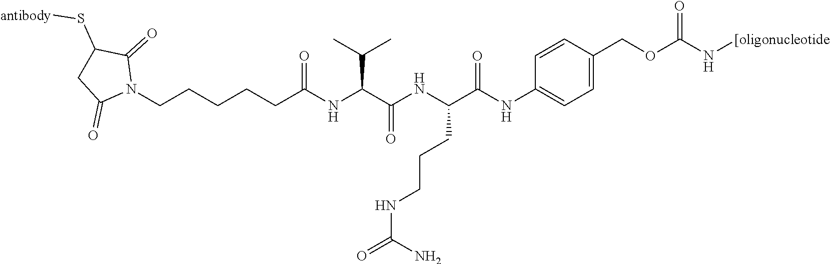

wherein the linker is linked to the 5′ end, the 3′ end, or internally of the oligonucleotide, and wherein the linker is linked to the antibody via a thiol-reactive linkage (e.g., via a cysteine in the antibody).

Another example of a structure of a complex comprising an anti-TfR antibody covalently linked to a molecular payload via a Val-cit linker is provided below:

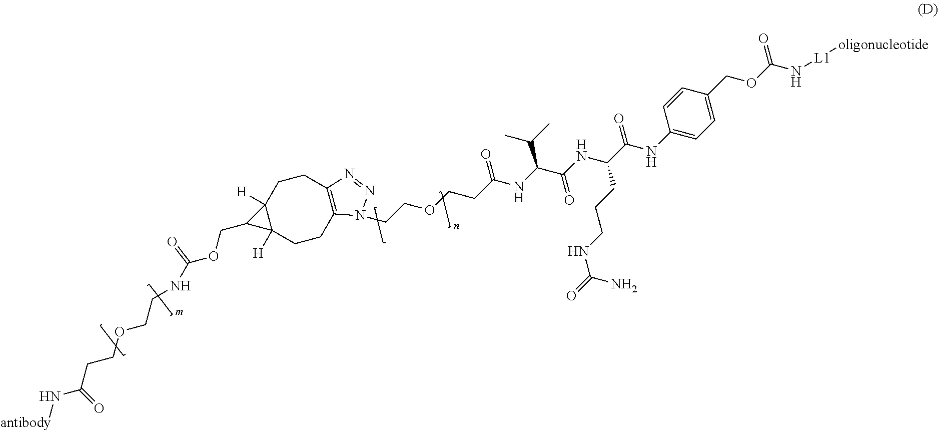

wherein n is a number between 0-10, wherein m is a number between 0-10, wherein the linker is linked to the antibody via an amine group (e.g., on a lysine residue), and/or (e.g., and) wherein the linker is linked to the oligonucleotide (e.g., at the 5′ end, 3′ end, or internally). In some embodiments, the linker is linked to the antibody via a lysine, the linker is linked to the oligonucleotide at the 5′ end, n is 3, and m is 4. In some embodiments, the molecular payload is an oligonucleotide (e.g., an oligonucleotide targeting a muscle atrophy gene listed in Table 1). In some embodiments, the oligonucleotide is a siRNA comprising an antisense strand and a sense strand, and is linked via the 3′ or 5′ end of either the sense strand or the antisense strand.

It should be appreciated that antibodies can be linked to oligonucleotides with different stochiometries, a property that may be referred to as a drug to antibody ratios (DAR) with the “drug” being the oligonucleotide. In some embodiments, one oligonucleotide is linked to an antibody (DAR=1). In some embodiments, two oligonucleotides are linked to an antibody (DAR=2). In some embodiments, three oligonucleotides are linked to an antibody (DAR=3). In some embodiments, four oligonucleotides are linked to an antibody (DAR=4). In some embodiments, a mixture of different complexes, each having a different DAR, is provided. In some embodiments, an average DAR of complexes in such a mixture may be in a range of 1 to 3, 1 to 4, 1 to 5 or more. DAR may be increased by conjugating oligonucleotides to different sites on an antibody and/or (e.g., and) by conjugating multimers to one or more sites on antibody. For example, a DAR of 2 may be achieved by conjugating a single oligonucleotide to two different sites on an antibody or by conjugating a dimer oligonucleotide to a single site of an antibody.

In some embodiments, the complex described herein comprises a transferrin receptor antibody (e.g., an antibody or any variant thereof as described herein) covalently linked to an oligonucleotide. In some embodiments, the complex described herein comprises a transferrin receptor antibody (e.g., an antibody or any variant thereof as described herein) covalently linked to an oligonucleotide via a linker (e.g., a Val-cit linker). In some embodiments, the linker (e.g., a Val-cit linker) is linked to the 5′ end, the 3′ end, or internally of the oligonucleotide. In some embodiments, the linker (e.g., a Val-cit linker) is linked to the antibody (e.g., an antibody or any variant thereof as described herein) via a thiol-reactive linkage (e.g., via a cysteine in the antibody). In some embodiments, the linker (e.g., a Val-cit linker) is linked to the antibody (e.g., an anti-TfR antibody described herein) via an amine group (e.g., via a lysine in the antibody).

In some embodiments, in any one of the examples of complexes described herein, the molecular payload is an oligonucleotide comprising a region of complementarity of at least 15 nucleotides to any one of the gene target sequences described herein (e.g., a target sequence for a muscle atrophy gene listed in Table 1).

In some embodiments, the complex described herein comprises an anti-TfR antibody covalently linked to a molecular payload (e.g., an oligonucleotide), wherein the anti-TfR antibody comprises a CDR-H1, a CDR-H2, and a CDR-H3 that are the same as the CDR-H1, CDR-H2, and CDR-H3 shown in Table 2, Table 4, Table 7, or Table 9; and a CDR-L1, a CDR-L2, and a CDR-L3 that are the same as the CDR-L1, CDR-L2, and CDR-L3 shown in Table 2, Table 4, Table 7, or Table 9. In some embodiments, the molecular payload is an oligonucleotide (e.g., an oligonucleotide targeting a muscle atrophy gene listed in Table 1).

In some embodiments, the complex described herein comprises an anti-TfR antibody covalently linked to a molecular payload (e.g., an oligonucleotide), wherein the anti-TfR antibody comprises:

(i) a CDR-H1 of SEQ ID NO: 1, a CDR-H2 of SEQ ID NO: 2, SEQ ID NO: 262, or SEQ ID NO: 80, a CDR-H3 of SEQ ID NO: 3, a CDR-L1 of SEQ ID NO: 4, a CDR-L2 of SEQ ID NO: 5, and a CDR-L3 of SEQ ID NO: 6;

(ii) a CDR-H1 of SEQ ID NO: 145, a CDR-H2 of SEQ ID NO: 146, SEQ ID NO: 263, or SEQ ID NO: 265, a CDR-H3 of SEQ ID NO: 147, a CDR-L1 of SEQ ID NO: 148, a CDR-L2 of SEQ ID NO: 149, and a CDR-L3 of SEQ ID NO: 6; or

(iii) a CDR-H1 of SEQ ID NO: 150, a CDR-H2 of SEQ ID NO: 151, SEQ ID NO: 270, or SEQ ID NO: 271, a CDR-H3 of SEQ ID NO: 152, a CDR-L1 of SEQ ID NO: 153, a CDR-L2 of SEQ ID NO: 5, and a CDR-L3 of SEQ ID NO: 154. In some embodiments, the molecular payload is an oligonucleotide (e.g., an oligonucleotide targeting a muscle atrophy gene listed in Table 1).

In some embodiments, the complex described herein comprises an anti-TfR antibody covalently linked to a molecular payload (e.g., an oligonucleotide), wherein the anti-TfR antibody comprises:

(i) a CDR-H1 of SEQ ID NO: 9, a CDR-H2 of SEQ ID NO: 10, a CDR-H3 of SEQ ID NO: 11, a CDR-L1 of SEQ ID NO: 12, a CDR-L2 of SEQ ID NO: 13, and a CDR-L3 of SEQ ID NO: 14;

(ii) a CDR-H1 of SEQ ID NO: 155, a CDR-H2 of SEQ ID NO: 156, a CDR-H3 of SEQ ID NO: 157, a CDR-L1 of SEQ ID NO: 158, a CDR-L2 of SEQ ID NO: 159, and a CDR-L3 of SEQ ID NO: 14; or

(iii) a CDR-H1 of SEQ ID NO: 160, a CDR-H2 of SEQ ID NO: 161, a CDR-H3 of SEQ ID NO: 162, a CDR-L1 of SEQ ID NO: 163, a CDR-L2 of SEQ ID NO: 13, and a CDR-L3 of SEQ ID NO: 164. In some embodiments, the molecular payload is an oligonucleotide (e.g., an oligonucleotide targeting a muscle atrophy gene listed in Table 1).

In some embodiments, the complex described herein comprises an anti-TfR antibody covalently linked to a molecular payload (e.g., an oligonucleotide), wherein the anti-TfR antibody comprises:

(i) a CDR-H1 of SEQ ID NO: 17, SEQ ID NO: 266, or SEQ ID NO: 268, a CDR-H2 of SEQ ID NO: 18, a CDR-H3 of SEQ ID NO: 19, a CDR-L1 of SEQ ID NO: 20, a CDR-L2 of SEQ ID NO: 21, and a CDR-L3 of SEQ ID NO: 22;

(ii) a CDR-H1 of SEQ ID NO: 165, SEQ ID NO: 267, or SEQ ID NO: 269, a CDR-H2 of SEQ ID NO: 166, a CDR-H3 of SEQ ID NO: 167, a CDR-L1 of SEQ ID NO: 168, a CDR-L2 of SEQ ID NO: 169, and a CDR-L3 of SEQ ID NO: 22; or

(iii) a CDR-H1 of SEQ ID NO: 170, a CDR-H2 of SEQ ID NO: 171, a CDR-H3 of SEQ ID NO: 172, a CDR-L1 of SEQ ID NO: 173, a CDR-L2 of SEQ ID NO: 21, and a CDR-L3 of SEQ ID NO: 174. In some embodiments, the molecular payload is an oligonucleotide (e.g., an oligonucleotide targeting a muscle atrophy gene listed in Table 1).

In some embodiments, the complex described herein comprises an anti-TfR antibody covalently linked to a molecular payload (e.g., an oligonucleotide), wherein the anti-TfR antibody comprises:

(i) a CDR-H1 of SEQ ID NO: 188, a CDR-H2 of SEQ ID NO: 189, a CDR-H3 of SEQ ID NO: 190, a CDR-L1 of SEQ ID NO: 191, a CDR-L2 of SEQ ID NO: 192, and a CDR-L3 of SEQ ID NO: 193;

(ii) a CDR-H1 of SEQ ID NO: 194, a CDR-H2 of SEQ ID NO: 195, a CDR-H3 of SEQ ID NO: 196, a CDR-L1 of SEQ ID NO: 197, a CDR-L2 of SEQ ID NO: 198, and a CDR-L3 of SEQ ID NO: 193; or

(iii) a CDR-H1 of SEQ ID NO: 199, a CDR-H2 of SEQ ID NO: 200, a CDR-H3 of SEQ ID NO: 201, a CDR-L1 of SEQ ID NO: 202, a CDR-L2 of SEQ ID NO: 192, and a CDR-L3 of SEQ ID NO: 203. In some embodiments, the molecular payload is an oligonucleotide (e.g., an oligonucleotide targeting a muscle atrophy gene listed in Table 1).

In some embodiments, the complex described herein comprises an anti-TfR antibody covalently linked to a molecular payload (e.g., an oligonucleotide), wherein the anti-TfR antibody comprises VH as shown in Table 2 or Table 7; and a VL as shown in Table 2 or Table 7. In some embodiments, the molecular payload is an oligonucleotide (e.g., an oligonucleotide targeting a muscle atrophy gene listed in Table 1).

In some embodiments, the complex described herein comprises an anti-TfR antibody covalently linked to a molecular payload (e.g., an oligonucleotide), wherein the anti-TfR antibody comprises a VH having the amino acid sequence of SEQ ID NO: 7 and a VL having the amino acid sequence of SEQ ID NO: 8. In some embodiments, the molecular payload is an oligonucleotide (e.g., an oligonucleotide targeting a muscle atrophy gene listed in Table 1).

In some embodiments, the complex described herein comprises an anti-TfR antibody covalently linked to a molecular payload (e.g., an oligonucleotide), wherein the anti-TfR antibody comprises a VH having the amino acid sequence of SEQ ID NO: 15 and a VL having the amino acid sequence of SEQ ID NO: 16. In some embodiments, the molecular payload is an oligonucleotide (e.g., an oligonucleotide targeting a muscle atrophy gene listed in Table 1).

In some embodiments, the complex described herein comprises an anti-TfR antibody covalently linked to a molecular payload (e.g., an oligonucleotide), wherein the anti-TfR antibody comprises a VH having the amino acid sequence of SEQ ID NO: 23 and a VL having the amino acid sequence of SEQ ID NO: 24. In some embodiments, the molecular payload is an oligonucleotide (e.g., an oligonucleotide targeting a muscle atrophy gene listed in Table 1).

In some embodiments, the complex described herein comprises an anti-TfR antibody covalently linked to a molecular payload (e.g., an oligonucleotide), wherein the anti-TfR antibody comprises a VH having the amino acid sequence of SEQ ID NO: 204 and a VL having the amino acid sequence of SEQ ID NO: 205. In some embodiments, the molecular payload is an oligonucleotide (e.g., an oligonucleotide targeting a muscle atrophy gene listed in Table 1).

In some embodiments, the complex described herein comprises an anti-TfR antibody covalently linked to a molecular payload (e.g., an oligonucleotide), wherein the anti-TfR antibody comprises a heavy chain having the amino acid sequence of SEQ ID NO: 178, SEQ ID NO: 185, SEQ ID NO: 300, SEQ ID NO: 301, SEQ ID NO: 304, or SEQ ID NO: 305, and a light chain having the amino acid sequence of SEQ ID NO: 179. In some embodiments, the molecular payload is an oligonucleotide (e.g., an oligonucleotide targeting a muscle atrophy gene listed in Table 1).

In some embodiments, the complex described herein comprises an anti-TfR antibody covalently linked to a molecular payload (e.g., an oligonucleotide) wherein the anti-TfR antibody comprises a heavy chain having the amino acid sequence of SEQ ID NO: 180, SEQ ID NO: 186, and a light chain having the amino acid sequence of SEQ ID NO: 181. In some embodiments, the molecular payload is an oligonucleotide (e.g., an oligonucleotide targeting a muscle atrophy gene listed in Table 1).

In some embodiments, the complex described herein comprises an anti-TfR antibody covalently linked to a molecular payload (e.g., an oligonucleotide), wherein the anti-TfR antibody comprises a heavy chain having the amino acid sequence of SEQ ID NO: 182, SEQ ID NO: 187, SEQ ID NO: 302, SEQ ID NO: 303, SEQ ID NO: 306, or SEQ ID NO: 307, and a light chain having the amino acid sequence of SEQ ID NO: 183. In some embodiments, the molecular payload is an oligonucleotide (e.g., an oligonucleotide targeting a muscle atrophy gene listed in Table 1).

In some embodiments, the complex described herein comprises an anti-TfR antibody covalently linked to a molecular payload (e.g., an oligonucleotide), wherein the anti-TfR antibody comprises a heavy chain having the amino acid sequence of SEQ ID NO: 210, SEQ ID NO: 211, SEQ ID NO: 213, or SEQ ID NO: 308, and a light chain having the amino acid sequence of SEQ ID NO: 212. In some embodiments, the molecular payload is an oligonucleotide (e.g., an oligonucleotide targeting a muscle atrophy gene listed in Table 1).

In some embodiments, the complex described herein comprises an anti-TfR antibody covalently linked to a molecular payload, wherein the antibody is a humanized antibody that comprises a VH that contains human framework regions with the CDR-H1, CDR-H2, and CDR-H3 of a murine antibody listed in Table 2 or Table 4 (e.g., 3A4, 3M12, or 5H12), and a VL that contains human framework regions with the CDR-L1, CDR-L2, and CDR-L3 of a murine antibody listed in Table 2 or Table 4 (e.g., 3A4, 3M12, or 5H12). In some embodiments, the molecular payload is an oligonucleotide (e.g., an oligonucleotide targeting a muscle atrophy gene listed in Table 1).

In some embodiments, the complex described herein comprises an anti-TfR antibody covalently linked to a molecular payload, wherein the antibody comprises a VH that contains human framework regions with the CDR-H1, CDR-H2, and CDR-H3 of a VH as set forth in SEQ ID NO: 7, and a VL that contains human framework regions with the CDR-L1, CDR-L2, and CDR-L3 of a VL as forth in SEQ ID NO: 8. In some embodiments, the molecular payload is an oligonucleotide (e.g., an oligonucleotide targeting a muscle atrophy gene listed in Table 1).

In some embodiments, the complex described herein comprises an anti-TfR antibody covalently linked to a molecular payload, wherein the antibody comprises a VH that contains human framework regions with the CDR-H1, CDR-H2, and CDR-H3 of a VH as set forth in SEQ ID NO: 15, and a VL that contains human framework regions with the CDR-L1, CDR-L2, and CDR-L3 of a VL as forth in SEQ ID NO: 16. In some embodiments, the molecular payload is an oligonucleotide (e.g., an oligonucleotide targeting a muscle atrophy gene listed in Table 1).

In some embodiments, the complex described herein comprises an anti-TfR antibody covalently linked to a molecular payload, wherein the antibody comprises a VH that contains human framework regions with the CDR-H1, CDR-H2, and CDR-H3 of a VH as set forth in SEQ ID NO: 23, and a VL that contains human framework regions with the CDR-L1, CDR-L2, and CDR-L3 of a VL as forth in SEQ ID NO: 24. In some embodiments, the molecular payload is an oligonucleotide (e.g., an oligonucleotide targeting a muscle atrophy gene listed in Table 1).

In some embodiments, the complex described herein comprises an anti-TfR antibody covalently linked to a molecular payload, wherein the antibody is an IgG1 kappa that comprises human framework regions with the CDRs of a murine antibody listed in Table 2 or Table 4 (e.g., 3A4, 3M12, or 5H12). In some embodiments, the molecular payload is an oligonucleotide (e.g., an oligonucleotide targeting a muscle atrophy gene listed in Table 1).

In some embodiments, the complex described herein comprises an anti-TfR antibody covalently linked to a molecular payload, wherein the antibody is a Fab′ fragment of an IgG1 kappa that comprises human framework regions with the CDRs of a murine antibody listed in Table 2 or Table 4 (e.g., 3A4, 3M12, or 5H12). In some embodiments, the molecular payload is an oligonucleotide (e.g., an oligonucleotide targeting a muscle atrophy gene listed in Table 1).

In some embodiments, the complex described herein comprises an anti-TfR antibody covalently linked to a molecular payload, wherein the antibody is a Fab′ fragment of an IgG1 kappa that comprises human framework regions with the CDRs of a murine antibody listed in Table 2 or Table 4 (e.g., 3A4, 3M12, or 5H12). In some embodiments, the molecular payload is an oligonucleotide (e.g., an oligonucleotide targeting a muscle atrophy gene listed in Table 1).

In some embodiments, the complex described herein comprises an anti-TfR antibody covalently linked via a lysine to the 5′ end of an oligonucleotide, wherein the antibody is a Fab′ fragment of an IgG1 kappa that comprises human framework regions with the CDRs of a murine antibody listed in Table 2 or Table 4 (e.g., 3A4, 3M12, or 5H12), wherein the complex has the structure of:

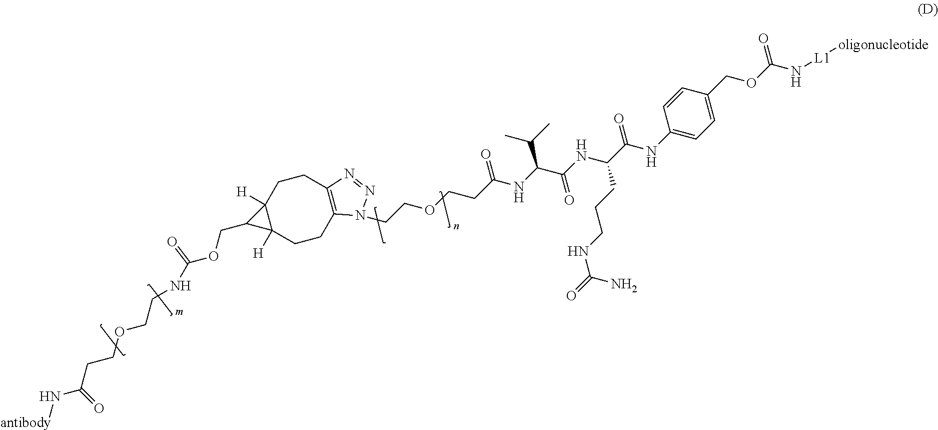

wherein n is 3 and m is 4. In some embodiments, the molecular payload is an oligonucleotide (e.g., an oligonucleotide targeting a muscle atrophy gene listed in Table 1).

In some embodiments, the complex described herein comprises an anti-TfR antibody covalently linked via a lysine to the 5′ end of an oligonucleotide, wherein the antibody is a Fab′ fragment of an IgG1 kappa that comprises a heavy chain comprising the amino acid sequence of SEQ ID NO: 308 and a light chain comprising the amino acid sequence of SEQ ID NO: 212, wherein the complex has the structure of:

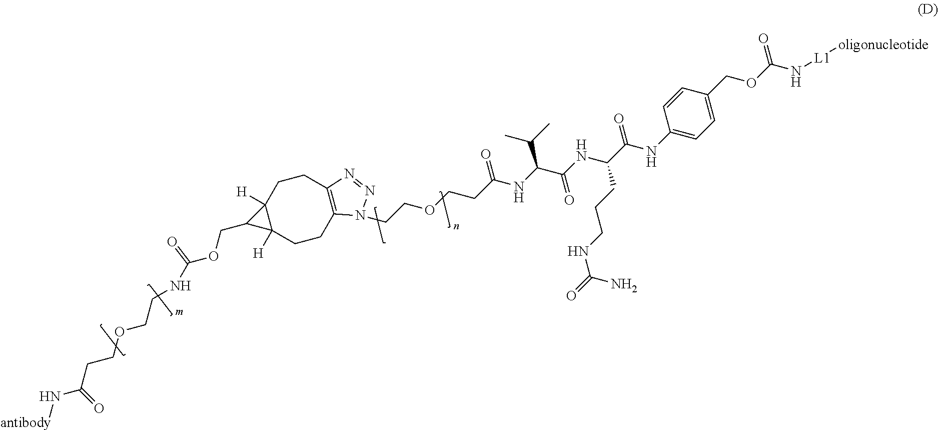

wherein n is 3 and m is 4. In some embodiments, the molecular payload is an oligonucleotide (e.g., an oligonucleotide targeting a muscle atrophy gene listed in Table 1).

In some embodiments, the complex described herein comprises an anti-TfR antibody covalently linked via a lysine to the 5′ end of an oligonucleotide, wherein the antibody is a Fab′ fragment of an IgG1 kappa that comprises a heavy chain comprising the amino acid sequence of SEQ ID NO: 213 and a light chain comprising the amino acid sequence of SEQ ID NO: 212, wherein the complex has the structure of:

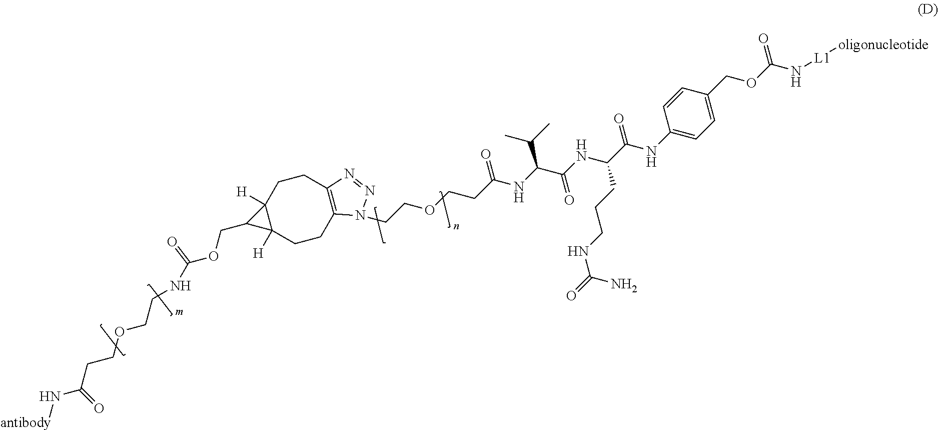

wherein n is 3 and m is 4. In some embodiments, the molecular payload is an oligonucleotide (e.g., an oligonucleotide targeting a muscle atrophy gene listed in Table 1).

In some embodiments, the complex described herein comprises an anti-TfR Fab covalently linked via a lysine to the 5′ end of an oligonucleotide (e.g., an oligonucleotide targeting a muscle atrophy gene listed in Table 1), wherein the anti-TfR Fab comprises a CDR-H1 as set forth in SEQ ID NO: 1, a CDR-H2 as set forth in SEQ ID NO: 2, a CDR-H3 as set forth in SEQ ID NO: 3, a CDR-L1 as set forth in SEQ ID NO: 4, a CDR-L2 as set forth in SEQ ID NO: 5, and a CDR-L3 as set forth in SEQ ID NO: 6; wherein the complex has the structure of:

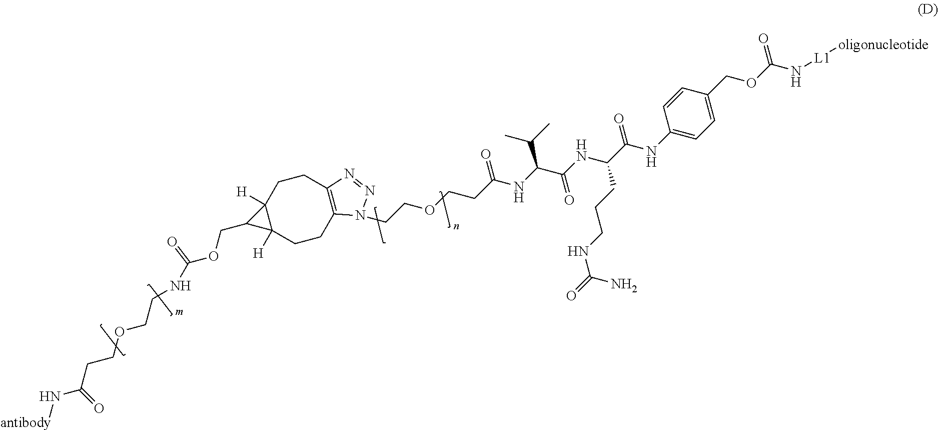

wherein n is 3 and m is 4, optionally wherein the oligonucleotide comprises a region of complementarity of at least 8 nucleotides to a pro-atrophy gene provided in Table 1.

In some embodiments, the complex described herein comprises an anti-TfR Fab covalently linked via a lysine to the 5′ end of an oligonucleotide (e.g., an oligonucleotide targeting a muscle atrophy gene listed in Table 1), wherein the anti-TfR Fab comprises a CDR-H1 as set forth in SEQ ID NO: 1, a CDR-H2 as set forth in SEQ ID NO: 262, a CDR-H3 as set forth in SEQ ID NO: 3, a CDR-L1 as set forth in SEQ ID NO: 4, a CDR-L2 as set forth in SEQ ID NO: 5, and a CDR-L3 as set forth in SEQ ID NO: 6; wherein the complex has the structure of:

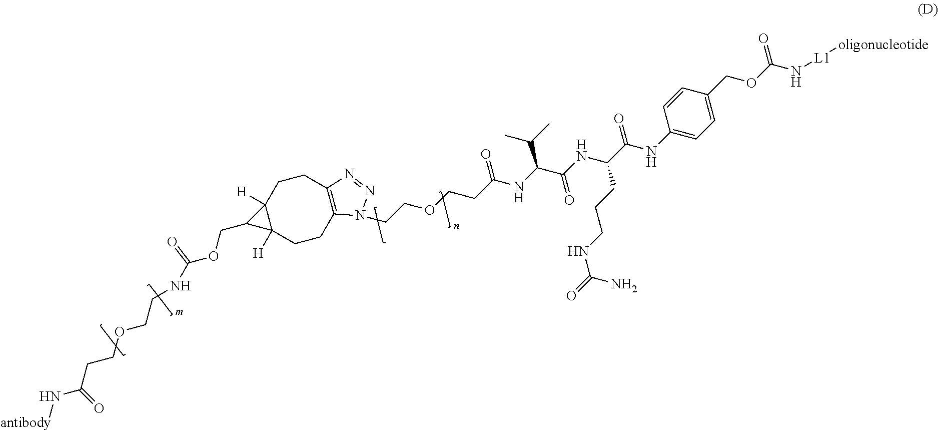

wherein n is 3 and m is 4, optionally wherein the oligonucleotide comprises a region of complementarity of at least 8 nucleotides to a pro-atrophy gene provided in Table 1.

In some embodiments, the complex described herein comprises an anti-TfR Fab covalently linked via a lysine to the 5′ end of an oligonucleotide (e.g., an oligonucleotide targeting a muscle atrophy gene listed in Table 1), wherein the anti-TfR Fab comprises a CDR-H1 as set forth in SEQ ID NO: 1, a CDR-H2 as set forth in SEQ ID NO: 80, a CDR-H3 as set forth in SEQ ID NO: 3, a CDR-L1 as set forth in SEQ ID NO: 4, a CDR-L2 as set forth in SEQ ID NO: 5, and a CDR-L3 as set forth in SEQ ID NO: 6; wherein the complex has the structure of:

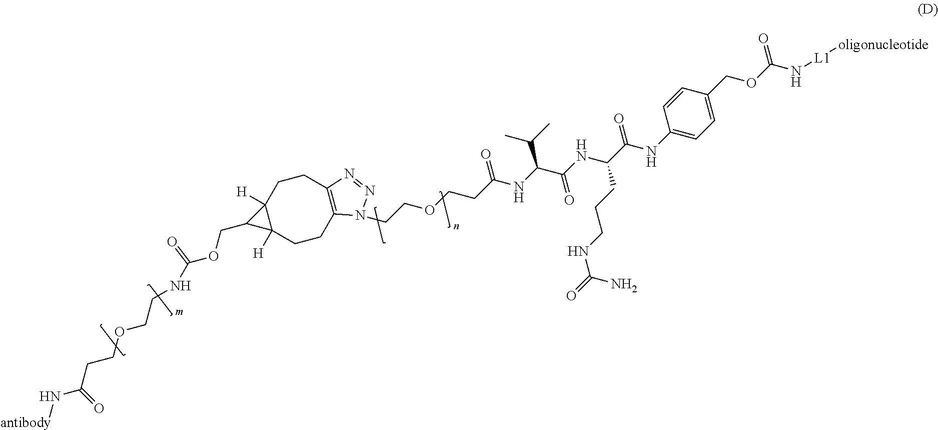

wherein n is 3 and m is 4, optionally wherein the oligonucleotide comprises a region of complementarity of at least 8 nucleotides to a pro-atrophy gene provided in Table 1.

In some embodiments, the complex described herein comprises an anti-TfR Fab covalently linked via a lysine to the 5′ end of an oligonucleotide (e.g., an oligonucleotide targeting a muscle atrophy gene listed in Table 1), wherein the anti-TfR Fab comprises a CDR-H1 as set forth in SEQ ID NO: 145, a CDR-H2 as set forth in SEQ ID NO: 146, a CDR-H3 as set forth in SEQ ID NO: 147, a CDR-L1 as set forth in SEQ ID NO: 148, a CDR-L2 as set forth in SEQ ID NO: 149, and a CDR-L3 as set forth in SEQ ID NO: 6; wherein the complex has the structure of:

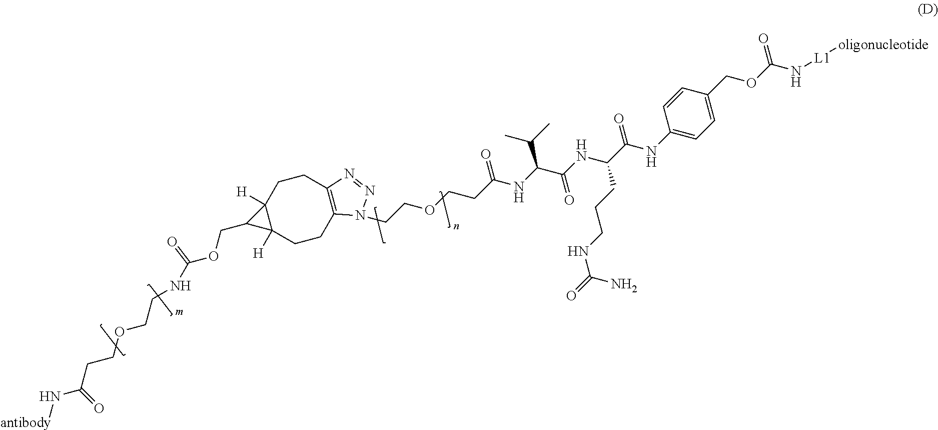

wherein n is 3 and m is 4, optionally wherein the oligonucleotide comprises a region of complementarity of at least 8 nucleotides to a pro-atrophy gene provided in Table 1.

In some embodiments, the complex described herein comprises an anti-TfR Fab covalently linked via a lysine to the 5′ end of an oligonucleotide (e.g., an oligonucleotide targeting a muscle atrophy gene listed in Table 1), wherein the anti-TfR Fab comprises a CDR-H1 as set forth in SEQ ID NO: 145, a CDR-H2 as set forth in SEQ ID NO: 263, a CDR-H3 as set forth in SEQ ID NO: 147, a CDR-L1 as set forth in SEQ ID NO: 148, a CDR-L2 as set forth in SEQ ID NO: 149, and a CDR-L3 as set forth in SEQ ID NO: 6; wherein the complex has the structure of:

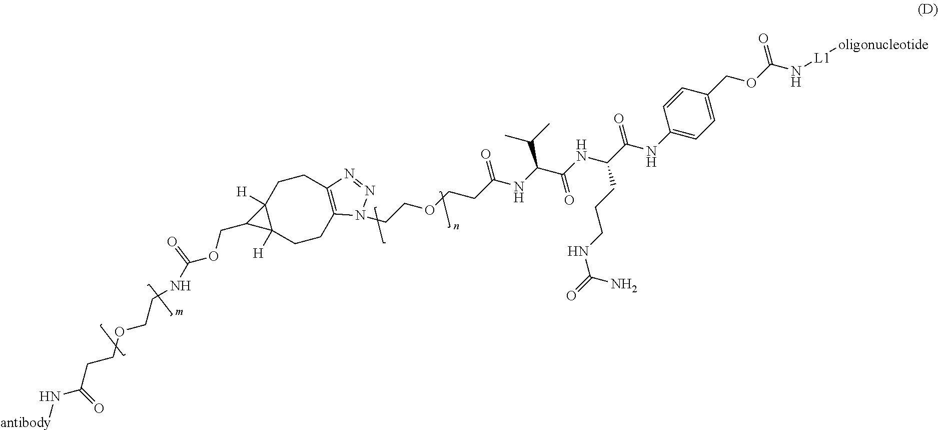

wherein n is 3 and m is 4, optionally wherein the oligonucleotide comprises a region of complementarity of at least 8 nucleotides to a pro-atrophy gene provided in Table 1.

In some embodiments, the complex described herein comprises an anti-TfR Fab covalently linked via a lysine to the 5′ end of an oligonucleotide (e.g., an oligonucleotide targeting a muscle atrophy gene listed in Table 1), wherein the anti-TfR Fab comprises a CDR-H1 as set forth in SEQ ID NO: 145, a CDR-H2 as set forth in SEQ ID NO: 265, a CDR-H3 as set forth in SEQ ID NO: 147, a CDR-L1 as set forth in SEQ ID NO: 148, a CDR-L2 as set forth in SEQ ID NO: 149, and a CDR-L3 as set forth in SEQ ID NO: 6; wherein the complex has the structure of:

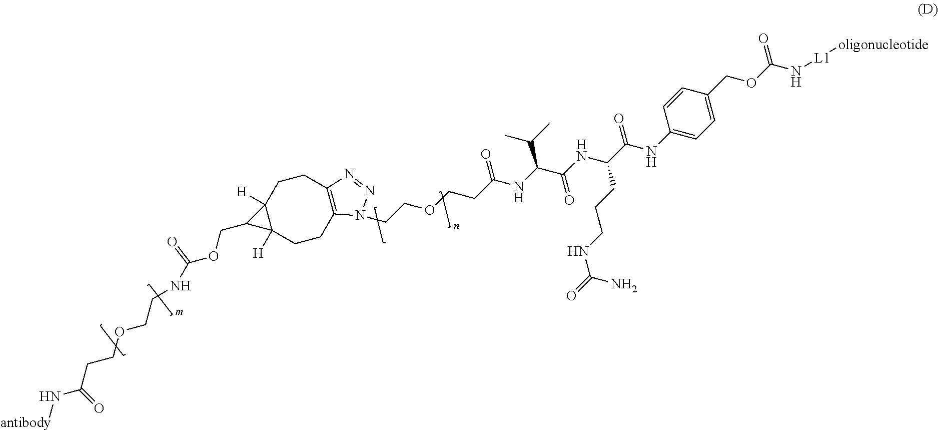

wherein n is 3 and m is 4, optionally wherein the oligonucleotide comprises a region of complementarity of at least 8 nucleotides to a pro-atrophy gene provided in Table 1.

In some embodiments, the complex described herein comprises an anti-TfR Fab covalently linked via a lysine to the 5′ end of an oligonucleotide (e.g., an oligonucleotide targeting a muscle atrophy gene listed in Table 1), wherein the anti-TfR Fab comprises a CDR-H1 as set forth in SEQ ID NO: 150, a CDR-H2 as set forth in SEQ ID NO: 151, a CDR-H3 as set forth in SEQ ID NO: 152, a CDR-L1 as set forth in SEQ ID NO: 153, a CDR-L2 as set forth in SEQ ID NO: 5, and a CDR-L3 as set forth in SEQ ID NO: 154; wherein the complex has the structure of:

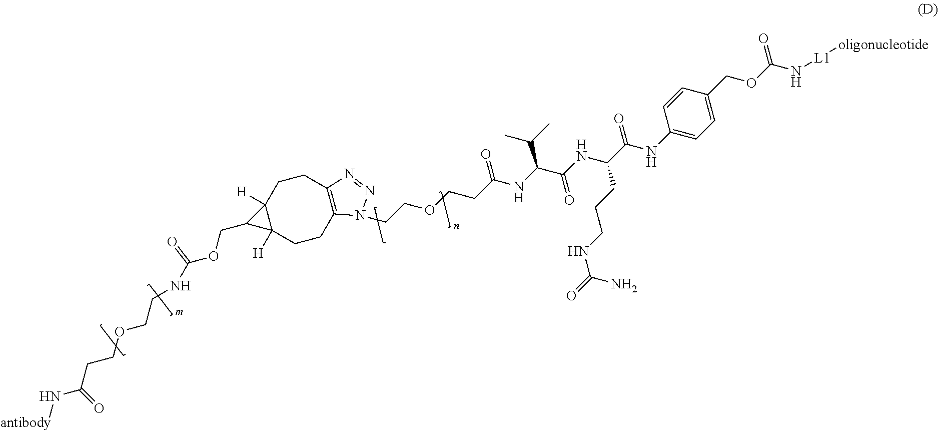

wherein n is 3 and m is 4, optionally wherein the oligonucleotide comprises a region of complementarity of at least 8 nucleotides to a pro-atrophy gene provided in Table 1.

In some embodiments, the complex described herein comprises an anti-TfR Fab covalently linked via a lysine to the 5′ end of an oligonucleotide (e.g., an oligonucleotide targeting a muscle atrophy gene listed in Table 1), wherein the anti-TfR Fab comprises a CDR-H1 as set forth in SEQ ID NO: 150, a CDR-H2 as set forth in SEQ ID NO: 270, a CDR-H3 as set forth in SEQ ID NO: 152, a CDR-L1 as set forth in SEQ ID NO: 153, a CDR-L2 as set forth in SEQ ID NO: 5, and a CDR-L3 as set forth in SEQ ID NO: 154; wherein the complex has the structure of:

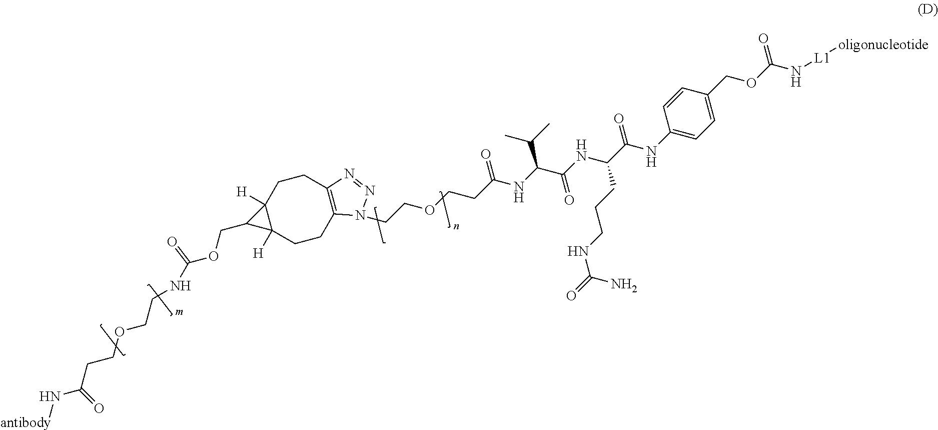

wherein n is 3 and m is 4, optionally wherein the oligonucleotide comprises a region of complementarity of at least 8 nucleotides to a pro-atrophy gene provided in Table 1.

In some embodiments, the complex described herein comprises an anti-TfR Fab covalently linked via a lysine to the 5′ end of an oligonucleotide (e.g., an oligonucleotide targeting a muscle atrophy gene listed in Table 1), wherein the anti-TfR Fab comprises a CDR-H1 as set forth in SEQ ID NO: 150, a CDR-H2 as set forth in SEQ ID NO: 271, a CDR-H3 as set forth in SEQ ID NO: 152, a CDR-L1 as set forth in SEQ ID NO: 153, a CDR-L2 as set forth in SEQ ID NO: 5, and a CDR-L3 as set forth in SEQ ID NO: 154; wherein the complex has the structure of:

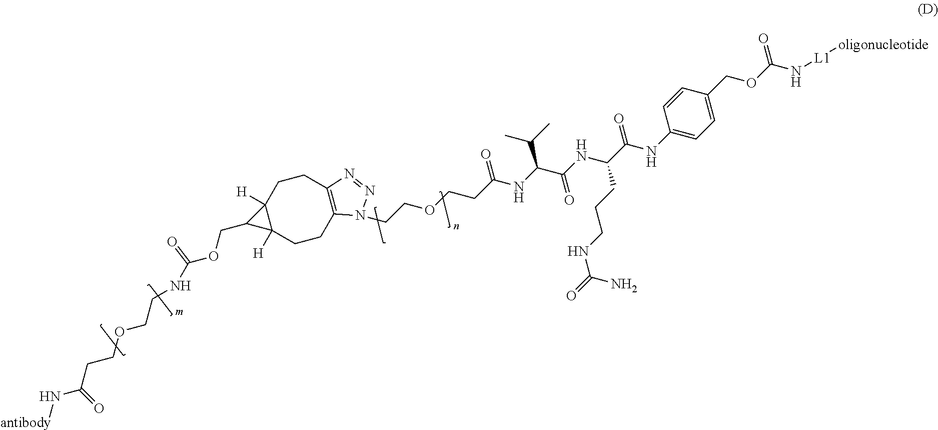

wherein n is 3 and m is 4, optionally wherein the oligonucleotide comprises a region of complementarity of at least 8 nucleotides to a pro-atrophy gene provided in Table 1.

In some embodiments, the complex described herein comprises an anti-TfR Fab covalently linked via a lysine to the 5′ end of an oligonucleotide (e.g., an oligonucleotide targeting a muscle atrophy gene listed in Table 1), wherein the anti-TfR Fab comprises a CDR-H1 as set forth in SEQ ID NO: 9, a CDR-H2 as set forth in SEQ ID NO: 10, a CDR-H3 as set forth in SEQ ID NO: 11, a CDR-L1 as set forth in SEQ ID NO: 12, a CDR-L2 as set forth in SEQ ID NO: 13, and a CDR-L3 as set forth in SEQ ID NO: 14; wherein the complex has the structure of:

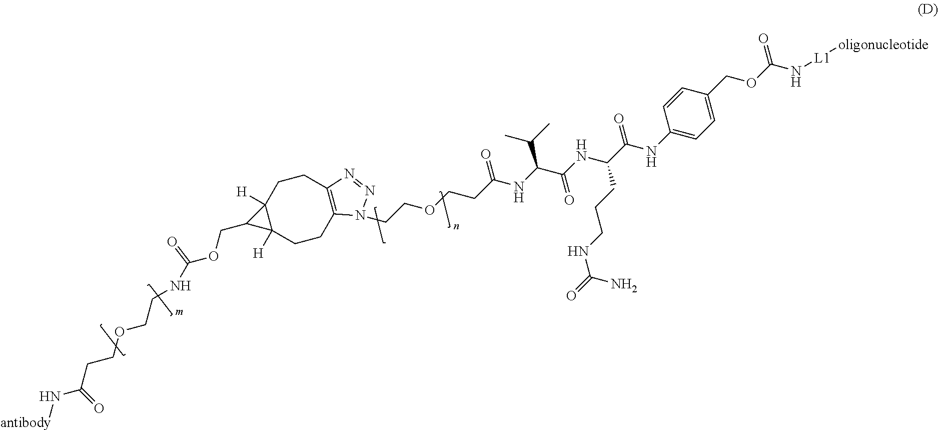

wherein n is 3 and m is 4, optionally wherein the oligonucleotide comprises a region of complementarity of at least 8 nucleotides to a pro-atrophy gene provided in Table 1.

In some embodiments, the complex described herein comprises an anti-TfR Fab covalently linked via a lysine to the 5′ end of an oligonucleotide (e.g., an oligonucleotide targeting a muscle atrophy gene listed in Table 1), wherein the anti-TfR Fab comprises a CDR-H1 as set forth in SEQ ID NO: 155, a CDR-H2 as set forth in SEQ ID NO: 156, a CDR-H3 as set forth in SEQ ID NO: 157, a CDR-L1 as set forth in SEQ ID NO: 158, a CDR-L2 as set forth in SEQ ID NO: 159, and a CDR-L3 as set forth in SEQ ID NO: 14; wherein the complex has the structure of:

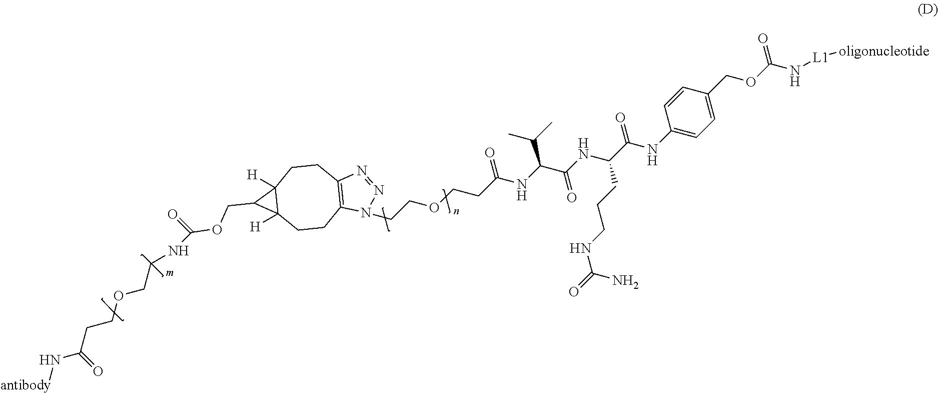

wherein n is 3 and m is 4, optionally wherein the oligonucleotide comprises a region of complementarity of at least 8 nucleotides to a pro-atrophy gene provided in Table 1.

In some embodiments, the complex described herein comprises an anti-TfR Fab covalently linked via a lysine to the 5′ end of an oligonucleotide (e.g., an oligonucleotide targeting a muscle atrophy gene listed in Table 1), wherein the anti-TfR Fab comprises a CDR-H1 as set forth in SEQ ID NO: 160, a CDR-H2 as set forth in SEQ ID NO: 161, a CDR-H3 as set forth in SEQ ID NO: 162, a CDR-L1 as set forth in SEQ ID NO: 163, a CDR-L2 as set forth in SEQ ID NO: 13, and a CDR-L3 as set forth in SEQ ID NO: 164; wherein the complex has the structure of:

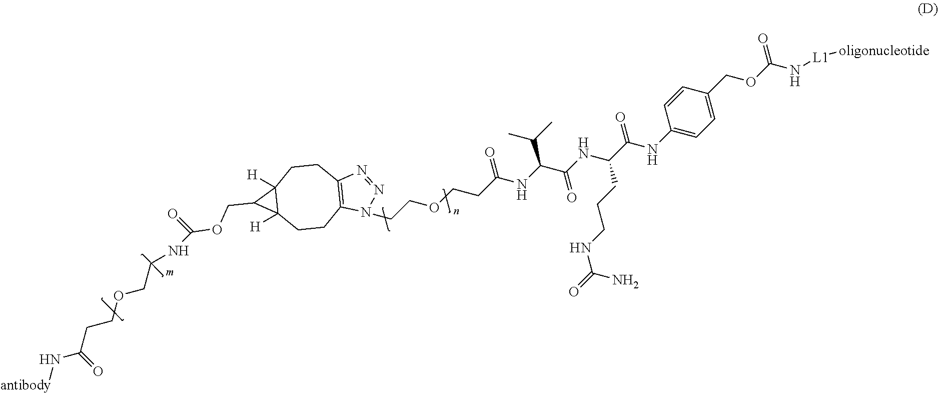

wherein n is 3 and m is 4, optionally wherein the oligonucleotide comprises a region of complementarity of at least 8 nucleotides to a pro-atrophy gene provided in Table 1.

In some embodiments, the complex described herein comprises an anti-TfR Fab covalently linked via a lysine to the 5′ end of an oligonucleotide (e.g., an oligonucleotide targeting a muscle atrophy gene listed in Table 1), wherein the anti-TfR Fab comprises a CDR-H1 as set forth in SEQ ID NO: 17, a CDR-H2 as set forth in SEQ ID NO: 18, a CDR-H3 as set forth in SEQ ID NO: 19, a CDR-L1 as set forth in SEQ ID NO: 20, a CDR-L2 as set forth in SEQ ID NO: 21, and a CDR-L3 as set forth in SEQ ID NO: 22; wherein the complex has the structure of:

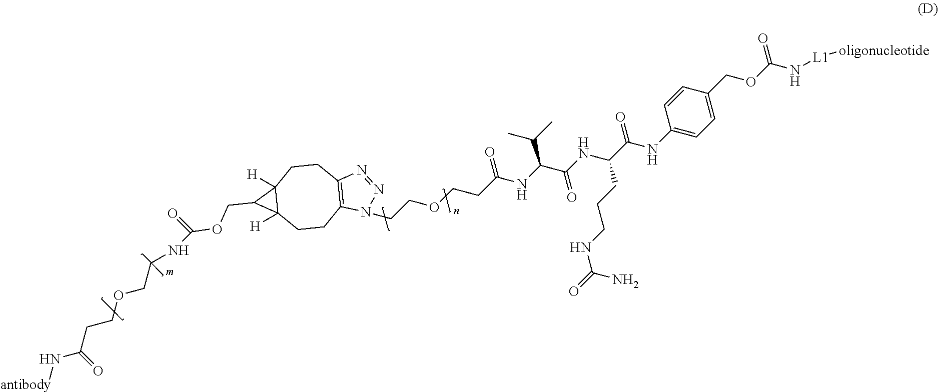

wherein n is 3 and m is 4, optionally wherein the oligonucleotide comprises a region of complementarity of at least 8 nucleotides to a pro-atrophy gene provided in Table 1.

In some embodiments, the complex described herein comprises an anti-TfR Fab covalently linked via a lysine to the 5′ end of an oligonucleotide (e.g., an oligonucleotide targeting a muscle atrophy gene listed in Table 1), wherein the anti-TfR Fab comprises a CDR-H1 as set forth in SEQ ID NO: 266, a CDR-H2 as set forth in SEQ ID NO: 18, a CDR-H3 as set forth in SEQ ID NO: 19, a CDR-L1 as set forth in SEQ ID NO: 20, a CDR-L2 as set forth in SEQ ID NO: 21, and a CDR-L3 as set forth in SEQ ID NO: 22; wherein the complex has the structure of:

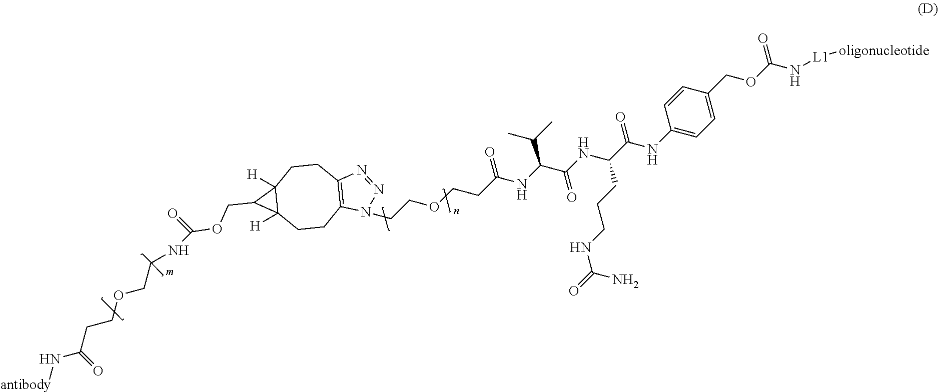

wherein n is 3 and m is 4, optionally wherein the oligonucleotide comprises a region of complementarity of at least 8 nucleotides to a pro-atrophy gene provided in Table 1.

In some embodiments, the complex described herein comprises an anti-TfR Fab covalently linked via a lysine to the 5′ end of an oligonucleotide (e.g., an oligonucleotide targeting a muscle atrophy gene listed in Table 1), wherein the anti-TfR Fab comprises a CDR-H1 as set forth in SEQ ID NO: 268, a CDR-H2 as set forth in SEQ ID NO: 18, a CDR-H3 as set forth in SEQ ID NO: 19, a CDR-L1 as set forth in SEQ ID NO: 20, a CDR-L2 as set forth in SEQ ID NO: 21, and a CDR-L3 as set forth in SEQ ID NO: 22; wherein the complex has the structure of:

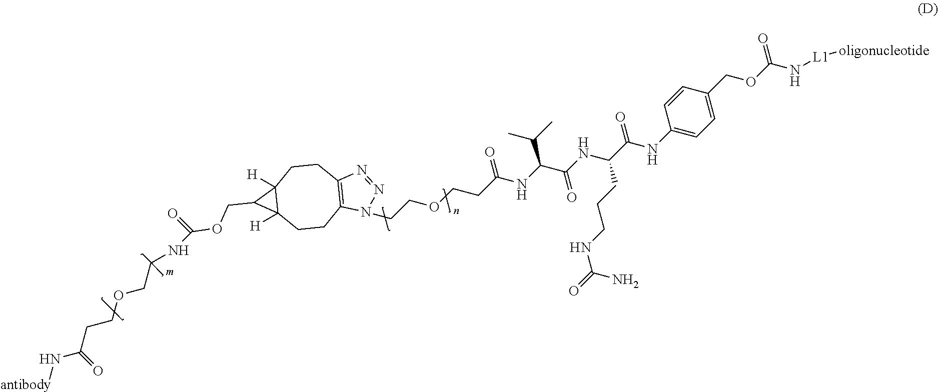

wherein n is 3 and m is 4, optionally wherein the oligonucleotide comprises a region of complementarity of at least 8 nucleotides to a pro-atrophy gene provided in Table 1.

In some embodiments, the complex described herein comprises an anti-TfR Fab covalently linked via a lysine to the 5′ end of an oligonucleotide (e.g., an oligonucleotide targeting a muscle atrophy gene listed in Table 1), wherein the anti-TfR Fab comprises a CDR-H1 as set forth in SEQ ID NO: 165, a CDR-H2 as set forth in SEQ ID NO: 166, a CDR-H3 as set forth in SEQ ID NO: 167, a CDR-L1 as set forth in SEQ ID NO: 168, a CDR-L2 as set forth in SEQ ID NO: 169, and a CDR-L3 as set forth in SEQ ID NO: 22; wherein the complex has the structure of:

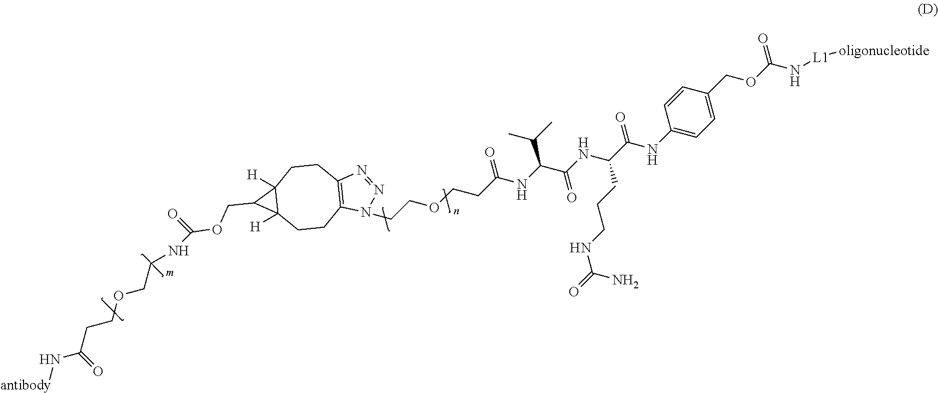

wherein n is 3 and m is 4, optionally wherein the oligonucleotide comprises a region of complementarity of at least 8 nucleotides to a pro-atrophy gene provided in Table 1.

In some embodiments, the complex described herein comprises an anti-TfR Fab covalently linked via a lysine to the 5′ end of an oligonucleotide (e.g., an oligonucleotide targeting a muscle atrophy gene listed in Table 1), wherein the anti-TfR Fab comprises a CDR-H1 as set forth in SEQ ID NO: 267, a CDR-H2 as set forth in SEQ ID NO: 166, a CDR-H3 as set forth in SEQ ID NO: 167, a CDR-L1 as set forth in SEQ ID NO: 168, a CDR-L2 as set forth in SEQ ID NO: 169, and a CDR-L3 as set forth in SEQ ID NO: 22; wherein the complex has the structure of:

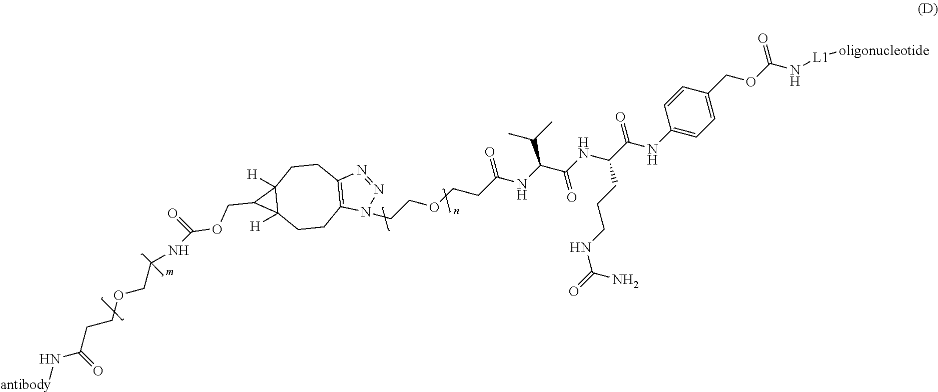

wherein n is 3 and m is 4, optionally wherein the oligonucleotide comprises a region of complementarity of at least 8 nucleotides to a pro-atrophy gene provided in Table 1.

In some embodiments, the complex described herein comprises an anti-TfR Fab covalently linked via a lysine to the 5′ end of an oligonucleotide (e.g., an oligonucleotide targeting a muscle atrophy gene listed in Table 1), wherein the anti-TfR Fab comprises a CDR-H1 as set forth in SEQ ID NO: 269, a CDR-H2 as set forth in SEQ ID NO: 166, a CDR-H3 as set forth in SEQ ID NO: 167, a CDR-L1 as set forth in SEQ ID NO: 168, a CDR-L2 as set forth in SEQ ID NO: 169, and a CDR-L3 as set forth in SEQ ID NO: 22; wherein the complex has the structure of:

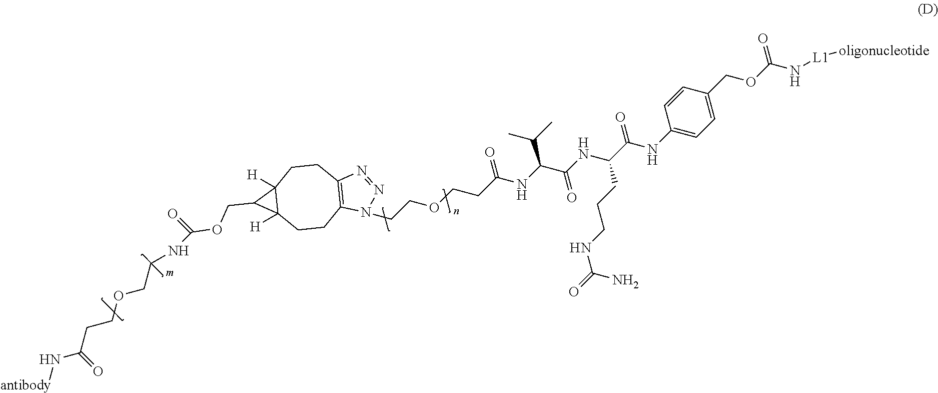

wherein n is 3 and m is 4, optionally wherein the oligonucleotide comprises a region of complementarity of at least 8 nucleotides to a pro-atrophy gene provided in Table 1.

In some embodiments, the complex described herein comprises an anti-TfR Fab covalently linked via a lysine to the 5′ end of an oligonucleotide (e.g., an oligonucleotide targeting a muscle atrophy gene listed in Table 1), wherein the anti-TfR Fab comprises a CDR-H1 as set forth in SEQ ID NO: 170, a CDR-H2 as set forth in SEQ ID NO: 171, a CDR-H3 as set forth in SEQ ID NO: 172, a CDR-L1 as set forth in SEQ ID NO: 173, a CDR-L2 as set forth in SEQ ID NO: 21, and a CDR-L3 as set forth in SEQ ID NO: 174; wherein the complex has the structure of:

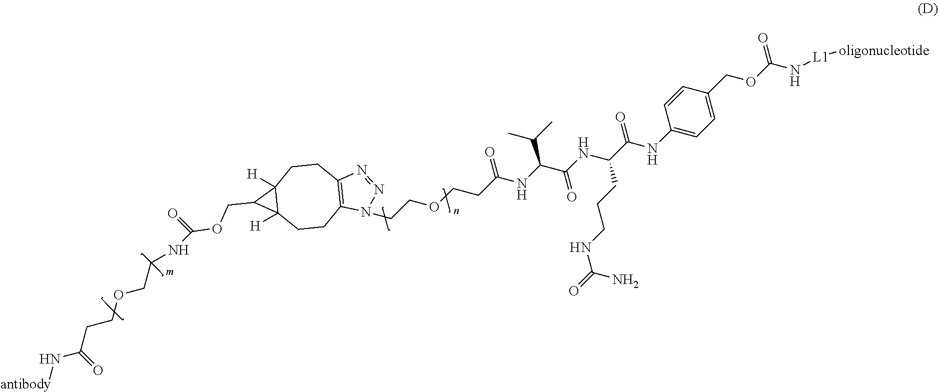

wherein n is 3 and m is 4, optionally wherein the oligonucleotide comprises a region of complementarity of at least 8 nucleotides to a pro-atrophy gene provided in Table 1.

In some embodiments, the complex described herein comprises an anti-TfR Fab covalently linked via a lysine to the 5′ end of an oligonucleotide (e.g., an oligonucleotide targeting a muscle atrophy gene listed in Table 1), wherein the anti-TfR Fab comprises a CDR-H1 as set forth in SEQ ID NO: 188, a CDR-H2 as set forth in SEQ ID NO: 189, a CDR-H3 as set forth in SEQ ID NO: 190, a CDR-L1 as set forth in SEQ ID NO: 191, a CDR-L2 as set forth in SEQ ID NO: 192, and a CDR-L3 as set forth in SEQ ID NO: 193; wherein the complex has the structure of:

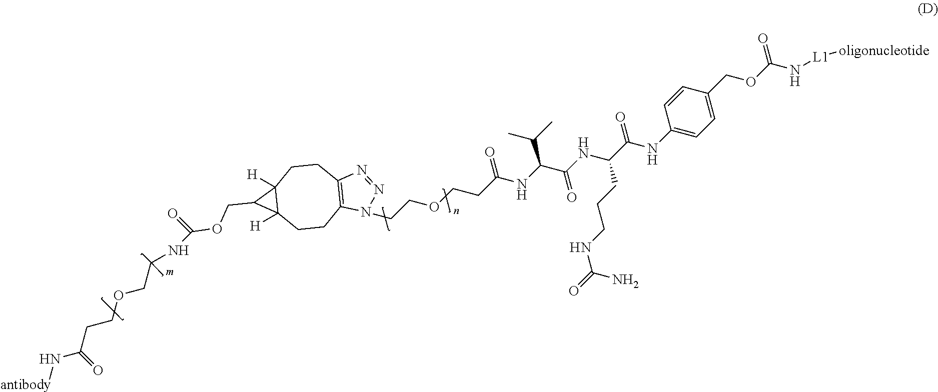

wherein n is 3 and m is 4, optionally wherein the oligonucleotide comprises a region of complementarity of at least 8 nucleotides to a pro-atrophy gene provided in Table 1.

In some embodiments, the complex described herein comprises an anti-TfR Fab covalently linked via a lysine to the 5′ end of an oligonucleotide (e.g., an oligonucleotide targeting a muscle atrophy gene listed in Table 1), wherein the anti-TfR Fab comprises a CDR-H1 as set forth in SEQ ID NO: 194, a CDR-H2 as set forth in SEQ ID NO: 195, a CDR-H3 as set forth in SEQ ID NO: 196, a CDR-L1 as set forth in SEQ ID NO: 197, a CDR-L2 as set forth in SEQ ID NO: 198, and a CDR-L3 as set forth in SEQ ID NO: 193; wherein the complex has the structure of:

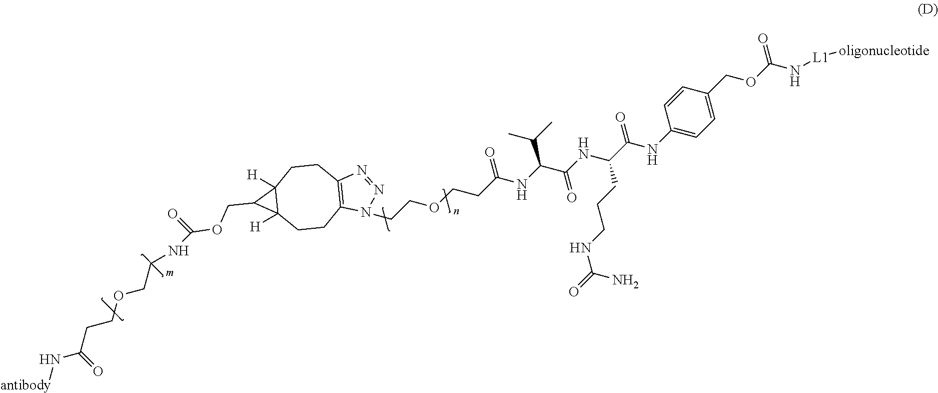

wherein n is 3 and m is 4, optionally wherein the oligonucleotide comprises a region of complementarity of at least 8 nucleotides to a pro-atrophy gene provided in Table 1.

In some embodiments, the complex described herein comprises an anti-TfR Fab covalently linked via a lysine to the 5′ end of an oligonucleotide (e.g., an oligonucleotide targeting a muscle atrophy gene listed in Table 1), wherein the anti-TfR Fab comprises a CDR-H1 as set forth in SEQ ID NO: 199, a CDR-H2 as set forth in SEQ ID NO: 200, a CDR-H3 as set forth in SEQ ID NO: 201, a CDR-L1 as set forth in SEQ ID NO: 202, a CDR-L2 as set forth in SEQ ID NO: 192, and a CDR-L3 as set forth in SEQ ID NO: 203; wherein the complex has the structure of:

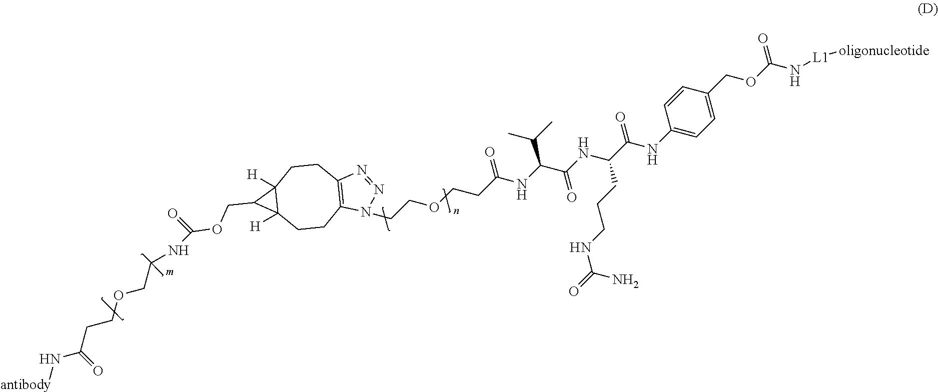

wherein n is 3 and m is 4, optionally wherein the oligonucleotide comprises a region of complementarity of at least 8 nucleotides to a pro-atrophy gene provided in Table 1.

In some embodiments, in any one of the examples of complexes described herein, L1 is any one of the spacers described herein.

In some embodiments, L1 is

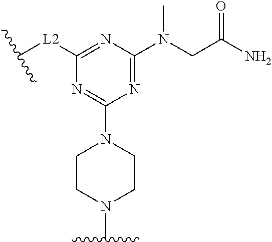

wherein the piperazine moiety links to the oligonucleotide, wherein L2 is

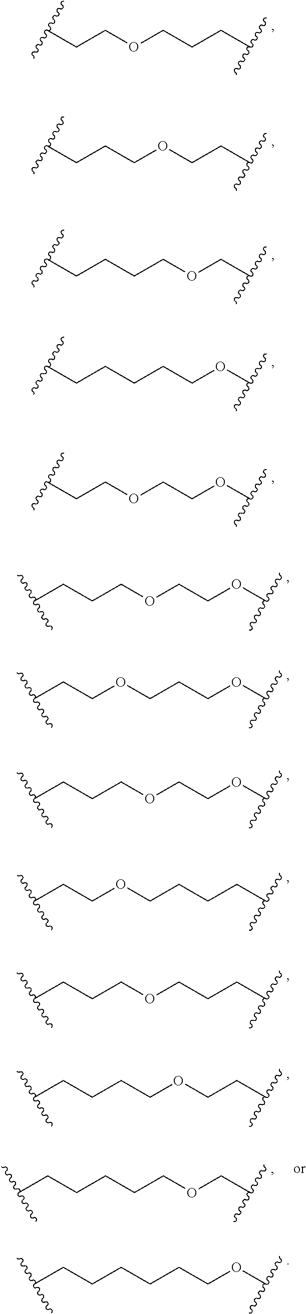

In some embodiments, L1 is:

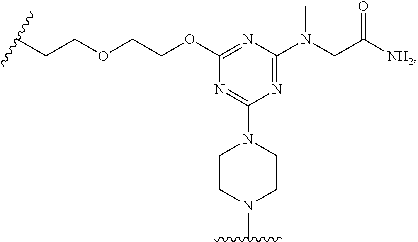 [SMILES: *CCOCCOc1nc(N(C)CC(N)=O)nc(N2CCN(*)CC2)n1]

wherein the piperazine is linked to the oligonucleotide.

In some embodiments, L1 is

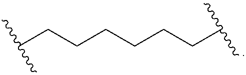 [SMILES: *CCCCCC*]

In some embodiments, L1 is linked to the 5′ phosphate of the oligonucleotide. In some embodiments, L1 is linked to the 5′ phosphorothioate of the oligonucleotide. In some embodiments, L1 is linked to the 5′ phosphonoamidate of the oligonucleotide.

In some embodiments, L1 is optional (e.g., need not be present).

### III. Formulations

Complexes provided herein may be formulated in any suitable manner. Generally, complexes provided herein are formulated in a manner suitable for pharmaceutical use. For example, complexes can be delivered to a subject using a formulation that minimizes degradation, facilitates delivery and/or (e.g., and) uptake, or provides another beneficial property to the complexes in the formulation. In some embodiments, provided herein are compositions comprising complexes and pharmaceutically acceptable carriers. Such compositions can be suitably formulated such that when administered to a subject, either into the immediate environment of a target cell or systemically, a sufficient amount of the complexes enter target muscle cells. In some embodiments, complexes are formulated in buffer solutions such as phosphate-buffered saline solutions, liposomes, micellar structures, and capsids.

It should be appreciated that, in some embodiments, compositions may include separately one or more components of complexes provided herein (e.g., muscle-targeting agents, linkers, molecular payloads, or precursor molecules of any one of them).

In some embodiments, complexes are formulated in water or in an aqueous solution (e.g., water with pH adjustments). In some embodiments, complexes are formulated in basic buffered aqueous solutions (e.g., PBS). In some embodiments, formulations as disclosed herein comprise an excipient. In some embodiments, an excipient confers to a composition improved stability, improved absorption, improved solubility and/or (e.g., and) therapeutic enhancement of the active ingredient. In some embodiments, an excipient is a buffering agent (e.g., sodium citrate, sodium phosphate, a tris base, or sodium hydroxide) or a vehicle (e.g., a buffered solution, petrolatum, dimethyl sulfoxide, or mineral oil).

In some embodiments, a complex or component thereof (e.g., oligonucleotide or antibody) is lyophilized for extending its shelf-life and then made into a solution before use (e.g., administration to a subject). Accordingly, an excipient in a composition comprising a complex, or component thereof, described herein may be a lyoprotectant (e.g., mannitol, lactose, polyethylene glycol, or polyvinyl pyrolidone), or a collapse temperature modifier (e.g., dextran, ficoll, or gelatin).

In some embodiments, a pharmaceutical composition is formulated to be compatible with its intended route of administration. Examples of routes of administration include parenteral, e.g., intravenous, intradermal, subcutaneous, administration. Typically, the route of administration is intravenous or subcutaneous. In some embodiments, the route of administration is extramuscular parenteral administration.

Pharmaceutical compositions suitable for injectable use include sterile aqueous solutions (where water soluble) or dispersions and sterile powders for the extemporaneous preparation of sterile injectable solutions or dispersions. The carrier can be a solvent or dispersion medium containing, for example, water, ethanol, polyol (for example, glycerol, propylene glycol, and liquid polyethylene glycol, and the like), and suitable mixtures thereof. In some embodiments, formulations include isotonic agents, for example, sugars, polyalcohols such as mannitol, sorbitol, and sodium chloride in the composition. Sterile injectable solutions can be prepared by incorporating the complexes in a required amount in a selected solvent with one or a combination of ingredients enumerated above, as required, followed by filtered sterilization.

In some embodiments, a composition may contain at least about 0.1% of the a complex, or component thereof, or more, although the percentage of the active ingredient(s) may be between about 1% and about 80% or more of the weight or volume of the total composition. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

### IV. Methods of Use/Treatment

Complexes comprising a muscle-targeting agent covalently linked to a molecular payload as described herein are effective in treating muscle atrophy (e.g., muscle atrophy due to a chronic illness, including AIDS, congestive heart failure, cancer, chronic obstructive pulmonary disease, and renal failure, or muscle disuse). In some embodiments, muscle atrophy is associated with the activity of one or more genes listed in Table 1.

In some embodiments, a subject may be a human subject, a non-human primate subject, a rodent subject, or any suitable mammalian subject. In some embodiments, a subject may have muscle atrophy, or be at risk of developing muscle atrophy.

An aspect of the disclosure includes a methods involving administering to a subject an effective amount of a complex as described herein. In some embodiments, an effective amount of a pharmaceutical composition that comprises a complex comprising a muscle-targeting agent covalently linked to a molecular payload can be administered to a subject in need of treatment. In some embodiments, a pharmaceutical composition comprising a complex as described herein may be administered by a suitable route, which may include intravenous administration, e.g., as a bolus or by continuous infusion over a period of time. In some embodiments, intravenous administration may be performed by intramuscular, intraperitoneal, intracerebrospinal, subcutaneous, intra-articular, intrasynovial, or intrathecal routes. In some embodiments, a pharmaceutical composition may be in solid form, aqueous form, or a liquid form. In some embodiments, an aqueous or liquid form may be nebulized or lyophilized. In some embodiments, a nebulized or lyophilized form may be reconstituted with an aqueous or liquid solution.

Compositions for intravenous administration may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, water soluble antibodies can be administered by the drip method, whereby a pharmaceutical formulation containing the antibody and a physiologically acceptable excipients is infused. Physiologically acceptable excipients may include, for example, 5% dextrose, 0.9% saline, Ringer's solution or other suitable excipients. Intramuscular preparations, e.g., a sterile formulation of a suitable soluble salt form of the antibody, can be dissolved and administered in a pharmaceutical excipient such as Water-for-Injection, 0.9% saline, or 5% glucose solution.

In some embodiments, a pharmaceutical composition that comprises a complex comprising a muscle-targeting agent covalently linked to a molecular payload is administered via site-specific or local delivery techniques. Examples of these techniques include implantable depot sources of the complex, local delivery catheters, site specific carriers, direct injection, or direct application.

In some embodiments, a pharmaceutical composition that comprises a complex comprising a muscle-targeting agent covalently linked to a molecular payload is administered at an effective concentration that confers therapeutic effect on a subject. Effective amounts vary, as recognized by those skilled in the art, depending on the severity of the disease, unique characteristics of the subject being treated, e.g. age, physical conditions, health, or weight, the duration of the treatment, the nature of any concurrent therapies, the route of administration and related factors. These related factors are known to those in the art and may be addressed with no more than routine experimentation. In some embodiments, an effective concentration is the maximum dose that is considered to be safe for the patient. In some embodiments, an effective concentration will be the lowest possible concentration that provides maximum efficacy.

Empirical considerations, e.g. the half-life of the complex in a subject, generally will contribute to determination of the concentration of pharmaceutical composition that is used for treatment. The frequency of administration may be empirically determined and adjusted to maximize the efficacy of the treatment.

Generally, for administration of any of the complexes described herein, an initial candidate dosage may be about 1 to 100 mg/kg, or more, depending on the factors described above, e.g. safety or efficacy. In some embodiments, a treatment will be administered once. In some embodiments, a treatment will be administered daily, biweekly, weekly, bimonthly, monthly, or at any time interval that provide maximum efficacy while minimizing safety risks to the subject. Generally, the efficacy and the treatment and safety risks may be monitored throughout the course of treatment

The efficacy of treatment may be assessed using any suitable methods. In some embodiments, the efficacy of treatment may be assessed by evaluation of observation of symptoms associated with muscle atrophy.

In some embodiments, a pharmaceutical composition that comprises a complex comprising a muscle-targeting agent covalently linked to a molecular payload described herein is administered to a subject at an effective concentration sufficient to inhibit activity or expression of a target gene by at least 10%, at least 20%, at least 30%, at least 40%, at least 50%, at least 60%, at least 70%, at least 80%, at least 90% or at least 95% relative to a control, e.g. baseline level of gene expression prior to treatment.

In some embodiments, a single dose or administration of a pharmaceutical composition that comprises a complex comprising a muscle-targeting agent covalently linked to a molecular payload described herein to a subject is sufficient to inhibit activity or expression of a target gene for at least 1-5, 1-10, 5-15, 10-20, 15-30, 20-40, 25-50, or more days. In some embodiments, a single dose or administration of a pharmaceutical composition that comprises a complex comprising a muscle-targeting agent covalently linked to a molecular payload described herein to a subject is sufficient to inhibit activity or expression of a target gene for at least 1, 2, 3, 4, 5, 6, 7, 8, 9, 10, 11, or 12 weeks. In some embodiments, a single dose or administration of a pharmaceutical composition that comprises a complex comprising a muscle-targeting agent covalently linked to a molecular payload described herein to a subject is sufficient to inhibit activity or expression of a target gene for at least 1, 2, 3, 4, 5, or 6 months.

In some embodiments, a pharmaceutical composition may comprises more than one complex comprising a muscle-targeting agent covalently linked to a molecular payload. In some embodiments, a pharmaceutical composition may further comprise any other suitable therapeutic agent for treatment of a subject, e.g. a human subject having muscle atrophy. In some embodiments, the other therapeutic agents may enhance or supplement the effectiveness of the complexes described herein. In some embodiments, the other therapeutic agents may function to treat a different symptom or disease than the complexes described herein.

### EXAMPLES

### Example 1: Targeting HPRT with Transfected Antisense Oligonucleotides

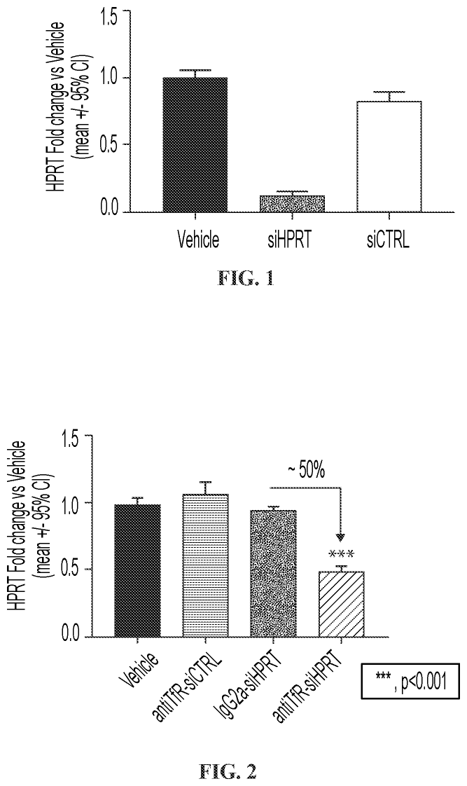

A siRNA that targets hypoxanthine phosphoribosyltransferase (HPRT) (Table 10) was tested in vitro for its ability to reduce expression levels of HPRT in an immortalized cell line. Briefly, Hepa 1-6 cells were transfected with either a control siRNA (siCTRL; 100 nM) or the siRNA that targets HPRT (siHPRT; 100 nM), formulated with lipofectamine 2000. HPRT expression levels were evaluated 48 hours following transfection. A control experiment was also performed in which vehicle (phosphate-buffered saline) was delivered to Hepa 1-6 cells in culture and the cells were maintained for 48 hours. As shown in FIG. 1, it was found that the HPRT siRNA reduced HPRT expression levels by ˜90% compared with controls.

TABLE 10
Sequences of siHPRT and siCTRL

| | | SEQ |
|---|---|---|
| | Sequence | ID NO: |
| siHPRT sense | 5′-UcCuAuGaCuGuAgA | 258 |
| strand | uUuUaU-(CH₂)₆NH₂-3′ | |
| siHPRT antisense | 5′-aUaAaAuCuAcAgUc | 259 |
| strand | AuAgGasAsu-3′ | |
| siCTRL sense | 5′-UgUaAuAaCcAuAuC | 260 |
| strand | uAcCuU-(CH₂)₆NH₂-3′ | |
| siCTRL antisense | 5′-aAgGuAgAuAuGgUu | 261 |
| strand | AuUaCasAsa-3′ | |

*Lower case-2'-O-Me modified nucleosides; Capital letter-2'-Fluoro modified nucleosides; s-phosphorothioate linkage

### Example 2: Targeting HPRT with a Muscle-Targeting Complex

A muscle-targeting complex was generated comprising the HPRT siRNA used in Example 1 (siHPRT) covalently linked, via a non-cleavable N-gamma-maleimidobutyryl-oxysuccinimide ester (GMBS) linker, to DTX-A-002, an anti-transferrin receptor antibody.

Briefly, the GMBS linker was dissolved in dry DMSO and coupled to the 3′ end of the sense strand of siHPRT through amide bond formation under aqueous conditions. Completion of the reaction was verified by Kaiser test. Excess linker and organic solvents were removed by gel permeation chromatography. The purified, maleimide functionalized sense strand of siHPRT was then coupled to DTX-A-002 antibody using a Michael addition reaction.

The product of the antibody coupling reaction was then subjected to hydrophobic interaction chromatography (HIC-HPLC). antiTfR-siHPRT complexes comprising one or two siHPRT molecules covalently attached to DTX-A-002 antibody were purified. Densitometry confirmed that the purified sample of complexes had an average siHPRT to antibody ratio of 1.46. SDS-PAGE analysis demonstrated that >90% of the purified sample of complexes comprised DTX-A-002 linked to either one or two siHPRT molecules.

Using the same methods as described above, a control IgG2a-siHPRT complex was generated comprising the HPRT siRNA used in Example 1 (siHPRT) covalently linked via the GMBS linker to an IgG2a (Fab) antibody (DTX-A-003). Densitometry confirmed that DTX-C-001 had an average siHPRT to antibody ratio of 1.46 and SDS-PAGE demonstrated that >90% of the purified sample of control complexes comprised DTX-A-003 linked to either one or two siHPRT molecules.

The antiTfR-siHPRT complex was then tested for cellular internalization and inhibition of HPRT in cellulo. Hepa 1-6 cells, which have relatively high expression levels of transferrin receptor, were incubated in the presence of vehicle (phosphate-buffered saline), IgG2a-siHPRT (100 nM), antiTfR-siCTRL (100 nM), or antiTfR-siHPRT (100 nM), for 72 hours. After the 72 hour incubation, the cells were isolated and assayed for expression levels of HPRT (FIG. 2). Cells treated with the antiTfR-siHPRT demonstrated a reduction in HPRT expression by —50% relative to the cells treated with the vehicle control. Meanwhile, cells treated with either of the IgG2a-siHPRT or antiTfR-siCTRL had HPRT expression levels comparable to the vehicle control (no reduction in HPRT expression). These data indicate that the anti-transferrin receptor antibody of the antiTfR-siHPRT enabled cellular internalization of the complex, thereby allowing the siHPRT to inhibit expression of HPRT.

### Example 3: Targeting HPRT in Mouse Muscle Tissues with a Muscle-Targeting Complex

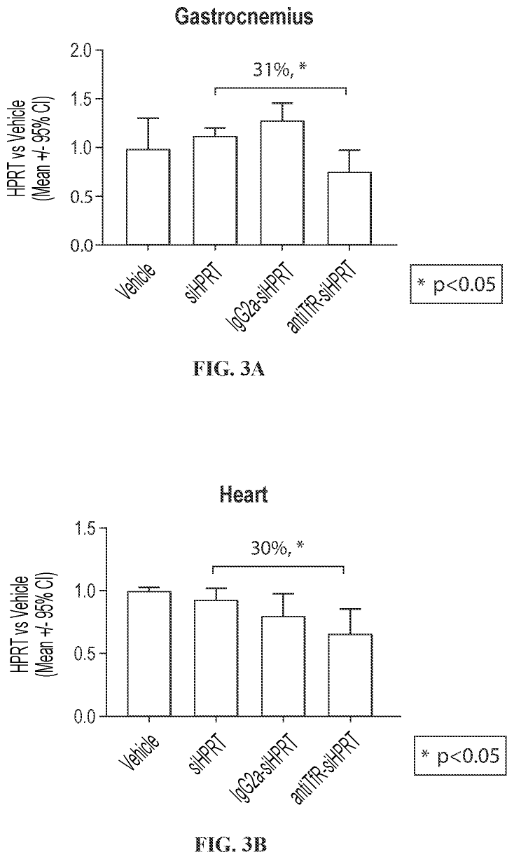

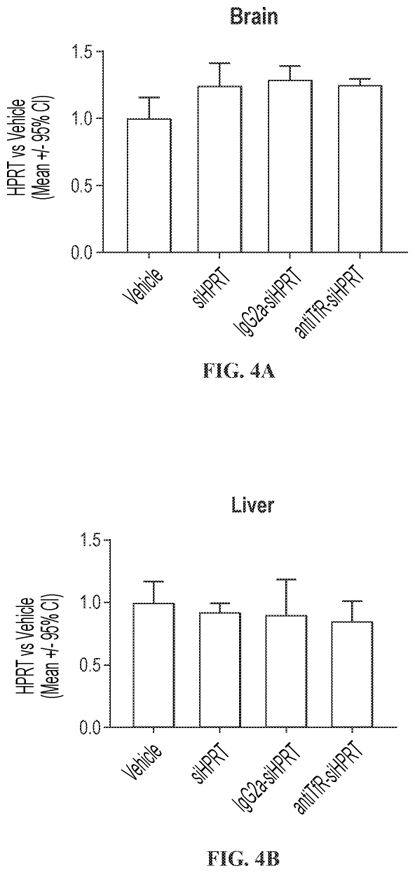

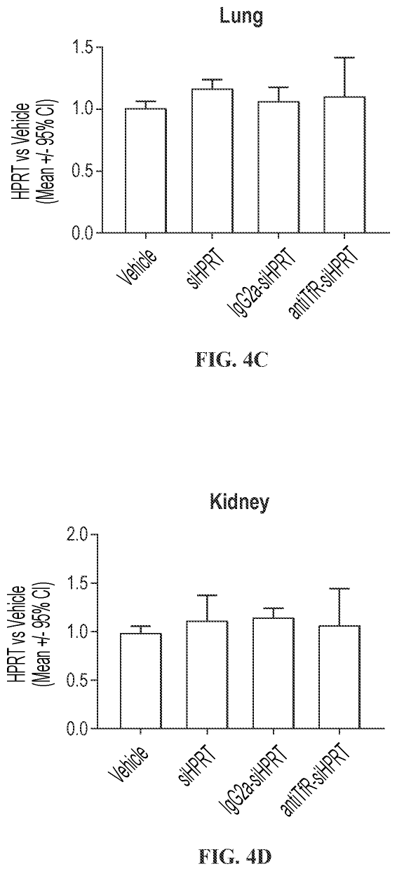

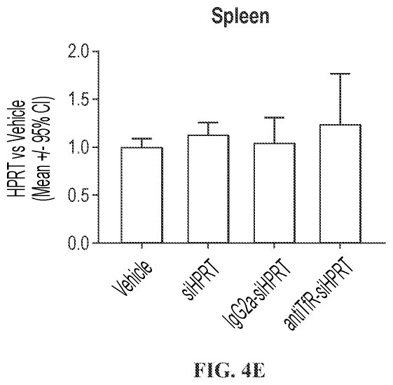

The muscle-targeting complex described in Example 2, antiTfR-siHPRT, was tested for inhibition of HPRT in mouse tissues. C57BL/6 wild-type mice were intravenously injected with a single dose of a vehicle control (phosphate-buffered saline); siHPRT (2 mg/kg of RNA); IgG2a-siHPRT (2 mg/kg of RNA, corresponding to 9 mg/kg antibody complex); or antiTfR-siHPRT (2 mg/kg of RNA, corresponding to 9 mg/kg antibody complex. Each experimental condition was replicated in four individual C57BL/6 wild-type mice. Following a three-day period after injection, the mice were euthanized and segmented into isolated tissue types. Individual tissue samples were subsequently assayed for expression levels of HPRT (FIGS. 3A-3B and 4A-4E).

Mice treated with the antiTfR-siHPRT complex demonstrated a reduction in HPRT expression in gastrocnemius (31% reduction; p<0.05) and heart (30% reduction; p<0.05), relative to the mice treated with the siHPRT control (FIGS. 3A-3B). Meanwhile, mice treated with the IgG2a-siHPRT complex had HPRT expression levels comparable to the siHPRT control (little or no reduction in HPRT expression) for all assayed muscle tissue types.

Mice treated with the antiTfR-siHPRT complex demonstrated no change in HPRT expression in non-muscle tissues such as brain, liver, lung, kidney, and spleen tissues (FIGS. 4A-4E).

These data indicate that the anti-transferrin receptor antibody of the antiTfR-siHPRT complex enabled cellular internalization of the complex into muscle-specific tissues in an in vivo mouse model, thereby allowing the siHPRT to inhibit expression of HPRT. These data further demonstrate that the antiTfR-oligonucleotide complexes of the current disclosure are capable of specifically targeting muscle tissues.

### Example 4: Targeting MSTN with a Muscle-Targeting Complex

A muscle-targeting complex is generated comprising an antisense oligonucleotide that targets an allele of MSTN (MSTN ASO) covalently linked, via a cathepsin cleavable linker, to an anti-transferrin receptor antibody (e.g. RI7 217 (Fab) or 15G11).

Briefly, a maleimidocaproyl-L-valine-L-citrulline-p-aminobenzyl alcohol p-nitrophenyl carbonate (MC-Val-Cit-PABC-PNP) linker molecule is coupled to NH2-C6-MSTN ASO using an amide coupling reaction. Excess linker and organic solvents are removed by gel permeation chromatography. The purified Val-Cit-linker-MSTN ASO is then coupled to a thiol-reactive anti-transferrin receptor antibody (e.g. RI7 217 (Fab) or 15G11).

The product of the antibody coupling reaction is then subjected to hydrophobic interaction chromatography (HIC-HPLC) to purify the muscle-targeting complex. Densitometry and SDS-PAGE analysis of the purified complex allow for determination of the average ratio of ASO-to-antibody and total purity, respectively.

Using the same methods as described above, a control complex is generated comprising MSTN ASO covalently linked via a Val-Cit linker to an IgG2a (Fab) antibody.

The purified muscle-targeting complex comprising anti-transferrin receptor antibody (e.g. RI7 217 (Fab) or 15G11) covalently linked to MSTN ASO is then tested for cellular internalization and inhibition of MSTN. Disease-relevant muscle cells that have relatively high expression levels of transferrin receptor, are incubated in the presence of vehicle control (saline), muscle-targeting complex (100 nM), or control complex (100 nM) for 72 hours. After the 72 hour incubation, the cells are isolated and assayed for expression levels of MSTN.

### Example 5: Targeting FBXO32 with a Muscle-Targeting Complex

A muscle-targeting complex is generated comprising an antisense oligonucleotide that targets an allele of FBXO32 (FBXO32 ASO) covalently linked, via a cathepsin cleavable linker, to an anti-transferrin receptor antibody (e.g. RI7 217 (Fab) or 15G11).

Briefly, a maleimidocaproyl-L-valine-L-citrulline-p-aminobenzyl alcohol p-nitrophenyl carbonate (MC-Val-Cit-PABC-PNP) linker molecule is coupled to NH2-C6-FBXO32 ASO using an amide coupling reaction. Excess linker and organic solvents are removed by gel permeation chromatography. The purified Val-Cit-linker-FBXO32 ASO is then coupled to a thiol-reactive anti-transferrin receptor antibody (e.g. RI7 217 (Fab) or 15G11).

The product of the antibody coupling reaction is then subjected to hydrophobic interaction chromatography (HIC-HPLC) to purify the muscle-targeting complex. Densitometry and SDS-PAGE analysis of the purified complex allow for determination of the average ratio of ASO-to-antibody and total purity, respectively.

Using the same methods as described above, a control complex is generated comprising FBXO32 ASO covalently linked via a Val-Cit linker to an IgG2a (Fab) antibody. The purified muscle-targeting complex comprising an anti-transferrin receptor antibody (e.g. RI7 217 (Fab) or 15G11) covalently linked to FBXO32 ASO is then tested for cellular internalization and inhibition of FBXO32. Disease-relevant muscle cells that have relatively high expression levels of transferrin receptor, are incubated in the presence of vehicle control (saline), muscle-targeting complex (100 nM), or control complex (100 nM) for 72 hours. After the 72 hour incubation, the cells are isolated and assayed for expression levels of FBXO32.

### Example 6: Targeting TRIM63 with a Muscle-Targeting Complex

A muscle-targeting complex is generated comprising an antisense oligonucleotide that targets an allele of TRIM63 (TRIM63 ASO) covalently linked, via a cathepsin cleavable linker, to an anti-transferrin receptor antibody (e.g. RI7 217 (Fab) or 15G11).

Briefly, a maleimidocaproyl-L-valine-L-citrulline-p-aminobenzyl alcohol p-nitrophenyl carbonate (MC-Val-Cit-PABC-PNP) linker molecule is coupled to NH2-C6-TRIM63 ASO using an amide coupling reaction. Excess linker and organic solvents are removed by gel permeation chromatography. The purified Val-Cit-linker-TRIM63 ASO is then coupled to a thiol-reactive anti-transferrin receptor antibody (e.g. RI7 217 (Fab) or 15G11).

The product of the antibody coupling reaction is then subjected to hydrophobic interaction chromatography (HIC-HPLC) to purify the muscle-targeting complex. Densitometry and SDS-PAGE analysis of the purified complex allow for determination of the average ratio of ASO-to-antibody and total purity, respectively.

Using the same methods as described above, a control complex is generated comprising TRIM63 ASO covalently linked via a Val-Cit linker to an IgG2a (Fab) antibody. The purified muscle-targeting complex comprising an anti-transferrin receptor antibody (e.g. RI7 217 (Fab) or 15G11) covalently linked to TRIM63 ASO is then tested for cellular internalization and inhibition of TRIM63. Disease-relevant muscle cells that have relatively high expression levels of transferrin receptor, are incubated in the presence of vehicle control (saline), muscle-targeting complex (100 nM), or control complex (100 nM) for 72 hours. After the 72 hour incubation, the cells are isolated and assayed for expression levels of TRIM63.

### Example 7: Targeting INHBA with a Muscle-Targeting Complex

A muscle-targeting complex is generated comprising an antisense oligonucleotide that targets an allele of INHBA (INHBA ASO) covalently linked, via a cathepsin cleavable linker, to an anti-transferrin receptor antibody (e.g. RI7 217 (Fab) or 15G11).

Briefly, a maleimidocaproyl-L-valine-L-citrulline-p-aminobenzyl alcohol p-nitrophenyl carbonate (MC-Val-Cit-PABC-PNP) linker molecule is coupled to NH2-C6-INHBA ASO using an amide coupling reaction. Excess linker and organic solvents are removed by gel permeation chromatography. The purified Val-Cit-linker-INHBA ASO is then coupled to a thiol-reactive anti-transferrin receptor antibody (e.g. RI7 217 (Fab) or 15G11).

The product of the antibody coupling reaction is then subjected to hydrophobic interaction chromatography (HIC-HPLC) to purify the muscle-targeting complex. Densitometry and SDS-PAGE analysis of the purified complex allow for determination of the average ratio of ASO-to-antibody and total purity, respectively.

Using the same methods as described above, a control complex is generated comprising INHBA ASO covalently linked via a Val-Cit linker to an IgG2a (Fab) antibody. The purified muscle-targeting complex comprising an anti-transferrin receptor antibody (e.g. RI7 217 (Fab) or 15G11) covalently linked to INHBA ASO is then tested for cellular internalization and inhibition of INHBA. Disease-relevant muscle cells that have relatively high expression levels of transferrin receptor, are incubated in the presence of vehicle control (saline), muscle-targeting complex (100 nM), or control complex (100 nM) for 72 hours. After the 72 hour incubation, the cells are isolated and assayed for expression levels of INHBA.

### Example 8: Targeting Gene Expression in Cynomolgus Monkey Muscle Tissues with a Muscle-Targeting Complex

A muscle-targeting complex (DTX-C-012) was generated. DTX-C-012 complex comprises an anti-transferrin receptor antibody (a 15G11 antibody comprising a heavy chain comprising the amino acid sequence of SEQ ID NO: 240 and a light chain comprising the amino acid sequence of SEQ ID NO: 237) that binds to the human transferrin receptor and the cynomolgus monkey transferrin receptor, covalently linked, via a cathepsin cleavable Val-Cit linker, to control DMPK-ASO, an antisense oligonucleotide that targets a gene that is expressed in muscle tissues (DMPK). Following HIC-HPLC purification, densitometry confirmed that DTX-C-012 had an average ASO to antibody ratio of 1.32, and SDS-PAGE revealed a purity of 92.3%.

DTX-C-012 was tested for dose-dependent inhibition of gene expression in male cynomolgus monkey tissues. Male cynomolgus monkeys (19-31 months; 2-3 kg) were intravenously injected with a single dose of a saline control, control DMPK-ASO (naked ASO) (10 mg/kg of RNA), or DTX-C-012 (10 mg/kg of RNA) on Day 0. Each experimental condition was replicated in three individual male cynomolgus monkeys. On Day 7 after injection, tissue biopsies (including muscle tissues) were collected. DMPK mRNA expression levels, ASO detection assays, serum clinical chemistries, tissue histology, clinical observations, and body weights were analyzed. The monkeys were euthanized on Day 14.

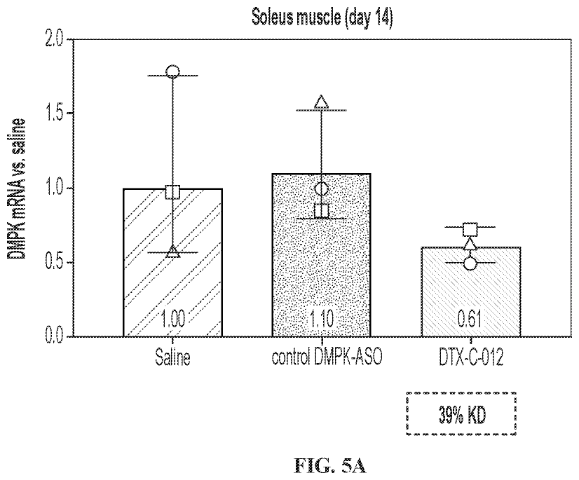

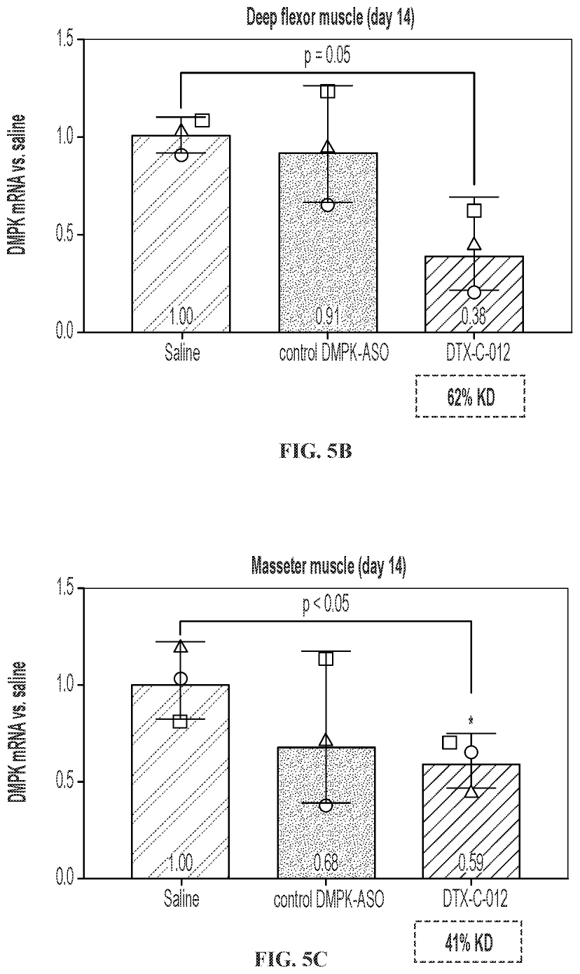

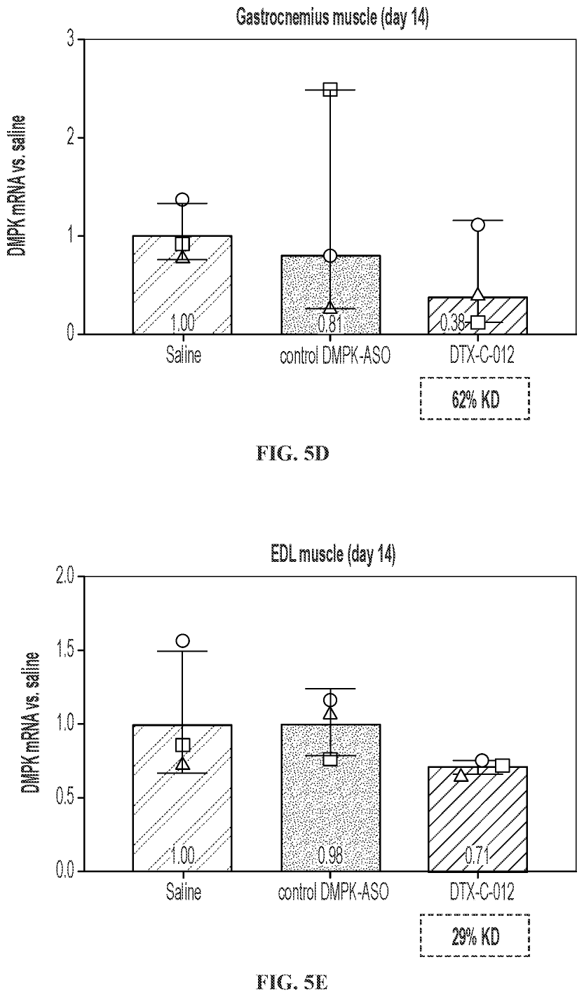

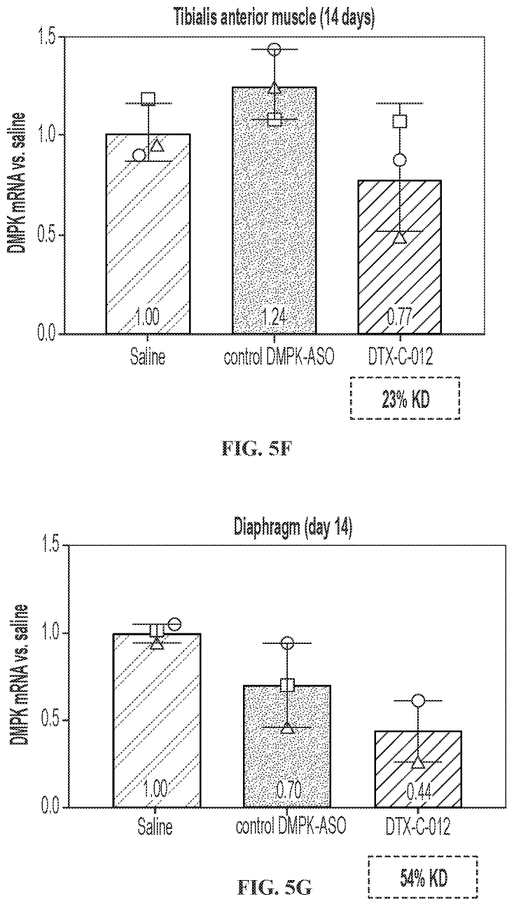

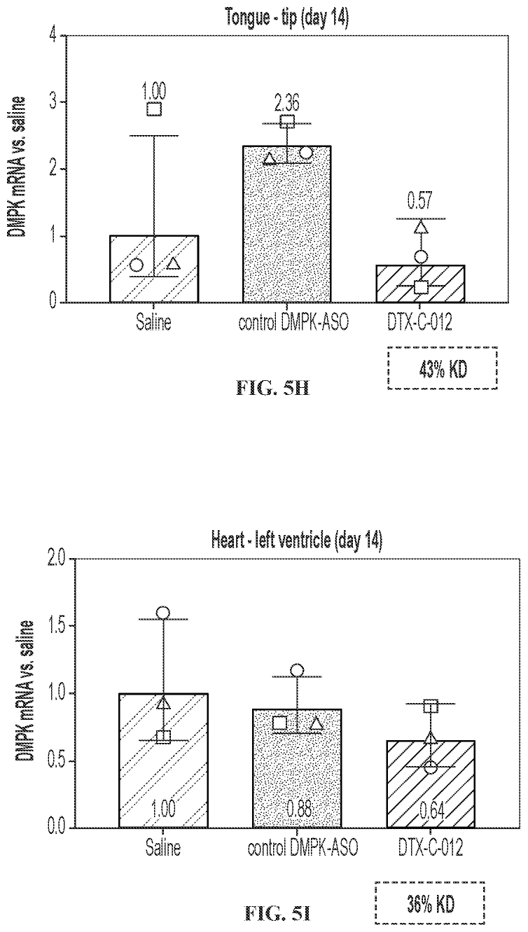

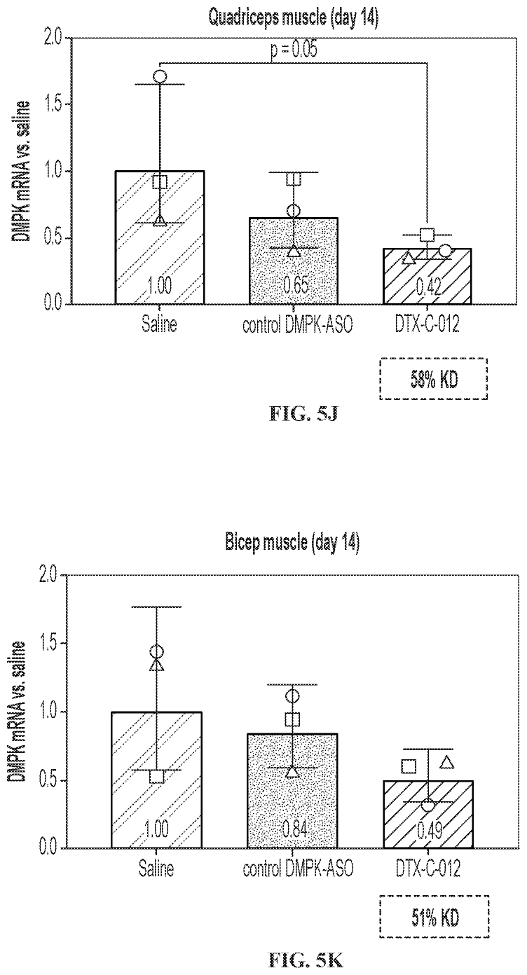

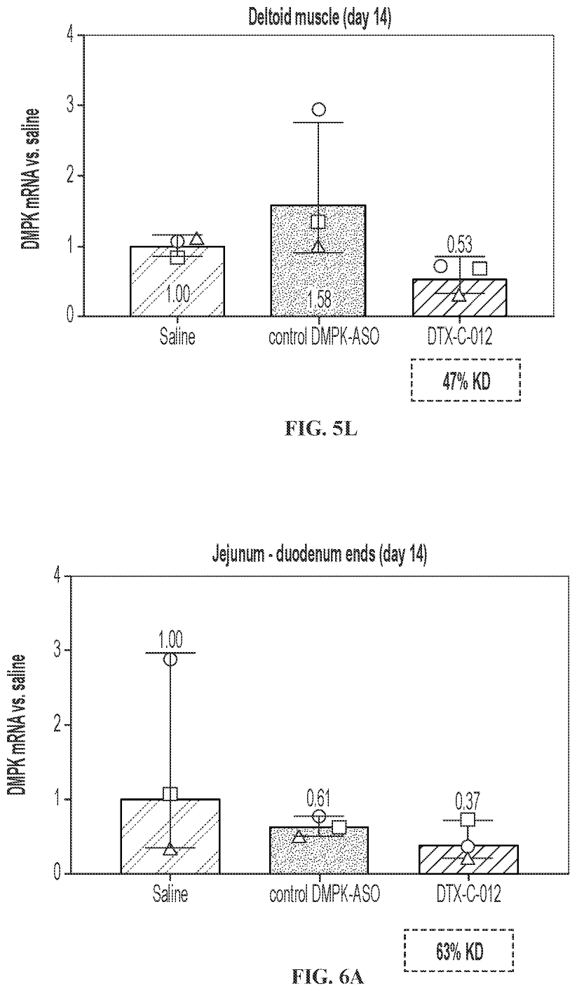

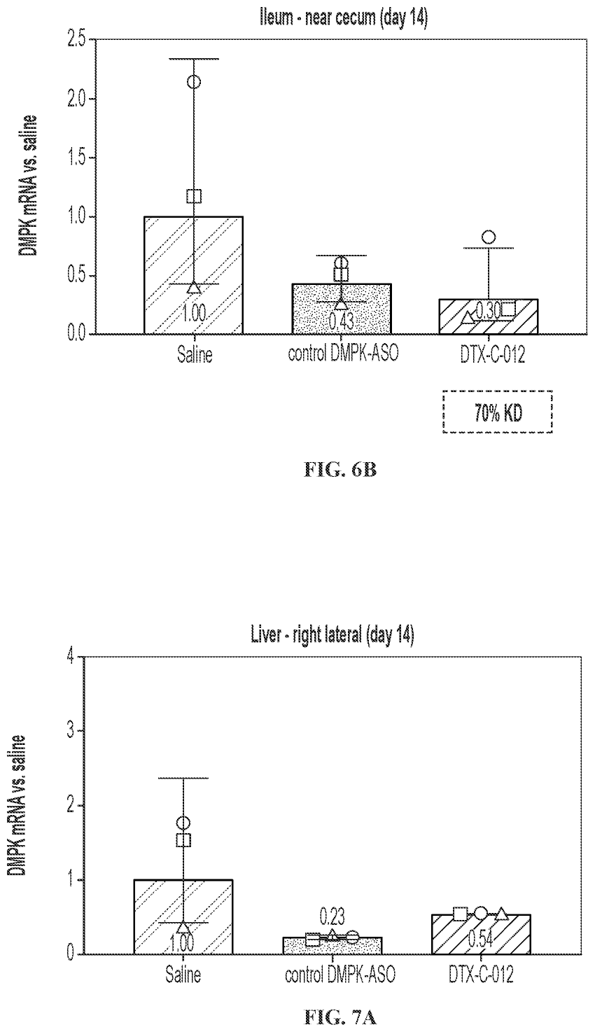

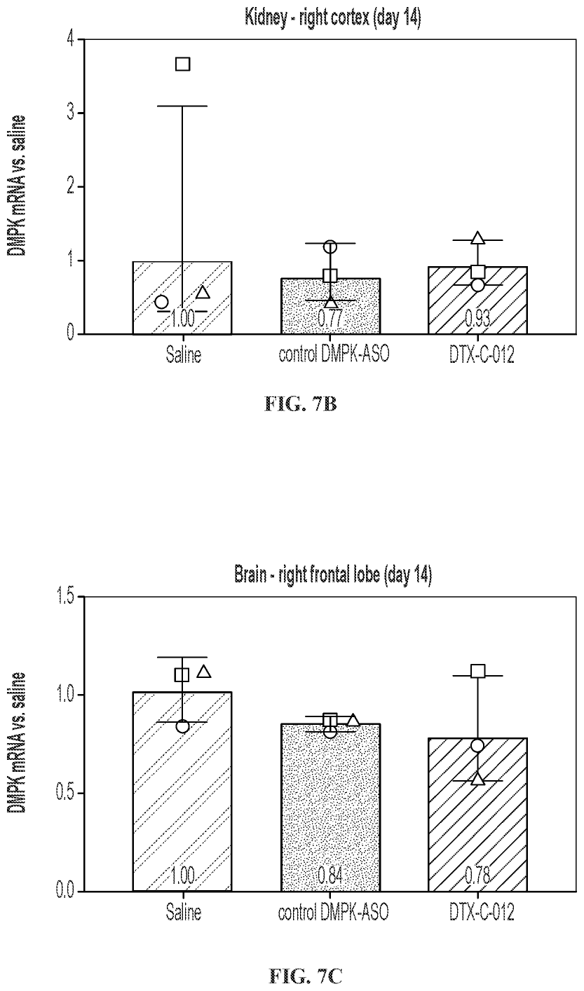

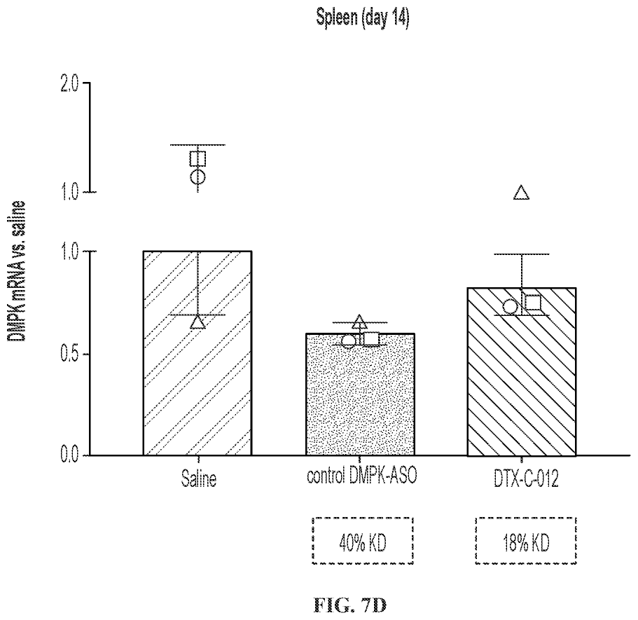

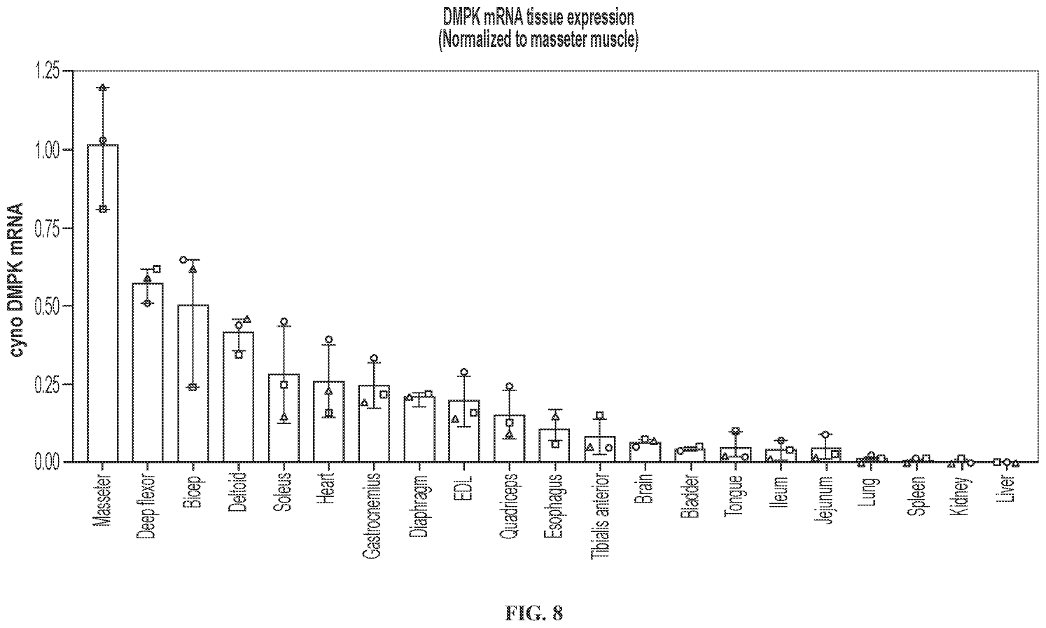

Significant knockdown (KD) of mRNA expression using DTX-C-012 was observed in soleus, deep flexor, and masseter muscles relative to saline control, with 39% KD, 62% KD, and 41% KD, respectively (FIGS. 5A-5C). Robust knockdown of mRNA expression DTX-C-012 was further observed in gastrocnemius (62% KD; FIG. 5D), EDL (29% KD; FIG. 5E), tibialis anterior muscle (23% KD; FIG. 5F), diaphragm (54% KD; FIG. 5G), tongue (43% KD; FIG. 5H), heart muscle (36% KD; FIG. 5I), quadriceps (58% KD; FIG. 5J), bicep (51% KD; FIG. 5K), and deltoid muscles (47% KD; FIG. 5L). Knockdown of mRNA expression DTX-C-012 in smooth muscle was also observed in the intestine, with 63% KD at jejunum-duodenum ends (FIG. 6A) and 70% KD in ileum (FIG. 6B). Notably, naked ASO (i.e., not linked to a muscle-targeting agent), control DMPK-ASO, had minimal effects on gene expression levels relative to the vehicle control (i.e., little or no reduction in DMPK expression) for all assayed muscle tissue types. Monkeys treated with the DTX-C-012 complex demonstrated no change in DMPK expression in non-muscle tissues, such as liver, kidney, brain, and spleen tissues (FIGS. 7A-7D). Additional tissues were examined, as depicted in FIG. 8, which shows normalized mRNA tissue expression levels across several tissue types in cynomolgus monkeys. (N=3 male cynomolgus monkeys)

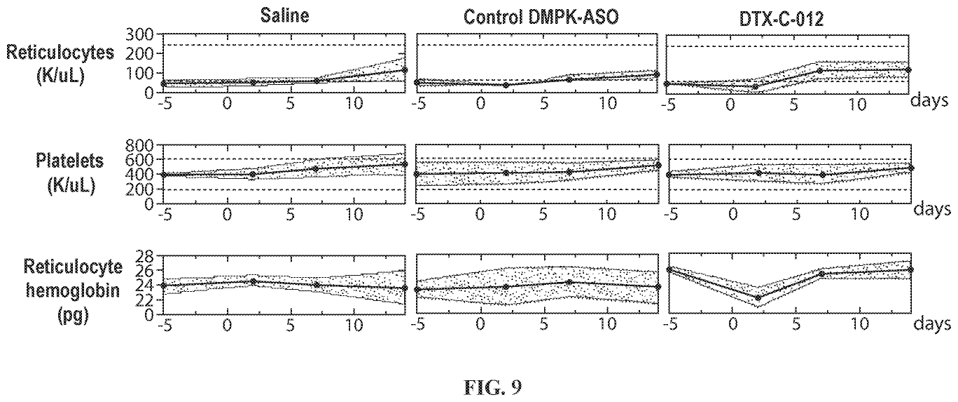

Prior to euthanization, all monkeys were tested for reticulocyte levels, platelet levels, hemoglobin expression, alanine aminotransferase (ALT) expression, aspartate aminotransferase (AST) expression, and blood urea nitrogen (BUN) levels on days 2, 7, and 14 after dosing. As shown in FIG. 9, monkeys dosed with antibody-oligonucleotide complex had normal reticulocyte levels, platelet levels, hemoglobin expression, alanine aminotransferase (ALT) expression, aspartate aminotransferase (AST) expression, and blood urea nitrogen (BUN) levels throughout the length of the experiment. These data show that a single dose of a complex comprising control DMPK-ASO is safe and tolerated in cynomolgus monkeys.

These data demonstrate that the anti-transferrin receptor 15G11 antibody of the DTX-C-012 complex enabled cellular internalization of the complex into muscle-specific tissues in an in vivo cynomolgus monkey model, thereby allowing the antisense oligonucleotide to be delivered to muscle cells. These data further demonstrate that the anti-transferrin receptor 15G11 antibody of the DTX-C-012 complex is capable of specifically targeting muscle tissues without substantially impacting non-muscle tissues. This is direct contrast with the limited ability of control DMPK-ASO, a naked ASO (i.e., not linked to a muscle-targeting agent), to inhibit gene expression in muscle tissues of an in vivo cynomolgus monkey model.

### Example 9: Binding Affinity of Selected Anti-TfR1 Antibodies in Table 2 to Human TfR1

Selected anti-TfR1 antibodies were tested for their binding affinity to human TfR1 for measurement of Ka (association rate constant), Kd (dissociation rate constant), and K_{D} (affinity). Two known anti-TfR1 antibodies were used as control, 15G11 and OKT9. The binding experiment was performed on Carterra LSA at 25C. An anti-mouse IgG and anti-human IgG antibody “lawn” was prepared on a HC30M chip by amine coupling. The tested IgGs were captured on the chip. Dilution series of hTfR1, cyTfR1, and hTfR2 were injected to the chip for binding (starting from 1000 nM, 1:3 dilution, 8 concentrations).

Binding data were referenced by subtracting the responses from a buffer analyte injection and globally fitting to a 1:1 Langmuir binding model for estimate of Ka (association rate constant), Kd (dissociation rate constant), and KD (affinity) using the Carterra™ Kinetics software. 5-6 concentrations were used for curve fitting.

The result showed that the mouse mAbs demonstrated binding to hTfR1 with KD values ranging from 13 pM to 50 nM. A majority of the mouse mAbs had KD values in the single digit nanomolar to sub-nanomolar range. The tested mouse mAbs showed cross-reactive binding to cyTfR1 with KD values ranging from 16 pM to 22 nM.

Ka, Kd, and K_{D} values of anti-TfR1 antibodies are provided in Table 11.

TABLE 11
Ka, Kd, and Kd values of anti-TfR1 antibodies

| Name | K_{D} (M) | Ka (M) | Kd (M) |
|---|---|---|---|
| ctrl-15G11 | 2.83E−10 | 3.70E+05 | 1.04E−04 |
| ctrl-OKT9 mIgG | 5.36E−10 | 7.74E+05 | 4.15E−04 |
| 3-A04 | 4.36E−10 | 4.47E+05 | 1.95E−04 |
| 3-M12 | 7.68E−10 | 1.66E+05 | 1.27E−04 |
| 5-H12 | 2.08E−07 | 6.67E+04 | 1.39E−02 |
| 10-H02 | 2.72E−09 | 1.26E+05 | 3.42E−04 |
| 10-P05 | 1.63E−09 | 1.70E+05 | 2.78E−04 |
| 2-H19 | 2.06E−09 | 2.22E+05 | 4.56E−04 |
| 3-E05 | 4.55E−10 | 2.20E+04 | 1.00E−05 |
| 3-F03 | 2.23E−09 | 1.38E+05 | 3.09E−04 |
| 3-M09 | 2.54E−09 | 1.50E+05 | 3.82E−04 |
| 3-P24 | 9.70E−10 | 6.72E+04 | 6.52E−05 |
| 4-C05 | 1.61E−09 | 3.01E+04 | 4.85E−05 |
| 4-H04 | 1.39E−08 | 6.17E+04 | 8.57E−04 |
| 4-O12 | 1.80E−09 | 7.98E+04 | 1.43E−04 |
| 6-D03 | 9.86E−10 | 1.08E+05 | 1.07E−04 |
| 8-D15 | 8.22E−09 | 3.13E+04 | 2.57E−04 |
| 8-K06 | 6.94E−11 | 1.44E+05 | 1.00E−05 |
| 8-O17 | 1.83E−09 | 4.99E+04 | 9.12E−05 |
| 9-C04 | 1.41E−08 | 4.10E+04 | 5.75E−04 |
| 9-D04 | 5.86E−09 | 4.20E+04 | 2.46E−04 |
| 9-K23 | 4.01E−10 | 5.40E+04 | 2.17E−05 |

### Example 10: Conjugation of Anti-TfR1 Antibodies with Oligonucleotides

Complexes containing an anti-TfR1 antibody listed in Table 2 covalently conjugated to a tool oligo (control DMPK-ASO) were generated. First, Fab′ fragments of anti-TfR antibody clones 3-A4, 3-M12, 5-H12, 8-K6, 9-K23, 3-E5, 6-D3, 4-012, 4-05, 10-P5, 2-H19, 3-F3, 8-017, 3-M9, 10-H2, 4-J22, 9-D4, 8-D15, 4-H4, and 9-C4 were prepared by cutting the mouse monoclonal antibodies with an enzyme in or below the hinge region of the full IgG followed by partial reduction. The Fab′s were confirmed to be comparable to mAbs in avidity or affinity.

Muscle-targeting complexes was generated by covalently linking the anti-TfR mAbs to the control DMPK-ASO via a cathepsin cleavable linker. Briefly, a Bicyclo[6.1.0]nonyne-PEG3-L-valine-L-citrulline-pentafluorophenyl ester (BCN-PEG3-Val-Cit-PFP) linker molecule was coupled to control DMPK-ASO through a carbamate bond. Excess linker and organic solvents were removed by tangential flow filtration (TFF). The purified Val-Cit-linker-ASO was then coupled to an azide functionalized anti-transferrin receptor antibody generated through modifying ε-amine on lysine with Azide-PEG4-PFP. A positive control muscle-targeting complex was also generated using 15G11.

The product of the antibody coupling reaction was then subjected to two purification methods to remove free antibody and free payload: 1) hydrophobic interaction chromatography (HIC-HPLC), and 2) Size exclusion chromatography (SEC). The HIC column utilized a decreasing salt gradient to separate free antibody from conjugate. During SEC, fractionation was performed based on A260/A280 traces to specifically collect conjugated material. Concentrations of the conjugates were determined by either Nanodrop A280 or BCA protein assay (for antibody) and Quant-It Ribogreen assay (for payload). Corresponding drug-antibody ratios (DARs) were calculated. DARs ranged between 0.8 and 2.0, and were standardized so that all samples receive equal amounts of payload.

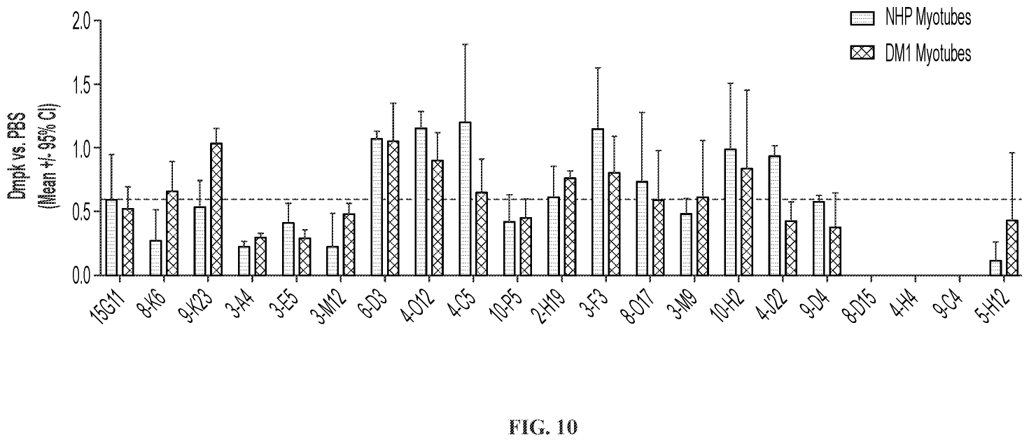

The purified complexes were then tested for cellular internalization and inhibition of the target gene, DMPK. Non-human primate (NHP) or DM1 (donated by DM1 patients) cells were grown in 96-well plates and differentiated into myotubes for 7 days. Cells were then treated with escalating concentrations (0.5 nM, 5 nM, 50 nM) of each complex for 72 hours. Cells were harvested, RNA was isolated, and reverse transcription was performed to generate cDNA. qPCR was performed using TaqMan kits specific for Ppib (control) and DMPK on the QuantStudio 7. The relative amounts of remaining DMPK transcript in treated vs non-treated cells was were calculated and the results are shown in Table 12 and FIG. 10.

The results demonstrated that the anti-TfR1 antibodies are able to target muscle cells, be internalized by the muscle cells with the molecular payload (the tool oligo control DMPK-ASO), and that the molecular payload (DMPK ASO) are able to target and knockdown the target gene (DMPK). Knockdown activity of a complex comprising the anti-TfR1 antibody conjugated to a molecular payload (e.g., an oligonucleotide) targeting any of the atrophy genes listed in Table 1 can be tested using the same assay described herein.

TABLE 12
Binding Affinity of anti-TfR1 Antibodies and Efficacy of Conjugates

| | | | | % knockdown of |
|---|---|---|---|---|
| | | | % knockdown of | DMPK in cells |
| | | | DMPK in NHP | from human DM1 |
| | huTfR1 Avg K_{D} | | cells using | patients using |
| | (M) | cyTfR1 Avg K_{D} (M) | Antibody-DMPK | Antibody-DMPK |
| Clone Name | (antibody alone) | (antibody alone) | ASO conjugate | ASO conjugate |
| 15G11 (control) | 8.0E−10 | 1.0E−09 | 36 | 46 |
| 3-A4 | 4.36E−10 | 2.32E−09 | 77 | 70 |
| 3-M12 | 7.68E−10 | 5.18E−09 | 77 | 52 |
| 5-H12 | 2.02316E−07 | 1.20E−08 | 88 | 57 |
| 8-K6 | 6.78121E−11 | 3.76E−10 | 73 | 34 |
| 9-K23 | 3.19783E−10 | 6.62E−10 | 47 | −4 |
| 3-E5 | 4.55E−10 | 6.71E−10 | 59 | 71 |
| 6-D3 | 9.86E−10 | 7.78E−10 | −8 | −5 |
| 4-O12 | 1.27416E−09 | 2.70E−07 | −16 | 10 |
| 4-C5 | 1.38324E−09 | 1.36E−08 | −20 | 35 |
| 10-P5 | 1.63E−09 | 1.10E−08 | 58 | 55 |
| 2-H19 | 2.06E−09 | 5.75E−09 | 39 | 24 |
| 3-F3 | 2.23E−09 | 1.84E−08 | −15 | 20 |
| 8-O17 | 2.24245E−09 | 1.10E−09 | 26 | 41 |
| 3-M9 | 2.50135E−09 | 4.37E−09 | 52 | 39 |
| 10-H2 | 2.72E−09 | 1.24E−08 | 2 | 16 |
| 4-J22 | 3.41E−09 | 1.37E−09 | 7 | 57 |
| 9-D4 | 5.79556E−09 | 8.68E−10 | 42 | 62 |
| 8-D15 | 9.15057E−09 | 1.11E−08 | * | * |
| 4-H4 | 1.39E−08 | 2.18E−08 | * | * |
| 9-C4 | 1.47657E−08 | 1.20E−08 | * | * |

* very low yield from expression/conjugation

Interestingly, the DMPK knockdown results showed a lack of correlation between the binding affinity of the anti-TfR to transferrin receptor and efficacy in delivering a DMPK ASO to cells for DMPK knockdown. Surprisingly, the anti-TfR antibodies provided herein (e.g., at least 3-A4, 3-M12, 5-H12, 8-K6, 3-E5, 10-P5, 3-M9, and 9-D4) demonstrated superior activity in delivering a payload (e.g., DMPK ASO) to the target cells and achieving the biological effect of the molecular payload (e.g., DMPK knockdown) in either cyno cells or human DM1 patient cells, compared to the control antibody 15G11, despite the comparable binding affinity (or, in certain instances, such as 5-H12, lower binding affinity) to human or cyno transferrin receptor between these antibodies and the control antibody 15G11.

Top attributes such as high huTfR1 affinity, >50% knockdown of DMPK in NHP and DM1 patient cell line, identified epitope binding with 3 unique sequences, low/no predicted PTM sites, and good expression and conjugation efficiency were considered for the selection of clones for humanization.

### Example 11. Binding Activities of the Anti-TfR1 Antibodies

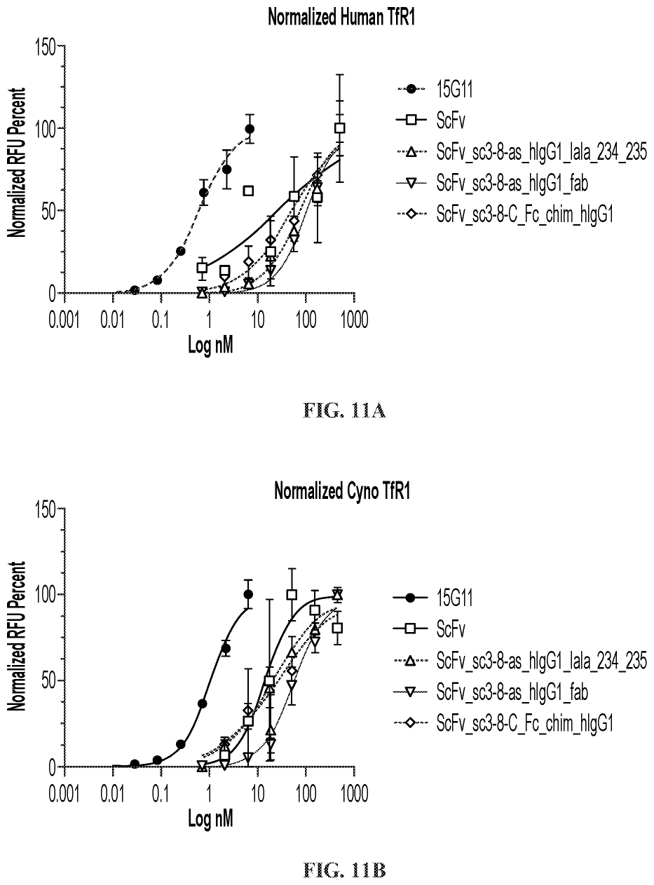

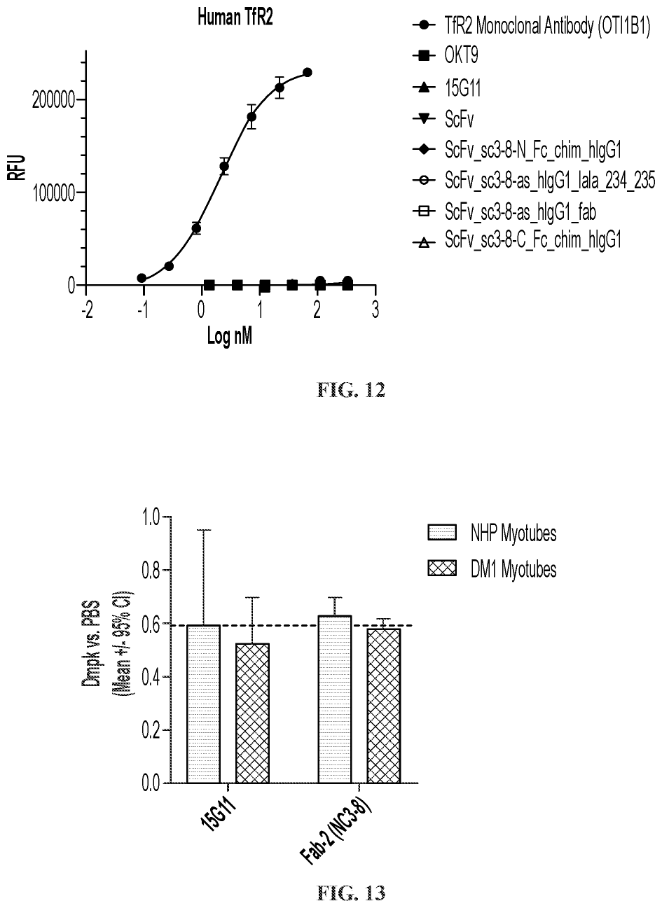

The screen identified 1 scFv clone (shown in Table 7), which was reformatted into different formats. The binding activity of selected formats were tested against human TfR1, cyno TfR1, and human TfR2 in an ELISA assay. 15G11 was used as control in this experiment. The results show that all tested antibodies bind to human TfR1 and cyno TfR1 (FIGS. 11A and 11B), but do not bind to human TfR2 (FIG. 12). The EC50 values for each tested antibody are provided in Table 13.

TABLE 13
EC50 (nM) values for anti-TfR antibodies

| | | | hIgG1 (with | | |
|---|---|---|---|---|---|
| | | | L234A/L235A | | |
| | | | mutations in HC | | |
| | 15G11 | ScFv | constant region) | FAB | scFv_C_Fc |
| Cyno TfR1 | 1.08 | 14.75 | 22.01 | 63.89 | 27.21 |
| Human TfR1 | 0.589 | 24.8 | 75.83 | 101.9 | 49.56 |

### Example 12: Conjugation of anti-TfR1 Antibodies with Oligonucleotides

Complexes containing an anti-TfR1 Fab covalently conjugated to a tool oligo, control DMPK-ASO (targeting DMPK), were generated. The TfR Fab tested comprises a VH of SEQ ID NO: 204 and a VL of SEQ ID NO: 205. A Fab′ fragment of a known anti-TfR antibody, 15G11 was generated and used to produce a complex as positive control.

Muscle-targeting complexes was generated by covalently linking the anti-TfR antibodies to control DMPK-ASO via a cathepsin cleavable linker. The purified Val-Cit-linker-ASO was coupled to functionalized anti-transferrin receptor antibodies generated through modifying ε-amine on lysine of the antibody.

The product of the antibody coupling reaction was then subjected to two purification methods to remove free antibody and free payload: 1) hydrophobic interaction chromatography (HIC-HPLC), and 2) Size exclusion chromatography (SEC). The HIC column utilized a decreasing salt gradient to separate free antibody from conjugate. During SEC, fractionation was performed based on A260/A280 traces to specifically collect conjugated material. Concentrations of the conjugates were determined by either Nanodrop A280 or BCA protein assay (for antibody) and Quant-It Ribogreen assay (for payload). Corresponding drug-antibody ratios (DARs) were calculated. DAR was about 2.05.

The purified complexes were then tested for cellular internalization and inhibition of DMPK. Non-human primate (NHP) or DM1 (donated by DM1 patients) cells were grown in 96-well plates and differentiated into myotubes for 7 days. Cells were then treated with escalating concentrations (0.5 nM, 5 nM, 50 nM) of each complex for 72 hours. Cells were harvested, RNA was isolated, and reverse transcription was performed to generate cDNA. qPCR was performed using TaqMan kits specific for Ppib (control) and DMPK on the QuantStudio 7. The relative amounts of remaining DMPK transcript in treated vs non-treated cells was were calculated and the results are shown in FIG. 13. The complex containing anti-TfR Fab described herein achieved comparable DMPK knockdown as the complex containing 15G11.

The results demonstrated that the anti-TfR1 antibodies are able to target muscle cells, be internalized by the muscle cells with the molecular payload (the tool oligo control DMPK-ASO), and that the molecular payload (DMPK ASO) are able to target and knockdown the target gene (DMPK). Knockdown activity of a complex comprising the anti-TfR1 antibody conjugated to a molecular payload (e.g., an oligonucleotide) targeting any of the atrophy genes listed in Table 1 can be tested using the same assay described herein.

### Example 13. Binding and Biological Activity of Anti-TfR-Oligonucleotide Conjugates

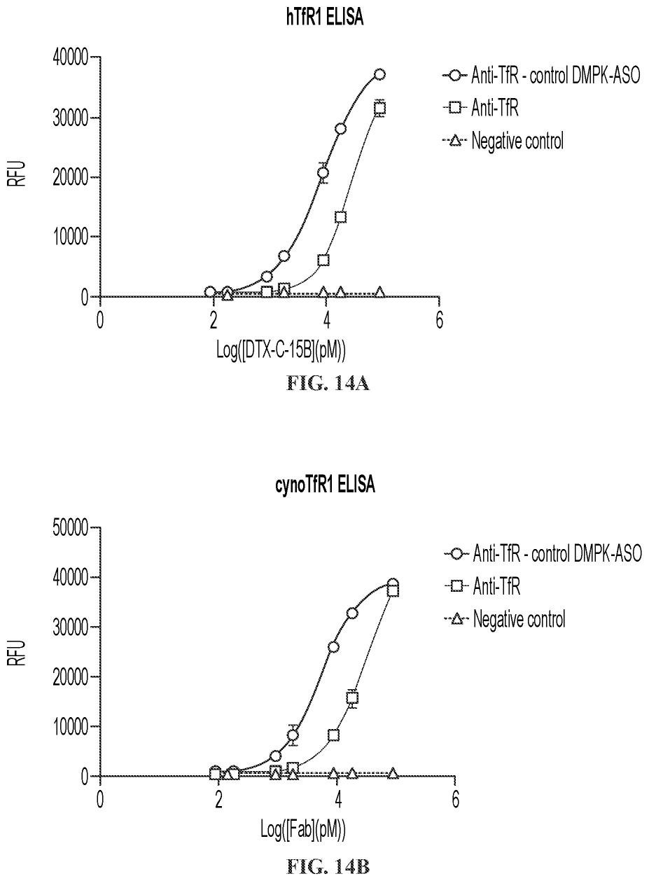

The anti-TfR antibody described herein (e.g., as in Table 7) alone or in a conjugate where the antibody was conjugated to a DMPK-targeting oligonucleotide (control DMPK-ASO) were tested for binding to human (FIG. 14A) and cynomolgus monkey (FIG. 14B) TfR1. Results demonstrate that binding of the anti-TfR antibody to both hTfR1 and cynoTfR1 increases 3-6-fold upon conjugation to DMPK-targeting oligonucleotide.

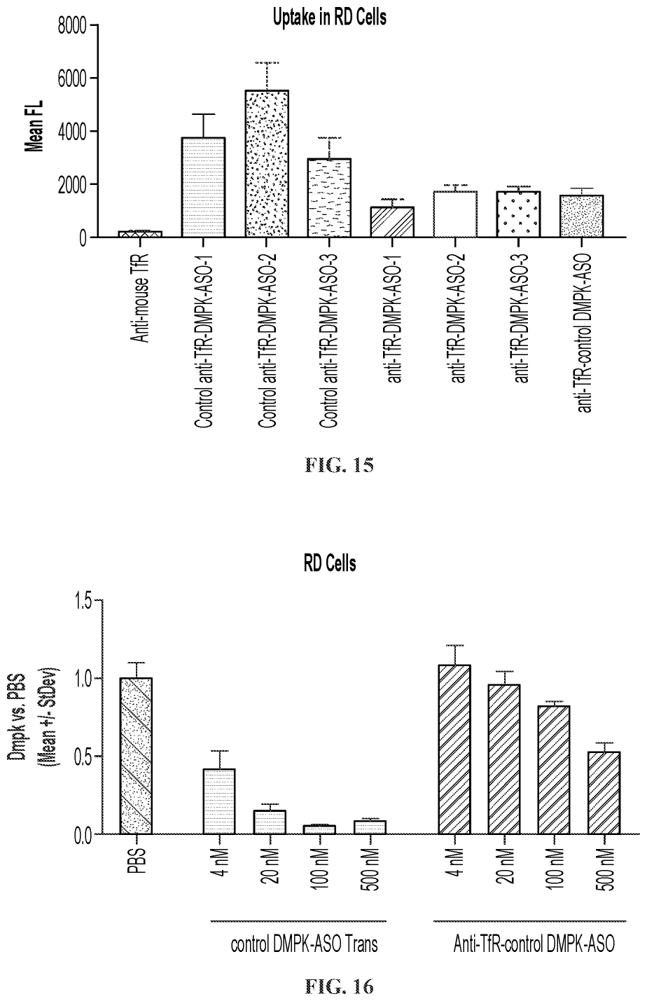

The conjugate was also tested in cellular uptake experiments to evaluate TfR1-mediated internalization. To measure such cellular uptake mediated by antibodies, the anti-TfR antibody was conjugated to several different DMPK-targeting oligonucleotides, and the conjugate were labeled with Cypher5e, a pH-sensitive dye. Rhabdomyosarcoma (RD) cells were treated for 4 hours with 100 nM of the conjugates, trypsinized, washed twice, and analyzed by flow cytometry. Mean Cypher5e fluorescence (representing uptake) was calculated using Attune NxT software. As shown in FIG. 15, the anti-TfR antibody show endosomal uptake. Similar internalization efficiency were observed for different oligonucleotide payloads. An anti-mouse TfR antibody was used as the negative control. Cold (non-internalizing) conditions abrogated the fluorescence signal of the positive control antibody-conjugate (data not shown), indicating that the positive signal in the positive control and humanized anti-TfR Fab-conjugates is due to internalization of the Fab-conjugates.

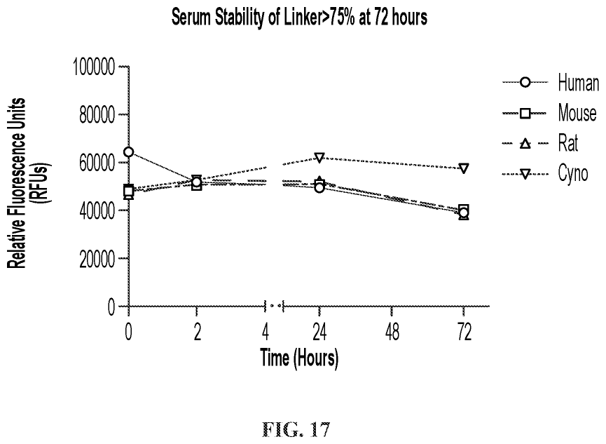

The activity of the conjugate containing the anti-TfR antibody and the DMPK-targeting oligonucleotide (control DMPK-ASO) in knocking down DMPK mRNA level in RD cells was also tested. The results showed that the conjugated achieved dose-dependent knock down of DMPK mRNA level (FIG. 16).

The results demonstrate that the anti-TfR1 antibody bind to TfR1 on muscles with high affinity, can mediate the internalized of a conjugated molecular payload (e.g., oligonucleotide) and that the molecular payload (DMPK-targeting oligonucleotide) are able to target and knockdown the target gene (DMPK). Molecular payloads targeting other genes can also be conjugated to the anti-TfR antibody described herein and used to target other genes specifically in muscle cells.

### Example 14. Serum Stability of the Linker Linking the Anti-TfR Antibody and the Molecular Payload

Oligonucleotides which were linked to antibodies in examples were conjugated via a cleavable linker shown in Formula (C). It is important that the linker maintain stability in serum and provide release kinetics that favor sufficient payload accumulation in the targeted muscle cell. This serum stability is important for systemic intravenous administration, stability of the conjugated oligonucleotide in the bloodstream, delivery to muscle tissue and internalization of the therapeutic payload in the muscle cells. The linker has been confirmed to facilitate precise conjugation of multiple types of payloads (including ASOs, siRNAs and PMOs) to Fabs. This flexibility enabled rational selection of the appropriate type of payload to address the genetic basis of each muscle disease. Additionally, the linker and conjugation chemistry allowed the optimization of the ratio of payload molecules attached to each Fab for each type of payload, and enabled rapid design, production and screening of molecules to enable use in various muscle disease applications.

FIG. 17 shows serum stability of the linker in vivo, which was comparable across multiple species over the course of 72 hours after intravenous dosing. At least 75% stability was measured in each case at 72 hours after dosing.

### Additional Embodiments

1. A method for treating a subject diagnosed as having muscle atrophy, the method comprising administering to the subject a complex comprising a muscle-targeting agent covalently linked to a molecular payload configured to inhibit expression or activity of a pro-atrophy gene, wherein the muscle-targeting agent specifically binds to an internalizing cell surface receptor on muscle cells of the subject,

wherein the muscle targeting agent is an antibody that binds to a transferrin receptor and comprises a heavy chain variable region (VH) comprising a CDR-H1, a CDR-H2, and a CDR-H3 of any one of the antibodies listed in Table 2, Table 4, or Table 7, and/or a light chain variable region (VL) comprising a CDR-L1, a CDR-L2, and a CDR-L3 of any one of the antibodies listed in Table 2, Table 4, or Table 7.

2. The method of embodiment 1, wherein the antibody comprises a VH that is at least 85% identical to the VH of any one of the antibodies listed in Table 2 or Table 7, and/or a VL that is at least 85% identical to the VL of any one of the antibodies listed in Table 2 or Table 7.

3. The method of embodiment 1, wherein the antibody is selected from:

(i) an antibody comprising a CDR-H1, a CDR-H2, and a CDR-H3 of a VH comprising the amino acid sequence of SEQ ID NO: 7, and/or a CDR-L1, a CDR-L2, and a CDR-L3 of a VL comprising the amino acid sequence of SEQ ID NO: 8;

(ii) an antibody comprising a CDR-H1, a CDR-H2, and a CDR-H3 of a VH comprising the amino acid sequence of SEQ ID NO: 15, and/or a CDR-L1, a CDR-L2, and a CDR-L3 of a VL comprising the amino acid sequence of SEQ ID NO: 16;

(iii) an antibody comprising a CDR-H1, a CDR-H2, and a CDR-H3 of a VH comprising the amino acid sequence of SEQ ID NO: 23, and/or a CDR-L1, a CDR-L2, and a CDR-L3 of a VL comprising the amino acid sequence of SEQ ID NO: 24; and

(iv) an antibody comprising a CDR-H1, a CDR-H2, and a CDR-H3 of a VH comprising the amino acid sequence of SEQ ID NO: 204, and/or a CDR-L1, a CDR-L2, and a CDR-L3 of a VL comprising the amino acid sequence of SEQ ID NO: 205.

4. The method of embodiment 1, wherein the antibody comprises:

(i) a CDR-H1 of SEQ ID NO: 1, a CDR-H2 of SEQ ID NO: 2, SEQ ID NO: 262, or SEQ ID NO: 80, a CDR-H3 of SEQ ID NO: 3, a CDR-L1 of SEQ ID NO: 4, a CDR-L2 of SEQ ID NO: 5, and a CDR-L3 of SEQ ID NO: 6;

(ii) a CDR-H1 of SEQ ID NO: 145, a CDR-H2 of SEQ ID NO: 146, SEQ ID NO: 263, or SEQ ID NO: 265, a CDR-H3 of SEQ ID NO: 147, a CDR-L1 of SEQ ID NO: 148, a CDR-L2 of SEQ ID NO: 149, and a CDR-L3 of SEQ ID NO: 6; or

(iii) a CDR-H1 of SEQ ID NO: 150, a CDR-H2 of SEQ ID NO: 151, SEQ ID NO: 270, or SEQ ID NO: 271, a CDR-H3 of SEQ ID NO: 152, a CDR-L1 of SEQ ID NO: 153, a CDR-L2 of SEQ ID NO: 5, and a CDR-L3 of SEQ ID NO: 154.

5. The method of embodiment 1, wherein the antibody comprises:

(i) a CDR-H1 of SEQ ID NO: 9, a CDR-H2 of SEQ ID NO: 10, a CDR-H3 of SEQ ID NO: 11, a CDR-L1 of SEQ ID NO: 12, a CDR-L2 of SEQ ID NO: 13, and a CDR-L3 of SEQ ID NO: 14;

(ii) a CDR-H1 of SEQ ID NO: 155, a CDR-H2 of SEQ ID NO: 156, a CDR-H3 of SEQ ID NO: 157, a CDR-L1 of SEQ ID NO: 158, a CDR-L2 of SEQ ID NO: 159, and a CDR-L3 of SEQ ID NO: 14; or

(iii) a CDR-H1 of SEQ ID NO: 160, a CDR-H2 of SEQ ID NO: 161, a CDR-H3 of SEQ ID NO: 162, a CDR-L1 of SEQ ID NO: 163, a CDR-L2 of SEQ ID NO: 13, and a CDR-L3 of SEQ ID NO: 164.

6. The method of embodiment 1, wherein the antibody comprises:

(i) a CDR-H1 of SEQ ID NO: 17, SEQ ID NO: 266, or SEQ ID NO: 268, a CDR-H2 of SEQ ID NO: 18, a CDR-H3 of SEQ ID NO: 19, a CDR-L1 of SEQ ID NO: 20, a CDR-L2 of SEQ ID NO: 21, and a CDR-L3 of SEQ ID NO: 22;

(ii) a CDR-H1 of SEQ ID NO: 165, SEQ ID NO: 267, or SEQ ID NO: 269, a CDR-H2 of SEQ ID NO: 166, a CDR-H3 of SEQ ID NO: 167, a CDR-L1 of SEQ ID NO: 168, a CDR-L2 of SEQ ID NO: 169, and a CDR-L3 of SEQ ID NO: 22; or

(iii) a CDR-H1 of SEQ ID NO: 170, a CDR-H2 of SEQ ID NO: 171, a CDR-H3 of SEQ ID NO: 172, a CDR-L1 of SEQ ID NO: 173, a CDR-L2 of SEQ ID NO: 21, and a CDR-L3 of SEQ ID NO: 174.

7. The method of embodiment 1, wherein the antibody comprises:

(i) a CDR-H1 of SEQ ID NO: 188, a CDR-H2 of SEQ ID NO: 189, a CDR-H3 of SEQ ID NO: 190, a CDR-L1 of SEQ ID NO: 191, a CDR-L2 of SEQ ID NO: 192, and a CDR-L3 of SEQ ID NO: 193;

(ii) a CDR-H1 of SEQ ID NO: 194, a CDR-H2 of SEQ ID NO: 195, a CDR-H3 of SEQ ID NO: 196, a CDR-L1 of SEQ ID NO: 197, a CDR-L2 of SEQ ID NO: 198, and a CDR-L3 of SEQ ID NO: 193; or

(iii) a CDR-H1 of SEQ ID NO: 199, a CDR-H2 of SEQ ID NO: 200, a CDR-H3 of SEQ ID NO: 201, a CDR-L1 of SEQ ID NO: 202, a CDR-L2 of SEQ ID NO: 192, and a CDR-L3 of SEQ ID NO: 203.

8. The method of any one of embodiments 1-7, wherein the antibody is selected from:

(i) an antibody comprising a VH comprising an amino acid sequence at least 85% identical to SEQ ID NO: 7, and/or a VL comprising an amino acid sequence at least 85% identical to SEQ ID NO: 8;

(ii) an antibody comprising a VH comprising an amino acid sequence at least 85% identical to SEQ ID NO: 15, and/or a VL comprising an amino acid sequence at least 85% identical to SEQ ID NO: 16;

(iii) an antibody comprising a VH comprising an amino acid sequence at least 85% identical to SEQ ID NO: 23, and/or a VL comprising an amino acid sequence at least 85% identical to SEQ ID NO: 24; and

(iv) an antibody comprising a VH comprising an amino acid sequence at least 85% identical to SEQ ID NO: 204, and/or a VL comprising an amino acid sequence at least 85% identical to SEQ ID NO: 205.

9. The method of any one of embodiments 1-8, wherein the subject has had ongoing muscle atrophy in excess of one month.

10. The method of any one of embodiments 1-9, wherein the muscle atrophy is not localized to the subject's lower or hind limb extremities.

11. The method of any one of embodiments 1 to 10, wherein the muscle atrophy is present in multiple different muscle groups of the subject.

12. The method of any one of embodiments 1 to 11, wherein the subject does not have peripheral artery disease.

13. The method of any one of embodiments 1 to 12, wherein the peripheral arterial system of the subject is intact.

14. The method of any one of embodiments 1 to 13, wherein the equilibrium dissociation constant (KD) of binding of the antibody to the transferrin receptor is in a range from 10⁻¹¹M to 10⁻⁶ M.

15. The method of any one of embodiments 1 to 14, wherein the antibody does not specifically bind to the transferrin binding site of the transferrin receptor and/or wherein the antibody does not inhibit binding of transferrin to the transferrin receptor.

16. The method of any one of embodiments 1 to 15, wherein the antibody is cross-reactive with extracellular epitopes of two or more of a human, non-human primate and rodent transferrin receptor.

17. The method of any one of embodiments 1 to 16, wherein the method is configured to promote transferrin receptor mediated internalization of the molecular payload into a muscle cell.

18. The method of any one of embodiments 1 to 17, wherein the antibody is a chimeric antibody, optionally wherein the chimeric antibody is a humanized monoclonal antibody.

19. The method of any one of embodiments 1 to 18, wherein the antibody is in the form of a ScFv, Fab fragment, F(ab′) fragment, F(ab′)₂ fragment, or Fv fragment.

20. The method of any one of embodiments 1 to 19, wherein the molecular payload is an oligonucleotide.

21. The method of embodiment 20, wherein the oligonucleotide comprises a region of complementarity to an INHBA, MSTN, TRIM63, or FBXO32 gene.

23. A method for treating a subject having muscle atrophy, the method comprising extramuscular parenteral administration of a complex comprising a muscle-targeting agent covalently linked to an oligonucleotide to the subject, wherein the muscle-targeting agent specifically binds to an internalizing cell surface receptor on muscle cells of the subject, and wherein the oligonucleotide comprises a region of complementarity to a pro-atrophy gene, wherein the muscle targeting agent is an antibody that binds to a transferrin receptor and comprises a heavy chain variable region (VH) comprising a CDR-H1, a CDR-H2, and a CDR-H3 of any one of the antibodies listed in Table 2, Table 4, or Table 7, and/or comprises a light chain variable region (VL) comprising a CDR-L1, a CDR-L2, and a CDR-L3 of any one of the antibodies listed in Table 2, Table 4, or Table 7.

24. The method of embodiment 23, wherein the method comprises intravenous administration of the complex to the subject.

25. The method of embodiment 23, wherein the method comprises subcutaneous administration of the complex to the subject.

26. A complex comprising a muscle-targeting agent linked to a single-stranded oligonucleotide, wherein the muscle-targeting agent specifically binds to an internalizing cell surface receptor on muscle cells, and wherein the oligonucleotide comprises a region of complementarity to a pro-atrophy gene, wherein the muscle targeting agent is an antibody that binds to a transferrin receptor and comprises a heavy chain variable region (VH) comprising a CDR-H1, a CDR-H2, and a CDR-H3 of any one of the antibodies listed in Table 2, Table 4, or Table 7, and/or comprises a light chain variable region (VL) comprising a CDR-L1, a CDR-L2, and a CDR-L3 of any one of the antibodies listed in Table 2, Table 4, or Table 7.

27. A composition comprising a plurality of complexes, each complex comprising a muscle-targeting agent covalently linked to at least three oligonucleotides, wherein the muscle-targeting agent specifically binds to an internalizing cell surface receptor on muscle cells of a subject, and wherein each oligonucleotide comprises a region of complementarity to a pro-atrophy gene, wherein the muscle targeting agent is an antibody that binds to a transferrin receptor and comprises a heavy chain variable region (VH) comprising a CDR-H1, a CDR-H2, and a CDR-H3 of any one of the antibodies listed in Table 2, Table 4, or Table 7, and/or comprises a light chain variable region (VL) comprising a CDR-L1, a CDR-L2, and a CDR-L3 of any one of the antibodies listed in Table 2, Table 4, or Table 7.

28. A complex comprising a muscle-targeting agent covalently linked to a molecular payload configured to inhibit expression or activity of a pro-atrophy gene that encodes a non-secreted product that functions within muscle cells, wherein the muscle-targeting agent specifically binds to an internalizing cell surface receptor on muscle cells, wherein the muscle targeting agent is an antibody that binds to a transferrin receptor and comprises a heavy chain variable region (VH) comprising a CDR-H1, a CDR-H2, and a CDR-H3 of any one of the antibodies listed in Table 2, Table 4, or Table 7. and/or a light chain variable region (VL) comprising a CDR-L1, a CDR-L2, and a CDR-L3 of any one of the antibodies listed in Table 2, Table 4, or Table 7.

29. The complex of embodiment 28, wherein the antibody comprises a VH that is at least 85% identical to the VH of any one of the antibodies listed in Table 2 or Table 7, and/or a VL that is at least 85% identical to the VL of any one of the antibodies listed in Table 2 or Table 7.

30. The complex of embodiment 28, wherein the antibody is selected from:

(i) an antibody comprising a CDR-H1, a CDR-H2, and a CDR-H3 of a VH comprising the amino acid sequence of SEQ ID NO: 7, and/or a CDR-L1, a CDR-L2, and a CDR-L3 of a VL comprising the amino acid sequence of SEQ ID NO: 8;

(ii) an antibody comprising a CDR-H1, a CDR-H2, and a CDR-H3 of a VH comprising the amino acid sequence of SEQ ID NO: 15, and/or a CDR-L1, a CDR-L2, and a CDR-L3 of a VL comprising the amino acid sequence of SEQ ID NO: 16;

(iii) an antibody comprising a CDR-H1, a CDR-H2, and a CDR-H3 of a VH comprising the amino acid sequence of SEQ ID NO: 23, and/or a CDR-L1, a CDR-L2, and a CDR-L3 of a VL comprising the amino acid sequence of SEQ ID NO: 24; and

(iv) an antibody comprising a CDR-H1, a CDR-H2, and a CDR-H3 of a VH comprising the amino acid sequence of SEQ ID NO: 204, and/or a CDR-L1, a CDR-L2, and a CDR-L3 of a VL comprising the amino acid sequence of SEQ ID NO: 205.

31. The complex of embodiment 28, wherein the antibody comprises:

(i) a CDR-H1 of SEQ ID NO: 1, a CDR-H2 of SEQ ID NO: 2, SEQ ID NO: 262, or SEQ ID NO: 80, a CDR-H3 of SEQ ID NO: 3, a CDR-L1 of SEQ ID NO: 4, a CDR-L2 of SEQ ID NO: 5, and a CDR-L3 of SEQ ID NO: 6;

(ii) a CDR-H1 of SEQ ID NO: 145, a CDR-H2 of SEQ ID NO: 146, SEQ ID NO: 263, or SEQ ID NO: 265, a CDR-H3 of SEQ ID NO: 147, a CDR-L1 of SEQ ID NO: 148, a CDR-L2 of SEQ ID NO: 149, and a CDR-L3 of SEQ ID NO: 6; or

(iii) a CDR-H1 of SEQ ID NO: 150, a CDR-H2 of SEQ ID NO: 151, SEQ ID NO: 270, or SEQ ID NO: 271, a CDR-H3 of SEQ ID NO: 152, a CDR-L1 of SEQ ID NO: 153, a CDR-L2 of SEQ ID NO: 5, and a CDR-L3 of SEQ ID NO: 154.

32. The complex of embodiment 28, wherein the antibody comprises:

(i) a CDR-H1 of SEQ ID NO: 9, a CDR-H2 of SEQ ID NO: 10, a CDR-H3 of SEQ ID NO: 11, a CDR-L1 of SEQ ID NO: 12, a CDR-L2 of SEQ ID NO: 13, and a CDR-L3 of SEQ ID NO: 14;

(ii) a CDR-H1 of SEQ ID NO: 155, a CDR-H2 of SEQ ID NO: 156, a CDR-H3 of SEQ ID NO: 157, a CDR-L1 of SEQ ID NO: 158, a CDR-L2 of SEQ ID NO: 159, and a CDR-L3 of SEQ ID NO: 14; or

(iii) a CDR-H1 of SEQ ID NO: 160, a CDR-H2 of SEQ ID NO: 161, a CDR-H3 of SEQ ID NO: 162, a CDR-L1 of SEQ ID NO: 163, a CDR-L2 of SEQ ID NO: 13, and a CDR-L3 of SEQ ID NO: 164.

33. The complex of embodiment 28, wherein the antibody comprises:

(i) a CDR-H1 of SEQ ID NO: 17, SEQ ID NO: 266, or SEQ ID NO: 268, a CDR-H2 of SEQ ID NO: 18, a CDR-H3 of SEQ ID NO: 19, a CDR-L1 of SEQ ID NO: 20, a CDR-L2 of SEQ ID NO: 21, and a CDR-L3 of SEQ ID NO: 22;

(ii) a CDR-H1 of SEQ ID NO: 165, SEQ ID NO: 267, or SEQ ID NO: 269, a CDR-H2 of SEQ ID NO: 166, a CDR-H3 of SEQ ID NO: 167, a CDR-L1 of SEQ ID NO: 168, a CDR-L2 of SEQ ID NO: 169, and a CDR-L3 of SEQ ID NO: 22; or

(iii) a CDR-H1 of SEQ ID NO: 170, a CDR-H2 of SEQ ID NO: 171, a CDR-H3 of SEQ ID NO: 172, a CDR-L1 of SEQ ID NO: 173, a CDR-L2 of SEQ ID NO: 21, and a CDR-L3 of SEQ ID NO: 174.

34. The complex of embodiment 28, wherein the antibody comprises:

(i) a CDR-H1 of SEQ ID NO: 188, a CDR-H2 of SEQ ID NO: 189, a CDR-H3 of SEQ ID NO: 190, a CDR-L1 of SEQ ID NO: 191, a CDR-L2 of SEQ ID NO: 192, and a CDR-L3 of SEQ ID NO: 193;

(ii) a CDR-H1 of SEQ ID NO: 194, a CDR-H2 of SEQ ID NO: 195, a CDR-H3 of SEQ ID NO: 196, a CDR-L1 of SEQ ID NO: 197, a CDR-L2 of SEQ ID NO: 198, and a CDR-L3 of SEQ ID NO: 193; or

(iii) a CDR-H1 of SEQ ID NO: 199, a CDR-H2 of SEQ ID NO: 200, a CDR-H3 of SEQ ID NO: 201, a CDR-L1 of SEQ ID NO: 202, a CDR-L2 of SEQ ID NO: 192, and a CDR-L3 of SEQ ID NO: 203.

35. The complex of any one of embodiments 28-34, wherein the antibody is selected from:

(i) an antibody comprising a VH comprising an amino acid sequence at least 85% identical to SEQ ID NO: 7, and/or a VL comprising an amino acid sequence at least 85% identical to SEQ ID NO: 8;

(ii) an antibody comprising a VH comprising an amino acid sequence at least 85% identical to SEQ ID NO: 15, and/or a VL comprising an amino acid sequence at least 85% identical to SEQ ID NO: 16;

(iii) an antibody comprising a VH comprising an amino acid sequence at least 85% identical to SEQ ID NO: 23, and/or a VL comprising an amino acid sequence at least 85% identical to SEQ ID NO: 24;

(iv) an antibody comprising a VH comprising an amino acid sequence at least 85% identical to SEQ ID NO: 204, and/or a VL comprising an amino acid sequence at least 85% identical to SEQ ID NO: 205.

36. The complex of any one of embodiments 28-35, wherein the equilibrium dissociation constant (Kd) of binding of the antibody to the transferrin receptor is in a range from 10⁻¹¹M to 10⁻⁶M.

37. The complex of any one of embodiments 28 to 36, wherein the antibody does not specifically bind to the transferrin binding site of the transferrin receptor and/or wherein the antibody does not inhibit binding of transferrin to the transferrin receptor.

38. The complex of any one of embodiments 28 to 37, wherein the antibody is cross-reactive with extracellular epitopes of two or more of a human, non-human primate and rodent transferrin receptor.

39. The complex of any one of embodiments 28 to 38, wherein the complex is configured to promote transferrin receptor mediated internalization of the molecular payload into a muscle cell.

40. The complex of any one of embodiments 28 to 39, wherein the antibody is a chimeric antibody, wherein optionally the chimeric antibody is a humanized monoclonal antibody.

41. The complex of any one of embodiments 28 to 40, wherein the antibody is in the form of a ScFv, Fab fragment, F(ab′) fragment, F(ab′)₂ fragment, or Fv fragment.

42. The complex of any one of embodiments 28 to 41, wherein the molecular payload is an oligonucleotide.

43. The complex of embodiment 42, wherein the oligonucleotide comprises a region of complementarity to the pro-atrophy gene.

44. The complex of embodiment 43, wherein the pro-atrophy gene is INHBA, MSTN, TRIM63 or FBXO32.

45. The complex of any one of embodiments 28 to 41, wherein the molecular payload is an polypeptide.

46. The complex of embodiment 45, wherein the polypeptide is an E3 ubiquitin ligase inhibitor peptide.

47. The complex of embodiment 42 or 43, wherein the oligonucleotide comprises at least one modified internucleotide linkage.

48. The complex of embodiment 47, wherein the at least one modified internucleotide linkage is a phosphorothioate linkage.

49. The complex of embodiment 48, wherein the oligonucleotide comprises phosphorothioate linkages in the Rp stereochemical conformation and/or in the Sp stereochemical conformation.

50. The complex of embodiment 49, wherein the oligonucleotide comprises phosphorothioate linkages that are all in the Rp stereochemical conformation or that are all in the Sp stereochemical conformation.

51. The complex of any one of embodiments 42, 43, or 47 to 50, wherein the oligonucleotide comprises one or more modified nucleotides.

52. The complex of embodiment 51, wherein the one or more modified nucleotides are 2′-modified nucleotides.

53. The complex of any one of embodiments 42, 43, or 47 to 52, wherein the oligonucleotide is a gapmer oligonucleotide that directs RNAse H-mediated cleavage of an mRNA transcript encoded by the pro-atrophy gene in a cell.

54. The complex of embodiment 53, wherein the gapmer oligonucleotide comprises a central portion of 5 to 15 deoxyribonucleotides flanked by wings of 2 to 8 modified nucleotides.

55. The complex of embodiment 54, wherein the modified nucleotides of the wings are 2′-modified nucleotides.

56. The complex of any one of embodiments 42, 43, or 47 to 52, wherein the oligonucleotide is a mixmer oligonucleotide.

57. The complex of embodiment 56, wherein the mixmer oligonucleotide comprises two or more different 2′ modified nucleotides.

58. The complex of any one of embodiments 42, 43, or 47 to 52, wherein the oligonucleotide is an RNAi oligonucleotide that promotes RNAi-mediated cleavage of a mRNA transcript encoded by the pro-atrophy gene.

59. The complex of embodiment 58, wherein the RNAi oligonucleotide is a double-stranded oligonucleotide of 19 to 25 nucleotides in length.

60. The complex of embodiment 58 or 59, wherein the RNAi oligonucleotide comprises at least one 2′ modified nucleotide.

61. The complex of embodiment 52, 55, 57, or 60, wherein each 2′ modified nucleotide is selected from the group consisting of: 2′-O-methyl, 2′-fluoro (2′-F), 2′-O-methoxyethyl (2′-MOE), and 2′,4′-bridged nucleotides.

62. The complex of embodiment 51, wherein the one or more modified nucleotides are bridged nucleotides.

63. The complex of any one of embodiments 52, 55, 57, or 60, wherein at least one 2′ modified nucleotide is a 2′,4′-bridged nucleotide selected from: 2′,4′-constrained 2′-O-ethyl (cEt) and locked nucleic acid (LNA) nucleotides.

64. The complex of any one of embodiments 42, 43, or 47 to 52, wherein the oligonucleotide comprises a guide sequence for a genome editing nuclease.

65. The complex of any one of embodiments 42, 43, or 47 to 52, wherein the oligonucleotide is phosphorodiamidite morpholino oligomer.

66. The complex of any one of embodiments 28 to 65, wherein the antibody is covalently linked to the molecular payload via a cleavable linker.

67. The complex of embodiment 66, wherein the cleavable linker is selected from: a protease-sensitive linker, pH-sensitive linker, and glutathione-sensitive linker.

68. The complex of embodiment 67, wherein the cleavable linker is a protease-sensitive linker.

69. The complex of embodiment 68, wherein the protease-sensitive linker comprises a sequence cleavable by a lysosomal protease and/or an endosomal protease.

70. The complex of embodiment 68, wherein the protease-sensitive linker comprises a valine-citrulline dipeptide sequence.

71. The complex of embodiment 67, wherein the linker is a pH-sensitive linker that is cleaved at a pH in a range of 4 to 6.

72. The complex of any one of embodiments 28 to 65, wherein the antibody is covalently linked to the molecular payload via a non-cleavable linker.

73. The complex of embodiment 72, wherein the non-cleavable linker is an alkane linker.

74. The complex of any one of embodiments 28 to 73, wherein the antibody comprises a non-natural amino acid to which the oligonucleotide is covalently linked.

75. The complex of any one of embodiments 28 to 74, wherein the antibody is covalently linked to the oligonucleotide via conjugation to a lysine residue or a cysteine residue of the antibody.

76. The complex of embodiment 75, wherein the oligonucleotide is conjugated to the cysteine of the antibody via a maleimide-containing linker, optionally wherein the maleimide-containing linker comprises a maleimidocaproyl or maleimidomethyl cyclohexane-1-carboxylate group.

77. The complex of embodiments 29 to 76, wherein the antibody is a glycosylated antibody that comprises at least one sugar moiety to which the oligonucleotide is covalently linked.

78. The complex of embodiment 77, wherein the sugar moiety is a branched mannose.

79. The complex of embodiment 77 or 78, wherein the antibody is a glycosylated antibody that comprises one to four sugar moieties each of which is covalently linked to a separate oligonucleotide.

80. The complex of embodiment 77, wherein the antibody is a fully-glycosylated antibody.

81. The complex of embodiment 77, wherein the antibody is a partially-glycosylated antibody.

82. The complex of embodiment 81, wherein the partially-glycosylated antibody is produced via chemical or enzymatic means.

83. The complex of embodiment 81, wherein the partially-glycosylated antibody is produced in a cell that is deficient for an enzyme in the N- or O-glycosylation pathway.

84. A method of delivering a molecular payload to a cell expressing transferrin receptor, the method comprising contacting the cell with the complex of any one of embodiments 29 to 83.

85. A method of inhibiting activity of a pro-atrophy gene in a cell, the method comprising contacting the cell with the complex of any one of embodiments 29 to 83 in an amount effective for promoting internalization of the molecular payload to the cell.

86. The method of embodiment 85, wherein the cell is in vitro.

87. The method of embodiment 85, wherein the cell is in a subject.

88. The method of embodiment 87, wherein the subject is a human.

89. The method of any one of embodiments 85 to 88, wherein the pro-atrophy gene is INHBA, MSTN, TRIM63 or FBX032.

90. A method of treating a subject having muscle atrophy, the method comprising administering to the subject an effective amount of the complex of any one of embodiments 29 to 83.

91. The method of embodiment 90, wherein the subject has had ongoing muscle atrophy in excess of one month.

92. The method of embodiment 90 or 91, wherein the muscle atrophy is not localized to the subject's lower or hind limb extremities.

93. The method of any one of embodiments 90 to 92, wherein the muscle atrophy is present in multiple different muscle groups of the subject.

94. The method of any one of embodiments 90 to 93, wherein the subject does not have peripheral artery disease.

95. The method of any one of embodiments 90 to 94, wherein the peripheral arterial system of the subject is intact.

### Equivalents and Terminology

The disclosure illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the disclosure. Thus, it should be understood that although the present disclosure has been specifically disclosed by preferred embodiments, optional features, modification and variation of the concepts herein disclosed may be resorted to by those skilled in the art, and that such modifications and variations are considered to be within the scope of this disclosure.

In addition, where features or aspects of the disclosure are described in terms of Markush groups or other grouping of alternatives, those skilled in the art will recognize that the disclosure is also thereby described in terms of any individual member or subgroup of members of the Markush group or other group.

It should be appreciated that, in some embodiments, sequences presented in the sequence listing may be referred to in describing the structure of an oligonucleotide or other nucleic acid. In such embodiments, the actual oligonucleotide or other nucleic acid may have one or more alternative nucleotides (e.g., an RNA counterpart of a DNA nucleotide or a DNA counterpart of an RNA nucleotide) and/or (e.g., and) one or more modified nucleotides and/or (e.g., and) one or more modified internucleotide linkages and/or (e.g., and) one or more other modification compared with the specified sequence while retaining essentially same or similar complementary properties as the specified sequence.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein. Variations of those embodiments may become apparent to those of ordinary skill in the art upon reading the foregoing description.

The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. Those skilled in the art will recognize, or be able to ascertain using no more than routine experimentation, many equivalents to the specific embodiments of the invention described herein. Such equivalents are intended to be encompassed by the following claims.

1. A complex comprising an anti-transferrin receptor antibody covalently linked to a molecular payload configured for to inhibit expression or activity of a pro-atrophy gene, wherein the antibody comprises a heavy chain complementarity determining region 1 (CDR-H1), a heavy chain complementarity determining region 2 (CDR-H2), a heavy chain complementarity determining region 3 (CDR-H3) of a heavy chain variable region (VH) comprising the amino acid sequence of SEQ ID NO: 54, and a light chain complementarity determining region 1 (CDR-L1), a light chain complementarity determining region 2 (CDR-L2), a light chain complementarity determining region 3 (CDR-L3) of a light chain variable region (VL) comprising the amino acid sequence of SEQ ID NO: 55.

2. The complex of claim 1, wherein the antibody comprises a CDR-H1 of SEQ ID NO: 49, a CDR-H2 of SEQ ID NO: 50, a CDR-H3 of SEQ ID NO: 51, a CDR-L1 of SEQ ID NO: 52, a CDR-L2 of SEQ ID NO: 29, and a CDR-L3 of SEQ ID NO: 53.

3. The complex of claim 1, wherein the antibody comprises human or humanized framework regions with the CDR-H1, the CDR-H2, the CDR-H3 of a VH as set forth in SEQ ID NO: 54, and the CDR-L1, the CDR-L2, the CDR-L3 of a VL as set forth in SEQ ID NO: 55.

4. The complex of claim 1, wherein the antibody comprises a VH comprising an amino acid sequence at least 80% identical to SEQ ID NO:
54, and a VL comprising an amino acid sequence at least 80% identical to SEQ ID NO: 55.

5. The complex of claim 1, wherein the equilibrium dissociation constant (K_{D}) of binding of the antibody to the transferrin receptor is in a range from 10⁻¹¹M to 10⁻⁶ M.

6. The complex of claim 1, wherein the antibody is selected from the group consisting of a full-length IgG, a Fab fragment, a F(ab′) fragment, a F(ab′)2 fragment, a scFv, and a Fv.

7. The complex of claim 1, wherein the molecular payload is an oligonucleotide.

8. The complex of claim 7, wherein the oligonucleotide comprises at least one modified internucleoside linkage.

9. The complex of claim 8, wherein the at least one modified internucleoside linkage is a phosphorothioate linkage.

10. The complex of claim 7, wherein the oligonucleotide comprises one or more modified nucleotides.

11. The complex of claim 10, wherein the one or more modified nucleotides are 2′-modified nucleotides.

12. The complex of claim 11, wherein the 2′ modified nucleotide is selected from the group consisting of: 2′-O-methyl (2′-O-Me), 2′-fluoro (2′-F), 2′-O-methoxyethyl (2′-MOE), and 2′,4′-bicyclic nucleosides.

13. The complex of claim 7, wherein the oligonucleotide is a gapmer oligonucleotide that directs RNAse H-mediated cleavage of an mRNA transcript encoded by the muscle disease gene in a cell.

14. The complex of claim 7, wherein the oligonucleotide is a mixmer oligonucleotide.

15. The complex of claim 7, wherein the oligonucleotide is an RNAi oligonucleotide that promotes RNAi-mediated cleavage of a mRNA transcript encoded by the muscle disease gene.

16. The complex of claim 7, wherein the oligonucleotide is a phosphorodiamidate morpholino oligomer (PMO).

17. The complex of claim 1, wherein the antibody is covalently linked to the molecular payload via a cleavable linker.

18. A method of delivering a molecular payload to a cell expressing transferrin receptor, the method comprising contacting the cell with the complex of claim 1.

19. A method of inhibiting expression or activity of a pro-atrophy gene in a cell, the method comprising contacting the cell with the complex of claim 1 in an amount effective for promoting internalization of the molecular payload to the cell.

20. A method of treating a subject having muscle atrophy, the method comprising administering to the subject an effective amount of the complex of claim 1.
